(19) United States
(12) Patent Application Publication
(10) Pub. No.: US 2007/0254882 A1
(43) Pub. Date: Nov. 1, 2007

(54) Sulfamide Derivatives and Pharmaceutical Composition for Upregulation of Lipid Metabolism Comprising Same

(75) Inventors: Joong Myung Cho, Seoul (KR); Tae Gyu Lee, Daejeon (KR); Seonggu Ro, Daejeon (KR); Jin Hwan Kim, Daejeon (KR); Young Ho Jeon, Daejeon (KR); Dong Kyu Shin, Seoul (KR); Young-Lan Hyun, Seoul (KR); Gyu Hwan Yon, Daejeon (KR); Young-Gwi Yoon, Yongin-si (KR); Eun Bok Choi, Daejeon (KR); Hyeon Kyu Lee, Daejeon (KR); Chwang Siek Pak, Daejeon (KR)

Correspondence Address: SUGHRUE MION, PLLC, 2100 PENNSYLVANIA AVENUE, N.W., SUITE 800, WASHINGTON, DC, 20037, US

(73) Assignees: CRYSTAL GENOMICS, INC., Seoul (KR); KOREA RESEARCH INSTITUTE OF CHEMICAL, Daejeon (KR)

(21) Appl. No.: 11/571,836

(22) PCT Filed: Jul. 14, 2005
(86) PCT No.: PCT/KR05/02266

§ 371 Date: Jan. 9, 2007

(30) Foreign Application Priority Data
Jul. 14, 2004 (KR) 10-2004-0054818

(51) Int. Cl. A61K 31/421; A61K 31/4709; A61K 31/496; A61K 31/5377; C07D 215/58; C07D 263/32; C07D 413/12

(52) U.S. Cl. 514/236.8; 514/254.02; 514/312; 514/374; 544/137; 544/369; 546/153; 548/235

(57) ABSTRACT

The present invention relates to a novel sulfamide derivative, a pharmaceutically acceptable salt thereof and a pharmaceutical composition for upregulation of lipid metabolism comprising same as an active ingredient.

### FIELD OF THE INVENTION

The present invention relates to a novel sulfamide derivative, a pharmaceutically acceptable salt thereof, and a pharmaceutical composition for upregulating the lipid metabolism comprising same as an active ingredient.

### BACKGROUND OF THE INVENTION

Essential lipids such as cholesterols and fatty acids required by organs in the body are transported in the form of lipoproteins e.g., triglycerides (TGs), cholesterol esters, phospholipids and apolipoproteins.

VLDLs (very low density lipoproteins) containing TGs are secreted from the liver and play the role of transporting fatty acids to each organ. The VLDLs transported to adipocyte is used for energy storage, and those transported to cardiac or skeletal muscles are used for energy production. VLDL releases a fatty acid, and it is converted to a LDL (low density lipoprotein) by the action of a lipoprotein lipase produced in adipocyte, skeletal muscle or macrophage, and the resulting LDL is transported to surrounding tissues to be used for steroidogenesis or maintaining cell membranes. Excess cholesterols present in many cells, on the other hand, are transported to the liver in the form of HDL (high density lipoprotein), and metabolized to nontoxic bile acids.

Thus, if the above energy homeostasis process breaks down, insulin resistance syndrome or metabolic syndrome (X-syndrome) appears as the result of lipids accumulating in the body, triggering obesity, diabetes, heart diseases, inflammation, insulin resistance, dislipidemia and hypertension (Lee et al., Endocrinology, 144, 2201-2207, 2003).

The human body contains PPARs (peroxisome proliferating-activated receptors) which function as a hormone system in charge of physiological control of lipids contained in food. PPARs belonging to the family of nuclear receptors, and consist of 2 zinc finger binding domain and hydrophobic ligand binding pocket, and they are divided into three subtypes, “PPAR α”, “PPAR δ” and “PPAR δ” (Willson et al., Journal of Med. Chem., 43, 527-550, 2000). PPAR α is expressed in the muscle, heart, kidney, and particularly in a high level in the liver which controls the disintegration of fatty acids. PPAR γ is expressed in adipocyte and macrophage, and controls the proliferation of adipocyte, lipid storage and glucose homeostasis, besides checking the proliferation of keratinocyte.

A study using PPAR CL null mice revealed that PPAR α is involved in the beta oxidation of a long chain fatty acid such as palmitic acid present in mitochondria (Costet et al., J. Bio. Chem., 273, 5678-5684, 1998). For example, the importance of PPAR α in the lipid homeostasis has been recognized by a study which measured the accumulation of lipids in the liver and heart of PPAR CL null mice, which showed hypercholesterolemia and obesity as a result of accumulated excess triglycerides (Costet et al., J. Bio. Chem., 273, 29577-29585, 1998). It is also known that PPAR α is a target protein of OEA (oleylethanolamide) which controls the appetite and body weight of null mice (Fu et al., Nature, 425 (6953), 90-93, 2000).

In addition, the activation of PPAR α causes lowering of the expression of apolipoprotein C-III which is known to inhibit the hydrolysis of TGs by LDL, leading to the inhibition of the expression of VCAM (vascular cell adhesion molecule)-1 to prevent arteriosclerosis (Marx et al., Circulation, 99, 3125-3131, 1999), IL-linterleukin-1 (interleukin-1)-induced secretion of IL-6 (interleukin-6), or the production of prostaglandin in vascular smooth muscles (Staels et al., Nature, 393, 790-793, 1998).

The tertiary structure of PPAR α has been reported to take the form of a complex with agonistGW409544 (Xu et al., PNAS, 98, 13919-13924, 2001), and also reported was the finding that the difference between Tyr (314 amino acid) of PPAR α and H is (323 amino acid) of PPAR γ determines the selectivity of PPAR. In addition, the fatty acid binding pockets of PPAR α and PPAR γ are much more pronounced than those of PPAR δ (Nolte et al., Nature, 395, 137-143, 1998). Thus, the tertiary structure of PPAR α is considered to the key in the development of PPAR α agonists.

Compounds which have been previously found to affect the activity of PPAR α are Wy-14643, clofibrate, fenofibrate, bezafibrate, GW2331, SW9578 and BM17.0744 (Willson et al., J. Med. Chem., 43, 527-550, 2000). These agonists of PPAR α bring about increased insulin sensitivity in mouse models by way of reducing TGs, adiposity and steatosis of the liver or muscle (Chou et al., JBC, 277, 24484-24489, 2002; Kim et al., Diabetes, 52, 1770-1778, 2003; and Peters et al., Mol. Cell. Biol., 20, 5119-5128, 2000).

Fenofibrate or gemfibrozil reduces TGs in blood and elevates the HDL level in hyperlipidemia patients (Lee et al., Endocrinology, 144, 2201-2207, 2003), but such an agonist requires a large daily dosage (300 to 1,200 mg/day), and therefore, there exists a need to develop more effective PPAR α agonists having excellent selectivity against PPAR α.

### SUMMARY OF THE INVENTION

Accordingly, it is an object of the present invention to provide a novel sulfamide derivative upregulating lipid metabolism and a process for the preparation same.

It is another object of the present invention to provide a pharmaceutical composition for upregulation of lipid metabolism comprising the sulfamide derivative or a pharmaceutically acceptable salt thereof.

In accordance with one aspect of the present invention, there is provided a sulfamide derivative of formula (I). 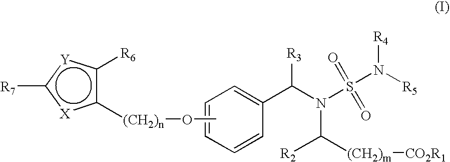
wherein,

R₁, R₂ and R₃ are each independently hydrogen or C₁₋₃ alkyl;

R₄ and R₅ are each independently hydrogen; C₁₋₅ alkyl; C₃₋₅ alkenyl; C₃₋₅ alkynyl; phenyl which is unsubstituted or substituted with C₁₋₃ alkyl, C₁₋₃ alkoxy, C₁₋₃ halogenated alkyl or halogen; or are fused together with the nitrogen atom to which they are attached to form a heterocyclic ring comprising a nitrogen atom;

R₆ is hydrogen or C₁₋₃ alkyl;

R₇ is phenyl which is unsubstituted or substituted with C₁₋₃ alkyl, C₁₋₃ halogenated alkyl or C₁₋₃ alkoxy; or thiophen;

X is nitrogen when Y is oxygen, and X is oxygen when Y is nitrogen;

m is 0 or 1; and

n is 1 or 2.

In accordance with another aspect of the present invention, there is provided a process for the preparation of the sulfamide derivative.

In accordance with further aspect of the present invention, there is provided a pharmaceutical composition for upregulation of lipid metabolism comprising the sulfamide derivative or a pharmaceutically acceptable salt thereof as an active ingredient.

### BRIEF DESCRIPTION OF THE DRAWINGS

The above and other objects and features of the present invention will become apparent from the following description of the invention taken in conjunction with the following accompanying drawings, which respectively show:

FIGS. 1a and 1b: graphs showing the time-dependant changes in the feed intake rate and cumulative amounts of total feed intake observed for a control group treated only with an excipient, a positive control group treated with fenofibrate, and test groups treated with the compounds of Examples 5, 62 and 64, respectively;

FIGS. 2a and 2b: graphs showing the time-dependant changes in terms of average body weight and body weight gain observed for a control group treated only with an excipient, a positive control group treated with fenofibrate, and test groups treated with the compounds of Examples 5, 62 and 64, respectively;

FIGS. 3a to 3c: graphs showing the results of oral glucose tolerance tests (OGTT) performed for a control group treated only with an excipient, a positive control group treated with fenofibrate, and test groups treated with the compounds of Examples 5, 62 and 64, respectively; and

FIGS. 4a to 4d: experimental findings regarding the weight of total fat, the adiposity index, the weight of subcutaneous fat and the weight of visceral fat determined after anatomy for a control group treated only with an excipient, a positive control group treated with fenofibrate, and test groups treated with the compounds of Examples 5, 62 and 64, respectively.

### DETAILED DESCRIPTION OF THE INVENTION

The sulfamide derivative of the present invention may comprise an asymmetric carbon, and therefore, it may be in the form of a stereo-specific isomer, a racemate or a mixture thereof. The sulfamide derivative of the present invention may also be used in the form of a pharmaceutically acceptable salt, hydrate or solvate. The pharmaceutically acceptable salt may be a salt formed with an inorganic or organic base, e.g., alkali metal or alkaline earth metal compounds, ammonia, organic amines such as methylamine, ethylamine, pyridine, guanidine and arginine; or with an inorganic or organic acid, e.g., hydrochloric acid, hydrobromic acid, sulfuric acid, phosphoric acid, citric acid, acetic acid, lactic acid, tartaric acid, maleic acid, fumaric acid, gluconic acid, methanesulfonic acid, glycolic acid, succinic acid, 4-toluenesulfonic acid, gluturonic acid, embonic acid, glutamic acid or aspartic acid.

Examples of the preferred compounds of formula (I) according to the present invention are:
1) [N-(aminosulfonyl)-N-[[4-(2-phenyl-5-methyloxazol-4-yl)ethoxy]benzyl]amino]acetic acid;
2) (S)-3-methyl-2-[N-(sulfamoyl)-N-[3-[(5-methyl-2-p-tolyloxazol-4-yl)methoxy]benzyl]amino]butyric acid;
3) [N—[(N,N-dimethylamino)sulfonyl]-N-[3-[(2-phenyl-5-methyloxazol-4-yl)methoxy]benzyl]amino]acetic acid;
4) [N—[(N,N-dimethylamino)sulfonyl]-N-[3-[[2-(4-methylphenyl)-5-methyloxazol-4-yl]methoxy]benzyl]amino]acetic acid;
5) [N—(N,N-dimethylamino)sulfonyl-N-[3-[[2-(4-trifluoromethylphenyl)-5-methyloxazol-4-yl]methoxy]benzyl]amino]acetic acid;
6) [N—(N-t-butylamino)sulfonyl-N-[3-[[2-(4-trifluoromethylphenyl)-5-methyloxazol-4-yl]methoxy]benzyl]amino]acetic acid;
7) [N—(N,N-diethylamino)sulfonyl-N-[3-[[2-(4-trifluoromethylphenyl)-5-methyloxazol-4-yl]methoxy]benzyl]amino]acetic acid;
8) [N—(N-isopropyl-N-methylamino)sulfonyl-N-[3-[[2-(4-trifluoromethyl phenyl)-5-methyloxazol-4-yl]methoxy]benzyl]amino]acetic acid;
9) [N—(N-allyl-N-methylamino)sulfonyl-N-[3-[[2-(4-trifluoromethyl phenyl)-5-methyloxazol-4-yl]methoxy]benzyl]amino]acetic acid;
10) [N—(N-methyl-N-propargyl)sulfonyl-N-[3-[[2-(4-trifluoromethyl phenyl)-5-methyloxazol-4-yl]methoxy]benzyl]amino]acetic acid;
11) [N-(piperidinyl)sulfonyl-N-[3-[[2-(4-trifluoromethylphenyl)-5-methyloxazol-4-yl]methoxy]benzyl]amino]acetic acid;
12) [N—(N,N-dimethylamino)sulfonyl-N-[3-[[2-(thiophen-2-yl)-5-methyl oxazol-4-yl]methoxy]benzyl]amino]acetic acid;
13) [N—(N,N-dimethylamino)sulfonyl-N-[3-[[2-(4-trifluoromethylphenyl)-4-methyloxazol-5-yl]methoxy]benzyl]amino]acetic acid;
14) [N—[(N-methyl-N-phenyl)amino]sulfonyl-N-[3-[(2-phenyl-5-methyl oxazol-4-yl)methoxy]benzyl]amino]acetic acid;
15) [N—[(N-methyl-N-phenyl)amino]sulfonyl-N-[3-[[2-(4-methylphenyl)-5-methyloxazol-4-yl]methoxy]benzyl]amino]acetic acid;
16) [N—[(N-methyl-N-phenyl)amino]sulfonyl-N-[3-[[2-(4-trifluoromethyl phenyl)-5-methyloxazol-4-yl]methoxy]benzyl]amino]acetic acid;
17) [N—[(N-methyl-N-phenyl)amino]sulfonyl-N-[3-[[2-(thiophen-2-yl)-5-methyloxazol-4-yl]methoxy]benzyl]amino]acetic acid;
18) [N—[[N-methyl-N-(4-chlorophenyl)]amino]sulfonyl-N-[3-[(2-phenyl-5-methyloxazol-4-yl)methoxy]benzyl]amino]acetic acid;
19) [N—[[N-methyl-N-(4-chlorophenyl)]amino]sulfonyl-N-[3-[[2-(4-methylphenyl)-5-methyloxazol-4-yl]methoxy]benzyl]amino]acetic acid;
20) [N—[[N-methyl-N-(4-chlorophenyl)]amino]sulfonyl-N-[3-[[2-(4-tri fluoromethylphenyl)-5-methyloxazol-4-yl]methoxy]benzyl]amino]acetic acid;
21) [N—(N-ethyl-N-m-tolylamino)sulfonyl-N-[3-[[2-(4-trifluoromethyl phenyl)-5-methyloxazol-4-yl]methoxy]benzyl]amino]acetic acid;
22) [N—(N-p-anisoyl-N-methylamino)sulfonyl-N-[3-[[2-(4-trifluoro methylphenyl)-5-methyloxazol-4-yl]methoxy]benzyl]amino]acetic acid;
23) [N—[N-(3-fluorophenyl)-N-methylamino]sulfonyl-N-[3-[[2-(4-tri fluoromethylphenyl)-5-methyloxazol-4-yl]methoxy]benzyl]amino]acetic acid;
24) [N—[[N-methyl-N-(p-chlorophenyl)]amino]sulfonyl-N-[3-[[2-(thiophen-2-yl)-5-methyloxazol-4-yl]methoxy]benzyl]amino]acetic acid;
25) [N-(pyrrolidinyl)sulfonyl-N-[3-[[2-(4-trifluoromethylphenyl)-5-methyloxazol-4-yl]methoxy]benzyl]amino]acetic acid;
26) [N-(pyrrolidinyl)sulfonyl-N-[3-[[2-(4-trifluoromethylphenyl)-4-methyloxazol-5-yl]methoxy]benzyl]amino]acetic acid;
27) [N-(4-methyl-1-piperazinyl)sulfonyl-N-[3-[[2-(4-trifluoromethyl phenyl)-5-methyloxazol-4-yl]methoxy]benzyl]amino]acetic acid;
28) [N-(morpholinyl)sulfonyl-N-[3-[[2-(4-methylphenyl)-5-methyl oxazol-4-yl]methoxy]benzyl]amino]acetic acid;
29) [N-(morpholinyl)sulfonyl-N-[3-[[2-(4-trifluoromethylphenyl)-4-methyloxazol-5-yl]methoxy]benzyl]amino]acetic acid;
30) [N-(indolinyl)sulfonyl-N-[3-[(2-phenyl-5-methyloxazol-4-yl)methoxy]benzyl]amino]acetic acid;
31) [N-(indolinyl)sulfonyl-N-[3-[[2-(4-methylphenyl)-5-methyloxazol-4-yl]methoxy]benzyl]amino]acetic acid;
32) [N-(indolinyl)sulfonyl-N-[3-[[2-(4-trifluoromethylphenyl)-5-methyl oxazol-4-yl]methoxy]benzyl]amino]acetic acid;
33) [N-(indolinyl)sulfonyl-N-[3-[[2-(thiophen-2-yl)-5-methyloxazol-4-yl]methoxy]benzyl]amino]acetic acid;
34) [N-(1,2,3,4-tetrahydroquinolinyl)sulfonyl-N-[3-[(2-phenyl-5-methyl oxazol-4-yl)methoxy]benzyl]amino]acetic acid;
35) [N-(1,2,3,4-tetrahydroquinolinyl)sulfonyl-N-[3-[[2-(4-methylphenyl)-5-methyloxazol-4-yl]methoxy]benzyl]amino]acetic acid;
36) [N-(1,2,3,4-tetraquinolinyl)sulfonyl-N-[3-[[2-(4-trifluoromethyl phenyl)-5-methyloxazol-4-yl]methoxy]benzyl]amino]acetic acid;
37) [N-(1,2,3,4-tetraquinolinyl)sulfonyl-N-[3-[[2-(thiophen-2-yl)-5-methyloxazol-4-yl]methoxy]benzyl]amino]acetic acid;
38) [N—(N,N-dimethylamino)sulfonyl-N-[4-[(2-phenyl-5-methyloxazol-4-yl)methoxy]benzyl]amino]acetic acid;
39) [N—(N,N-dimethylamino)sulfonyl-N-[4-[[2-(4-methylphenyl)-5-methyloxazol-4-yl]methoxy]benzyl]amino]acetic acid;
40) [N—(N,N-dimethylamino)sulfonyl-N-[4-[[2-(4-trifluoromethylphenyl)-5-methyloxazol-4-yl]methoxy]benzyl]amino]acetic acid;
41) [N—(N,N-dimethylamino)sulfonyl-N-[4-[[2-(thiophen-2-yl)-5-methyl oxazol-4-yl]methoxy]benzyl]amino]acetic acid;
42) [N-(pyrrolidinyl)sulfonyl-N-[4-[[2-(4-methylphenyl)-5-methyloxazol-4-yl]methoxy]benzyl]amino]acetic acid;
43) [N-(4-methyl-1-piperazinyl)sulfonyl-N-[4-[[2-(4-methylphenyl)-5-methyloxazol-4-yl]methoxy]benzyl]amino]acetic acid;
44) [N-(morpholinyl)sulfonyl-N-[4-[[2-(4-methylphenyl)-5-methyl oxazol-4-yl]methoxy]benzyl]amino]acetic acid;
45) [N—[(N-methyl-N-phenyl)amino]sulfonyl-N-[4-[(2-phenyl-5-methyl oxazol-4-yl)methoxy]benzyl]amino]acetic acid;
46) [N—[(N-methyl-N-phenyl)amino]sulfonyl-N-[4-[[2-(4-methylphenyl)-5-methyloxazol-4-yl]methoxy]benzyl]amino]acetic acid;
47) [N—[(N-methyl-N-phenyl)amino]sulfonyl-N-[4-[[2-(4-trifluoromethyl phenyl)-5-methyloxazol-4-yl]methoxy]benzyl]amino]acetic acid;
48) [N—[(N-methyl-N-phenyl)amino]sulfonyl-N-[4-[[2-(thiophen-2-yl)-5-methyloxazol-4-yl]methoxy]benzyl]amino]acetic acid;
49) [N—[[N-methyl-N-(4-chlorophenyl)]amino]sulfonyl-N-[4-[(2-phenyl-5-methyloxazol-4-yl)methoxy]benzyl]amino]acetic acid;
50) [N—[[N-methyl-N-(4-chlorophenyl)]amino]sulfonyl-N-[4-[[2-(4-methylphenyl)-5-methyloxazol-4-yl]methoxy]benzyl]amino]acetic acid;
51) [N—[[N-methyl-N-(4-chlorophenyl)]amino]sulfonyl-N-[4-[[2-(4-trifluoromethylphenyl)-5-methyloxazol-4-yl]methoxy]benzyl]amino]acetic acid;
52) [N—[[N-methyl-N-(4-chlorophenyl)]amino]sulfonyl-N-[4-[[2-(thiophen-2-yl)-5-methyloxazol-4-yl]methoxy]benzyl]amino]acetic acid;
53) [N-(indolinyl)sulfonyl-N-[4-[(2-phenyl-5-methyloxazol-4-yl)methoxy]benzyl]amino]acetic acid;
54) [N-(indolinyl)sulfonyl-N-[4-[[2-(4-methylphenyl)-5-methyloxazol-4-yl]methoxy]benzyl]amino]acetic acid;
55) [N-(indolinyl)sulfonyl-N-[4-[[2-(4-trifluoromethylphenyl)-5-methyl oxazol-4-yl]methoxy]benzyl]amino]acetic acid;
56) [N-(indolinyl)sulfonyl-N-[4-[[2-(thiophen-2-yl)-5-methyloxazol-4-yl]methoxy]benzyl]amino]acetic acid;
57) [N-(1,2,3,4-tetrahydroquinolinyl)sulfonyl-N-[4-[(2-phenyl-5-methyl oxazol-4-yl)methoxy]benzyl]amino]acetic acid;
58) [N-(1,2,3,4-tetraquinolinyl)sulfonyl-N-[4-[[2-(4-methylphenyl)-5-methyloxazol-4-yl]methoxy]benzyl]amino]acetic acid;
59) [N-(1,2,3,4-tetrahydroquinolinyl)sulfonyl-N-[4-[[2-(4-trifluoro methylphenyl)-5-methyloxazol-4-yl]methoxy]benzyl]amino]acetic acid;
60) [N-(1,2,3,4-tetrahydroquinolinyl)sulfonyl-N-[4-[[2-(thiophen-2-yl)-5-methyloxazol-4-yl]methoxy]benzyl]amino]acetic acid;
61) [N—(N,N-dimethylamino)sulfonyl-N-[3-[2-(2-phenyl-5-methyloxazol-4-yl)ethoxy]benzyl]amino]acetic acid;
62) [N—(N,N-dimethylamino)sulfonyl-N-[3-[2-[2-(4-methylphenyl)-5-methyloxazol-4-yl]ethoxy]benzyl]amino]acetic acid;
63) [N—(N,N-diethylamino)sulfonyl-N-[3-[2-[2-(4-methylphenyl)-5-methyloxazol-4-yl]ethoxy]benzyl]amino]acetic acid;
64) [N—(N-isopropyl-N-methylamino)sulfonyl-N-[3-[2-[2-(4-methyl phenyl)-5-methyloxazol-4-yl]ethoxy]benzyl]amino]acetic acid;
65) [N—(N,N-dimethylamino)sulfonyl-N-[3-[2-[2-(4-trifluoromethyl phenyl)-5-methyloxazol-4-yl]ethoxy]benzyl]amino]acetic acid;
66) [N—(N-t-butylamino)sulfonyl-N-[3-[2-[2-(4-trifluoromethylphenyl)-5-methyloxazol-4-yl]ethoxy]benzyl]amino]acetic acid;
67) [N—(N,N-diethylamino)sulfonyl-N-[3-[2-[2-(4-trifluoromethylphenyl)-5-methyloxazol-4-yl]ethoxy]benzyl]amino]acetic acid;
68) [N—(N-isopropyl-N-methylamino)sulfonyl-N-[3-[2-[2-(4-trifluoro methylphenyl)-5-methyloxazol-4-yl]ethoxy]benzyl]amino]acetic acid;
69) [N—(N-allyl-N-methylamino)sulfonyl-N-[3-[2-[2-(4-trifluoromethyl phenyl)-5-methyloxazol-4-yl]ethoxy]benzyl]amino]acetic acid;
70) [N—(N-methyl-N-propargylamino)sulfonyl-N-[3-[2-[2-(4-trifluoro methylphenyl)-5-methyloxazol-4-yl]ethoxy]benzyl]amino]acetic acid;
71) [N-(piperidinyl)sulfonyl-N-[3-[2-[2-(4-trifluoromethylphenyl)-5-methyloxazol-4-yl]ethoxy]benzyl]amino]acetic acid;
72) [N—(N,N-dimethylamino)sulfonyl-N-[3-[2-[2-(thiophen-2-yl)-5-methyloxazol-4-yl]ethoxy]benzyl]amino]acetic acid;
73) [N—[(N-methyl-N-phenyl)amino]sulfonyl-N-[3-[2-(2-phenyl-5-methyloxazol-4-yl)ethoxy]benzyl]amino]acetic acid;
74) [N—[(N-methyl-N-phenyl)amino]sulfonyl-N-[3-[2-[2-(4-methyl phenyl)-5-methyloxazol-4-yl]ethoxy]benzyl]amino]acetic acid;
75) [N—[(N-methyl-N-phenyl)amino]sulfonyl-N-[3-[2-[2-(4-trifluoro methylphenyl)-5-methyloxazol-4-yl]ethoxy]benzyl]amino]acetic acid;
76) [N—[(N-methyl-N-phenyl)amino]sulfonyl-N-[3-[2-[2-(thiophen-2-yl)-5-methyloxazol-4-yl]ethoxy]benzyl]amino]acetic acid;
77) [N—[[N-methyl-N-(4-chlorophenyl)]amino]sulfonyl-N-[3-[2-(2-phenyl-5-methyloxazol-4-yl)ethoxy]benzyl]amino]acetic acid;
78) [N—[[N-methyl-N-(4-chlorophenyl)]amino]sulfonyl-N-[3-[2-[2-(4-methylphenyl)-5-methyloxazol-4-yl]ethoxy]benzyl]amino]acetic acid;
79) [N—[[N-methyl-N-(4-chlorophenyl)]amino]sulfonyl-N-[3-[2-[2-(4-trifluoromethylphenyl)-5-methyloxazol-4-yl]ethoxy]benzyl]amino]acetic acid;
80) [N—(N-ethyl-N-m-tolylamino)sulfonyl-N-[3-[2-[2-(4-trifluoromethyl phenyl)-5-methyloxazol-4-yl]ethoxy]benzyl]amino]acetic acid;
81) [N—(N-anisoyl-N-methylamino)sulfonyl-N-[3-[2-[2-(4-trifluoro methylphenyl)-5-methyloxazol-4-yl]ethoxy]benzyl]amino]acetic acid;
82) [N—[N-(3-fluorophenyl)-N-methylamino]sulfonyl-N-[3-[2-[2-(4-trifluoromethylphenyl)-5-methyloxazol-4-yl]ethoxy]benzyl]amino]acetic acid;
83) [N—[[N-methyl-N-(4-chlorophenyl)]amino]sulfonyl-N-[3-[2-[2-(thiophen-2-yl)-5-methyloxazol-4-yl]ethoxy]benzyl]amino]acetic acid;
84) [N-(pyrrolidinyl)sulfonyl-N-[3-[2-[2-(4-methylphenyl)-5-methyl oxazol-4-yl]ethoxy]benzyl]amino]acetic acid;
85) [N-(pyrrolidinyl)sulfonyl-N-[3-[2-[2-(4-trifluoromethylphenyl)-5-methyloxazol-4-yl]ethoxy]benzyl]amino]acetic acid;
86) [N-(morpholinyl)sulfonyl-N-[3-[2-[2-(4-methylphenyl)-5-methyl oxazol-4-yl]ethoxy]benzyl]amino]acetic acid;
87) [N-(morpholinyl)sulfonyl-N-[3-[2-[2-(4-trifluoromethylphenyl)-5-methyloxazol-4-yl]ethoxy]benzyl]amino]acetic acid;
88) [N-(4-methyl-1-piperazinyl)sulfonyl-N-[3-[2-[2-(4-methylphenyl)-5-methyloxazol-4-yl]ethoxy]benzyl]amino]acetic acid;
89) [N-(4-methyl-1-piperazinyl)sulfonyl-N-[3-[2-[2-(4-trifluoromethyl phenyl)-5-methyloxazol-4-yl]ethoxy]benzyl]amino]acetic acid;
90) [N-(indolinyl)sulfonyl-N-[3-[2-(2-phenyl-5-methyloxazol-4-yl)ethoxy]benzyl]amino]acetic acid;
91) [N-(indolinyl)sulfonyl-N-[3-[2-[2-(4-methylphenyl)-5-methyloxazol-4-yl]ethoxy]benzyl]amino]acetic acid;
92) [N-(indolinyl)sulfonyl-N-[3-[2-[2-(4-trifluoromethylphenyl)-5-methyloxazol-4-yl]ethoxy]benzyl]amino]acetic acid;
93) [N-(indolinyl)sulfonyl-N-[3-[2-[2-(thiophen-2-yl)-5-methyloxazol-4-yl]ethoxy]benzyl]amino]acetic acid;
94) [N-(1,2,3,4-tetrahydroquinolinyl)sulfonyl-N-[3-[2-(2-phenyl-5-methyloxazol-4-yl)ethoxy]benzyl]amino]acetic acid;
95) [N-(1,2,3,4-tetraquinolinyl)sulfonyl-N-[3-[2-[2-(4-methylphenyl)-5-methyloxazol-4-yl]ethoxy]benzyl]amino]acetic acid;
96) [N-(1,2,3,4-tetrahydroquinolinyl)sulfonyl-N-[3-[2-[2-(4-trifluoro methylphenyl)-5-methyloxazol-4-yl]ethoxy]benzyl]amino]acetic acid;
97) [N-(1,2,3,4-tetrahydroquinolinyl)sulfonyl-N-[3-[2-[2-(thiophen-2-yl)-5-methyloxazol-4-yl]ethoxy]benzyl]amino]acetic acid;
98) [N—(N,N-dimethylamino)sulfonyl-N-[4-[2-(2-phenyl-5-methyloxazol-4-yl)ethoxy]benzyl]amino]acetic acid;
99) [N—(N,N-dimethylamino)sulfonyl-N-[4-[2-[2-(4-methylphenyl)-5-methyloxazol-4-yl]ethoxy]benzyl]amino]acetic acid;
100) [N—(N-t-butylamino)sulfonyl-N-[4-[2-[2-(4-methylphenyl)-5-methyl oxazol-4-yl]ethoxy]benzyl]amino]acetic acid;
101) [N—(N,N-diethylamino)sulfonyl-N-[4-[2-[2-(4-methylphenyl)-5-methyloxazol-4-yl]ethoxy]benzyl]amino]acetic acid;
102) [N—(N-isopropyl-N-methylamino)sulfonyl-N-[4-[2-[2-(4-methyl phenyl)-5-methyloxazol-4-yl]ethoxy]benzyl]amino]acetic acid;
103) [N—(N,N-dimethylamino)sulfonyl-N-[4-[2-[2-(4-trifluoromethyl phenyl)-5-methyloxazol-4-yl]ethoxy]benzyl]amino]acetic acid;
104) [N—(N,N-dimethylamino)sulfonyl-N-[4-[2-[2-(thiophen-2-yl)-5-methyloxazol-4-yl]ethoxy]benzyl]amino]acetic acid;
105) [N—[(N-methyl-N-phenyl)amino]sulfonyl-N-[4-[2-(2-phenyl-5-methyloxazol-4-yl)ethoxy]benzyl]amino]acetic acid;
106) [N—[(N-methyl-N-phenyl)amino]sulfonyl-N-[4-[2-[2-(4-methyl phenyl)-5-methyloxazol-4-yl]ethoxy]benzyl]amino]acetic acid;
107) [N—[(N-methyl-N-phenyl)amino]sulfonyl-N-[4-[2-[2-(4-trifluoro methylphenyl)-5-methyloxazol-4-yl]ethoxy]benzyl]amino]acetic acid;
108) [N—[(N-methyl-N-phenyl)amino]sulfonyl-N-[4-[2-[2-(thiophen-2-yl)-5-methyloxazol-4-yl]ethoxy]benzyl]amino]acetic acid;
109) [N—[[N-methyl-N-(4-chlorophenyl)]amino]sulfonyl-N-[4-[2-(2-phenyl-5-methyloxazol-4-yl)ethoxy]benzyl]amino]acetic acid;
110) [N—[[N-methyl-N-(4-chlorophenyl)]amino]sulfonyl-N-[4-[2-[2-(4-methylphenyl)-5-methyloxazol-4-yl]ethoxy]benzyl]amino]acetic acid;
111) [N—(N-ethyl-N-m-tolylamino)sulfonyl-N-[4-[2-[2-(4-methylphenyl)-5-methyloxazol-4-yl]ethoxy]benzyl]amino]acetic acid;
112) [N—(N-anisoyl-N-methylamino)sulfonyl-N-[4-[2-[2-(4-methyl phenyl)-5-methyloxazol-4-yl]ethoxy]benzyl]amino]acetic acid;
113) [N—[N-(3-fluorophenyl)-N-methylamino]sulfonyl-N-[4-[2-[2-(4-methylphenyl)-5-methyloxazol-4-yl]ethoxy]benzyl]amino]acetic acid;
114) [N—[[N-methyl-N-(4-chlorophenyl)]amino]sulfonyl-N-[4-[2-[2-(4-trifluoromethylphenyl)-5-methyloxazol-4-yl]ethoxy]benzyl]amino]acetic acid;
115) [N—[[N-methyl-N-(4-chlorophenyl)]amino]sulfonyl-N-[4-[2-[2-(thiophen-2-yl)-5-methyloxazol-4-yl]ethoxy]benzyl]amino]acetic acid;
116) [N-(indolinyl)sulfonyl-N-[4-[2-[(2-phenyl-5-methyloxazol-4-yl)ethoxy]benzyl]amino]acetic acid;
117) [N-(indolinyl)sulfonyl-N-[4-[2-[2-(4-methylphenyl)-5-methyl oxazol-4-yl]ethoxy]benzyl]amino]acetic acid;
118) [N-(indolinyl)sulfonyl-N-[4-[2-[2-(4-trifluoromethylphenyl)-5-methyloxazol-4-yl]ethoxy]benzyl]amino]acetic acid;
119) [N-(indolinyl)sulfonyl-N-[4-[2-[2-(thiophen-2-yl)-5-methyloxazol-4-yl]ethoxy]benzyl]amino]acetic acid;
120) [N-(1,2,3,4-tetrahydroquinolinyl)sulfonyl-N-[4-[2-(2-phenyl-5-methyloxazol-4-yl)ethoxy]benzyll]amino]acetic acid;
121) [N-(1,2,3,4-tetrahydroquinolinyl)sulfonyl-N-[4-[2-[2-(4-methyl phenyl)-5-methyloxazol-4-yl]ethoxy]benzyl]amino]acetic acid;
122) [N-(1,2,3,4-tetrahydroquinolinyl)sulfonyl-N-[4-[2-[2-(4-trifluoro methylphenyl)-5-methyloxazol-4-yl]ethoxy]benzyl]amino]acetic acid;
123) [N-(1,2,3,4-tetrahydroquinolinyl)sulfonyl-N-[4-[2-[2-(thiophen-2-yl)-5-methoxyoxazol-4-yl]ethoxy]benzyl]amino]acetic acid;
124) (S)-2-[N—(N,N-dimethylamino)sulfonyl-N-[3-[(5-methyl-2-p-tolyl oxazol-4-yl)methoxy]benzyl]amino]propionic acid;
125) (S)-3-methyl-2-[N—(N,N-dimethylamino)sulfonyl-N-[3-[(5-methyl-2-p-tolyloxazol-4-yl)methoxy]benzyl]amino]butyric acid;
126) [N—(N,N-dimethylamino)sulfonyl-N-[1-[[3-(5-methyl-2-p-tolyl oxazol-4-yl)methoxy]phenyl]ethyl]amino]acetic acid;
127) [N-(pyrrolidinyl)sulfonyl-N-[1-[[3-(5-methyl-2-p-tolyloxazol-4-yl)methoxy]phenyl]ethyl]amino]acetic acid;
128) [N—(N,N-diethylamino)sulfonyl-N-[1-[[3-(5-methyl-2-p-tolyl oxazol-4-yl)methoxy]phenyl]ethyl]amino]acetic acid;
129) [N—(N-isopropyl-N-methylamino)sulfonyl-N-[1-[[3-(5-methyl-2-p-tolyloxazol-4-yl)methoxy]phenyl]ethyl]amino]acetic acid;
130) 3-[N—(N,N-dimethylamino)sulfonyl-N-[3-[(5-methyl-2-p-tolyl oxazol-4-yl)methoxy]benzyl]amino]propionic acid;
131) [N—(N,N-dimethylamino)sulfonyl-N-[3-[(2-phenyloxazol-4-yl)ethoxy]benzyl]amino]acetic acid;
132) [N—(N,N-dimethylamino)sulfonyl-N-[4-[(2-phenyloxazol-4-yl)ethoxy]benzyl]amino]acetic acid;
133) [N—[[N-(4-chlorophenyl)-N-methyl]amino]sulfonyl-N-[4-[(2-phenyl oxazol-4-yl)ethoxy]benzyl]amino]acetic acid; and
134) [N—[(N,N-dimethylamino)sulfonyl]-N-[4-[(2-phenyl-5-isopropyl oxazol-4-yl)methoxy]benzyl]amino]acetic acid.

More preferred compounds of formula (I) of the present invention are those wherein R₁ is hydrogen; R₂ is hydrogen or methyl; R₃ is hydrogen or C₁₋₃ alkyl; R₄ and R₅ are each independently hydrogen, methyl, ethyl, isopropyl, propargyl, piperidinyl, or phenyl which is unsubstituted or substituted with methyl, methoxy, fluoro or chloro; or are fused together with the nitrogen atom to which they are attached to form piperidine or quinolone ring; R₆ is methyl; R₇ is phenyl, which is unsubstituted or substitutied with CF₃ or CH₃; X is nitrogen; Y is oxygen; m is 0 or 1; and n is 1 or 2.

Examples of the more preferred compounds of formula (I) according to the present invention are:
2) (S)-3-methyl-2-[N-(sulfamoyl)-N-[3-[(5-methyl-2-p-tolyloxazol-4-yl)methoxy]benzyl]amino]butyric acid;
7) [N—(N,N-diethylamino)sulfonyl-N-[3-[[2-(4-trifluoromethylphenyl)-5-methyloxazol-4-yl]methoxy]benzyl]amino]acetic acid;
8) [N—(N-isopropyl-N-methylamino)sulfonyl-N-[3-[[2-(4-trifluoromethyl phenyl)-5-methyloxazol-4-yl]methoxy]benzyl]amino]acetic acid;
9) [N—(N-allyl-N-methylamino)sulfonyl-N-[3-[[2-(4-trifluoromethyl phenyl)-5-methyloxazol-4-yl]methoxy]benzyl]amino]acetic acid;
10) [N—(N-methyl-N-propargyl)sulfonyl-N-[3-[[2-(4-trifluoromethyl phenyl)-5-methyloxazol-4-yl]methoxy]benzyl]amino]acetic acid;
11) [N-(piperidinyl)sulfonyl-N-[3-[[2-(4-trifluoromethylphenyl)-5-methyloxazol-4-yl]methoxy]benzyl]amino]acetic acid;
21) [N—(N-ethyl-N-m-tolylamino)sulfonyl-N-[3-[[2-(4-trifluoromethyl phenyl)-5-methyloxazol-4-yl]methoxy]benzyl]amino]acetic acid;
22) [N—(N-p-anisoyl-N-methylamino)sulfonyl-N-[3-[[2-(4-trifluoro methylphenyl)-5-methyloxazol-4-yl]methoxy]benzyl]amino]acetic acid;
23) [N—[N-(3-fluorophenyl)-N-methylamino]sulfonyl-N-[3-[[2-(4-tri fluoromethylphenyl)-5-methyloxazol-4-yl]methoxy]benzyl]amino]acetic acid;
58) [N-(1,2,3,4-tetraquinolinyl)sulfonyl-N-[4-[[2-(4-methylphenyl)-5-methyloxazol-4-yl]methoxy]benzyl]amino]acetic acid;
62) [N—(N,N-dimethylamino)sulfonyl-N-[3-[2-[2-(4-methylphenyl)-5-methyloxazol-4-yl]ethoxy]benzyl]amino]acetic acid;
63) [N—(N,N-diethylamino)sulfonyl-N-[3-[2-[2-(4-methylphenyl)-5-methyloxazol-4-yl]ethoxy]benzyl]amino]acetic acid;
65) [N—(N,N-dimethylamino)sulfonyl-N-[3-[2-[2-(4-trifluoromethyl phenyl)-5-methyloxazol-4-yl]ethoxy]benzyl]amino]acetic acid;
67) [N—(N,N-diethylamino)sulfonyl-N-[3-[2-[2-(4-trifluoromethylphenyl)-5-methyloxazol-4-yl]ethoxy]benzyl]amino]acetic acid;
70) [N—(N-methyl-N-propargylamino)sulfonyl-N-[3-[2-[2-(4-trifluoro methylphenyl)-5-methyloxazol-4-yl]ethoxy]benzyl]amino]acetic acid;
71) [N-(piperidinyl)sulfonyl-N-[3-[2-[2-(4-trifluoromethylphenyl)-5-methyloxazol-4-yl]ethoxy]benzyl]amino]acetic acid;
80) [N—(N-ethyl-N-m-tolylamino)sulfonyl-N-[3-[2-[2-(4-trifluoromethyl phenyl)-5-methyloxazol-4-yl]ethoxy]benzyl]amino]acetic acid;
81) [N—(N-anisoyl-N-methylamino)sulfonyl-N-[3-[2-[2-(4-trifluoro methylphenyl)-5-methyloxazol-4-yl]ethoxy]benzyl]amino]acetic acid;
82) [N—[N-(3-fluorophenyl)-N-methylamino]sulfonyl-N-[3-[2-[2-(4-tri fluoromethylphenyl)-5-methyloxazol-4-yl]ethoxy]benzyl]amino]acetic acid;
101) [N—(N,N-diethylamino)sulfonyl-N-[4-[2-[2-(4-methylphenyl)-5-methyloxazol-4-yl]ethoxy]benzyl]amino]acetic acid;
102) [N—(N-isopropyl-N-methylamino)sulfonyl-N-[4-[2-[2-(4-methyl phenyl)-5-methyloxazol-4-yl]ethoxy]benzyl]amino]acetic acid;
111) [N—(N-ethyl-N-m-tolylamino)sulfonyl-N-[4-[2-[2-(4-methylphenyl)-5-methyloxazol-4-yl]ethoxy]benzyl]amino]acetic acid;
112) [N—(N-anisoyl-N-methylamino)sulfonyl-N-[4-[2-[2-(4-methyl phenyl)-5-methyloxazol-4-yl]ethoxy]benzyl]amino]acetic acid;
113) [N—[N-(3-fluorophenyl)-N-methylamino]sulfonyl-N-[4-[2-[2-(4-methylphenyl)-5-methyloxazol-4-yl]ethoxy]benzyl]amino]acetic acid;
124) (S)-2-[N—(N,N-dimethylamino)sulfonyl-N-[3-[(5-methyl-2-p-tolyl oxazol-4-yl)methoxy]benzyl]amino]propionic acid;
125) (S)-3-methyl-2-[N—(N,N-dimethylamino)sulfonyl-N-[3-[(5-methyl-2-p-tolyloxazol-4-yl)methoxy]benzyl]amino]butyric acid;
126) [N—(N,N-dimethylamino)sulfonyl-N-[1-[[3-(5-methyl-2-p-tolyl oxazol-4-yl)methoxy]phenyl]ethyl]amino]acetic acid;
127) [N-(pyrrolidinyl)sulfonyl-N-[1-[[3-(5-methyl-2-p-tolyloxazol-4-yl)methoxy]phenyl]ethyl]amino]acetic acid;
129) [N—(N-isopropyl-N-methylamino)sulfonyl-N-[1-[[3-(5-methyl-2-p-tolyloxazol-4-yl)methoxy]phenyl]ethyl]amino]acetic acid;
130) 3-[N—(N,N-dimethylamino)sulfonyl-N-[3-[(5-methyl-2-p-tolyl oxazol-4-yl)methoxy]benzyl]amino]propionic acid;
131) [N—(N,N-dimethylamino)sulfonyl-N-[3-[(2-phenyloxazol-4-yl)ethoxy]benzyl]amino]acetic acid;
132) [N—(N,N-dimethylamino)sulfonyl-N-[4-[(2-phenyloxazol-4-yl)ethoxy]benzyl]amino]acetic acid; and
133) [N—[[N-(4-chlorophenyl)-N-methyl]amino]sulfonyl-N-[4-[(2-phenyl oxazol-4-yl)ethoxy]benzyl]amino]acetic acid.

The sulfamide derivative of formula (I) may be prepared by the procedure shown in Reaction Scheme (I): 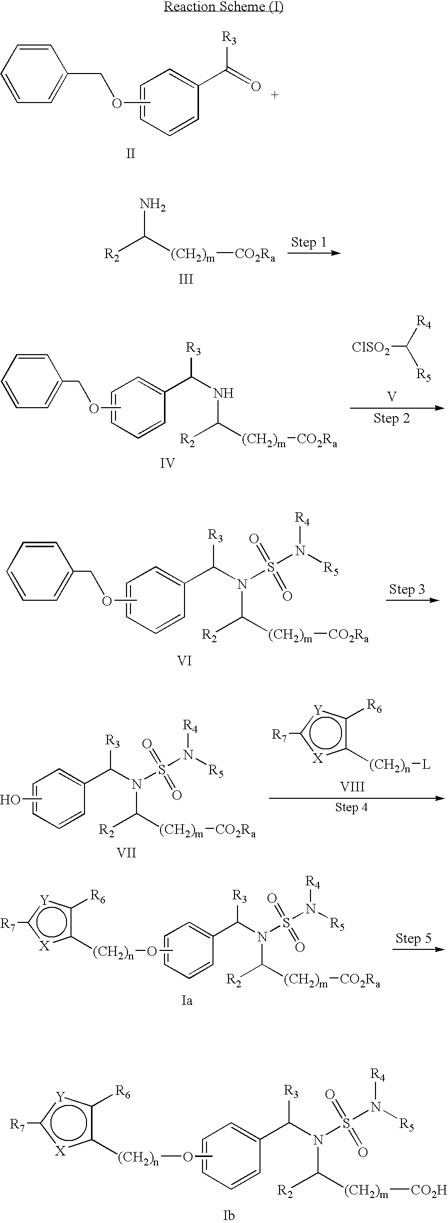
wherein,

R₂ to R₇, m and n have the same meanings as defined above; Rₐ is C₁₋₃ alkyl; and L is Cl, Br, mesylate (OMs), tosylate (OTs) or hydroxy.

As shown in Reaction Scheme (I), the inventive compound of formula (I) may be prepared by a process comprising the following steps:

Step 1: compounds of formula II and formula III are subjected to reductive amination to obtain a secondary amine compound of formula IV (yield: 80 to 98%). The reducing agents used in this step may be sodium triacetoxyborohydride or sodium borohydride, and if the compound of formula III used in this step is an acid additional salt, the reaction can be conducted in the presence of an organic base such as triethylamine. The solvent may be a halogenated organic solvents such as dichloromethane, chloroform or dichloroethane, and the reaction can be carried out at a temperature ranging from 0 to 40° C.

Step 2: The compound of formula IV is subjected to a condensation reaction with a compound of formula V in the presence of a base to obtain a compound of formula V1 (yield: 40 to 90%). The compound of formula V can be prepared in accordance with any of the known methods [Kloek, J. A et al., J. Org. Chem., 41, 4028, 1976; Matier et al., J. Med. Chem., 15, 538, 1972]. The base used in this step may be an inorganic or organic base such as sodium hydride, potassium t-butoxide, lithium bistrimethylsilylamide, lithium diisopropylamide, sodium carbonate, potassium carbonate, potassium hydroxide, sodium hydroxide, triethylamine and pyridine; the solvent, dimethylformamide, tetrahydrofuran, ethylether, dimethylsulfoxide, dichloromethane, chloroform, acetonitrile or acetone; and the reaction temperature, 0 to 40° C.

Step 3: the compound of formula V1 is allowed to react with gaseous hydrogen in the presence of a palladium catalyst/active carbon catalyst to obtain a phenol derivative of formula VII. The solvent used in this step may be ethyl alcohol or tetrahydrofuran, and the reaction is preferably carried out at room temperature under a hydrogen pressure of 1 to 5 atm.

Step 4: a compound of formula VIII, in which L is a leaving group suce as Cl, Br, OMs, or OTs, is allowed to undergo alkylation with the compound of formula VII in the presence of a base to obtain the inventive compound of formula Ia. The base may be sodium hydride, potassium t-butoxide, butyl lithium, potassium carbonate or sodium carbonate; the solvent, dimethylformamide or tetrahydrofuran, and the reaction temperature, −20 to 100° C.

Alternatively, when L of the compound of formula VIII is hydroxy, the compound of formula VIII is subjected to Mitsunobu reaction (see: Mitsunobu, O., Synthesis, 1, 1981) with the compound of formula VII to obtain the inventive compound of formula Ia. The Mitsunobu reaction is conducted in the presence of triphenylphosphine, diisopropyl azocarboxylate or diethyl azocarboxylate, using toluene or tetrahydrofuran as the solvent.

Step 5: the ester derivative of formula Ia is hydrolyzed in the presence of a base to obtain the inventive compound of formula Ib. The base may be lithium hydroxide, potassium hydroxide, sodium hydroxide, potassium carbonate and sodium carbonate, and the solvent used in this step may be methanol, ethanol, tetrahydrofuran or a mixture thereof with water.

The inventive compound of formula (I) may be prepared by another process shown in Reaction Scheme (II): 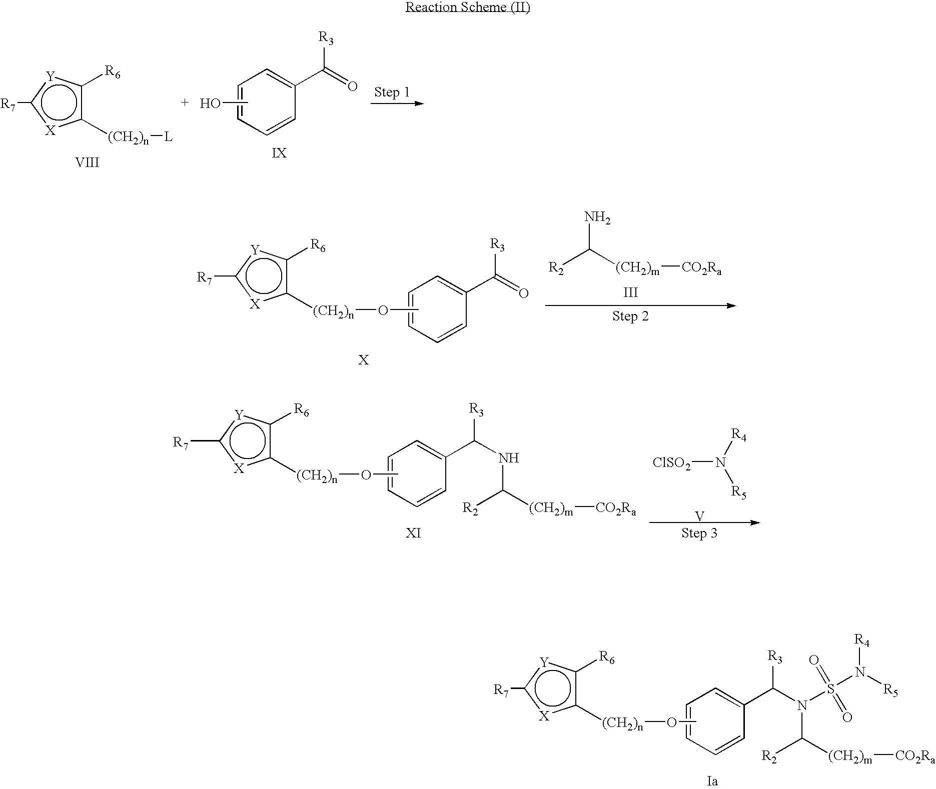
wherein,

Rₐ, R₂ to R₇, L, m or n have the same meanings as defined above.

Step 1: a compound of formula IX is subjected to a reaction which is same as that of step 4 of Reaction Scheme I to obtain a compound of formula X. In case L of the compound of formula VIII is a leaving group such as Cl, Br, mesylate or tosylate, the compound of formula VIII is subjected to an alkylation with the compound of formula IX in the presence of a base to obtain the compound of formula X. The base may be a base such as sodium hydride, potassium t-butoxide, butyl lithium, potassium carbonate or sodium carbonate; the solvent, dimethylformamide or tetrahydrofuran, and the reaction temperature, −20 to 100° C.

In case when L is hydroxy, the compound of formula VIII is subjected to Mitsunobu reaction (see: Mitsunobu, O., Synthesis, 1, 1981) with the compound of formula IX to obtain the compound of formula X. The Mitsunobu reaction is carried out in the presence of triphenylphosphine, diisopropyl azocarboxylate or diethyl azocarboxylate, using toluene or tetrahydrofuran as the solvent.

Step 2: the compound of formula X and a compound of formula III are subjected to reductive amination to obtain a secondary amine compound of formula XI in accordance with the method of step 1 of Reaction Scheme I.

Step 3: the compound of formula XI and a compound of formula V are subjected to condensation reaction in the presence of a base to obtain the inventive compound of formula Ia in accordance with the method of step 2 of Reaction Scheme I.

The inventive sulfamide derivative of formula (I) or a pharmaceutically acceptable salt thereof is effective in upregulating the lipid metabolism. Thus, the present invention also includes within its scope a pharmaceutical composition for upregulation of lipid metabolism comprising a therapeutically effective amount of a sulfamide derivative of formula (I), as defined above, or a pharmaceutically acceptable salt thereof as an active ingredient together with a pharmaceutically acceptable carrier.

The inventive pharmaceutical composition is useful for the treatment and prevention of metabolic syndrome X including obesity, NIDDM (non-insulin dependent diabetes mellitus), hyperlipidemia, arteriosclerosis, steatosis of the liver or muscle and diseases caused by lipid accumulation.

The pharmaceutical compositions of the invention may be formulated for administration orally or parenterally, including intravenous, intraperitoneal, subcutaneous, rectal and topical routes of administration in accordance with conventional methods. The composition for administration may take various forms such as tablets, powder, soft and hard gelatin capsules, aqueous solutions, suspensions, emulsions, syrups, granules, aerosol elixirs, sterilized aqueous solution, sterilized powder, non-aqueous solution and lyophilized agent, and additionally includes conventional additives such as a diluent, lubricant, filler, extender, wetting agent, absorbent, colorant, flavor, sweetener, preservative, emulsifier and the like. The pharmaceutical compositions of the invention may also be formulated for fast, continuous or delayed release of an active ingredient after administration in accordance with conventional methods.

The compound of formula (I) or a pharmaceutically acceptable salt thereof may be administered orally or parenterally as an active ingredient in an effective amount ranging from about 0.1 to 1,000 mg/kg body weight in case of mammals including human, preferably from about 1 to 100 mg/kg per day in a single dose or in divided doses. However, the foregoing dosage should be monitored, and change in consideration of idiosyncrasy and weight of the patient, kind and seriousness of illnesses, characteristics of the drug and interval and duration of drug.

The following Examples are intended to further illustrate the present invention without limiting its scope.

### EXAMPLE 1

### Preparation of [N-(aminosulfonyl)-N-[[4-(2-phenyl-5-methyl oxazol-4-yl)ethoxy]benzyl]amino]acetic acid

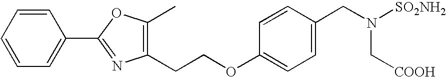

### Step 1: Preparation of [N-(4-benzyloxybenzyl)amino]acetic acid ethyl ester

21.2 g (0.1 mol) of 4-benzyloxybenzaldehyde, 15.4 g (0.11 mol) of glycine ethyl ester hydrochloride, and 11.4 g (0.11 mol) of triethylamine were dissolved in 600 ml of 1,2-dichloroethane, and 31.8 g (0.15 mol) of sodium triacetoxyborohydride was added thereto. The mixture was stirred at room temperature for 5 hours, and 500 ml of saturated sodium bicarbonylic acid was added thereto. The resulting mixture was extracted with methylene chloride, dried over anhydrous magnesium sulfate, concentrated under a reduced pressure, and the residue was subjected to silica gel (Merck, silica gel 60, 230-400 mesh) column chromatography (column: diameter×length=8 cm×50 cm, eluent: hexane/ethylacetate=3/1) to obtain the title compound (25.45 g, 85%).

¹H-NMR (CDCl₃, 300 MHz): δ (ppm) 7.43-7.30 (m, 5H), 7.24 (d, J=8.7 Hz, 2H), 6.93 (d, J=8.7 Hz, 2H), 5.04 (s, 2H), 4.17 (q, J=7.2 Hz, 2H), 3.73 (s, 2H), 3.38 (s, 2H), 1.83 (br s, 1H), 1.26 (t, J=7.2 Hz, 3H).

### Step 2: Preparation of [N-(4-benzyloxybenzyl)-N-[(t-butoxycarbonylamino) sulfonyl]amino]acetic acid ethyl ester

16.98 g (0.12 mol) of chlorosulfonyl isocyanic acid was dissolved in 120 ml of methylene chloride, and 14.82 g (0.2 mol) of t-butanol was added slowly thereto at a temperature lower than 0° C. The mixture was stirred for 30 min, 25.4 g (0.084 mol) of the compound obtained in Step 1 dissolved in 250 ml of methylene chloride was added to the reaction mixture, stirred at room temperature for overnight, and subjected to silica gel column chromatography (eluent: hexane/ethylacetate=5/1) to obtain the title compound (38.6 g, 96%).

### Step 3: Preparation of [N-(4-hydroxybenzyl)-N-[(t-butoxycarbonylamino) sulfonyl]amino]acetic acid ethyl ester

23.93 g (0.05 mol) of the compound obtained in Step 2 was dissolved in 250 ml of tetrahydrofuram, then a 10% Pd/C catalyst was added thereto, and stirred at room temperature for 12 hours under 60 psi hydrogen. The mixture was filtered to remove the catalyst, and concentrated under a reduced pressure to obtain the title compound (19.23 g, 99%).

¹H-NMR (CDCl₃, 200 MHz): δ (ppm) 7.17 (d, J=8.6 Hz, 2H), 6.80 (d, J=8.6 Hz, 2H), 4.54 (s, 2H), 4.18 (q, J=7.2 Hz, 2H), 3.98 (s, 2H), 1.51 (s, 9H), 1.26 (t, J=7.2 Hz, 3H).

### Step 4: Preparation of [N-[(t-butoxycarbonylamino)sulfonyl]-N-[[4-(2-phenyl-5-methyloxazol-4-yl)ethoxy]benzyl]amino]acetic acid ethyl ester

0.39 g (0.001 mol) of the compound obtained in Step 3, 0.31 g (0.0015 mol) of 2-(5-methyl-2-phenyl-oxazol-4-yl)ethanol and 0.45 g (0.0017 mol) of triphenylphosphine were dissolved in 5 ml of toluene, and 0.35 g (0.017 mol) of diisopropyl azodicarboxylic acid was added slowly thereto. The mixture was stirred at room temperature for 12 hours, concentrated under a reduced pressure to remove the solvent, and the residue was subjected to silica gel column chromatography (eluent: hexane/ethylacetate=3/1) to obtain the title compound (0.5 g, 89%).

¹H-NMR (CDCl₃, 300 MHz): δ (ppm) 8.19 (br s, 1H), 7.99-7.96 (m, 2H), 7.45-7.36 (m, 3H), 7.18 (dd, J=1.89 Hz, J=4.99 Hz, 2H), 6.86 (dd, J=1.89 Hz, J=4.99 Hz, 2H), 4.54 (s, 2H), 4.23 (t, J=6.6 Hz, 2H), 4.16 (q, J=7.2 Hz, 2H), 3.96 (s, 2H), 2.98 (t, J=6.6 Hz, 2H), 2.37 (s, 3H), 1.54 (s, 9H), 1.24 (t, J=7.2 Hz, 3H).

### Step 5: Preparation of [N-(aminosulfonyl)-N-[[4-(2-phenyl-5-methyloxazol-4-yl)ethoxy]benzyl]amino]acetic acid ethyl ester

0.5 g (0.9 mmol) of the compound obtained in Step 4 was dissolved in 3 ml of ethanol, 0.2 g (2.6 mmol) of acetylchloride was added slowly thereto at room temperature, and stirred for 1 day. The resulting mixture was concentrated under a reduced pressure to remove the solvent, 3 g of ice and 3 ml of saturated sodium bicarbonate aqueous solution were added thereto, extracted with methylene chloride, dried over anhydrous magnesium sulfate, and the residue was subjected to silica gel column chromatography (eluent: hexane/ethylacetate=3/1) to obtain the title compound (0.32 g, 76%).

¹H-NMR (CDCl₃, 300 MHz): δ (ppm) 7.99-7.96 (m, 2H), 7.45-7.36 (m, 3H), 7.25 (dd, J=1.89 Hz, J=4.89, 2H), 6.86 (dd, J=1.89 Hz, J=4.89 Hz, 2H), 5.29 (s, 2H), 4.30 (s, 2H), 4.23 (t, J=6.6 Hz, 2H), 4.15 (q, J=7.2 Hz, 2H), 3.87 (s, 3H), 2.98 (t, J=6.6 Hz, 2H), 2.37 (s, 3H), 1.24 (t, J=7.2 Hz, 3H).

### Step 6: Preparation of [N-(aminosulfonyl)-N-[[4-(2-phenyl-5-methyloxazol-4-yl)ethoxy]benzyl]amino]acetic acid

0.32 g (0.7 mmol) of the compound obtained in Step 5 was dissolved in 3 ml of a mixture of methanol/water (3/1, v/v), 0.34 g (0.0081 mol) of lithium hydroxide monohydrate was added thereto, and stirred at 30 to 35° C. for 3 hours. The resulting mixture was concentrated under a reduced pressure to remove methanol, diluted with 3 ml of ethylacetate, and extracted with methylenechlroride after treating with 2 ml of 1M HCl. The resulting mixture was dried over anhydrous magnesium sulfate, and the residue was subjected to silica gel column chromatography (eluent: hexane/ethylacetate=3/1) to obtain the title compound (0.3 g, 99%).

¹H-NMR (CDCl₃, 300 MHz): δ (ppm) 7.97-7.94 (m, 2H), 7.42-7.40 (m, 3H), 7.25 (d, J=8.6 Hz, 2H), 6.85 (d, J=8.6 Hz, 2H), 4.30-4.16 (m, 7H), 3.57 (s, 2H), 2.96 (t, J=6.6 Hz, 2H), 2.37 (s, 3H).

### EXAMPLE 2

### Preparation of (S)-3-methyl-2-[N-(sulfamoyl)-N-[3-[(5-methyl-2-p-tolyloxazol-4-yl)methoxy]benzyl]amino]butyric acid

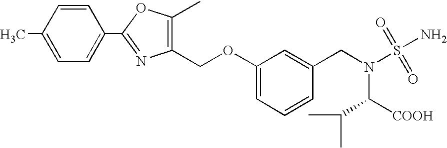

### Step 1: Preparation of 3-methyl-2-[N-(sulfamoyl)-N-3-[(5-methyl-2-p-tolyl oxazol-4-yl)methoxy]benzyl]amino]butyrate methyl ester

The procedure of Steps 2 and 5 of Example 1 was repeated except for using 442 mg (1 mmol) of (S)-3-methyl-[[2-[3-(5-methyl-2-p-tolyloxazol-4-yl)methoxy]benzyl]amino]butyrate methyl ester, 111 mg (1.1 mmol) of triethylamine and 260 mg (1.2 mmol) of t-butoxycarbonylamino sulfonyl chloride to obtain the title compound (335 mg, 65%).

¹H-NMR (CDCl₃, 300 MHz): δ (ppm) 7.89 (d, J=8.1 Hz, 2H), 7.25-7.21 (m, 3H), 7.12 (s, 1H), 7.00-6.91 (m, 2H), 4.99 (s, 2H), 4.77 (s, 2H), 4.42 (q, J=15.6 Hz, J=14.1 Hz, 2H), 3.96 (d, J=10.5 Hz, 1H), 3.69 (s, 3H), 2.42 (s, 3H), 2.39 (s, 3), 2.30-2.15 (m, 1H), 0.88-0.83 (m, 6H).

### Step 2: Preparation of (S)-3-methyl-2-[N-(sulfamoyl)-N-[3-[(5-methyl-2-p-tolyl oxazol-4-yl)methoxy]benzyl]amino]butyric acid

The procedure of Step 6 of Example 1 was repeated except for using 100 mg (0.245 mmol) of (S)-3-methyl-2-[N-(sulfamoyl)-N-3-[(5-methyl-2-p-tolyloxazol-4-yl)methoxy]benzyl]amino]butyrate methyl ester and 15 mg (0.368 mmol) of lithium hydroxy monohydrate to obtain the title compound (313 mg, 99%).

¹H-NMR (CDCl₃, 300 MHz): δ (ppm) 8.52 (br s, 1H), 7.89 (d, J=8.1 Hz, 2H), 7.30 (s, 1H), 7.01-6.96 (m, 2H), 5.02 (s, 2H), 4.42 (s, 2H), 3.81 (d, J=3.6 Hz, 1H), 2.42 (s, 3H), 2.38 (s, 3H), 2.20-2.04 (m, 1H), 1.02-0.83 (m, 6H).

### EXAMPLE 3

### Preparation of [N—[(N,N-dimethylamino)sulfonyl]-N-[3-[(2-phenyl-5-methyloxazol-4-yl)methoxy]benzyl]amino]acetic acid

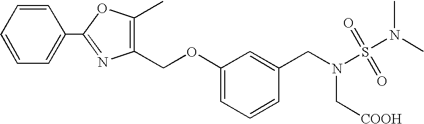

### Step 1: Preparation of [N-(3-benzyloxybenzyl)amino]acetic acid ethyl ester

106 g (0.5 mol) of 3-benzyloxybenzaldehyde, 77 g (0.55 mol) of glycine ethyl ester hydrochloride and 57 g (0.55 mol) of triethylamine were dissolved in 3 L of 1,2-dichloroethane, and 159 g (0.75 mol) of sodium triacetoxyborohydride was added thereto at room temperature, followed by stirring for 10 hours. The resulting mixture was extracted with 1 L of methylene chloride after adding 1.5 L of sodium bicarbonate, dried over anhydrous magnesium sulfate, concentrated under a reduced pressure, and the residue was subjected to silica gel column chromatography (eluent: hexane/ethylacetate=1/2) to obtain the title compound (120 g, 80%).

### Step 2: Preparation of [[N—(N,N-dimethylsulfonyl)-N-(3-benzyloxy benzyl)]amino]acetic acid ethyl ester

14.97 g (0.05 mol) of the compound obtained in Step 1 and 10.77 g (0.075 mol) of N,N-dimethylsulfamoylchloride were dissolved in 250 ml of dichloromethane, and 8.1 g (0.08 mol) of triethylamine was added thereto at room temperature, followed by stirring for 2 days. The resulting mixture was concentrated under a reduced pressure, and the residue was subjected to silica gel column chromatography (eluent: hexane/ethylacetate=3/1) to obtain the title compound (18.3 g, 90%).

### Step 3: Preparation of [[N—(N,N-dimethylsulfonyl)-N-(3-hydroxybenzyl)]amino]acetic acid ethyl ester

18.3 g (0.0475 mol) of the compound obtained in Step 2 was dissolved in 250 ml of THF, 4 g of 10% Pd/C catalyst was added thereto, and stirred for 12 hours under gaseous hydrogen (60 psi). The resulting mixture was filtered using cellite 545 (Dondyang chemicals), and the residue was concentrated under a reduced pressure to obtain the title compound (14.1 g, 99%).

### Step 4: Preparation of [N—[(N,N-dimethylamino)sulfonyl]-N-[3-[(2-phenyl-5-methyloxazol-4-yl)methoxy]benzyl]amino]acetic acid ethyl ester

0.316 g (0.001 mol) of the compound obtained in Step 3 and 0.25 g (0.0012 mol) of (4-chloromethyl-5-methyl-2-phenyl)oxazol were dissolved in 10 ml of DMF, 0.056 g (0.0014 mol) of 60% sodium hydride was added thereto, and stirred for 12 hours. After adding 5 g of ice and 3 ml of water thereto, 2 ml of 1 M HCl was added to the resulting mixture followed by extracting the mixture with 20 ml of dichloromethane. The extract was dried over 1 g of anhydrous sodium sulfate, concentrated under a reduced pressure, and the residue was subjected to silica gel column chromatography (eluent: hexane/ethylacetate=3/1) to obtain the title compound (0.34 g, 70%).

¹H-NMR (CDCl₃, 300 MHz): δ (ppm) 8.04-7.99 (m, 2H), 7.45-7.41 (m, 3H), 7.27 (t, J=7.7 Hz, 1H), 7.01-6.91 (m, 3H), 4.98 (s, 2H), 4.54 (s, 2H), 4.16 (q, J=7.2 Hz, 2H), 3.85 (s, 2H), 2.87 (s, 6H), 2.44 (s, 3H), 1.25 (t, J=7.2 Hz, 3H).

### Step 5: Preparation of [N—[(N,N-dimethylamino)sulfonyl]-N-[3-[(2-phenyl-5-methyloxazol-4-yl)methoxy]benzyl]amino]acetic acid

0.34 g (0.7 mmol) of the compound obtained in Step 4 was dissolved in 1 ml of a mixture of methanol/water (3/1, v/v), 46 mg (1.1 mmol) of lithium hydroxy monohydrate was added thereto, and stirred at room temperature for 3 hours. The resulting mixture was diluted with 1 ml of ethylacetate, and extracted with 10 ml of dichloromethane after treating with 1 ml of 1M HCl. The extract was dried over 1 g of anhydrous magnesium sulfate, concentrated under a reduced pressure and the residue was subjected to silica gel column chromatography (eluent: methanol/dichloromethane=1/20) to obtain the title compound (0.32 g, 99%).

¹H-NMR (CDCl₃, 300 MHz): δ (ppm) 9.67 (br s, 1H), 8.02-7.98 (m, 2H), 7.45-7.41 (m, 3H), 7.25-7.21 (m, 1H), 6.99-6.88 (m, 3H), 5.00 (s, 2H), 4.47 (s, 2H), 3.87 (s, 2H), 2.83 (s, 6H), 2.43 (s, 3H).

### EXAMPLE 4

### Preparation of [N—[(N,N-dimethylamino)sulfonyl]-N-[3-[[2-(4-methylphenyl)-5-methyloxazol-4-yl]methoxy]benzyl]amino]acetic acid

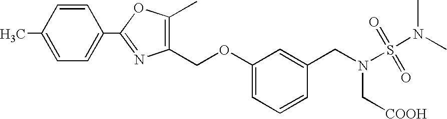

### Step 1: Preparation of [N—[(N,N-dimethylamino)sulfonyl]-N-[3-[[2-(4-methyl phenyl)-5-methyloxazol-4-yl]methoxy]benzyl]amino]acetic acid ethyl ester

The procedure of Step 4 of Example 3 was repeated except for using 316 mg (1 mmol) of the compound obtained in Step 3 of Example 3, 266 mg (1.2 mmol) of [4-chloromethyl-5-methyl-2-(4-methylphenyl)]oxazol and 56 mg (1.4 mmol) of 60% sodium hydride to obtain the title compound (416 mg, 83%).

¹H-NMR (CDCl₃, 300 MHz): δ (ppm) 7.90 (d, J=8.1 Hz, 2H), 7.31-7.22 (m, 3H), 7.00-6.91 (m, 3H), 4.97 (s, 2H), 4.53 (s, 2H), 4.15 (q, J=7.2 Hz, 2H), 3.85 (s, 2H), 2.86 (s, 6H), 2.43 (s, 3H), 2.39 (s, 3H), 1.25 (t, J=7.2 Hz, 3H).

### Step 2: Preparation of [N—[(N,N-dimethylamino)sulfonyl]-N-[3-[[2-(4-methyl phenyl)-5-methyloxazol-4-yl]methoxy]benzyl]amino]acetic acid

The procedure of Step 5 of Example 3 was repeated except for using 416 mg (0.83 mmol) of the compound obtained in Step 1 and 54 mg (1.3 mmol) of lithium hydroxide monohydrate to obtain the title compound (389 mg, 99%).

¹H-NMR (CDCl₃, 300 MHz): δ (ppm) 10.21 (br s, 1H), 7.87 (d, J=8.1 Hz, 2H), 7.26-7.20 (m, 3H), 6.99-6.87 (m, 3H), 4.99 (s, 2H), 4.46 (s, 2H), 3.86 (s, 2H), 2.82 (s, 6H), 2.42 (s, 3H), 2.38 (s, 3H).

### EXAMPLE 5

### Preparation of [N—(N,N-dimethylamino)sulfonyl-N-[3-[[2-(4-trifluoromethylphenyl)-5-methyloxazol-4-yl]methoxy]benzyl]amino]acetic acid

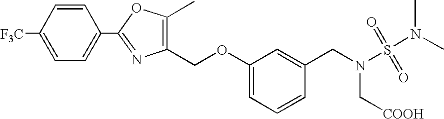

### Step 1: Preparation of [N—(N,N-dimethylamino)sulfonyl-N-[3-[[2-(4-trifluoro methylphenyl)-5-methyloxazol-4-yl]methoxy]benzyl]amino]acetic acid ethyl ester

The procedure of Step 4 of Example 3 was repeated except for using 316 mg (1 mmol) of the compound obtained in Step 3 of Example 3 and 56 mg (1.4 mmol) of 60% sodium hydride to obtain the title compound (444 mg, 80%).

¹H-NMR (CDCl₃, 300 MHz): δ (ppm) 8.13 (d, J=8.1 Hz, 2H), 7.69 (d, J=8.5 Hz, 2H), 7.32-7.24 (m, 1H), 7.03-6.91 (m, 3H), 5.00 (s, 2H), 4.55 (s, 2H), 4.16 (q, J=7.2 Hz, 2H), 3.85 (s, 2H), 2.87 (s, 6H), 2.47 (s, 3H), 1.25 (t, J=7.2 Hz, 3H).

### Step 2: Preparation of [N—(N,N-dimethylamino)sulfonyl-N-[3-[[2-(4-trifluoromethylphenyl)-5-methyloxazol-4-yl]methoxy]benzyl]amino]acetic acid

The procedure of Step 5 of Example 3 was repeated except for using 444 mg (0.8 mmol) of the compound obtained in Step 1 and 50 mg (1.2 mmol) of lithium hydroxide monohydrate to obtain the title compound (444 mg, 99%).

¹H-NMR (CDCl₃, 300 MHz): δ (ppm) 9.29 (br s, 1H), 8.12 (d, J=8.1 Hz, 2H), 7.70 (d, J=8.1 Hz, 2H), 7.28-7.24 (m, 1H), 7.02-6.90 (m, 3H), 5.01 (s, 2H), 4.49 (s, 2H), 3.89 (s, 2H), 2.84 (s, 6H), 2.47 (s, 3H).

### EXAMPLE 6

### Preparation of [N—(N-t-butylamino)sulfonyl-N-[3-[[2-(4-tri fluoromethylphenyl)-5-methyloxazol-4-yl]methoxy]benzyl]amino]acetic acid

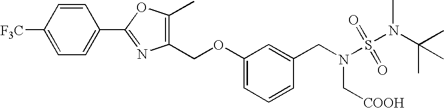

### Step 1: Preparation of [N—(N-t-butylamino)sulfonyl-N-[3-[[2-(4-trifluoromethyl phenyl)-5-methyloxazol-4-yl]methoxy]benzyl]amino]acetic acid ethyl ester

450 mg (1 mmol) of [N-[3-[[2-(4-trifluoromethylphenyl)-5-methyloxazol-4-yl]methoxy]benzyl]amino]acetic acid ethyl ester, 111 mg (1.1 mmol) of triethylamine and 210 mg (1.2 mmol) of t-butylaminosulfonylchloride were stirred together at room temperature for overnight, and the resulting mixture was subjected to silica gel column chromatography (eluent: hexane/ethylacetate=6/1) to obtain the title compound (0.35 g, 60%).

¹H-NMR (CDCl₃, 200 MHz): δ (ppm) 8.13 (d, J=8.7 Hz, 2H), 7.68 (d, J=8.7 Hz, 2H), 7.31-7.23 (m, 1H), 7.05-6.92 (m, 3H), 5.26 (s, 1H), 4.99 (s, 2H), 4.37 (s, 2H), 4.18 (q, J=7.2 Hz, 2H), 3.91 (s, 2H), 2.47 (s, 3H), 1.42 (s, 9H), 1.26 (t, J=7.2 Hz, 3H).

### Step 2: Preparation of [N—(N-t-butylamino)sulfonyl-N-[3-[[2-(4-trifluoromethyl phenyl)-5-methyloxazol-4-yl]methoxy]benzyl]amino]acetic acid

The procedure of Step 5 of Example 3 was repeated except for using 350 mg (0.6 mmol) of the compound obtained in Step 1 and 40 mg (0.9 mmol) of lithium hydroxide monohydrate to obtain the title compound (330 mg, 99%).

¹H-NMR (CDCl₃, 200 MHz): δ (ppm) 8.11 (d, J=8.8 Hz, 2H), 7.70 (d, J=8.8 Hz, 2H), 7.29-7.21 (m, 1H), 7.03-6.89 (m, 3H), 5.00 (s, 2H), 4.35 (s, 2H), 3.91 (s, 2H), 2.47 (s, 3H), 1.37 (s, 9H).

### EXAMPLE 7

### Preparation of [N—(N,N-diethylamino)sulfonyl-N-[3-[[2-(4-trifluoromethylphenyl)-5-methyloxazol-4-yl]methoxy]benzyl]amino]acetic acid

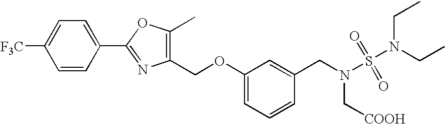

### Step 1: Preparation of [N—(N,N-diethylamino)sulfonyl-N-[3-[[2-(4-trifluoro methylphenyl)-5-methyloxazol-4-yl]methoxy]benzyl]amino]acetic acid ethyl ester

The procedure of Step 2 of Example 3 was repeated except for using 447 mg (1 mmol) of [N-[3-[[2-(4-trifluoromethylphenyl)-5-methyloxazol-4-yl]methoxy]benzyl]amino]acetic acid ethyl ester, 189 mg (1.1 mmol) of N,N-diethylsulfamoyl chloride and 111 mg (1.1 mmol) of triethylamine to obtain the title compound (472 mg, 81%).

¹H-NMR (CDCl₃, 200 MHz): δ (ppm) 8.13 (d, J=8.1 Hz, 2H), 7.69 (d, J=8.1 Hz, 2H), 7.31-7.23 (m, 1H), 7.05-6.93 (m, 3H), 5.00 (s, 2H), 4.49 (s, 2H), 4.14 (q, J=7.1 Hz, 2H), 3.81 (s, 2H), 3.33 (q, J=7.1 Hz, 4H), 2.47 (s, 3H), 1.28-1.17 (m, 9H).

### Step 2: Preparation of [N—(N,N-diethylamino)sulfonyl-N-[3-[[2-(4-trifluoro methylphenyl)-5-methyloxazol-4-yl]methoxy]benzyl]amino]acetic acid

The procedure of Step 5 of Example 3 was repeated except for using 472 mg (0.81 mmol) of the compound obtained in Step 1 and 51 mg (1.2 mmol) of lithium hydroxide monohydrate to obtain the title compound (445 mg, 99%).

¹H-NMR (CDCl₃, 200 MHz): δ (ppm) 9.19 (br s, 1H), 8.11 (d, J=8.1 Hz, 2H), 7.68 (d, J=8.1 Hz, 2H), 7.29-7.21 (m, 1H), 7.03-6.89 (m, 3H), 5.00 (s, 2H), 4.45 (s, 2H), 3.85 (s, 2H), 3.29 (q, J=7.1 Hz, 4H), 2.45 (s, 3H), 1.16 (t, J=7.1 Hz, 6H).

### EXAMPLE 8

### Preparation of [N—(N-isopropyl-N-methylamino)sulfonyl-N-[3-[[2-(4-trifluoromethylphenyl)-5-methyloxazol-4-yl]methoxy]benzyl]amino]acetic acid

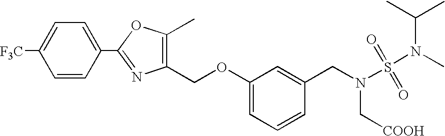

### Step 1: Preparation of [N—(N-isopropyl-N-methylamino)sulfonyl-N-[3-[[2-(4-trifluoromethylphenyl)-5-methyloxazol-4-yl]methoxy]benzyl]amino]acetic acid ethyl ester

The procedure of Step 2 of Example 3 was repeated except for using 447 mg (1 mmol) of [N-[3-[[2-(4-trifluoromethylphenyl)-5-methyloxazol-4-yl]methoxy]benzyl]amino]acetic acid ethyl ester, 189 mg (1.1 mmol) of N-isopropyl-N-methyl sulfamoyl chloride and 111 mg (1.1 mmol) of triethylamine to obtain the title compound (461 mg, 79%).

¹H-NMR (CDCl₃, 200 MHz): δ (ppm) 8.14 (d, J=8.1 hz, 2H), 7.69 (d, J=8.1 Hz, 2H), 7.31-7.23 (m, 1H), 7.05-6.93 (m, 3H), 4.99 (s, 2H), 4.48 (s, 2H), 4.24-4.09 (m, 3H), 3.81 (s, 2H), 2.74 (s, 3H), 2.47 (s, 3H), 1.28-1.17 (m, 9H).

### Step 2: Preparation of [N—(N-isopropyl-N-methylamino)sulfonyl-N-[3-[[2-(4-trifluoromethylphenyl)-5-methyloxazol-4-yl]methoxy]benzyl]amino]acetic acid

The procedure of Step 5 of Example 3 was repeated except for using 461 mg (0.79 mmol) of the compound obtained in Step 1 and 44 mg (1.05 mmol) of lithium hydroxide monohydrate to obtain the title compound (403 mg, 99%).

¹H-NMR (CDCl₃, 200 MHz): δ (ppm) 8.12 (d, J=8.1 Hz, 2H), 8.02 (br s, 1H), 7.69 (d, J=8.1 Hz, 2H), 7.30-7.22 (m, 1H), 7.04-6.91 (m, 3H), 5.00 (s, 2H), 4.44 (s, 2H), 4.21-4.15 (m, 1H), 3.85 (s, 2H), 2.72 (s, 3H), 2.47 (s, 3H), 1.19 (s, 3H), 1.15 (s, 3H).

### EXAMPLE 9

### Preparation of [N—(N-allyl-N-methylamino)sulfonyl-N-[3-[[2-(4-trifluoromethylphenyl)-5-methyloxazol-4-yl]methoxy]benzyl]amino]acetic acid

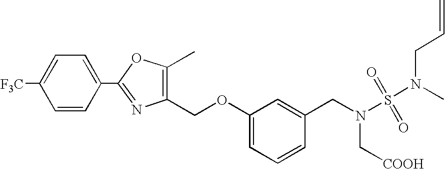

### Step 1: Preparation of [N—(N-allyl-N-methylamino)sulfonyl-N-[3-[[2-(4-trifluoromethylphenyl)-5-methyloxazol-4-yl]methoxy]benzyl]amino]acetic acid ethyl ester

The procedure of Step 2 of Example 3 was repeated except for using 447 mg (1 mmol) of [N-[3-[[2-(4-trifluoromethylphenyl)-5-methyloxazol-4-yl]methoxy]benzyl]amino]acetic acid ethyl ester, 187 mg (1.1 mmol) of N-allyl-N-methyl sulfamoyl chloride and 111 mg (1.1 mmol) of triethylamine to obtain the title compound (442 mg, 76%).

¹H-NMR (CDCl₃, 200 MHz): δ (ppm) 8.13 (d, J=8.1 Hz, 2H), 7.70 (d, J=8.1 Hz, 2H), 7.32-724 (m, 1H), 7.03-6.91 (m, 3H), 5.89-5.73 (m, 1H), 5.31-5.21 (m, 2H), 4.99 (s, 2H), 4.53 (s, 2H), 4.17 (q, J=7.1 Hz, 2H), 3.86-3.66 (m, 4H), 2.82 (s, 3H), 2.47 (s, 3H), 1.25 (t, J=7.1 Hz, 3H).

### Step 2: Preparation of [N—(N-allyl-N-methylamino)sulfonyl-N-[3-[[2-(4-trifluoromethylphenyl)-5-methyloxazol-4-yl]methoxy]benzyl]amino]acetic acid

The procedure of Step 5 of Example 3 was repeated except for using 442 mg (0.76 mmol) of the compound obtained in Step 1 and 48 mg (1.14 mmol) of lithium hydroxide monohydrate to obtain the title compound (416 mg, 99%).

¹H-NMR (CDCl₃, 200 MHz): δ (ppm) 9.20 (br s, 1H), 8.11 (d, J=8.1 Hz, 2H), 7.69 (d, J=8.1 Hz, 2H), 7.30-7.22 (m, 1H), 7.02-6.89 (m, 3H), 5.88-5.74 (m, 1H), 5.29-5.19 (m, 2H), 5.01 (s, 2H), 4.48 (s, 2H), 3.89 (s, 2H), 3.79 (d, J=6.3 Hz, 2H), 2.79 (s, 3H), 2.47 (s, 3H).

### EXAMPLE 10

### Preparation of [N—(N-methyl-N-propargylamino)sulfonyl-N-[3-[[2-(4-trifluoromethylphenyl)-5-methyloxazol-4-yl]methoxy]benzyl]amino]acetic acid

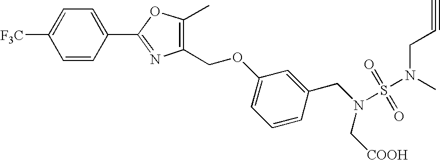

### Step 1: Preparation of [N—(N-methyl-N-propargylamino)sulfonyl-N-[3-[[2-(4-trifluoromethylphenyl)-5-methyloxazol-4-yl]methoxy]benzyl]amino]acetic acid ethyl ester

The procedure of Step 2 of Example 3 was repeated except for using 447 mg (1 mmol) of [N-[3-[[2-(4-trifluoromethylphenyl)-5-methyloxazol-4-yl]methoxy]benzyl]amino]acetic acid ethyl ester, 184 mg (1.1 mmol) of N-propargyl-N-methyl sulfamoyl chloride and 111 mg (1.1 mmol) of triethylamine to obtain the title compound (446 mg, 77%).

¹H-NMR (CDCl₃, 200 MHz): δ (ppm) 8.13 (d, J=8.1 Hz, 2H), 7.70 (d, J=8.1 Hz, 2H), 7.32-7.24 (m, 1H), 7.03-6.92 (m, 3H), 5.00 (s, 2H), 4.53 (s, 2H), 4.16 (q, J=7.1 Hz, 2H), 4.07 (d, J=2.6 Hz, 2H), 3.84 (s, 2H), 2.97 (s, 3H), 2.47 (s, 3H), 2.35 (t, J=2.6 Hz, 1H), 1.25 (t, J=7.1 Hz, 3H).

### Step 2: Preparation of [N—(N-methyl-N-propargylamino)sulfonyl-N-[3-[[2-(4-trifluoromethylphenyl)-5-methyloxazol-4-yl]methoxy]benzyl]amino]acetic acid

The procedure of Step 5 of Example 3 was repeated except for using 446 mg (0.77 mmol) of the compound obtained in Step 1 and 49 mg (1.16 mmol) of lithium hydroxide monohydrate to obtain the title compound (420 mg, 99%).

¹H-NMR (CDCl₃, 200 MHz): δ (ppm) 9.09 (br s, 1H), 8.11 (d, J=8.1 Hz, 2H), 7.70 (d, J=8.1 Hz, 2H), 7.30-7.04 (m, 1H), 6.97-6.90 (m, 3H), 5.01 (s, 2H), 4.48 (s, 2H), 4.03 (d, J=2.2 Hz, 2H), 3.88 (s, 2H), 2.94 (s, 3H), 2.47 (s, 3H), 2.35 (t, J=2.2 Hz, 1H).

### EXAMPLE 11

### Preparation of [N-(piperidinyl)sulfonyl-N-[3-[[2-(4-trifluoro methylphenyl)-5-methyloxazol-4-yl]methoxy]benzyl]amino]acetic acid

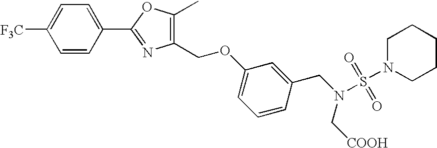

### Step 1: Preparation of [N-(piperidinyl)sulfonyl-N-[3-[[2-(4-trifluoromethyl phenyl)-5-methyloxazol-4-yl]methoxy]benzyl]amino]acetic acid ethyl ester

The procedure of Step 2 of Example 3 was repeated except for using 447 mg (1 mmol) of [N-[3-[[2-(4-trifluoromethylphenyl)-5-methyloxazol-4-yl]methoxy]benzyl]amino]acetic acid ethyl ester, 202 mg (1.1 mmol) of (piperidinyl)sulfamoyl chloride and 111 mg (1.1 mmol) of triethylamine to obtain the title compound (512 mg, 86%).

¹H-NMR (CDCl₃, 200 MHz): δ (ppm) 8.13 (d, J=8.1 Hz, 2H), 7.70 (d, J=8.1 Hz, 2H), 7.32-7.24 (m, 1H), 7.03-6.92 (m, 3H), 5.00 (s, 2H), 4.54 (s, 2H), 4.16 (q, J=7.1 Hz, 2H), 3.83 (s, 2H), 3.29-3.17 (m, 4H), 2.47 (s, 3H), 1.71-1.43 (m, 6H), 1.25 (t, J=7.1 Hz, 3H).

### Step 2: Preparation of [N-(piperidinyl)sulfonyl-N-[3-[[2-(4-trifluoromethyl phenyl)-5-methyloxazol-4-yl]methoxy]benzyl]amino]acetic acid

The procedure of Step 5 of Example 3 was repeated except for using 202 mg (0.86 mmol) of the compound obtained in Step 1 and 54 mg (1.29 mmol) of lithium hydroxide monohydrate to obtain the title compound (483 mg, 99%).

¹H-NMR (CDCl₃, 200 MHz): δ (ppm) 9.00 (br s, 1H), 8.12 (d, J=8.1 Hz, 2H), 7.69 (d, J=8.1 Hz, 2H), 7.30-7.22 (m, 1H), 7.02-6.90 (m, 3H), 5.00 (s, 2H), 4.49 (s, 2H), 3.88 (s, 2H), 3.27-3.17 (m, 4H), 2.47 (s, 3H), 1.65-1.44 (m, 6H).

### EXAMPLE 12

### Preparation of [N—(N,N-dimethylamino)sulfonyl-N-[3-[[2-(thiophen-2-yl)-5-methyloxazol-4-yl]methoxy]benzyl]amino]acetic acid

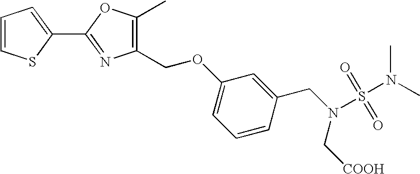

### Step 1: [N—(N,N-dimethylamino)sulfonyl-N-[3-[[2-(thiophen-2-yl)-5-methyl oxazol-4-yl]methoxy]benzyl]amino]acetic acid ethyl ester

The procedure of Step 4 of Example 3 was repeated except for using 316 mg (1 mmol) of the compound obtained in Step 3 of Example 3 and 56 mg (1.4 mmol) of 60% sodium hydride to obtain the title compound (375 mg, 76%).

¹H-NMR (CDCl₃, 300 MHz): δ (ppm) 7.65-7.62 (m, 1H), 7.41-7.39 (m, 1H), 7.27 (t, J=7.5 Hz, 1H), 7.11-7.07 (m, 1H), 6.98-6.92 (m, 3H), 4.96 (s, 2H), 4.53 (s, 2H), 4.16 (q, J=7.2 Hz, 2H), 3.85 (s, 2H), 2.87 (s, 6H), 2.42 (s, 3H), 1.25 (t, J=7.2 Hz, 3H).

### Step 2: Preparation of [N—(N,N-dimethylamino)sulfonyl-N-[3-[[2-(thiophen-2-yl)-5-methyloxazol-4-yl]methoxy]benzyl]amino]acetic acid

The procedure of Step 5 of Example 3 was repeated except for using 375 mg (0.76 mmol) of the compound obtained in Step 1 and 48 mg (1.14 mmol) of lithium hydroxide monohydrate to obtain the title compound (350 mg, 99%).

¹H-NMR (CDCl₃, 300 MHz): δ (ppm) 9.93 (br s, 1H), 7.66-7.64 (m, 1H), 7.42-7.40 (m, 1H), 7.27-7.21 (m, 1H), 6.98-6.89 (m, 3H), 4.97 (s, 2H), 4.48 (s, 2H), 3.88 (s, 2H), 2.83 (s, 6H), 2.41 (S, 3H).

### EXAMPLE 13

### Preparation of [N—(N,N-dimethylamino)sulfonyl-N-[3-[[2-(4-trifluoromethylphenyl)-4-methyloxazol-5-yl]methoxy]benzyl]amino]acetic acid

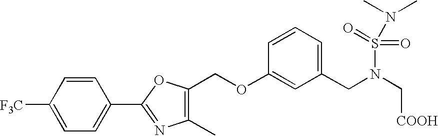

### Step 1: [N—(N,N-dimethylamino)sulfonyl-N-[3-[[2-(4-trifluoromethylphenyl)-4-methyloxazol-5-yl]methoxy]benzyl]amino]acetic acid ethyl ester

The procedure of Step 4 of Example 3 was repeated except for using 316 mg (1 mmol) of [[N—(N,N-dimethylsulfonyl)-N-(3-hydroxybenzyl)]amino]acetic acid ethyl ester, 330 mg (1.2 mmol) of [4-methyl-5-chloromethyl-2-(4-trifluoromethylphenyl)]oxazol and 56 mg (1.4 mmol) of 60% sodium hydride to obtain the title compound (450 mg, 81%).

¹H-NMR (CDCl₃, 200 MHz): δ (ppm) 8.15 (d, J=8.2 Hz, 2H), 7.71 (d, J=8.2 Hz, 2H), 7.33-7.25 (m, 1H), 7.04 (s, 1H), 6.97-6.93 (m, 2H), 5.09 (s, 2H), 4.56 (s, 2H), 4.17 (q, J=7.2 Hz, 2H), 3.86 (s, 2H), 2.88 (s, 6H), 2.31 (s, 3H), 1.25 (t, J=7.2 Hz, 3H).

### Step 2: Preparation of [N—(N,N-dimethylamino)sulfonyl-N-[3-[[2-(4-trifluoro methylphenyl)-4-methyloxazol-4-yl]methoxy]benzyl]amino]acetic acid

The procedure of Step 5 of Example 3 was repeated except for using 450 mg (0.81 mmol) of the compound obtained in Step 1 and 51 mg (1.22 mmol) of lithium hydroxide monohydrate to obtain the title compound (423 mg, 99%).

¹H-NMR (CDCl₃, 300 MHz): δ (ppm) 8.14 (d, J=8.1 Hz, 2H), 7.71 (d, J=8.1 Hz, 2H), 7.03-7.25 (m, 1H), 7.03 (s, 1H), 6.98-6.93 (m, 2H), 5.08 (s, 2H), 4.54 (s, 2H), 3.91 (s, 2H), 2.87 (s, 6H), 2.30 (s, 3H).

### EXAMPLE 14

### Preparation of [N—[(N-methyl-N-phenyl)amino]sulfonyl-N-[3-[(2-phenyl-5-methyloxazol-4-yl)methoxy]benzyl]amino]acetic acid

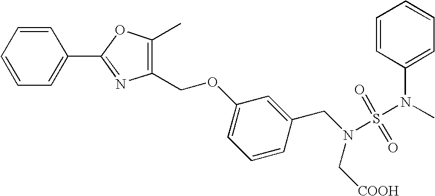

### Step 1: Preparation of [[N—[(N-methyl-N-phenyl)amino]sulfonyl-N-(3-benzyl oxybenzyl)]amino]acetic acid ethyl ester

14.97 g (0.05 mol) of [N-(3-benzyloxybenzyl)amino]acetic acid ethyl ester was dissolved in 250 ml of dichloromethane, and 15.42 g (0.075 mol) of [(N-methyl-N-phenyl)amino]sulfonyl chloride was added thereto after adding 8.1 g (0.08 mol) of triethylamine, followed by stirring at room temperature for 3 days. The resulting mixture was concentrated under a reduced pressure to remove the solvent, and the residue was subjected to silica gel column chromatography (eluent: hexane/ethylacetate=3/1) to obtain the title compound (18.3 g, 84%).

### Step 2: Preparation of [[N—[(N-methyl-N-phenyl)amino]sulfonyl-N-(3-hydroxy benzyl)]amino]acetic acid ethyl ester

The procedure of Step 3 of Example 3 was repeated except for using 18.3 g (0.042 mol) of the compound obtained in Step 1 and 4 g of 10% Pd/C catalyst to obtain the title compound (14.6 g, 99%).

### Step 3: Preparation of [N—[(N-methyl-N-phenyl)amino]sulfonyl-N-[3-[(2-phenyl-5-methyloxazol-4-yl)methoxy]benzyl]amino]acetic acid ethyl ester

The procedure of Step 4 of Example 3 was repeated except for using 379 mg (1 mmol) of the compound obtained in Step 2, 250 mg (1.2 mmol) of (4-chloromethyl-5-methyl-2-phenyl)oxazol and 56 mg (1.4 mmol) of 60% sodium hydride to obtain the title compound (434 mg, 79%).

¹H-NMR (CDCl₃, 300 MHz): δ (ppm) 8.03-7.99 (m, 2H), 7.47-7.34 (m, 7H), 7.28-7.23 (m, 2H), 6.95-6.93 (m, 2H), 6.83-6.82 (m, 1H), 4.95 (s, 2H), 4.49 (s, 2H), 4.15 (q, J=7.2 Hz, 2H), 3.85 (s, 2H), 3.31 (s, 3H), 2.43 (s, 3H), 1.24 (t, J=7.2 Hz, 3H).

### Step 4: Preparation of [N—[(N-methyl-N-phenyl)amino]sulfonyl-N-[3-[(2-phenyl-5-methyloxazol-4-yl)methoxy]benzyl]amino]acetic acid

The procedure of Step 5 of Example 3 was repeated except for using 434 mg (0.79 mmol) of the compound obtained in Step 3 and 50 mg (1.2 mmol) of lithium hydroxide monohydrate to obtain the title compound (408 mg, 99%).

¹H-NMR (CDCl₃, 200 MHz): δ (ppm) 8.01-7.96 (m, 2H), 7.45-7.14 (m, 9H), 6.95-6.78 (m, 3H), 6.22 (br s, 1H), 4.96 (s, 2H), 4.43 (s, 2H), 3.85 (s, 2H), 3.26 (s, 3H), 2.42 (s, 3H).

### EXAMPLE 15

### Preparation of [N—[(N-methyl-N-phenyl)amino]sulfonyl-N-[3-[[2-(4-methylphenyl)-5-methyloxazol-4-yl]methoxy]benzyl]amino]acetic acid

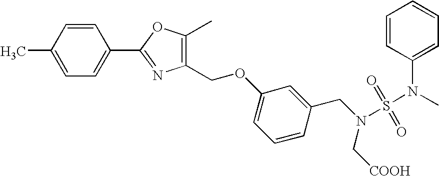

### Step 1: Preparation of [N—[(N-methyl-N-phenyl)amino]sulfonyl-N-[3-[[2-(4-methylphenyl)-5-methyloxazol-4-yl]methoxy]benzyl]amino]acetic acid ethyl ester

The procedure of Step 4 of Example 3 was repeated except for using 316 mg (1 mmol) of the compound obtained in Step 2 of Example 14, 266 mg (1.2 mmol) of [4-chloromethyl-5-methyl-2-(4-methylphenyl)]oxazol and 56 mg (1.4 mmol) of 60% sodium hydride to obtain the title compound (428 mg, 76%).

¹H-NMR (CDCl₃, 300 MHz): δ (ppm) 7.89 (d, J=8.1 Hz, 2H), 7.47-7.44 (m, 2H), 7.39-7.34 (m, 2H), 7.28-7.20 (m, 4H), 6.95-6.83 (m, 3H), 4.95 (s, 2H), 4.49 (s, 2H), 4.15 (q, J=7.2 Hz, 2H), 3.85 (s, 2H), 3.30 (s, 3H), 2.42 (s, 3H), 2.39 (s, 3H), 1.26 (t, J=7.2 Hz, 3H).

### Step 2: Preparation of [N—[(N-methyl-N-phenyl)amino]sulfonyl-N-[3-[[2-(4-methylphenyl)-5-methyloxazol-4-yl]methoxy]benzyl]amino]acetic acid

The procedure of Step 5 of Example 3 was repeated except for using 428 mg (0.76 mmol) of the compound obtained in Step 1 and 48 mg (1.14 mmol) of lithium hydroxide monohydrate to obtain the title compound (403 mg, 99%).

¹H-NMR (CDCl₃, 300 MHz): δ (ppm) 7.87 (d, J=8.1 Hz, 2H), 7.46-7.14 (m, 8H), 6.95-6.78 (m, 3H), 5.91 (br s, 1H), 4.95 (s, 2H), 4.42 (s, 2H), 3.84 (s, 2H), 3.26 (s, 3H), 2.41 (s, 3H), 2.38 (s, 3H).

### EXAMPLE 16

### Preparation of [N—[(N-methyl-N-phenyl)amino]sulfonyl-N-[3-[[2-(4-trifluoromethylphenyl)-5-methyloxazol-4-yl]methoxy]benzyl]amino]acetic acid

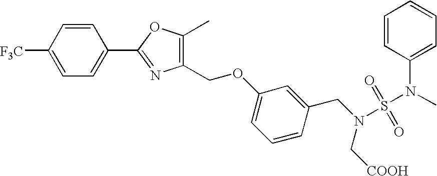

### Step 1: Preparation of [N—[(N-methyl-N-phenyl)amino]sulfonyl-N-[3-[[2-(4-trifluoromethylphenyl)-5-methyloxazol-4-yl]methoxy]benzyl]amino]acetic acid ethyl ester

The procedure of Step 4 of Example 3 was repeated except for using 316 mg (1 mmol) of the compound obtained in Step 2 of Example 14, 330 mg (1.2 mmol) of [4-chloromethyl-5-methyl-2-(4-trifluoromethylphenyl)]oxazol and 56 mg (1.4 mmol) of 60% sodium hydride to obtain the title compound (476 mg, 77%).

¹H-NMR (CDCl₃, 300 MHz): δ (ppm) 8.13 (d, J=8.4 Hz, 2H), 7.69 (d, J=8.4 Hz, 2H), 7.46-7.21 (m, 6H), 6.96-6.84 (m, 3H), 4.98 (s, 2H), 4.49 (s, 2H), 4.15 (q, J=7.2 Hz, 2H), 3.85 (s, 2H), 3.31 (s, 3H), 2.46 (s, 3H), 1.25 (t, J=7.2 Hz, 3H).

### Step 2: Preparation of [N—[(N-methyl-N-phenyl)amino]sulfonyl-N-[3-[[2-(4-trifluoromethylphenyl)-5-methyloxazol-4-yl]methoxy]benzyl]amino]acetic acid

The procedure of Step 5 of Example 3 was repeated except for using 476 mg (0.77 mmol) of the compound obtained in Step 1 and 49 mg (1.16 mmol) of lithium hydroxide monohydrate to obtain the title compound (449 mg, 99%).

¹H-NMR (CDCl₃, 300 MHz): δ (ppm) 7.10 (d, J=8.4 Hz, 2H), 7.68 (d, J=8.4 Hz, 2H), 7.46-7.17 (m, 6H), 6.97-6.81 (m, 3H), 5.34 (br s, 1H), 4.97 (s, 2H), 4.45 (s, 2H), 3.86 (s, 2H), 3.28 (s, 3H), 2.45 (s, 3H).

### EXAMPLE 17

### Preparation of [N—[(N-methyl-N-phenyl)amino]sulfonyl-N-[3-[[2-(thiophen-2-yl)-5-methyloxazol-4-yl]methoxy]benzyl]amino]acetic acid

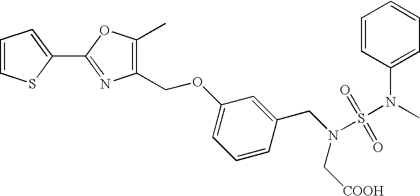

### Step 1: Preparation of [N—[(N-methyl-N-phenyl)amino]sulfonyl-N-[3-[[2-(thiophen-2-yl)-5-methyloxazol-4-yl]methoxy]benzyl]amino]acetic acid ethyl ester

The procedure of Step 4 of Example 3 was repeated except for using 316 mg (1 mmol) of the compound obtained in Step 2 of Example 14, 256 mg (1.2 mmol) of [4-chloromethyl-5-methyl-2-(thiophen-2-yl)]oxazol and 56 mg (1.4 mmol) of 60% sodium hydride to obtain the title compound (422 mg, 76%).

¹H-NMR (CDCl₃, 300 MHz): δ (ppm) 7.64-7.62 (m, 1H), 7.47-7.36 (m, 7H), 7.25-7.21 (m, 2H), 7.09-7.07 (m, 1H), 6.93-6.83 (m, 3H), 4.92 (s, 2H), 4.48 (s, 2H), 4.15 (q, J=7.2 Hz, 2H), 3.84 (s, 2H), 3.31 (s, 3H), 2.40 (s, 3H), 1.24 (t, J=7.2 Hz, 3H).

### Step 2: Preparation of [N—[(N-methyl-N-phenyl)amino]sulfonyl-N-[3-[[2-(thiophen-2-yl)-5-methyloxazol-4-yl]methoxy]benzyl]amino]acetic acid

The procedure of Step 5 of Example 3 was repeated except for using 422 mg (0.76 mmol) of the compound obtained in Step 1 and 48 mg (1.14 mmol) of lithium hydroxide monohydrate to obtain the title compound (397 mg, 99%).

¹H-NMR (CDCl₃, 300 MHz): δ (ppm) 7.67-7.64 (m, 1H), 7.46-7.06 (m, 7H), 6.94-6.79 (m, 4H), 5.79 (br s, 1H), 4.93 (s, 2H), 4.44 (s, 2H), 3.86 (s, 2H), 3.27 (s, 3H), 2.39 (s, 3H).

### EXAMPLE 18

### Preparation of [N—[[N-methyl-N-(4-chlorophenyl)]amino]sulfonyl-N-[3-[(2-phenyl-5-methyloxazol-4-yl)methoxy]benzyl]amino]acetic acid

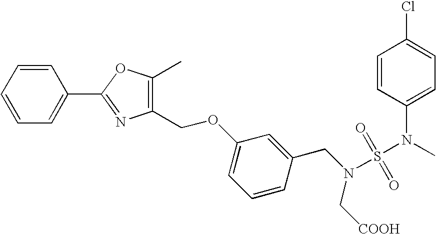

### Step 1: Preparation of [[N—[[N-methyl-N-(4-chlorophenyl)]amino]sulfonyl-N-(3-benzyloxybenzyl)]amino]acetic acid ethyl ester

14.97 g (0.05 mol) of the compound obtained in Step 1 of Example 3 and 18 g (0.075 mol) of [[N-methyl-N-(4-chlorophenyl)]amino]sulfamoylchloride were dissolved in 250 ml of dichloromethane, and 8.1 g (0.08 mol) of triethylamine was added thereto at room temperature, followed by stirring for 3 days. The resulting mixture was concentrated under a reduced pressure to remove the solvent, and the residue was subjected to silica gel column chromatography (eluent: hexane/ethylacetate=3/1) to obtain the title compound (21.1 g, 84%).

### Step 2: Preparation of [[N—[[N-methyl-N-(4-chlorophenyl)]amino]sulfonyl-N-(3-hydroxybenzyl)]amino]acetic acid ethyl ester

The procedure of Step 3 of Example 3 was repeated except for using 21.1 g (0.042 mol) of the compound obtained in Step 1 and 4 g of 10% Pd/C catalyst to obtain the title compound (17.2 g, 99%).

### Step 3: Preparation of [N—[[N-methyl-N-(4-chlorophenyl)]amino]sulfonyl-N-[3-[(2-phenyl-5-methyloxazol-4-yl)methoxy]benzyl]amino]acetic acid ethyl ester

The procedure of Step 4 of Example 3 was repeated except for using 413 mg (1 mmol) of the compound obtained in Step 2, 250 mg (1.2 mmol) of (4-chloromethyl-5-methyl-2-phenyl)oxazol and 56 mg (1.4 mmol) of 60% sodium hydride to obtain the title compound (438 mg, 75%).

¹H-NMR (CDCl₃, 300 MHz): δ (ppm) 8.03-7.99 (m, 2H), 7.44-7.22 (m, 8H), 6.96-6.94 (m, 2H), 6.86-6.83 (m, 1H), 4.96 (s, 2H), 4.47 (s, 2H), 4.15 (q, J=7.2 Hz, 2H), 3.85 (s, 2H), 3.27 (s, 3H), 2.43 (s, 3H), 1.24 (t, J=7.2 Hz, 3H).

### Step 4: Preparation of [N—[[N-methyl-N-(4-chlorophenyl)]amino]sulfonyl-N-[3-[(2-phenyl-5-methyloxazol-4-yl)methoxy]benzyl]amino]acetic acid

The procedure of Step 5 of Example 3 was repeated except for using 438 mg (0.75 mmol) of the compound obtained in Step 3 and 47 mg (1.13 mmol) of lithium hydroxide monohydrate to obtain the title compound (413 mg, 99%).

¹H-NMR (CDCl₃, 300 MHz): δ (ppm) 8.00-7.98 (m, 2H), 7.45-7.16 (m, 8H), 6.92-6.89 (m, 2H), 6.81-6.78 (m, 1H), 4.98 (s, 2H), 4.39 (s, 2H), 3.85 (s, 2H), 3.23 (s, 3H), 2.44 (s, 3H).

### EXAMPLE 19

### Preparation of [N—[[N-methyl-N-(4-chlorophenyl)]amino]sulfonyl-N-[3-[[2-(4-methylphenyl)-5-methyloxazol-4-yl]methoxy]benzyl]amino]acetic acid

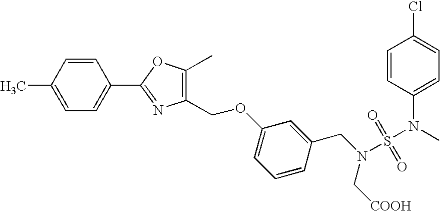

### Step 1: Preparation of [N—[[N-methyl-N-(4-chlorophenyl)]amino]sulfonyl-N-[3-[[2-(4-methylphenyl)-5-methyloxazol-4-yl]methoxy]benzyl]amino]acetic acid ethyl ester

The procedure of Step 4 of Example 3 was repeated except for using 413 mg (1 mmol) of the compound obtained in Step 3 of Example 18, 266 mg (1.2 mmol) of [4-chloromethyl-5-methyl-2-(4-methylphenyl)]oxazol and 56 mg (1.4 mmol) of 60% sodium hydride to obtain the title compound (443 mg, 74%).

¹H-NMR (CDCl₃, 300 MHz): δ (ppm) 7.90 (d, J=8.1 Hz, 2H), 7.41-7.21 (m, 7H), 6.96-6.93 (m, 2H), 6.85-6.83 (m, 1H), 4.94 (s, 2H), 4.46 (s, 2H), 4.15 (q, J=7.2 Hz, 2H), 3.84 (s, 2H), 3.27 (s, 3H), 2.42 (s, 3H), 2.39 (s, 3H), 1.24 (t, J=7.2 Hz, 3H).

### Step 2: Preparation of [N—[[N-methyl-N-(4-chlorophenyl)]amino]sulfonyl-N-[3-[[2-(4-methylphenyl)-5-methyloxazol-4-yl]methoxy]benzyl]amino]acetic acid

The procedure of Step 5 of Example 3 was repeated except for using 443 mg (0.74 mmol) of the compound obtained in Step 1 and 47 mg (1.11 mmol) of lithium hydroxide monohydrate to obtain the title compound (418 mg, 99%).

¹H-NMR (CDCl₃, 300 MHz): δ (ppm) 7.87 (d, J=8.1 Hz, 2H), 7.38-7.19 (m, 7H), 6.92-6.89 (m, 2H), 6.81-6.78 (m, 1H), 4.97 (s, 2H), 4.95 (br s, 1H), 4.38 (s, 2H), 3.84 (s, 2H), 3.24 (s, 3H), 2.43 (s, 3H), 2.39 (s, 3H).

### EXAMPLE 20

### Preparation of [N—[[N-methyl-N-(4-chlorophenyl)]amino]sulfonyl-N-[3-[[2-(4-trifluoromethylphenyl)-5-methyloxazol-4-yl]methoxy]benzyl]amino]acetic acid

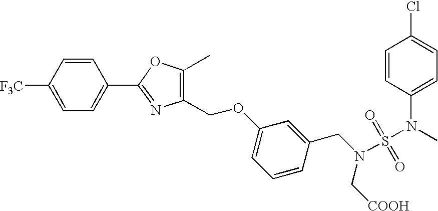

### Step 1: Preparation of [N—[[N-methyl-N-(4-chlorophenyl)]amino]sulfonyl-N-[3-[[2-(4-trifluoromethylphenyl)-5-methyloxazol-4-yl]methoxy]benzyl]amino]acetic acid ethyl ester

The procedure of Step 4 of Example 3 was repeated except for using 413 mg (1 mmol) of the compound obtained in Step 3 of Example 18, 330 mg (1.2 mmol) of [4-chloromethyl-5-methyl-2-(4-trifluoromethylphenyl)]oxazol and 56 mg (1.4 mmol) of 60% sodium hydride to obtain the title compound (496 mg, 76%).

¹H-NMR (CDCl₃, 300 MHz): δ (ppm) 8.13 (d, J=8.1 Hz, 2H), 7.70 (d, J=8.1 Hz, 2H), 7.42-7.22 (m, 5H), 6.96-6.94 (m, 2H), 6.86-6.84 (m, 2H), 4.97 (s, 2H), 4.47 (s, 2H), 4.15 (q, J=7.2 Hz, 2H), 3.85 (s, 2H), 3.28 (s, 3H), 2.46 (s, 3H), 1.24 (t, J=7.2 Hz, 3H).

### Step 2: Preparation of [N—[[N-methyl-N-(4-chlorophenyl)]amino]sulfonyl-N-[3-[[2-(4-trifluoromethylphenyl)-5-methyloxazol-4-yl]methoxy]benzyl]amino]acetic acid

The procedure of Step 5 of Example 3 was repeated except for using 496 mg (0.76 mmol) of the compound obtained in Step 1 and 48 mg (1.14 mmol) of lithium hydroxide monohydrate to obtain the title compound (470 mg, 99%).

¹H-NMR (CDCl₃, 300 MHz): δ (ppm) 8.11 (d, J=8.1 Hz, 2H), 7.69 (d, J=8.1 Hz, 2H), 7.38-7.28 (m, 5H), 6.94-6.90 (m, 2H), 6.82-6.79 (m, 1H), 5.45 (br s, 1H), 4.98 (s, 2H), 4.43 (s, 2H), 3.88 (s, 2H), 3.26 (s, 3H), 2.47 (s, 3H).

### EXAMPLE 21

### Preparation of [N—(N-ethyl-N-m-tolylamino)sulfonyl-N-[3-[[2-(4-trifluoromethylphenyl)-5-methyloxazol-4-yl]methoxy]benzyl]amino]acetic acid

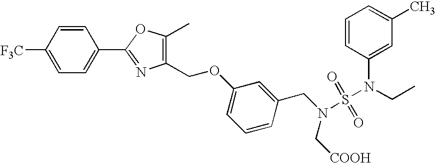

### Step 1: Preparation of [N—(N-ethyl-N-m-tolylamino)sulfonyl-N-[3-[[2-(4-trifluoromethylphenyl)-5-methyloxazol-4-yl]methoxy]benzyl]amino]acetic acid ethyl ester

The procedure of Step 2 of Example 3 was repeated except for using 447 mg (1 mmol) of [N-[3-[[2-(4-trifluoromethylphenyl)-5-methyloxazol-4-yl]methoxy]benzyl]amino]acetic acid ethyl ester, 257 mg (1.1 mmol) of (N-ethyl-N-m-tolylamino)sulfonylchloride and 111 mg (1.1 mmol) of triethylamine to obtain the title compound (310 mg, 48%).

¹H-NMR (CDCl₃, 200 MHz): δ (ppm) 8.13 (d, J=8.1 Hz, 2H), 7.69 (d, J=8.1 Hz, 2H), 7.26-7.20 (m, 4H), 7.11 (d, J=7.6 Hz, 1H), 6.95-6.92 (m, 2H), 6.83 (d, J=7.6 Hz, 2H), 4.96 (s, 2H), 4.50 (s, 2H), 4.14 (q, J=7.2 Hz, 2H), 3.83 (s, 2H), 3.70 (q, J=7.2 Hz, 2H), 2.46 (s, 3H), 2.35 (s, 3H), 1.24 (t, J=7.2 Hz, 3H), 1.08 (t, J=7.2 Hz, 3H).

### Step 2: Preparation of [N—(N-ethyl-N-m-tolylamino)sulfonyl-N-[3-[[2-(4-trifluoromethylphenyl)-5-methyloxazol-4-yl]methoxy]benzyl]amino]acetic acid

The procedure of Step 5 of Example 3 was repeated except for using 310 mg (0.48 mmol) of the compound obtained in Step 1 and 31 mg (0.72 mmol) of lithium hydroxide monohydrate to obtain the title compound (294 mg, 99%).

¹H-NMR (CDCl₃, 200 MHz): δ (ppm) 8.11 (d, J=8.1 Hz, 2H), 7.68 (d, J=8.1 Hz, 2H), 7.58 (br s, 1H), 7.29-7.09 (m, 5H), 6.96-6.89 (m, 2H), 6.79 (d, J=7.7 Hz, 1H), 4.96 (s, 2H), 4.45 (s, 2H), 3.84 (s, 2H), 3.69 (q, J=7.2 Hz, 2H), 2.45 (s, 3H), 2.34 (s, 3H), 1.08 (t, J=7.2 Hz, 3H).

### EXAMPLE 22

### Preparation of [N—(N-p-anisoyl-N-methylamino)sulfonyl-N-[3-[[2-(4-trifluoromethylphenyl)-5-methyloxazol-4-yl]methoxy]benzyl]amino]acetic acid

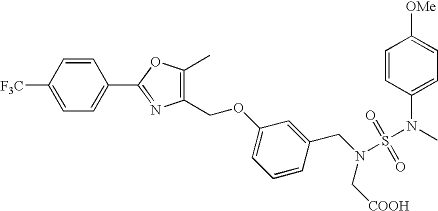

### Step 1: Preparation of [N—(N-p-anisoyl-N-methylamino)sulfonyl-N-[3-[[2-(4-trifluoromethylphenyl)-5-methyloxazol-4-yl]methoxy]benzyl]amino]acetic acid ethyl ester

The procedure of Step 2 of Example 3 was repeated except for using 447 mg (1 mmol) of [N-[3-[[2-(4-trifluoromethylphenyl)-5-methyloxazol-4-yl]methoxy]benzyl]amino]acetic acid ethyl ester, 259 mg (1.1 mmol) of (N-anisoyl-N-methylamino)sulfonyl chloride and 111 mg (1.1 mmol) of triethylamine to obtain the title compound (557 mg, 86%).

¹H-NMR (CDCl₃, 200 MHz): δ (ppm) 8.12 (d, J=8.1 Hz, 2H), 7.69 (d, J=8.1 Hz, 2H), 7.39 (d, J=8.9 Hz, 2H), 7.24-7.16 (m, 1H), 6.97-6.82 (m, 5H), 4.97 (s, 2H), 4.48 (s, 2H), 4.16 (q, J=7.0 Hz, 2H), 3.86 (s, 2H), 3.79 (s, 3H), 3.26 (s, 3H), 2.46 (s, 3H), 1.25 (t, J=7.2 Hz, 3H).

### Step 2: Preparation of [N—(N-p-anisoyl-N-methylamino)sulfonyl-N-[3-[[2-(4-trifluoromethylphenyl)-5-methyloxazol-4-yl]methoxy]benzyl]amino]acetic acid

The procedure of Step 5 of Example 3 was repeated except for using 557 mg (0.86 mmol) of the compound obtained in Step 1 and 54 mg (1.29 mmol) of lithium hydroxide monohydrate to obtain the title compound (527 mg, 99%).

¹H-NMR (CDCl₃, 200 MHz): δ (ppm) 8.35 (br s, 1H), 8.18 (d, J=8.1 Hz, 2H), 7.66 (d, J=8.1 Hz, 2H), 7.28 (d, J=8.9 Hz, 2H), 7.21-7.12 (m, 3H), 6.93-78 (m, 5H), 4.94 (s, 2H), 4.43 (s, 2H), 3.84 (s, 2H), 3.72 (s, 3H), 3.17 (s, 3H), 2.42 (s, 3H).

### EXAMPLE 23

### Preparation of [N—[N-(3-fluorophenyl)-N-methylamino]sulfonyl-N-[3-[[2-(4-trifluoromethylphenyl)-5-methyloxazol-4-yl]methoxy]benzyl]amino]acetic acid

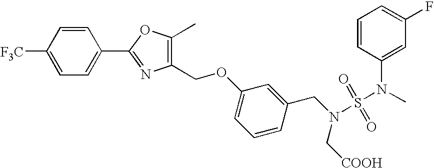

### Step 1: Preparation of [N—[N-(3-fluorophenyl)-N-methylamino]sulfonyl-N-[3-[[2-(4-trifluoromethylphenyl)-5-methyloxazol-4-yl]methoxy]benzyl]amino]acetic acid

The procedure of Step 2 of Example 3 was repeated except for using 447 mg (1 mmol) of [N-[3-[[2-(4-trifluoromethylphenyl)-5-methyloxazol-4-yl]methoxy]benzyl]amino]acetic acid ethyl ester, 246 mg (1.1 mmol) of [N-(3-fluorophenyl)-N-methylamino]sulfonyl chloride and 111 mg (1.1 mmol) of triethylamine to obtain the title compound (547 mg, 86%).

¹H-NMR (CDCl₃, 200 MHz): δ (ppm) 8.12 (d, J=8.1 Hz, 2H), 7.69 (d, J=8.1 Hz, 2H), 7.34-7.17 (m, 4H), 7.00-6.83 (m, 4H), 4.96 (s, 2H), 4.49 (s, 2H), 4.14 (q, J=7.2 Hz, 2H), 3.85 (s, 2H), 3.31 (s, 3H), 2.46 (s, 3H), 1.24 (t, J=7.2 Hz, 3H).

### Step 2: Preparation of [N—[N-(3-fluorophenyl)-N-methylamino]sulfonyl-N-[3-[[2-(4-trifluoromethylphenyl)-5-methyloxazol-4-yl]methoxy]benzyl]amino]acetic acid

The procedure of Step 5 of Example 3 was repeated except for using 547 mg (0.86 mmol) of the compound obtained in Step 1 and 54 mg (1.29 mmol) of lithium hydroxide monohydrate to obtain the title compound (517 mg, 99%).

¹H-NMR (CDCl₃, 200 MHz): δ (ppm) 8.65 (br s, 1H), 8.10 (d, J=8.1 Hz, 2H), 7.69 (d, J=8.1 Hz, 2H), 7.31-7.12 (m, 4H), 6.99-6.80 (m, 4H), 4.98 (s, 2H), 4.44 (s, 2H), 3.88 (s, 2H), 3.27 (s, 3H), 2.46 (s, 3H).

### EXAMPLE 24

### Preparation of [N—[[N-methyl-N-(p-chlorophenyl)]amino]sulfonyl-N-[3-[[2-(thiophen-2-yl)-5-methyloxazol-4-yl]methoxy]benzyl]amino]acetic acid

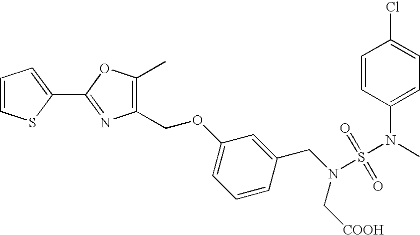

### Step 1: Preparation of [N—[[N-methyl-N-(4-chlorophenyl)]amino]sulfonyl-N-[3-[[2-(thiophen-2-yl)-5-methyloxazol-4-yl]methoxy]benzyl]amino]acetic acid ethyl ester

The procedure of Step 4 of Example 3 was repeated except for using 256 mg (1.2 mmol) of the compound obtained in Step 3 of Example 18 and 56 mg (1.4 mmol) of 60% sodium hydride to obtain the title compound (425 mg, 72%).

¹H-NMR (CDCl₃, 300 MHz): δ (ppm) 7.64-7.62 (m, 1H), 7.42-7.24 (m, 6H), 7.10-7.07 (m, 1H), 6.94-6.91 (m, 2H), 6.85-6.82 (m, 1H), 4.92 (s, 2H), 4.46 (s, 2H), 4.15 (q, J=7.2 Hz, 2H), 3.85 (s, 2H), 3.27 (s, 3H), 2.41 (s, 3H), 1.25 (t, J=7.2 Hz, 3H).

### Step 2: Preparation of [N—[[N-methyl-N-(4-chlorophenyl)]amino]sulfonyl-N-[3-[[2-(thiophen-2-yl)-5-methyloxazol-4-yl]methoxy]benzyl]amino]acetic acid

The procedure of Step 5 of Example 3 was repeated except for using 425 mg (0.72 mmol) of the compound obtained in Step 1 and 45 mg (1.08 mmol) of lithium hydroxide monohydrate to obtain the title compound (401 mg, 99%).

¹H-NMR (CDCl₃, 300 MHz): δ (ppm) 7.67-7.65 (m, 1H), 7.42-7.28 (m, 6H), 6.91-6.89 (m, 2H), 6.84-6.81 (m, 1H), 6.10 (br s, 1H), 4.95 (s, 2H), 4.41 (s, 2H), 3.86 (s, 2H), 3.25 (s, 3H), 2.41 (s, 3H).

### EXAMPLE 25

### Preparation of [N-(pyrrolidinyl)sulfonyl-N-[3-[[2-(4-trifluoro methylphenyl)-5-methyloxazol-4-yl]methoxy]benzyl]amino]acetic acid

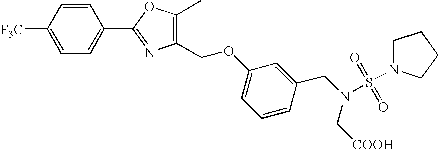

### Step 1: Preparation of [N-(pyrrolidinyl)sulfonyl-N-(3-benzyloxybenzyl)]amino]acetic acid ethyl ester

3 g (10 mmol) of [N-(3-benzyloxybenzyl)amino]acetic acid ethyl ester and 2.55 g (15 mmol) of N-(pyrrolidinyl)sulfonyl chloride were dissolved in 50 ml of dichloromethane, and 1.62 g (16 mmol) of triethylamine was added thereto at room temperature, followed by stirring for 2 days. The resulting mixture was concentrated under a reduced pressure to remove the solvent, and the residue was subjected to silica gel column chromatography (eluent: hexane/ethylacetate=3/1) to obtain the title compound (3.85 g, 89%).

### Step 2: Preparation of [N-(pyrrolidinyl)sulfonyl-N-(3-hydroxybenzyl)]amino]acetic acid ethyl ester

The procedure of Step 3 of Example 3 was repeated except for using 3.85 g (8.9 mmol) of the compound obtained in Step 1 and 0.8 g of Pd/C catalyst to obtain the title compound (3.02 g, 99%).

### Step 3: Preparation of [N-(pyrrolidinyl)sulfonyl-N-[3-[[2-(4-trifluoromethyl phenyl)-5-methyloxazol-4-yl]methoxy]benzyl]amino]acetic acid ethyl ester

The procedure of Step 4 of Example 3 was repeated except for using 342 mg (1 mmol) of the compound obtained in Step 2, 330 mg (1.2 mmol) of [4-chloromethyl-5-methyl-2-(4-trifluoromethylphenyl)]oxazol and 56 mg (1.4 mmol) of 60% sodium hydride to obtain the title compound (436 mg, 75%).

¹H-NMR (CDCl₃, 300 MHz): δ (ppm) 8.13 (d, J=8.1 Hz, 2H), 7.69 (d, J=8.1 Hz, 2H), 7.31-7.23 (m, 1H), 7.04 (s, 1H), 6.99-6.93 (m, 2H), 5.00 (s, 2H), 4.56 (s, 2H), 4.13 (q, J=7.2 Hz, 2H), 3.87 (s, 2H), 3.41-3.27 (m, 4H), 2.47 (s, 3H), 1.98-1.87 (m, 4H), 1.25 (t, J=7.2 Hz, 3H).

### Step 4: Preparation of [N-(pyrrolidinyl)sulfonyl-N-[3-[[2-(4-trifluoromethyl phenyl)-5-methyloxazol-4-yl]methoxy]benzyl]amino]acetic acid

The procedure of Step 5 of Example 3 was repeated except for using 436 mg (0.75 mmol) of the compound obtained in Step 3 and 47 mg (1.13 mmol) of lithium hydroxide monohydrate to obtain the title compound (411 mg, 99%).

¹H-NMR (CDCl₃, 300 MHz): δ (ppm) 8.12 (d, J=8.2 Hz, 2H), 7.70 (d, J=8.2 Hz, 2H), 7.29-7.23 (m, 2H), 7.05 (s, 1H), 6.96-6.92 (m, 2H), 5.00 (s, 2H), 4.51 (s, 2H), 4.50 (br s, 1H), 3.91 (s, 2H), 3.38-3.29 (m, 4H), 2.47 (s, 3H), 1.23-1.18 (m, 4H).

### EXAMPLE 26

### Preparation of [N-(pyrrolidinyl)sulfonyl-N-[3-[[2-(4-trifluoro methylphenyl)-4-methyloxazol-5-yl]methoxy]benzyl]amino]acetic acid

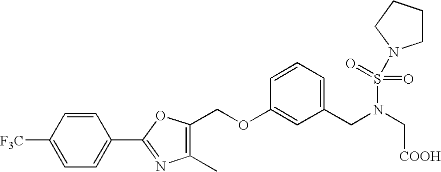

### Step 1: Preparation of [N-(pyrrolidinyl)sulfonyl-N-[3-[[2-(4-trifluoromethylphenyl)-4-methyloxazol-5-yl]methoxy]benzyl]amino]acetic acid ethyl ester

The procedure of Step 4 of Example 3 was repeated except for using 342 mg (1 mmol) of [N-(pyrrolidinyl)sulfonyl-N-(3-hydroxybenzyl)]amino]acetic acid ethyl ester, 330 mg (1.2 mmol) of [5-chloromethyl-4-methyl-2-(4-trifluoromethylphenyl)]oxazol and 56 mg (1.4 mmol) of 60% sodium hydride to obtain the title compound (413 mg, 71%).

¹H-NMR (CDCl₃, 300 MHz): δ (ppm) 8.15 (d, J=8.1 Hz, 2H), 7.71 (d, J=8.1 Hz, 2H), 7.69-7.25 (m, 1H), 7.07 (s, 1H), 6.98-6.91 (m, 3H), 5.09 (s, 2H), 4.57 (s, 2H), 4.16 (q, J=7.2 Hz, 2H), 3.87 (s, 2H), 3.41-3.35 (m, 4H), 2.31 (s, 3H), 1.94-1.88 (m, 4H), 1.25 (t, J=7.2 Hz, 2H).

### Step 2: Preparation of [N-(pyrrolidinyl)sulfonyl-N-[3-[[2-(4-trifluoromethyl phenyl)-4-methyloxazol-5-yl]methoxy]benzyl]amino]acetic acid

The procedure of Step 5 of Example 3 was repeated except for using 413 mg (0.71 mmol) of the compound obtained in Step 1 and 45 mg (1.07 mmol) of lithium hydroxide monohydrate to obtain the title compound (389 mg, 99%).

¹H-NMR (CDCl₃, 300 MHz): δ (ppm) 8.14 (d, J=8.3 Hz, 2H), 7.71 (d, J=8.3 Hz, 2H), 7.30-7.25 (m, 1H), 7.04-6.95 (m, 3H), 5.08 (s, 2H), 4.54 (s, 2H), 3.92 (s, 2H), 3.91-3.75 (m, 4H), 2.30 (s, 3H), 1.92-1.88 (m, 4H).

### EXAMPLE 27

### Preparation of [N-(4-methyl-1-piperazinyl)sulfonyl-N-[3-[[2-(4-trifluoromethylphenyl)-5-methyloxazol-4-yl]methoxy]benzyl]amino]acetic acid

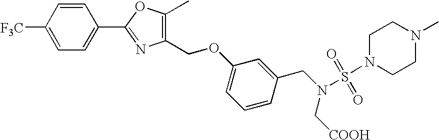

### Step 1: Preparation of [N-(4-methyl-1-piperazinyl)sulfonyl-N-(3-benzyloxy benzyl)]amino]acetic acid ethyl ester

3 g (10 mmol) of [N-(3-benzyloxybenzyl)amino]acetic acid ethyl ester and 2.98 g (15 mmol) of (4-methyl-1-piperazinyl)sulfonyl chloride were dissolved in 50 ml of dichloromethane, and 1.62 g (16 mmol) of triethylamine was added thereto at room temperature, followed by stirring for 2 days. The resulting mixture was concentrated under a reduced pressure to remove the solvent, and the residue was subjected to silica gel column chromatography (eluent: dichloromethane/ethylacetate=1/1) to obtain the title compound (4.06 g, 88%).

### Step 2: Preparation of [N-(4-methyl-1-piperazinyl)sulfonyl-N-(3-hydroxy benzyl)]amino]acetic acid ethyl ester

The procedure of Step 3 of Example 3 was repeated except for using 4.06 g (8.8 mmol) of [N-(4-methyl-1-piperazinyl)sulfonyl-N-(3-benzyloxybenzyl)]amino]acetic acid ethyl ester and 0.8 g of Pd/C catalyst to obtain the title compound (3.23 g, 99%).

### Step 3: Preparation of [N-(4-methyl-1-piperazinyl)sulfonyl-N-[3-[[2-(4-trifluoro methylphenyl)-5-methyloxazol-4-yl]methoxy]benzyl]amino]acetic acid

The procedure of Step 4 of Example 3 was repeated except for using 371 mg (1 mmol) of the compound obtained in Step 2, 330 mg (1.2 mmol) of [4-chloromethyl-5-methyl-2-(4-trifluoromethylphenyl)]oxazol and 56 mg (1.4 mmol) of 60% sodium hydride to obtain the title compound (470 mg, 77%).

¹H-NMR (CDCl₃, 300 MHz): δ (ppm) 8.13 (d, J=8.2 Hz, 2H), 7.69 (d, J=8.2 Hz, 2H), 7.32-7.24 (m, 1H), 7.02-6.92 (m, 3H), 5.00 (s, 2H), 4.55 (s, 2H), 4.16 (q, J=7.2 Hz, 2H), 3.84 (s, 2H), 3.37-3.32 (m, 4H), 2.47 (s, 3H), 2.46-2.43 (m, 4H), 2.30 (s, 3H), 1.25 (t, J=7.2 Hz, 3H).

### Step 4: Preparation of [N-(4-methyl-1-piperazinyl)sulfonyl-N-[3-[[2-(4-trifluoro methylphenyl)-5-methyloxazol-4-yl]methoxy]benzyl]amino]acetic acid

The procedure of Step 5 of Example 3 was repeated except for using 470 mg (0.77 mmol) of the compound obtained in Step 3 and 49 mg (1.16 mmol) of lithium hydroxide monohydrate to obtain the title compound (444 mg, 90%).

¹H-NMR (DMSO-d₆, 300 MHz): δ (ppm) 8.09 (d, J=8.2 Hz, 2H), 7.82 (d, J=8.2 Hz, 2H), 7.28-7.22 (m, 1H), 6.95-6.85 (m, 3H), 4.96 (s, 2H), 4.39 (s, 2H), 3.70 (s, 3H), 3.08-3.05 (m, 4H), 2.45 (s, 3H), 2.43-2.42 (m, 4H).

### EXAMPLE 28

### Preparation of [N-(morpholinyl)sulfonyl-N-[3-[[2-(4-methyl phenyl)-5-methyloxazol-4-yl]methoxy]benzyl]amino]acetic acid

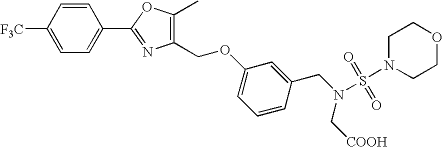

### Step 1: Preparation of [N-(morpholinyl)sulfonyl-N-(3-benzyloxybenzyl)]amino]acetic acid ethyl ester

3 g (10 mmol) of [N-(3-benzyloxybenzyl)amino]acetic acid ethyl ester and 2.78 g (15 mmol) of N-(morpholinyl)sulfonyl chloride were dissolved in 50 ml of dichloromethane, and 1.62 g (16 mmol) of triethylamine was added thereto at room temperature, followed by stirring for 2 days. The resulting mixture was concentrated under a reduced pressure to remove the solvent, and the residue was subjected to silica gel column chromatography (eluent: hexane/ethylacetate=1/1) to obtain the title compound (3.77 g, 84%).

### Step 2: Preparation of [N-(morpholinyl)sulfonyl-N-(3-hydroxybenzyl)]amino]acetic acid ethyl ester

The procedure of Step 3 of Example 3 was repeated except for using 3.77 g (8.4 mmol) of the compound obtained in Step 1 and 0.8 g of Pd/C catalyst to obtain the title compound (2.98 g, 99%).

### Step 3: Preparation of [N-(morpholinyl)sulfonyl-N-[3-[[2-(4-methylphenyl)-5-methyloxazol-4-yl]methoxy]benzyl]amino]acetic acid ethyl ester

The procedure of Step 4 of Example 3 was repeated except for using 330 mg (1.2 mmol) of the compound obtained in Step 2 and 56 mg (1.4 mmol) of 60% sodium hydride to obtain the title compound (466 mg, 78%).

¹H-NMR (CDCl₃, 300 MHz): δ (ppm) 8.13 (d, J=8.1 Hz, 2H), 7.69 (d, J=8.1 Hz, 2H), 7.33-7.25 (m, 1H), 7.02-6.91 (m, 3H), 5.00 (s, 2H), 4.57 (s, 2H), 4.17 (q, J=7.2 Hz, 2H), 3.86 (s, 2H), 3.74-3.69 (m, 4H), 3.32-3.27 (m, 4H), 2.47 (s, 3H), 1.25 (t, J=7.2 Hz, 3H).

### Step 4: Preparation of [N-(morpholinyl)sulfonyl-N-[3-[[2-(4-methylphenyl)-5-methyloxazol-4-yl]methoxy]benzyl]amino]acetic acid

The procedure of Step 5 of Example 3 was repeated except for using 466 mg (0.78 mmol) of the compound obtained in Step 3 and 49 mg (1.17 mmol) of lithium hydroxide monohydrate to obtain the title compound (440 mg, 99%).

¹H-NMR (CDCl₃, 300 MHz): δ (ppm) 8.12 (d, J=8.2 Hz, 2H), 7.70 (d, J=8.2 Hz, 2H), 7.27-7.24 (m, 1H), 7.02 (s, 1H), 6.98-6.89 (m, 2H), 5.01 (s, 2H), 4.53 (s, 2H), 3.90 (s, 2H), 3.73-3.70 (m, 4H), 3.51 (br s, 1H), 3.30-3.28 (m, 4H), 2.48 (s, 3H).

### EXAMPLE 29

### Preparation of [N-(morpholinyl)sulfonyl-N-[3-[[2-(4-trifluoromethylphenyl)-4-methyloxazol-5-yl]methoxy]benzyl]amino]acetic acid

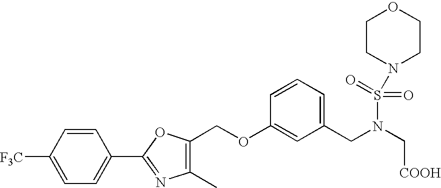

### Step 1: Preparation of [N-(morpholinyl)sulfonyl-N-[3-[[2-(4-trifluoromethyl phenyl)-4-methyloxazol-4-yl]methoxy]benzyl]amino]acetic acid ethyl ester

The procedure of Step 4 of Example 3 was repeated except for using 454 mg (1.0 mmol) of the compound obtained in Step 2 of Example 28, 330 mg (1.2 mmol) of [5-chloromethyl-4-methyl-2-(4-trifluoromethylphenyl)]oxazol and 56 mg (1.4 mmol) of 60% sodium hydride to obtain the title compound.

¹H-NMR (CDCl₃, 300 MHz): δ (ppm) 8.15 (d, J=8.1 Hz, 2H), 7.71 (d, J=8.1 Hz, 2H), 7.34-7.26 (m, 1H), 7.02-6.93 (m, 3H), 5.08 (s, 2H), 4.57 (s, 2H), 4.18 (q, J=7.2 Hz, 2H), 3.87 (s, 2H), 3.74-3.70 (m, 4H), 3.32-3.28 (m, 4H), 2.31 (s, 3H), 1.26 (t, J=7.2 Hz, 3H).

### Step 2: Preparation of [N-(morpholinyl)sulfonyl-N-[3-[[2-(4-trifluoromethyl phenyl)-4-methyloxazol-5-yl]methoxy]benzyl]amino]acetic acid

The procedure of Step 5 of Example 3 was repeated except for using 454 mg (0.76 mmol) of the compound obtained in Step 1 and 49 mg (1.17 mmol) of lithium hydroxide monohydrate to obtain the title compound (429 mg, 99%). ¹H-NMR (CDCl₃, 300 MHz): δ (ppm) 8.14 (d, J=8.3 Hz, 2H), 7.71 (d, J=8.3 Hz, 2H), 7.31-7.26 (m, 1H), 7.01-6.95 (m, 3H), 5.08 (s, 2H), 4.57 (s, 2H), 3.92 (s, 2H), 3.74-3.71 (m, 4H), 3.31-3.28 (m, 4H), 2.30 (s, 3H).

### EXAMPLE 30

### Preparation of [N-(indolinyl)sulfonyl-N-[3-[(2-phenyl-5-methyloxazol-4-yl)methoxy]benzyl]amino]acetic acid

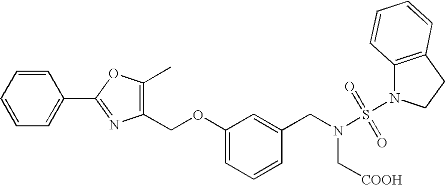

### Step 1: Preparation of [[N-(indolinyl)sulfonyl-N-(3-benzyloxybenzyl)]amino]acetic acid ethyl ester

14.97 g (0.05 mol) of [N-(3-benzyloxybenzyl)amino]acetic acid ethyl ester and 16.33 g (0.075 mol) of (indolinyl)sulfamoyl chloride were dissolved in 250 ml of dichloromethane, and 8.1 g (0.08 mol) of triethylamine was added thereto at room temperature, followed by stirring for 3 days. The resulting mixture was concentrated under a reduced pressure, and the residue was subjected to silica gel column chromatography (eluent: hexane/ethylacetate=3/1) to obtain the title compound (20.18 g, 84%).

### Step 2: Preparation of [[N-(indolinyl)sulfonyl-N-(3-hydroxybenzyl)]amino]acetic acid ethyl ester

The procedure of Step 3 of Example 3 was repeated except for using 20.18 g (0.042 mol) of the compound obtained in Step 1 and 0.8 g of Pd/C catalyst to obtain the title compound (16.23 g, 99%).

### Step 3: Preparation of [N-(indolinyl)sulfonyl-N-[3-[(2-phenyl-5-methyloxazol-4-yl)methoxy]benzyl]amino]acetic acid ethyl ester

The procedure of Step 4 of Example 3 was repeated except for using 390 mg (1 mmol) of the compound obtained in Step 2, 250 mg (1.2 mmol) of (4-chloromethyl-5-methyl-2-phenyl)oxazol and 56 mg (1.4 mmol) of 60% sodium hydride to obtain the title compound (438 mg, 78%).

¹H-NMR (CDCl₃, 200 MHz): δ (ppm) 8.02-7.99 (m, 2H), 7.43-7.41 (m, 4H), 7.25-7.12 (m, 3H), 6.95-6.82 (m, 4H), 4.88 (s, 2H), 4.62 (s, 2H), 4.11-3.89 (m, 6H), 3.12 (t, J=8.5 Hz, 2H), 2.43 (s, 3H), 1.12 (t, J=7.2 Hz, 3H).

### Step 4: Preparation of [N-(indolinyl)sulfonyl-N-[3-[(2-phenyl-5-methyloxazol-4-yl)methoxy]benzyl]amino]acetic acid

The procedure of Step 5 of Example 3 was repeated except for using 438 mg (0.78 mmol) of the compound obtained in Step 1 and 49 mg (1.17 mmol) of lithium hydroxide monohydrate to obtain the title compound (412 mg, 99%).

¹H-NMR (CDCl₃, 200 MHz): δ (ppm) 8.00-7.96 (m, 2H), 7.45-7.39 (m, 4H), 7.14-7.08 (m, 3H), 6.91-6.88 (m, 4H), 5.29 (s, 1H), 4.85 (s, 2H), 4.47 (s, 2H), 3.96 (t, J=8.5 Hz, 2H), 3.93 (s, 2H), 3.05 (t, J=8.5 Hz, 2H), 2.43 (s, 3H).

### EXAMPLE 31

### Preparation of [N-(indolinyl)sulfonyl-N-[3-[[2-(4-methyl phenyl)-5-methyloxazol-4-yl]methoxy]benzyl]amino]acetic acid

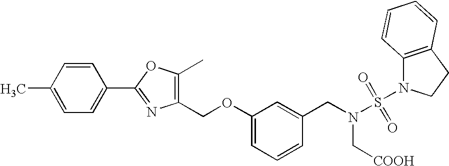

### Step 1: Preparation of [N-(indolinyl)sulfonyl-N-[3-[[2-(4-methylphenyl)-5-methyloxazol-4-yl]methoxy]benzyl]amino]acetic acid ethyl ester

The procedure of Step 4 of Example 3 was repeated except for using 412 mg of the compound obtained in Step 2 of Example 30, 266 mg (1.2 mmol) of [4-chloromethyl-5-methyl-2-(4-methylphenyl)]oxazol and 56 mg (1.4 mmol) of 60% sodium hydride to obtain the title compound (409 mg, 71%).

¹H-NMR (CDCl₃, 200 MHz): δ (ppm) 7.90 (d, J=8.1 Hz, 2H), 7.41 (d, J=7.9 Hz, 1H), 7.25-7.13 (m, 5H), 6.95-6.82 (m, 4H), 4.88 (s, 2H), 4.62 (s, 2H), 4.10-3.94 (m, 4H), 3.88 (s, 2H), 3.12 (t, J=8.5 Hz, 2H), 2.42 (s, 3H), 2.39 (s, 3H), 1.23 (t, J=7.2 Hz, 3H).

### Step 2: Preparation of [N-(indolinyl)sulfonyl-N-[3-[[2-(4-methylphenyl)-5-methyloxazol-4-yl]methoxy]benzyl]amino]acetic acid

The procedure of Step 5 of Example 3 was repeated except for using 409 mg (0.71 mmol) of the compound obtained in Step 1 and 45 mg (1.07 mmol) of lithium hydroxide monohydrate to obtain the title compound (385 mg, 99%).

¹H-NMR (CDCl₃, 200 MHz): δ (ppm) 7.86 (d, J=8.1 Hz, 2H), 7.42 (d, J=7.9 Hz, 1H), 7.25-7.08 (m, 5H), 6.94-6.76 (m, 4H), 5.37 (s, 1H), 4.87 (s, 2H), 4.54 (s, 2H), 4.00 (t, J=8.5 Hz, 2H), 3.92 (s, 2H), 3.04 (t, J=8.5 Hz, 2H), 2.41 (s, 3H), 2.39 (s, 3H).

### EXAMPLE 32

### Preparation of [N-(indolinyl)sulfonyl-N-[3-[[2-(4-trifluoro methylphenyl)-5-methyloxazol-4-yl]methoxy]benzyl]amino]acetic acid

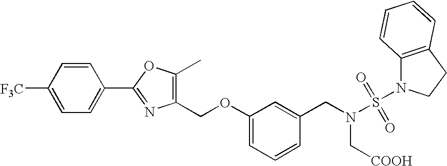

### Step 1: Preparation of [N-(indolinyl)sulfonyl-N-[3-[[2-(4-trifluoromethyl phenyl)-5-methyloxazol-4-yl]methoxy]benzyl]amino]acetic acid ethyl ester

The procedure of Step 4 of Example 3 was repeated except for using 390 mg (1 mmol) of the compound obtained in Step 2 of Example 30, 330 mg (1.2 mmol) of [4-chloromethyl-5-methyl-2-(4-trifluoromethylphenyl)]oxazol and 56 mg (1.4 mmol) of 60% sodium hydride to obtain the title compound (441 mg, 70%).

¹H-NMR (CDCl₃, 200 MHz): δ (ppm) 8.13 (d, J=8.1 Hz, 2H), 7.70 (d, J=8.1 Hz, 2H), 7.41 (d, J=9.1 Hz, 1H), 7.25-7.14 (m, 3H), 6.97-6.84 (m, 4H), 4.91 (s, 2H), 4.63 (s, 2H), 4.07 (t, J=8.5 Hz, 2H), 3.93 (q, J=7.2 Hz, 2H), 3.89 (s, 2H), 3.13 (t, J=8.5 Hz, 2H), 2.46 (s, 3H), 1.12 (t, J=7.2 Hz, 3H).

### Step 2: Preparation of [N-(indolinyl)sulfonyl-N-[3-[[2-(4-trifluoromethyl phenyl)-5-methyloxazol-4-yl]methoxy]benzyl]amino]acetic acid

The procedure of Step 5 of Example 3 was repeated except for using 441 mg (0.70 mmol) of the compound obtained in Step 1 and 44 mg (1.05 mmol) of lithium hydroxide monohydrate to obtain the title compound (417 mg, 99%).

¹H-NMR (CDCl₃, 200 MHz): δ (ppm) 8.10 (d, J=8.7 Hz, 2H), 7.75 (d, J=8.7 Hz, 2H), 7.39 (d, J=9.1 Hz, 1H), 7.25-7.09 (m, 3H), 6.95-6.78 (m, 4H), 4.87 (s, 2H), 4.83 (s, 1H), 4.53 (s, 2H), 4.03 (t, J=8.3 Hz, 2H), 3.95 (s, 2H), 3.08 (t, J=8.3 Hz, 2H), 2.45 (s, 3H).

### EXAMPLE 33

### Preparation of [N-(indolinyl)sulfonyl-N-[3-[[2-(thiophen-2-yl)-5-methyloxazol-4-yl]methoxy]benzyl]amino]acetic acid

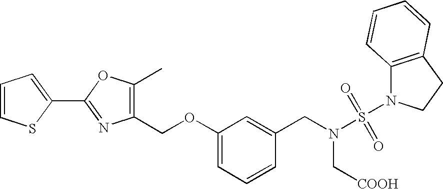

### Step 1: Preparation of [N-(indolinyl)sulfonyl-N-[3-[[2-(thiophen-2-yl)-5-methyloxazol-4-yl]methoxy]benzyl]amino]acetic acid ethyl ester

The procedure of Step 4 of Example 3 was repeated except for using 390 mg (1 mmol) of the compound obtained in Step 2 of Example 30, 256 mg (1.2 mmol) of [4-chloromethyl-5-methyl-2-(thiophen-2-yl)]oxazol and 56 mg (1.4 mmol) of 60% sodium hydride to obtain the title compound (392 mg, 69%).

¹H-NMR (CDCl₃, 200 MHz): δ (ppm) 7.64-7.62 (m, 1H), 7.41-7.38 (m, 2H), 7.22-7.07 (m, 4H), 6.93-6.86 (m, 4H), 4.86 (s, 2H), 4.62 (s, 2H), 4.12-3.95 (m, 4H), 3.89 (s, 2H), 3.12 (t, J=8.5 Hz, 2H), 2.41 (s, 3H), 1.13 (t, J=7.2 Hz, 3H).

### Step 2: Preparation of [N-(indolinyl)sulfonyl-N-[3-[[2-(thiophen-2-yl)-5-methyl oxazol-4-yl]methoxy]benzyl]amino]acetic acid

The procedure of Step 5 of Example 3 was repeated except for using 392 mg (0.69 mmol) of the compound obtained in Step 1 and 44 mg (1.04 mmol) of lithium hydroxide monohydrate to obtain the title compound (369 mg, 99%).

¹H-NMR (CDCl₃, 200 MHz): δ (ppm) 7.66-7.63 (m, 1H), 7.44-7.39 (m, 2H), 7.19-7.07 (m, 4H), 6.95-6.77 (m, 4H), 5.29 (s, 1H), 4.83 (s, 2H), 4.50 (s, 2H), 4.02 (t, J=8.3 Hz, 2H), 3.94 (s, 2H), 3.07 (t, J=8.3 Hz, 2H), 2.40 (s, 3H).

### EXAMPLE 34

### Preparation of [N-(1,2,3,4-tetrahydroquinolinyl)sulfonyl-N-[3-[(2-phenyl-5-methyloxazol-4-yl)methoxy]benzyl]amino]acetic acid

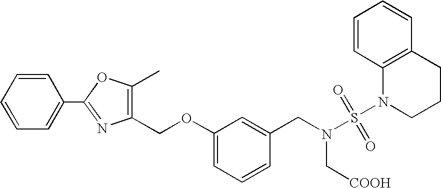

### Step 1: Preparation of [[N-(1,2,3,4-tetrahydroquinolinyl)sulfonyl-N-(3-benzyloxybenzyl)]amino]acetic acid ethyl ester

14.97 g (0.05 mol) of [N-(3-benzyloxybenzyl)amino]acetic acid ethyl ester and 17.38 g (0.075 mol) of (1,2,3,4-tetrahydroquinolinyl)sulfamoyl chloride were dissolved in 250 ml of dichloromethane, and 8.1 g (0.08 mol) of triethylamine was added thereto at room temperature, followed by stirring for 3 days. The resulting mixture was concentrated under a reduced pressure, and the residue was subjected to silica gel column chromatography (eluent: hexane/ethylacetate=3/1) to obtain the title compound (20.03 g, 81%).

### Step 2: Preparation of [[N-(1,2,3,4-tetraquinolinyl)sulfonyl-N-(3-hydroxy benzyl)]amino]acetic acid ethyl ester

The procedure of Step 3 of Example 3 was repeated except for using 19.78 g (0.04 mol) of the compound obtained in Step 1 and 0.8 g of Pd/C catalyst to obtain the title compound (16.02 g, 99%).

### Step 3: Preparation of [N-(1,2,3,4-tetrahydroquinolinyl)sulfonyl-N-[3-[(2-phenyl-5-methyloxazol-4-yl)methoxy]benzyl]amino]acetic acid ethyl ester

The procedure of Step 4 of Example 3 was repeated except for using 404 mg (1 mmol) of the compound obtained in Step 2, 250 mg (1.2 mmol) of (4-chloromethyl-5-methyl-2-phenyl)oxazol and 56 mg (1.4 mmol) of 60% sodium hydride to obtain the title compound (438 mg, 76%).

¹H-NMR (CDCl₃, 200 MHz): δ (ppm) 8.04-7.99 (m, 2H), 7.63 (d, J=7.7 Hz, 1H), 7.45-7.42 (m, 3H), 7.25-6.96 (m, 5H), 6.94-6.76 (m, 2H), 4.86 (s, 2H), 4.55 (s, 2H), 4.10 (q, J=7.2 Hz, 2H), 3.86 (s, 2H), 3.79 (t, J=5.8 Hz, 2H), 2.79 (t, J=6.8 Hz, 2H), 2.43 (s, 3H), 2.09-2.03 (m, 2H), 1.20 (t, J=7.2 Hz, 3H).

### Step 4: Preparation of [N-(1,2,3,4-tetrahydroquinolinyl)sulfonyl-N-[3-[(2-phenyl-5-methyloxazol-4-yl)methoxy]benzyl]amino]acetic acid

The procedure of Step 5 of Example 3 was repeated except for using 438 mg (0.76 mmol) of the compound obtained in Step 3 and 48 mg (1.14 mmol) of lithium hydroxide monohydrate to obtain the title compound (412 mg, 99%).

¹H-NMR (CDCl₃, 200 MHz): δ (ppm) 8.04-7.99 (m, 2H), 7.61 (d, J=7.7 Hz, 1H), 7.45-7.42 (m, 3H), 7.18-6.98 (m, 4H), 6.90-6.73 (m, 3H), 4.86 (s, 2H), 4.45 (s, 2H), 3.89 (s, 2H), 3.75 (t, J=5.8 Hz, 2H), 2.78 (t, J=6.8 Hz, 2H), 2.42 (s, 3H), 2.08-2.02 (m, 2H).

### EXAMPLE 35

### Preparation of [N-(1,2,3,4-tetrahydroquinolinyl)sulfonyl-N-[3-[[2-(4-methylphenyl)-5-methyloxazol-4-yl]methoxy]benzyl]amino]acetic acid

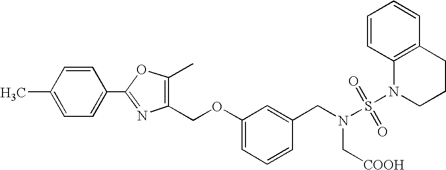

### Step 1: Preparation of [N-(1,2,3,4-tetrahydroquinolinyl)sulfonyl-N-[3-[[2-(4-methylphenyl)-5-methyloxazol-4-yl]methoxy]benzyl]amino]acetic acid ethyl ester

The procedure of Step 4 of Example 3 was repeated except for using 404 mg (1 mmol) of the compound obtained in Step 2 of Example 34, 266 mg (1.2 mmol) of [(4-chloromethyl-5-methyl-2-(4-methylphenyl)oxazol and 56 mg (1.4 mmol) of 60% sodium hydride to obtain the title compound (442 mg, 75%).

¹H-NMR (CDCl₃, 200 MHz): δ (ppm) 7.90 (d, J=8.0 Hz, 2H), 7.62 (d, J=8.3 Hz, 1H), 7.24-6.84 (m, 7H), 6.78-6.75 (m, 2H), 4.84 (s, 2H), 4.55 (s, 2H), 4.10 (q, J=7.2 Hz, 2H), 3.86 (s, 2H), 3.79 (t, J=5.8 Hz, 2H), 2.81 (t, J=6.8 Hz, 2H), 2.42 (s, 3H), 2.39 (s, 3H), 2.08-2.03 (m, 2H), 1.20 (t, J=7.2 Hz, 3H).

### Step 2: Preparation of [N-(1,2,3,4-tetrahydroquinolinyl)sulfonyl-N-[3-[[2-(4-methylphenyl)-5-methyloxazol-4-yl]methoxy]benzyl]amino]acetic acid

The procedure of Step 5 of Example 3 was repeated except for using 442 mg (0.75 mmol) of the compound obtained in Step 1 and 47 mg (1.14 mmol) of lithium hydroxide monohydrate to obtain the title compound (417 mg, 99%).

¹H-NMR (CDCl₃, 200 MHz): δ (ppm) 7.87 (d, J=8.0 Hz, 2H), 7.62 (d, J=8.3 Hz, 1H), 7.25-6.98 (m, 6H), 6.89-6.73 (m, 3H), 4.88 (br s, 1H), 4.44 (s, 2H), 3.89 (s, 2H), 3.76 (t, J=5.8 Hz, 2H), 2.79 (t, J=6.8 Hz, 2H), 2.42 (s, 3H), 2.38 (s, 3H), 2.08-2.03 (m, 2H).

### EXAMPLE 36

### Preparation of [N-(1,2,3,4-tetraquinolinyl)sulfonyl-N-[3-[[2-(4-trifluoromethylphenyl)-5-methyloxazol-4-yl]methoxy]benzyl]amino]acetic acid

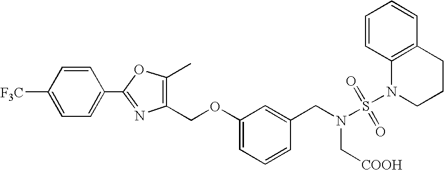

### Step 1: Preparation of [N-(1,2,3,4-tetraquinolinyl)sulfonyl-N-[3-[[2-(4-trifluoro methylphenyl)-5-methyloxazol-4-yl]methoxy]benzyl]amino]acetic acid ethyl ester

The procedure of Step 4 of Example 3 was repeated except for using 398 mg (1 mmol) of the compound obtained in Step 2 of Example 34, 330 mg (1.2 mmol) of [4-chloromethyl-5-methyl-2-(4-trifluoromethylphenyl)]oxazol and 56 mg (1.4 mmol) of 60% sodium hydride to obtain the title compound (476 mg, 74%).

¹H-NMR (CDCl₃, 200 MHz): δ (ppm) 8.13 (d, J=8.7 Hz, 2H), 7.70 (d, J=8.7 Hz, 2H), 7.63 (d, J=8.3 Hz, 1H), 7.27-6.77 (m, 7H), 4.88 (s, 2H), 4.56 (s, 2H), 4.10 (q, J=7.2 Hz, 2H), 3.86 (s, 2H), 3.80 (t, J=5.8 Hz, 2H), 2.83 (t, J=6.8 Hz, 2H), 2.46 (s, 3H), 2.09-2.04 (m, 2H), 1.20 (t, J=7.2 Hz, 3H).

### Step 2: Preparation of [N-(1,2,3,4-tetraquinolinyl)sulfonyl-N-[3-[[2-(4-trifluoro methylphenyl)-5-methyloxazol-4-yl]methoxy]benzyl]amino]acetic acid

The procedure of Step 5 of Example 3 was repeated except for using 476 mg (0.74 mmol) of the compound obtained in Step 1 and 47 mg (1.11 mmol) of lithium hydroxide monohydrate to obtain the title compound (451 mg, 99%).

¹H-NMR (CDCl₃, 300 MHz): δ (ppm) 8.11 (d, J=8.7 Hz, 2H), 7.68 (d, J=8.7 Hz, 2H), 7.61 (d, J=8.3 hz, 1H), 7.21-6.92 (m, 4H), 6.89-6.75 (m, 3H), 5.84 (br s, 1H), 4.88 (s, 2H), 4.49 (s, 2H), 3.90 (s, 2H), 3.65 (t, J=5.8 Hz, 2H), 2.80 (t, J=6.8 Hz, 2H), 2.45 (s, 3H), 2.09-2.03 (m, 2H).

### EXAMPLE 37

### Preparation of [N-(1,2,3,4-tetraquinolinyl)sulfonyl-N-[3-[[2-(thiophen-2-yl)-5-methyloxazol-4-yl]methoxy]benzyl]amino]acetic acid

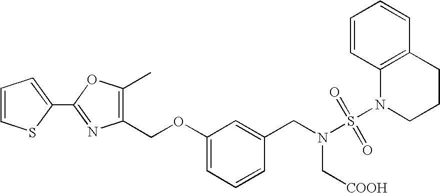

### Step 1: Preparation of [N-(1,2,3,4-tetraquinolinyl)sulfonyl-N-[3-[[2-(thiophen-2-yl)-5-methyloxazol-4-yl]methoxy]benzyl]amino]acetic acid ethyl ester

The procedure of Step 4 of Example 3 was repeated except for using 404 mg (1 mmol) of the compound obtained in Step 2 of Example 34, 256 mg (1.2 mmol) of [4-chloromethyl-5-methyl-2-(thiophen-2-yl)]oxazol and 56 mg (1.4 mmol) of 60% sodium hydride to obtain the title compound (407 mg, 70%).

¹H-NMR (CDCl₃, 200 MHz): δ (ppm) 7.65-7.61 (m, 2H), 7.40 (d, J=8.3 Hz, 1H), 7.27-7.01 (m, 5H), 6.93-6.76 (m, 3H), 4.83 (s, 2H), 4.54 (s, 2H), 4.10 (q, J=7.2 Hz, 2H), 3.86 (s, 2H), 3.79 (t, J=5.8 Hz, 2H), 2.82 (t, J=6.8 Hz, 2H), 2.41 (s, 3H), 2.09-2.03 (m, 2H), 1.20 (t, J=7.2 Hz, 3H).

### Step 2: Preparation of [N-(1,2,3,4-tetraquinolinyl)sulfonyl-N-[3-[[2-(thiophen-2-yl)-5-methyloxazol-4-yl]methoxy]benzyl]amino]acetic acid

The procedure of Step 5 of Example 3 was repeated except for using 407 mg (0.7 mmol) of the compound obtained in Step 1 and 44 mg (1.05 mmol) of lithium hydroxide monohydrate to obtain the title compound (384 mg, 99%).

¹H-NMR (CDCl₃, 300 MHz): δ (ppm) 7.65-7.60 (m, 2H), 7.39 (d, J=8.3 Hz, 1H), 7.19-6.98 (m, 5H), 6.89-6.74 (m, 3H), 6.72 (br s, 1H), 4.84 (s, 2H), 4.47 (s, 2H), 3.90 (s, 2H), 3.75 (t, J=5.8 Hz, 2H), 2.79 (t, J=6.8 Hz, 2H), 2.39 (s, 3H), 2.09-2.03 (m, 2H).

### EXAMPLE 38

### Preparation of [N—(N,N-dimethylamino)sulfonyl-N-[4-[(2-phenyl-5-methyloxazol-4-yl)methoxy]benzyl]amino]acetic acid

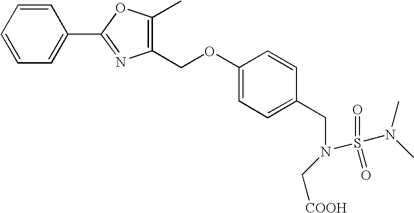

### Step 1: Preparation of [N-(4-benzyloxybenzyl)amino]acetic acid ethyl ester

The procedure of Step 1 of Example 3 was repeated except for using 106 g (0.5 mol) of 4-benzyloxybenzaldehyde, 77 g (0.55 mol) of glycine ethyl ester hydrochloride, 57 g (0.55 mol) of triethylamine and 159 g (0.75 mol) of sodium triacetoxyborohydride to obtain the title compound (127 g, 85%).

### Step 2: Preparation of [[N—(N,N-dimethylsulfamoyl)-N-(4-benzyloxybenzyl)]amino]acetic acid ethyl ester

The procedure of Step 2 of Example 3 was repeated except for using 14.97 g (0.05 mol) of the compound obtained in Step 1, 10.77 g (0.075 mol) of N,N-dimethylsulfamoyl chloride and 8.1 g (0.08 mol) of triethylamine to obtain the title compound (18.09 g, 89%).

### Step 3: Preparation of [[N—(N,N-dimethylamino)sulfonyl-N-(4-hydroxybenzyl)]amino]acetic acid ethyl ester

The procedure of Step 3 of Example 3 was repeated except for using 17.89 g (0.044 mol) of the compound obtained in Step 2 and 4 g of Pd/C catalyst to obtain the title compound (13.78 g, 99%).

### Step 4: Preparation of [N—(N,N-dimethylamino)sulfonyl-N-[4-[(2-phenyl-5-methyloxazol-4-yl)methoxy]benzyl]amino]acetic acid ethyl ester

The procedure of Step 4 of Example 3 was repeated except for using 316 mg (1 mmol) of the compound obtained in Step 3, 250 mg (1.2 mmol) of (4-chloromethyl-5-methyl-2-phenyl)oxazol and 56 mg (1.4 mmol) of 60% sodium hydride to obtain the title compound (373 mg, 78%).

¹H-NMR (CDCl₃, 300 MHz): δ (ppm) 8.03-8.00 (m, 2H), 7.47-7.43 (m, 3H), 7.25 (d, J=8.6 Hz, 2H), 7.00 (d, J=8.6 Hz, 2H), 4.98 (s, 2H), 4.49 (s, 2H), 4.16 (q, J=7.2 Hz, 2H), 3.82 (s, 2H), 2.86 (s, 6H), 2.43 (s, 3H), 1.25 (t, J=7.2 Hz, 3H).

### Step 5: Preparation of [N—(N,N-dimethylamino)sulfonyl-N-[4-[(2-phenyl-5-methyloxazol-4-yl)methoxy]benzyl]amino]acetic acid

The procedure of Step 5 of Example 3 was repeated except for using 373 mg (0.78 mmol) of the compound obtained in Step 4 and 50 mg (1.17 mmol) of lithium hydroxide monohydrate to obtain the title compound (355 mg, 99%).

¹H-NMR (CDCl₃, 300 MHz): δ (ppm) 8.02-7.99 (m, 2H), 7.46-7.44 (m, 3H), 7.22 (d, J=8.6 Hz, 2H), 6.96 (d, J=8.6 Hz, 2H), 5.00 (s, 2H), 4.46 (s, 2H), 3.82 (s, 2H), 2.84 (s. 6H), 2.45 (s, 3H).

### EXAMPLE 39

### Preparation of [N—(N,N-dimethylamino)sulfonyl-N-[4-[[2-(4-methylphenyl)-5-methyloxazol-4-yl]methoxy]benzyl]amino]acetic acid

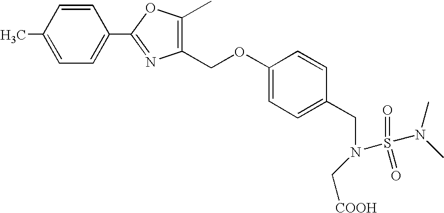

### Step 1: Preparation of [N—(N,N-dimethylamino)sulfonyl-N-[4-[[2-(4-methyl phenyl)-5-methyloxazol-4-yl]methoxy]benzyl]amino]acetic acid ethyl ester

The procedure of Step 4 of Example 3 was repeated except for using 316 mg (1 mmol) of the compound obtained in Step 3 of Example 38, 266 mg (1.2 mmol) of [(4-chloromethyl-5-methyl-2-(4-methylphenyl))oxazol and 56 mg (1.4 mmol) of 60% sodium hydride to obtain the title compound (386 mg, 77%).

¹H-NMR (CDCl₃, 200 MHz): δ (ppm) 7.89 (d, J=8.3 Hz, 2H), 7.28-7.22 (m, 4H), 7.01 (d, J=8.7 Hz, 2H), 4.97 (s, 2H), 4.49 (s, 2H), 4.15 (q, J=7.2 Hz, 2H), 3.82 (s, 2H), 2.86 (s, 6H), 2.42 (s, 3H), 2.39 (s, 3H), 1.25 (t, J=7.2 Hz, 3H).

### Step 2: Preparation of [N—(N,N-dimethylamino)sulfonyl-N-[4-[[2-(4-methyl phenyl)-5-methyloxazol-4-yl]methoxy]benzyl]amino]acetic acid

The procedure of Step 5 of Example 3 was repeated except for using 386 mg (0.77 mmol) of the compound obtained in Step 1 and 49 mg (1.16 mmol) of lithium hydroxide monohydrate to obtain the title compound (361 mg, 99%).

¹H-NMR (CDCl₃, 300 MHz): δ (ppm) 9.15 (br s, 1H), 7.88 (d, J=8.1 Hz, 2H), 7.24-7.18 (m, 4H), 6.94 (d, J=8.4 hz, 2H), 4.99 (s, 2H), 4.46 (s, 2H), 3.81 (s, 2H), 2.84 (s, 6H), 2.44 (s, 3H), 2.39 (s, 3H).

### EXAMPLE 40

### Preparation of [N—(N,N-dimethylamino)sulfonyl-N-[4-[[2-(4-trifluoromethylphenyl)-5-methyloxazol-4-yl]methoxy]benzyl]amino]acetic acid

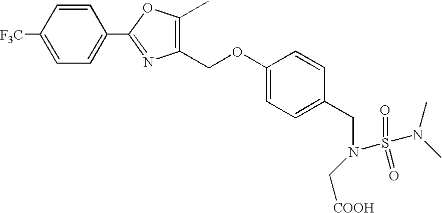

### Step 1: Preparation of [N—(N,N-dimethylamino)sulfonyl-N-[4-[[2-(4-trifluoro methylphenyl)-5-methyloxazol-4-yl]methoxy]benzyl]amino]acetic acid ethyl ester

The procedure of Step 4 of Example 3 was repeated except for using 316 mg (1 mmol) of the compound obtained in Step 3 of Example 38, 330 mg (1.2 mmol) of [4-chloromethyl-5-methyl-2-(4-trifluoromethylphenyl)]oxazol and 56 mg (1.4 mmol) of 60% sodium hydride to obtain the title compound (417 mg, 75%).

¹H-NMR (CDCl₃, 300 MHz): δ (ppm) 8.13 (d, J=8.1 Hz, 2H), 7.70 (d, J=8.1 Hz, 2H), 7.25 (d, J=8.7 Hz, 2H), 7.01 (d, J=8.7 Hz, 2H), 4.99 (s, 2H), 4.49 (s, 2H), 4.16 (q, J=7.2 Hz, 2H), 3.82 (s, 2H), 2.87 (s, 6H), 2.46 (s, 3H), 1.25 (t, J=7.2 Hz, 3H).

### Step 2: Preparation of [N—(N,N-dimethylamino)sulfonyl-N-[4-[[2-(4-trifluoro methylphenyl)-5-methyloxazol-4-yl]methoxy]benzyl]amino]acetic acid

The procedure of Step 5 of Example 3 was repeated except for using 417 mg (0.75 mmol) of the compound obtained in Step 1 and 53 mg (1.13 mmol) of lithium hydroxide monohydrate to obtain the title compound (361 mg, 99%).

¹H-NMR (CDCl₃, 300 MHz): δ (ppm) 9.39 (br s, 1H), 8.12 (d, J=8.4 Hz, 2H), 7.71 (d, J=8.4 Hz, 2H), 7.24 (d, J=8.4 Hz, 2H), 6.97 (d, J=8.4 Hz, 2H), 5.01 (s, 2H), 4.46 (s, 2H), 3.85 (s, 2H), 2.84 (s, 6H), 2.47 (s, 3H).

### EXAMPLE 41

### Preparation of [N—(N,N-dimethylamino)sulfonyl-N-[4-[[2-(thiophen-2-yl)-5-methyloxazol-4-yl]methoxy]benzyl]amino]acetic acid

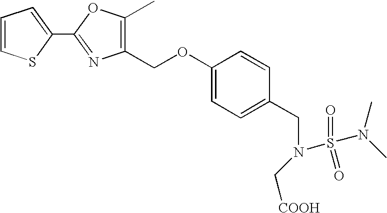

### Step 1: Preparation of [N—(N,N-dimethylamino)sulfonyl-N-[4-[[2-(thiophen-2-yl)-5-methyloxazol-4-yl]methoxy]benzyl]amino]acetic acid ethyl ester

The procedure of Step 4 of Example 3 was repeated except for using 316 mg (1 mmol) of the compound obtained in Step 3 of Example 38, 256 mg (1.2 mmol) of [4-chloromethyl-5-methyl-2-(thiophen-2-yl)]oxazol and 56 mg (1.4 mmol) of 60% sodium hydride to obtain the title compound (365 mg, 74%).

¹H-NMR (CDCl₃, 200 MHz): δ (ppm) 7.63 (dd, J=1.1 Hz, J=2.4 Hz, 1H), 7.39 (dd, J=1.1 Hz, J=3.9 Hz, 1H), 7.25 (d, J=8.5 Hz, 2H), 7.12-7.09 (m, 1H), 6.97 (d, J=8.5 Hz, 2H), 4.96 (s, 2H), 4.49 (s, 2H), 4.16 (q, J=7.2 Hz, 2H), 3.82 (s, 2H), 2.86 (s, 6H), 2.41 (s, 3H), 1.25 (t, J=7.2 Hz, 3H).

### Step 2: Preparation of [N—(N,N-dimethylamino)sulfonyl-N-[4-[[2-(thiophen-2-yl)-5-methyloxazol-4-yl]methoxy]benzyl]amino]acetic acid

The procedure of Step 5 of Example 3 was repeated except for using 365 mg (1.07 mmol) of the compound obtained in Step 1 and 47 mg (1.11 mmol) of lithium hydroxide monohydrate to obtain the title compound (341 mg, 99%).

¹H-NMR (CDCl₃, 300 MHz): δ (ppm) 9.76 (br s, 1H), 7.67 (dd, J=1.1 Hz, J=2.4 Hz, 1H), 7.42 (dd, J=1.1 Hz, J=3.9 Hz, 1H), 7.22 (d, J=8.7 Hz, 2H), 7.12-7.09 (m, 1H), 6.95 (d, J=8.7 Hz, 2H), 5.04 (s, 2H), 4.46 (s, 2), 3.83 (s, 2H), 2.83 (s, 6H), 2.42 (s, 3H).

### EXAMPLE 42

### Preparation of [N-(pyrrolidinyl)sulfonyl-N-[4-[[2-(4-methyl phenyl)-5-methyloxazol-4-yl]methoxy]benzyl]amino]acetic acid

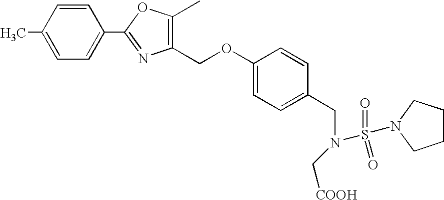

### Step 1: Preparation of [[N-(pyrrolidinyl)sulfonyl-N-(4-benzyloxybenzyl)]amino]acetate methyl ester

The procedure of Step 1 of Example 3 was repeated except for using 21.2 g (0.1 mol) of 4-benzyloxybenzaldehyde, 13.81 g (0.11 mol) of glycine methyl ester hydrochloride and 11.13 g (0.11 mol) of triethylamine to obtain [N-(4-benzyloxybenzyl)amino]acetate methyl ester (22.5 g, 79%). The procedure of Step 2 of Example 3 was repeated except for using 2.85 g (10 mmol) of the obtained compound, 2.04 g (12 mmol) of (pyrrolidinyl)sulfonyl chloride and 1.62 g (16 mmol) of triethylamine to obtain the title compound (3.6 g, 86%).

### Step 2: Preparation of [[N-(pyrrolidinyl)sulfonyl-N-(4-hydroxybenzyl)]amino]acetate methyl ester

The procedure of Step 3 of Example 3 was repeated except for using 3.6 g (8.6 mmol) of [[N-(pyrrolidinyl)sulfonyl-N-(4-benzyloxybenzyl)]amino]acetate methyl ester and 0.8 g of Pd/C catalyst to obtain the title compound (2.79 g, 99%).

### Step 3: Preparation of [N-(pyrrolidinyl)sulfonyl-N-[4-[[2-(4-methylphenyl)-5-methyloxazol-4-yl]methoxy]benzyl]amino]acetate methyl ester

The procedure of Step 4 of Example 3 was repeated except for using 328 mg (1 mmol) of the compound obtained in Step 2, 266 mg (1.2 mmol) of [4-chloromethyl-5-methyl-2-(4-methylphenyl)]oxazol and 56 mg (1.4 mmol) of 60% sodium hydride to obtain the title compound (390 mg, 76%).

¹H-NMR (CDCl₃, 200 MHz): δ (ppm) 7.89 (d, J=8.2 Hz, 2H), 7.28-7.21 (m, 4H), 6.98 (d, J=8.2 Hz, 2H), 4.96 (s, 2H), 4.48 (s, 2H), 3.85 (s, 2H), 3.69 (s, 3H), 3.38-3.32 (m, 4H), 2.41 (s, 3H), 2.38 (s, 3H), 1.98-1.86 (m, 4H).

### Step 4: Preparation of [N-(pyrrolidinyl)sulfonyl-N-[4-[[2-(4-methylphenyl)-5-methyloxazol-4-yl]methoxy]benzyl]amino]acetic acid

The procedure of Step 5 of Example 3 was repeated except for using 390 mg (0.76 mmol) of the compound obtained in Step 3 and 48 mg (1.14 mmol) of lithium hydroxide monohydrate to obtain the title compound (375 mg, 99%).

¹H-NMR (CDCl₃, 200 MHz): δ (ppm) 7.89 (d, J=8.1 Hz, 2H), 7.26-7.20 (m, 4H), 6.95 (d, J=8.1 Hz, 2H), 4.98 (s, 2H), 4.47 (s, 2H), 3.83 (s, 2H), 3.37-3.32 (m, 4H), 2.43 (s, 3H), 2.40 (s, 3H), 1.89-1.85 (m, 4H).

### EXAMPLE 43

### Preparation of [N-(4-methyl-1-piperazinyl)sulfonyl-N-[4-[[2-(4-methylphenyl)-5-methyloxazol-4-yl]methoxy]benzyl]amino]acetic acid

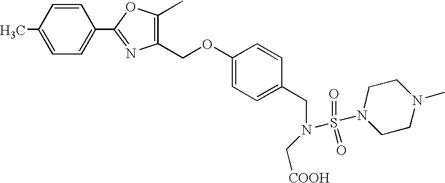

### Step 1: Preparation of [[N-(4-methyl-1-piperazinyl)sulfonyl-N-(4-benzyloxy benzyl)]amino]acetate methyl ester

The procedure of Step 2 of Example 3 was repeated except for using 2.85 g (10 mmol) of the compound obtained in Step 1 of Example 42, 2.38 g (12 mmol) of (4-methyl-1-piperazinyl)sulfonyl chloride and 1.62 g (16 mmol) of triethylamine to obtain the title compound (3.67 g, 82%).

### Step 2: Preparation of [[N-(4-methyl-1-piperazinyl)sulfonyl-N-(4-hydroxy benzyl)]amino]acetate methyl ester

The procedure of Step 3 of Example 3 was repeated except for using 3.67 g (8.2 mmol) of the compound obtained in Step 1 and 0.8 g of Pd/C catalyst to obtain the title compound (2.90 g, 99%).

### Step 3: Preparation of [N-(4-methyl-1-piperazinyl)sulfonyl-N-[4-[[2-(4-methyl phenyl)-5-methyloxazol-4-yl]methoxy]benzyl]amino]acetate methyl ester

The procedure of Step 4 of Example 3 was repeated except for using 357 mg (1 mmol) of the compound obtained in Step 2, 266 mg (1.2 mmol) of [(4-chloromethyl-5-methyl-2-(4-methylphenyl))oxazol and 56 mg (1.4 mmol) of 60% sodium hydride to obtain the title compound (407 mg, 75%).

¹H-NMR (CDCl₃, 300 MHz): δ (ppm) 7.90 (d, J=8.1 Hz, 2H), 7.25 (d, J=8.6 Hz, 2H), 7.24 (d, J=8.1 Hz, 2H), 6.99 (d, J=8.6 Hz, 2H), 4.97 (s, 2H), 4.48 (s, 2H), 3.82 (s, 2H), 3.70 (s, 3H), 3.35-3.32 (m, 4H), 2.48-2.44 (m, 4H), 2.43 (s, 3H), 2.40 (s, 3H), 2.31 (s, 3H).

### Step 4: Preparation of [N-(4-methyl-1-piperazinyl)sulfonyl-N-[4-[[2-(4-methyl phenyl)-5-methyloxazol-4-yl]methoxy]benzyl]amino]acetic acid

The procedure of Step 5 of Example 3 was repeated except for using 407 mg (0.75 mmol) of the compound obtained in Step 1 and 47 mg (1.13 mmol) of lithium hydroxide monohydrate to obtain the title compound (393 mg, 99%).

¹H-NMR (DMSO-d₆, 300 MHz): δ (ppm) 7.83 (d, J=8.1 Hz, 2H), 7.33 (d, J=8.1 Hz, 2H), 7.27 (d, J=8.5 Hz, 2H), 7.05 (d, J=8.5 Hz, 2H), 4.99 (s, 2H), 4.40 (s, 2H), 3.74 (s, 2H), 3.50 (br s, 1H), 3.40-3.00 (m, 4H), 2.62-2.58 (m, 4H), 2.44 (s, 3H), 2.37 (s, 3H).

### EXAMPLE 44

### Preparation of [N-(morpholinyl)sulfonyl-N-[4-[[2-(4-methyl phenyl)-5-methyloxazol-4-yl]methoxy]benzyl]amino]acetic acid

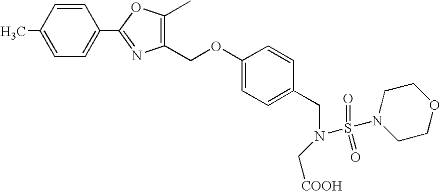

### Step 1: Preparation of [[N-(morpholinyl)sulfonyl-N-(4-benzyloxybenzyl)]amino]acetate methyl ester

The procedure of Step 2 of Example 3 was repeated except for using 2.85 g (10 mmol) of the compound obtained in Step 1 of Example 42, 2.23 g (12 mmol) of (morpholinyl)sulfonyl chloride and 1.62 g (16 mmol) of triethylamine to obtain the title compound (3.69 g, 85%).

### Step 2: Preparation of [[N-(morpholinyl)sulfonyl-N-(4-hydroxybenzyl)]amino]acetate methyl ester

The procedure of Step 3 of Example 3 was repeated except for using 3.69 g (8.5 mmol) of the compound obtained in Step 1 and 0.8 g of Pd/C catalyst to obtain the title compound (2.90 g, 99%).

### Step 3: Preparation of [N-(morpholinyl)sulfonyl-N-[4-[[2-(4-methylphenyl)-5-methyloxazol-4-yl]methoxy]benzyl]amino]acetate methyl ester

The procedure of Step 4 of Example 3 was repeated except for using 344 mg (1 mmol) of the compound obtained in Step 2, 266 mg (1.2 mmol) of [(4-chloromethyl-5-methyl-2-(4-methylphenyl))oxazol and 56 mg (1.4 mmol) of 60% sodium hydride to obtain the title compound (392 mg, 74%).

¹H-NMR (CDCl₃, 200 MHz): δ (ppm) 7.90 (d, J=8.1 Hz, 2H), 7.26-7.22 (m, 4H), 6.99 (d, J=8.1 Hz, 2H), 4.98 (s, 2H), 4.50 (s, 2H), 3.85-3.70 (m, 4H), 3.71 (s, 2H), 3.31-3.26 (m, 4H), 2.43 (s, 3H), 2.40 (s, 3H).

### Step 4: Preparation of [N-(morpholinyl)sulfonyl-N-[4-[[2-(4-methylphenyl)-5-methyloxazol-4-yl]methoxy]benzyl]amino]acetic acid

The procedure of Step 5 of Example 3 was repeated except for using 392 mg (0.74 mmol) of the compound obtained in Step 3 and 47 mg (1.11 mmol) of lithium hydroxide monohydrate to obtain the title compound (378 mg, 99%).

¹H-NMR (CDCl₃, 300 MHz): δ (ppm) 7.89 (d, J=8.1 Hz, 2H), 7.26 (d, J=8.1 Hz, 2H), 7.17 (d, J=8.4 Hz, 2H), 6.93 (d, J=8.4 Hz, 2H), 6.98 (s, 2H), 4.48 (s, 2H), 3.79 (s, 2H), 3.70-3.67 (m, 4H), 3.29-3.25 (m, 4H), 2.44 (s, 3H), 2.40 (s, 3H).

### EXAMPLE 45

### Preparation of [N—[(N-methyl-N-phenyl)amino]sulfonyl-N-[4-[(2-phenyl-5-methyloxazol-4-yl)methoxy]benzyl]amino]acetic acid

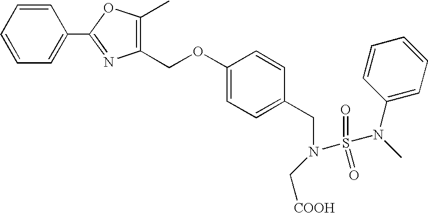

### Step 1: Preparation of [N—[(N-methyl-N-phenyl)amino]sulfonyl-N-(4-benzyloxy benzyl)]amino]acetic acid ethyl ester

The procedure of Step 2 of Example 3 was repeated except for using 14.97 g (0.05 mol) of the compound obtained in Step 1 of Example 38, 15.42 g (0.075 mol) of [(N-methyl-N-phenyl)amino]sulfonyl chloride and 8.1 g (0.08 mol) of triethylamine to obtain the title compound (19.21 g, 79%).

### Step 2: Preparation of [N—[(N-methyl-N-phenyl)amino]sulfonyl-N-(4-hydroxy benzyl)]amino]acetic acid ethyl ester

The procedure of Step 3 of Example 3 was repeated except for using 18.27 g (0.039 mol) of the compound obtained in Step 1 and 4 g of Pd/C catalyst to obtain the title compound (14.61 g, 99%).

### Step 3: Preparation of [N—[(N-methyl-N-phenyl)amino]sulfonyl-N-[4-[(2-phenyl-5-methyloxazol-4-yl)methoxy]benzyl]amino]acetic acid ethyl ester

The procedure of Step 4 of Example 3 was repeated except for using 378 mg (1 mmol) of the compound obtained in Step 2, 250 mg (1.2 mmol) of (4-chloromethyl-5-methyl-2-phenyl)oxazol and 56 mg (1.4 mmol) of 60% sodium hydride to obtain the title compound (423 mg, 77%).

¹H-NMR (CDCl₃, 300 MHz): δ (ppm) 8.01 (m, 2H), 7.47-7.29 (m, 7H), 7.28-7.23 (m, 1H), 7.16 (d, J=8.7 Hz, 2H), 6.95 (d, J=8.7 Hz, 2H), 4.97 (s, 2H), 4.44 (s, 2H), 4.15 (q, J=7.2 Hz, 2H), 3.81 (s, 2H), 3.29 (s, 3H), 2.43 (s, 3H), 1.24 (t, J=7.2 Hz, 3H).

### Step 4: Preparation of [N—[(N-methyl-N-phenyl)amino]sulfonyl-N-[4-[(2-phenyl-5-methyloxazol-4-yl)methoxy]benzyl]amino]acetic acid

The procedure of Step 5 of Example 3 was repeated except for using 423 mg (0.77 mmol) of the compound obtained in Step 3 and 49 mg (1.16 mmol) of lithium hydroxide monohydrate to obtain the title compound (398 mg, 99%).

¹H-NMR (CDCl₃, 300 MHz): δ (ppm) 8.01-7.97 (m, 2H), 7.44 (m, 5H), 7.36-7.15 (m, 3H), 7.10 (d, J=8.6 hz, 2H), 6.90 (d, J=8.6 Hz, 2H), 4.98 (s, 2H), 4.42 (s, 2H), 3.91 (s, 2H), 3.27 (s, 3H), 2.44 (s, 3H).

### EXAMPLE 46

### Preparation of [N—[(N-methyl-N-phenyl)amino]sulfonyl-N-[4-[[2-(4-methylphenyl)-5-methyloxazol-4-yl]methoxy]benzyl]amino]acetic acid

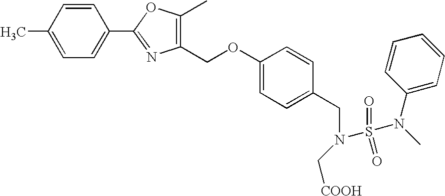

### Step 1: Preparation of [N—[(N-methyl-N-phenyl)amino]sulfonyl-N-[4-[[2-(4-methylphenyl)-5-methyloxazol-4-yl]methoxy]benzyl]amino]acetic acid

The procedure of Step 4 of Example 3 was repeated except for using 378 mg (1 mmol) of the compound obtained in Step 2 of Example 45, 266 mg (1.2 mmol) of [(4-chloromethyl-5-methyl-2-(4-methylphenyl))oxazol and 56 mg (1.4 mmol) of 60% sodium hydride to obtain the title compound (406 mg, 72%).

¹H-NMR (CDCl₃, 300 MHz): δ (ppm) 7.89 (d, J=8.1 Hz, 2H), 7.47-7.23 (m, 9H), 7.16 (d, J=8.7 Hz, 2H), 6.95 (d, J=8.7 Hz, 2H), 4.95 (s, 2H), 4.44 (s, 2H), 4.14 (q, J=7.2 Hz, 2H), 3.81 (s, 2H), 3.28 (s, 3H), 2.41 (s, 3H), 2.39 (s, 3H), 1.23 (t, J=7.2 Hz, 3H).

### Step 2: Preparation of [N—[(N-methyl-N-phenyl)amino]sulfonyl-N-[4-[[2-(4-methylphenyl)-5-methyloxazol-4-yl]methoxy]benzyl]amino]acetic acid

The procedure of Step 5 of Example 3 was repeated except for using 406 mg (0.72 mmol) of the compound obtained in Step 1 and 45 mg (1.08 mmol) of lithium hydroxide monohydrate to obtain the title compound (382 mg, 99%).

¹H-NMR (CDCl₃, 200 MHz): δ (ppm) 7.84 (d, J=8.1 Hz, 2H), 7.47-7.22 (m, 9H), 7.18 (d, J=8.7 Hz, 2H), 6.89 (d, J=8.7 Hz, 2H), 4.85 (s, 2H), 4.39 (s, 2H), 3.76 (s, 2H), 3.14 (s, 3H), 2.36 (s, 3H), 2.34 (s, 3H).

### EXAMPLE 47

### Preparation of [N—[(N-methyl-N-phenyl)amino]sulfonyl-N-[4-[[2-(4-trifluoromethylphenyl)-5-methyloxazol-4-yl]methoxy]benzyl]amino]acetic acid

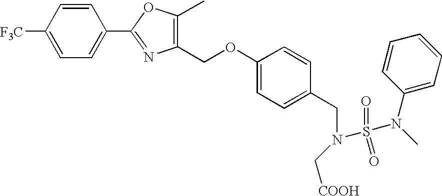

### Step 1: Preparation of [N—[(N-methyl-N-phenyl)amino]sulfonyl-N-[4-[[2-(4-trifluoromethylphenyl)-5-methyloxazol-4-yl]methoxy]benzyl]amino]acetic acid ethyl ester

The procedure of Step 4 of Example 3 was repeated except for using 364 mg (1 mmol) of the compound obtained in Step 2 of Example 45, 330 mg (1.2 mmol) of [4-chloromethyl-5-methyl-2-(4-trifluoromethylphenyl)]oxazol and 56 mg (1.4 mmol) of 60% sodium hydride to obtain the title compound (463 mg, 75%).

¹H-NMR (CDCl₃, 300 MHz): δ (ppm) 8.12 (d, J=8.3 Hz, 2H), 7.86 (d, J=8.3 hz, 2H), 7.71-7.23 (m, 5H), 7.17 (d, J=8.5 Hz, 2H), 6.95 (d, J=8.5 Hz, 2H), 4.98 (s, 2H), 4.44 (s, 2H), 4.15 (q, J=7.2 Hz, 2H), 3.81 (s, 2H), 3.30 (s, 3H), 2.45 (s, 3H), 1.24 (t, J=7.2 Hz, 3H).

### Step 2: Preparation of [N—[(N-methyl-N-phenyl)amino]sulfonyl-N-[4-[[2-(4-trifluoromethylphenyl)-5-methyloxazol-4-yl]methoxy]benzyl]amino]acetic acid

The procedure of Step 5 of Example 3 was repeated except for using 463 mg (0.75 mmol) of the compound obtained in Step 1 and 47 mg (1.13 mmol) of lithium hydroxide monohydrate to obtain the title compound (438 mg, 99%).

¹H-NMR (CDCl₃, 200 MHz): δ (ppm) 8.07 (d, J=8.3 Hz, 2H), 7.65 (d, J=8.3 hz, 2H), 7.37-7.18 (m, 5H), 7.05 (d, J=8.5 Hz, 2H), 6.82 (d, J=8.5 Hz, 2H), 4.89 (s, 2H), 4.41 (s, 2H), 3.78 (s, 2H), 3.18 (s, 3H), 2.39 (s, 3H).

### EXAMPLE 48

### Preparation of [N—[(N-methyl-N-phenyl)amino]sulfonyl-N-[4-[[2-(thiophen-2-yl)-5-methyloxazol-4-yl]methoxy]benzyl]amino]acetic acid

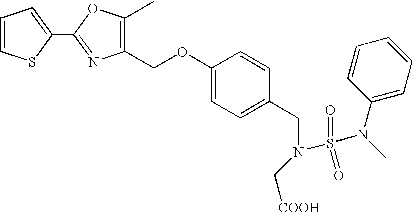

### Step 1: Preparation of [N—[(N-methyl-N-phenyl)amino]sulfonyl-N-[4-[[2-(thiophen-2-yl)-5-methyloxazol-4-yl]methoxy]benzyl]amino]acetic acid ethyl ester

The procedure of Step 4 of Example 3 was repeated except for using 378 mg (1 mmol) of the compound obtained in Step 2 of Example 45, 256 mg (1.2 mmol) of [4-chloromethyl-5-methyl-2-(thiophen-2-yl)]oxazol and 56 mg (1.4 mmol) of 60% sodium hydride to obtain the title compound (422 mg, 76%).

¹H-NMR (CDCl₃, 300 MHz): δ (ppm) 7.63-7.62 (m, 1H), 7.47-7.24 (m, 2H), 7.16 (d, J=8.5 Hz, 2H), 7.10-7.07 (m, 1H), 6.93 (J=8.5 Hz, 2H), 4.94 (s, 2H), 4.43 (s, 2H), 4.15 (q, J=7.2 Hz, 2H), 3.81 (s, 2H), 3.29 (s, 3H), 2.40 (s, 3H), 1.24 (t, J=7.2 Hz, 3H).

### Step 2: Preparation of [N—[(N-methyl-N-phenyl)amino]sulfonyl-N-[4-[[2-(thiophen-2-yl)-5-methyloxazol-4-yl]methoxy]benzyl]amino]acetic acid

The procedure of Step 5 of Example 3 was repeated except for using 422 mg (0.76 mmol) of the compound obtained in Step 1 and 48 mg (1.14 mmol) of lithium hydroxide monohydrate to obtain the title compound (438 mg, 99%).

¹H-NMR (CDCl₃, 200 MHz): δ (ppm) 7.63-7.62 (m, 1H), 7.47-7.24 (m, 2H), 7.16 (d, J=8.5 Hz, 2H), 7.10-7.07 (m, 1H), 6.85 (J=8.5 Hz, 2H), 4.87 (s, 2H), 4.40 (s, 2H), 3.78 (s, 2H), 3.19 (s, 3H), 2.36 (s, 3H).

### EXAMPLE 49

### Preparation of [N—[[N-methyl-N-(4-chlorophenyl)]amino]sulfonyl-N-[4-[(2-phenyl-5-methyloxazol-4-yl)methoxy]benzyl]amino]acetic acid

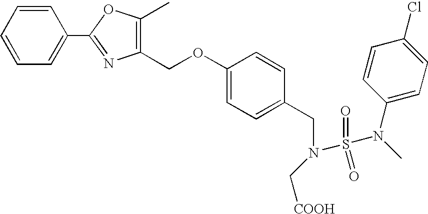

### Step 1: Preparation of [N—[[N-methyl-N-(4-chlorophenyl)]amino]sulfonyl-N-(4-benzyloxybenzyl)]amino]acetic acid ethyl ester

The procedure of Step 2 of Example 3 was repeated except for using 14.97 g (0.05 mol) of the compound obtained in Step 1 of Example 38, 18.01 g (0.075 mol) of [[N-methyl-N-(p-chlorophenyl)]amino]sulfonyl chloride and 8.1 g (0.08 mol) of triethylamine to obtain the title compound (18.86 g, 75%).

### Step 2: Preparation of [N—[[N-methyl-N-(4-chlorophenyl)]amino]sulfonyl-N-(4-hydroxybenzyl)]amino]acetic acid ethyl ester

The procedure of Step 3 of Example 3 was repeated except for using 18.61 g (0.37 mol) of the compound obtained in Step 1 and 4 g of Pd/C catalyst to obtain the title compound (15.12 g, 99%).

### Step 3: Preparation of [N—[[N-methyl-N-(4-chlorophenyl)]amino]sulfonyl-N-[4-[(2-phenyl-5-methyloxazol-4-yl)methoxy]benzyl]amino]acetic acid ethyl ester

The procedure of Step 4 of Example 3 was repeated except for using 413 mg (1 mmol) of the compound obtained in Step 2, 250 mg (1.2 mmol) of (4-chloromethyl-5-methyl-2-phenyl)oxazol and 56 mg (1.4 mmol) of 60% sodium hydride to obtain the title compound (421 mg, 72%).

¹H-NMR (CDCl₃, 300 MHz): δ (ppm) 8.03-8.00 (m, 2H), 7.47-7.30 (m, 7H), 7.17 (d, J=8.4 Hz, 2H), 6.96 (d, J=8.4 Hz, 2H), 4.97 (s, 2H), 4.42 (s, 2H), 4.15 (q, J=7.2 Hz, 2H), 3.81 (s, 2H), 3.27 (s, 3H), 1.24 (t, J=7.2 Hz, 3H).

### Step 4: Preparation of [N—[[N-methyl-N-(4-chlorophenyl)]amino]sulfonyl-N-[4-[(2-phenyl-5-methyloxazol-4-yl)methoxy]benzyl]amino]acetic acid

The procedure of Step 5 of Example 3 was repeated except for using 421 mg (0.72 mmol) of the compound obtained in Step 3 and 45 mg (1.08 mmol) of lithium hydroxide monohydrate to obtain the title compound (397 mg, 99%).

¹H-NMR (CDCl₃, 200 MHz): δ (ppm) 8.03-7.97 (m, 2H), 7.47-7.19 (m, 7H), 7.06 (d, J=8.4 Hz, 2H), 6.89 (d, J=8.4 Hz, 2H), 4.92 (s, 2H), 4.38 (s, 2H), 3.78 (s, 2H), 3.16 (s, 3H).

### EXAMPLE 50

### Preparation of [N—[[N-methyl-N-(4-chlorophenyl)]amino]sulfonyl-N-[4-[[2-(4-methylphenyl)-5-methyloxazol-4-yl]methoxy]benzyl]amino]acetic acid

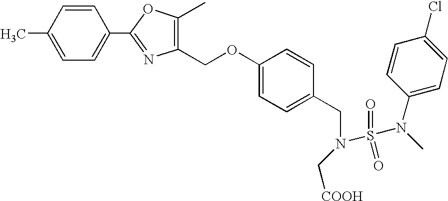

### Step 1: Preparation of [N—[[N-methyl-N-(4-chlorophenyl)]amino]sulfonyl-N-[4-[[2-(4-methylphenyl)-5-methyloxazol-4-yl]methoxy]benzyl]amino]acetic acid ethyl ester

The procedure of Step 4 of Example 3 was repeated except for using 413 mg (1 mmol) of the compound obtained in Step 2 of Example 49, 266 mg (1.2 mmol) of [(4-chloromethyl-5-methyl-2-(4-methylphenyl))oxazol and 56 mg (1.4 mmol) of 60% sodium hydride to obtain the title compound (416 mg, 77%).

¹H-NMR (CDCl₃, 300 MHz): δ (ppm) 7.90 (d, J=8.4 Hz, 2H), 7.42-7.23 (m, 6H), 7.16 (d, J=8.5 Hz, 2H), 6.95 (d, J=8.5 Hz, 2H), 4.96 (s, 2H), 4.42 (s, 2H), 4.15 (q, J=7.2 Hz, 2H), 3.81 (s, 2H), 3.27 (s, 3H), 2.42 (s, 3H), 2.39 (s, 3H), 1.24 (t, J=7.2 Hz, 3H).

### Step 2: Preparation of [N—[[N-methyl-N-(4-chlorophenyl)]amino]sulfonyl-N-[4-[[2-(4-methylphenyl)-5-methyloxazol-4-yl]methoxy]benzyl]amino]acetic acid

The procedure of Step 5 of Example 3 was repeated except for using 461 mg (0.77 mmol) of the compound obtained in Step 1 and 48 mg (1.16 mmol) of lithium hydroxide monohydrate to obtain the title compound (435 mg, 99%).

¹H-NMR (CDCl₃, 300 MHz): δ (ppm) 7.88 (d, J=8.4 Hz, 2H), 7.39-7.23 (m, 6H), 7.08 (d, J=8.5 Hz, 2H), 6.89 (d, J=8.5 Hz, 2H), 4.97 (s, 2H), 4.39 (s, 2H), 3.78 (s, 2H), 3.23 (s, 3H), 2.44 (s, 3H), 2.39 (s, 3H).

### EXAMPLE 51

### Preparation of [N—[[N-methyl-N-(4-chlorophenyl)]amino]sulfonyl-N-[4-[[2-(4-trifluoromethylphenyl)-5-methyloxazol-4-yl]methoxy]benzyl]amino]acetic acid

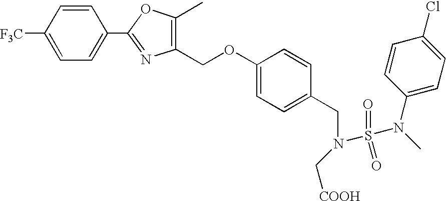

### Step 1: Preparation of [N—[[N-methyl-N-(4-chlorophenyl)]amino]sulfonyl-N-[4-[[2-(4-trifluoromethylphenyl)-5-methyloxazol-4-yl]methoxy]benzyl]amino]acetic acid ethyl ester

The procedure of Step 4 of Example 3 was repeated except for using 413 mg (1 mmol) of the compound obtained in Step 2 of Example 49, 330 mg (1.2 mmol) of [4-chloromethyl-5-methyl-2-(4-trifluoromethylphenyl)]oxazol and 56 mg (1.4 mmol) of 60% sodium hydride to obtain the title compound (498 mg, 75%).

¹H-NMR (CDCl₃, 300 MHz): δ (ppm) 8.12 (d, J=8.1 Hz, 2H), 7.07 (d, J=8.1 Hz, 2H), 7.42-7.31 (m, 4H), 7.18 (d, J=8.5 Hz, 2H), 6.96 (d, J=8.5 Hz, 2H), 4.98 (s, 2H), 4.43 (s, 2H), 4.15 (q, J=7.2 Hz, 2H), 3.82 (s, 2H), 3.27 (s, 3H), 2.46 (s, 3H), 1.25 (t, J=7.2 Hz, 3H).

### Step 2: Preparation of [N—[[N-methyl-N-(4-chlorophenyl)]amino]sulfonyl-N-[4-[[2-(4-trifluoromethylphenyl)-5-methyloxazol-4-yl]methoxy]benzyl]amino]acetic acid

The procedure of Step 5 of Example 3 was repeated except for using 489 mg (0.75 mmol) of the compound obtained in Step 1 and 47 mg (1.13 mmol) of lithium hydroxide monohydrate to obtain the title compound (463 mg, 99%).

¹H-NMR (CDCl₃, 300 MHz): δ (ppm) 8.06 (d, J=8.1 Hz, 2H), 7.65 (d, J=8.1 Hz, 2H), 7.25-7.21 (m, 4H), 7.03 (d, J=8.5 Hz, 2H), 6.85 (d, J=8.5 Hz, 2H), 4.89 (s, 2H), 4.36 (s, 2H), 3.78 (s, 2H), 3.11 (s, 3H), 2.40 (s, 3H).

### EXAMPLE 52

### Preparation of [N—[[N-methyl-N-(4-chlorophenyl)]amino]sulfonyl-N-[4-[[2-(thiophen-2-yl)-5-methyloxazol-4-yl]methoxy]benzyl]amino]acetic acid

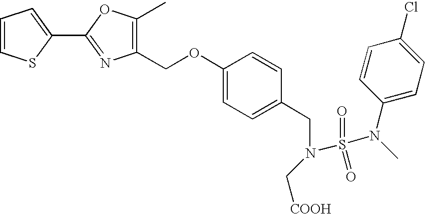

### Step 1: Preparation of [N—[[N-methyl-N-(4-chlorophenyl)]amino]sulfonyl-N-[4-[[2-(thiophen-2-yl)-5-methyloxazol-4-yl]methoxy]benzyl]amino]acetic acid ethyl ester

The procedure of Step 4 of Example 3 was repeated except for using 413 mg (1 mmol) of the compound obtained in Step 2 of Example 49, 256 mg (1.2 mmol) of [4-chloromethyl-5-methyl-2-(thiophen-2-yl)]oxazol and 56 mg (1.4 mmol) of 60% sodium hydride to obtain the title compound (425 mg, 72%).

¹H-NMR (CDCl₃, 300 MHz): δ (ppm) 7.62 (m, 1H), 7.42-7.31 (m, 5H), 7.15 (d, J=8.5 Hz, 2H), 7.11-7.08 (m, 1H), 6.94 (d, J=8.5 Hz, 2H), 4.95 (s, 2H), 4.42 (s, 2H), 4.15 (q, J=7.2 Hz, 2H), 3.82 (s, 2H), 3.27 (s, 3H), 2.41 (s, 3H), 1.25 (t, J=7.2 Hz, 3H).

### Step 2: Preparation of [N—[[N-methyl-N-(4-chlorophenyl)]amino]sulfonyl-N-[4-[[2-(thiophen-2-yl)-5-methyloxazol-4-yl]methoxy]benzyl]amino]acetic acid

The procedure of Step 5 of Example 3 was repeated except for using 425 mg (0.72 mmol) of the compound obtained in Step 1 and 45 mg (1.08 mmol) of lithium hydroxide monohydrate to obtain the title compound (401 mg, 99%).

¹H-NMR (CDCl₃, 300 MHz): δ (ppm) 7.61-7.59 (m, 1H), 7.38-7.36 (m, 1H), 7.27-7.18 (m, 4H), 7.07-7.00 (m, 3H), 6.82 (d, J=8.5 Hz, 2H), 5.29 (br s, 1H), 4.86 (s, 2H), 4.35 (s, 2H), 3.77 (s, 2H), 3.08 (s, 3H), 2.35 (s, 3H).

### EXAMPLE 53

### Preparation of [N-(indolinyl)sulfonyl-N-[4-[(2-phenyl-5-methyloxazol-4-yl)methoxy]benzyl]amino]acetic acid

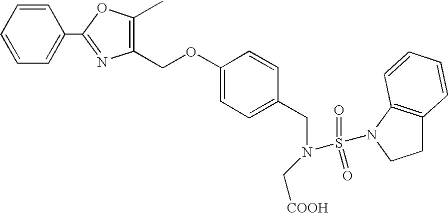

### Step 1: Preparation of [N-(indolinyl)sulfonyl-N-(4-benzyloxybenzyl)amino]acetic acid ethyl ester

The procedure of Step 2 of Example 4 was repeated except for using 14.97 g (0.05 mol) of the compound obtained in Step 1 of Example 38, 16.33 g (0.075 mol) of (indolinyl)sulfonyl chloride and 8.1 g (0.08 mol) of triethylamine to obtain the title compound (18.5 g, 77%).

### Step 2: Preparation of [N-(indolinyl)sulfonyl-N-(4-hydroxybenzyl)amino]acetic acid ethyl ester

The procedure of Step 3 of Example 3 was repeated except for using 18.26 g (0.038 mol) of the compound obtained in Step 1 and 4 g of Pd/C catalyst to obtain the title compound (14.69 g, 99%).

### Step 3: Preparation of [N-(indolinyl)sulfonyl-N-[4-[(2-phenyl-5-methyloxazol-4-yl)methoxy]benzyl]amino]acetic acid ethyl ester

The procedure of Step 4 of Example 3 was repeated except for using 390 mg (1 mmol) of the compound obtained in Step 2, 250 mg (1.2 mmol) of (4-chloromethyl-5-methyl-2-phenyl)oxazol and 56 mg (1.4 mmol) of 60% sodium hydride to obtain the title compound (395 mg, 72%).

¹H-NMR (CDCl₃, 300 MHz): δ (ppm) 8.03-7.98 (m, 2H), 7.47-7.42 (m, 4H), 7.17-7.11 (m, 4H), 6.96-6.92 (m, 3H), 4.97 (s, 2H), 4.57 (s, 2H), 4.06 (t, J=8.6 Hz, 2H), 3.95 (q, J=7.2 Hz, 2H), 3.85 (s, 2H), 3.12 (t, J=8.6 Hz, 2H), 2.43 (s, 3H), 1.12 (t, J=7.2 Hz, 3H).

### Step 4: Preparation of [N-(indolinyl)sulfonyl-N-[4-[(2-phenyl-5-methyloxazol-4-yl)methoxy]benzyl]amino]acetic acid

The procedure of Step 5 of Example 3 was repeated except for using 395 mg (0.72 mmol) of the compound obtained in Step 3 and 45 mg (1.08 mmol) of lithium hydroxide monohydrate to obtain the title compound (380 mg, 99%).

¹H-NMR (CDCl₃, 300 MHz): δ (ppm) 8.00-7.97 (m, 2H), 7.46-7.42 (m, 4H), 7.14-7.08 (m, 4H), 6.96-6.89 (m, 3H), 4.96 (s, 2H), 4.48 (s, 2H), 4.02 (t, J=8.4 Hz, 2H), 3.88 (s, 2H), 3.08 (t, J=8.4 Hz, 2H), 2.44 (s, 3H).

### EXAMPLE 54

### Preparation of [N-(indolinyl)sulfonyl-N-[4-[[2-(4-methyl phenyl)-5-methyloxazol-4-yl]methoxy]benzyl]amino]acetic acid

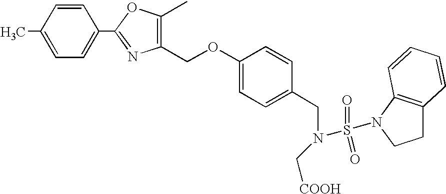

### Step 1: Preparation of [N-(indolinyl)sulfonyl-N-[4-[[2-(4-methylphenyl)-5-methyloxazol-4-yl]methoxy]benzyl]amino]acetic acid ethyl ester

The procedure of Step 4 of Example 3 was repeated except for using 390 mg (1 mmol) of the compound obtained in Step 2 of Example 53, 266 mg (1.2 mmol) of [(4-chloromethyl-5-methyl-2-(4-methylphenyl))oxazol and 56 mg (1.4 mmol) of 60% sodium hydride to obtain the title compound (432 mg, 75%).

¹H-NMR (CDCl₃, 300 MHz): δ (ppm) 7.87 (d, J=8.7 Hz, 2H), 7.43 (d, J=8.4 Hz, 1H), 7.24 (d, J=8.7 Hz, 2H), 7.17-7.04 (m, 4H), 6.97-6.91 (m, 3H), 4.99 (s, 2H), 4.57 (s, 2H), 4.06 (t, J=8.4 Hz, 2H), 3.95 (q, J=7.2 Hz, 2H), 3.86 (s, 2H), 3.12 (t, J=8.4 Hz, 2H), 2.41 (s, 3H), 2.39 (s, 3H), 1.12 (t, J=7.2 Hz, 3H).

### Step 2: Preparation of [N-(indolinyl)sulfonyl-N-[4-[[2-(4-methylphenyl)-5-methyloxazol-4-yl]methoxy]benzyl]amino]acetic acid

The procedure of Step 5 of Example 3 was repeated except for using 432 mg (0.75 mmol) of the compound obtained in Step 1 and 47 mg (1.13 mmol) of lithium hydroxide monohydrate to obtain the title compound (407 mg, 99%).

¹H-NMR (CDCl₃, 300 MHz): δ (ppm) 7.86 (d, J=8.1 Hz, 2H), 7.43 (d, J=8.1 Hz 1H), 7.25-7.23 (m, 2H), 7.16-7.06 (m, 4H), 6.95-6.07 (m, 3H), 4.94 (s, 2H), 4.46 (s, 2H), 4.01 (t, J=8.4 Hz, H), 3.85 (s, 2H), 3.06 (t, J=8.4 Hz, 2H), 2.43 (s, 3H), 2.40 (s, 3H).

### EXAMPLE 55

### Preparation of [N-(indolinyl)sulfonyl-N-[4-[[2-(4-trifluoromethylphenyl)-5-methyloxazol-4-yl]methoxy]benzyl]amino]acetic acid

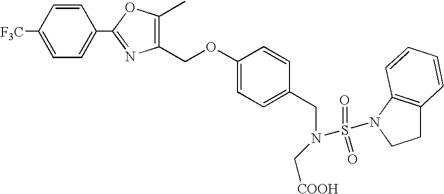

### Step 1: Preparation of [N-(indolinyl)sulfonyl-N-[4-[[2-(4-trifluoromethyl phenyl)-5-methyloxazol-4-yl]methoxy]benzyl]amino]acetic acid ethyl ester

The procedure of Step 4 of Example 3 was repeated except for using 390 mg (1 mmol) of the compound obtained in Step 2 of Example 53, 330 mg (1.2 mmol) of [(4-chloromethyl-5-methyl-2-(4-trifluoromethylphenyl))oxazol and 56 mg (1.4 mmol) of 60% sodium hydride to obtain the title compound (447 mg, 71%).

¹H-NMR (CDCl₃, 300 MHz): δ (ppm) 8.13 (d, J=8.3 Hz, 2H), 7.85 (d, J=8.3 Hz, 2H), 7.70 (d, J=8.4 Hz, 1H), 7.19-7.11 (m, 4H), 6.97-6.93 (m, 3H), 5.01 (s, 2H), 4.59 (s, 2H), 4.07 (t, J=8.4 Hz, 2H), 3.95 (q, J=7.2 Hz, 2H), 3.86 (s, 2H), 3.13 (t, J=8.4 Hz, 2H), 2.46 (s, 3H), 1.12 (t, J=7.2 Hz, 3H).

### Step 2: Preparation of [N-(indolinyl)sulfonyl-N-[4-[[2-(4-trifluoromethyl phenyl)-5-methyloxazol-4-yl]methoxy]benzyl]amino]acetic acid

The procedure of Step 5 of Example 3 was repeated except for using 447 mg (0.71 mmol) of the compound obtained in Step 1 and 45 mg (1.07 mmol) of lithium hydroxide monohydrate to obtain the title compound (423 mg, 99%).

¹H-NMR (CDCl₃, 300 MHz): δ (ppm) 8.10 (d, J=8.4 Hz, 2H), 7.70 (d, J=8.4 hz, 2H), 7.43 (d, J=8.4 Hz, 1H), 7.17-7.12 (m, 4H), 6.97-6.91 (m, 3H), 4.98 (s, 2H), 4.51 (s, 2H), 4.03 (t, J=8.4 Hz, 2H), 3.90 (s, 2H), 3.10 (t, J=8.4 Hz, 2H), 2.46 (s, 3H).

### EXAMPLE 56

### Preparation of [N-(indolinyl)sulfonyl-N-[4-[[2-(thiophen-2-yl)-5-methyloxazol-4-yl]methoxy]benzyl]amino]acetic acid

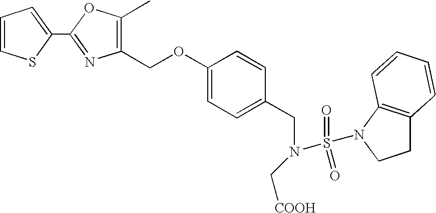

### Step 1: Preparation of [N-(indolinyl)sulfonyl-N-[4-[[2-(thiophen-2-yl)-5-methyloxazol-4-yl]methoxy]benzyl]amino]acetic acid ethyl ester

The procedure of Step 4 of Example 3 was repeated except for using 390 mg (1 mmol) of the compound obtained in Step 2 of Example 53, 256 mg (1.2 mmol) of [4-chloromethyl-5-methyl-2-(thiophen-2-yl)]oxazol and 56 mg (1.4 mmol) of 60% sodium hydride to obtain the title compound (414 mg, 73%).

¹H-NMR (CDCl₃, 300 MHz): δ (ppm) 7.63-7.62 (m, 1H), 7.44-7.39 (m, 2H), 7.17-7.08 (m, 5H), 6.97-6.91 (m, 3H), 4.94 (s, 2H), 4.57 (s, 2H), 4.06 (t, J=8.7 Hz, 2H), 3.95 (q, J=7.2 Hz, 2H), 3.85 (s, 2H), 3.12 (t, J=8.7 Hz, 2H), 2.40 (s, 3H), 1.12 (t, J=7.2 Hz, 3H).

### Step 2: Preparation of [N-(indolinyl)sulfonyl-N-[4-[[2-(thiophen-2-yl)-5-methyl oxazol-4-yl]methoxy]benzyl]amino]acetic acid

The procedure of Step 5 of Example 3 was repeated except for using 414 mg (0.73 mmol) of the compound obtained in Step 1 and 46 mg (1.10 mmol) of lithium hydroxide monohydrate to obtain the title compound (390 mg, 99%).

¹H-NMR (CDCl₃, 300 MHz): δ (ppm) 7.66-7.64 (m, 1H), 7.42-7.40 (m, 2H), 7.14-7.07 (m, 5H), 6.94-6.87 (m, 3H), 4.93 (s, 2H), 4.49 (s, 2H), 4.02 (t, J=8.4 Hz, 2H), 3.88 (s, 2H), 3.08 (t, J=8.4 Hz, 2H), 2.41 (s, 3H).

### EXAMPLE 57

### Preparation of [N-(1,2,3,4-tetrahydroquinolinyl)sulfonyl-N-[4-[(2-phenyl-5-methyloxazol-4-yl)methoxy]benzyl]amino]acetic acid

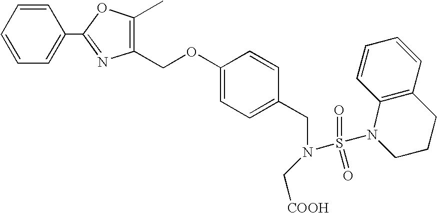

### Step 1: Preparation of [N-(1,2,3,4-tetrahydroquinolinyl)sulfonyl-N-(4-benzyl oxybenzyl)amino]acetic acid ethyl ester

The procedure of Step 2 of Example 3 was repeated except for using 14.97 g (0.05 mol) of the compound obtained in Step 1 of Example 38, 17.38 g (0.075 mol) of (1,2,3,4-tetrahydroquinolinyl)sulfonyl chloride and 8.1 g (0.08 mol) of triethylamine to obtain the title compound (18.79 g, 76%).

### Step 2: Preparation of [N-(1,2,3,4-tetrahydroquinolinyl)sulfonyl-N-(4-hydroxy benzyl)amino]acetic acid ethyl ester

The procedure of Step 3 of Example 3 was repeated except for using 18.79 g (0.038 mol) of the compound obtained in Step 1 and 4 g of Pd/C catalyst to obtain the title compound (15.22 g, 99%).

### Step 3: Preparation of [N-(1,2,3,4-tetrahydroquinolinyl)sulfonyl-N-[4-[(2-phenyl-5-methyloxazol-4-yl)methoxy]benzyl]amino]acetic acid ethyl ester

The procedure of Step 4 of Example 3 was repeated except for using 404 mg (1 mmol) of the compound obtained in Step 2, 250 mg (1.2 mmol) of (4-chloromethyl-5-methyl-2-phenyl)oxazol and 56 mg (1.4 mmol) of 60% sodium hydride to obtain the title compound (432 mg, 75%).

¹H-NMR (CDCl₃, 300 MHz): δ (ppm) 8.02 (m, 2H), 7.59 (d, J=8.1 Hz, 1H), 7.44 (m, 3H), 7.12-7.06 (m, 5H), 6.92 (d, J=8.7 Hz, 2H), 4.97 (s, 2H), 4.49 (s, 2H), 4.07 (q, J=7.2 Hz, 2H), 3.78 (m, 4H), 2.82 (t, J=6.7 Hz, 2H), 2.43 (s, 3H), 2.09 (m, 2H), 1.20 (t, J=7.2 Hz, 3H).

### Step 4: Preparation of [N-(1,2,3,4-tetrahydroquinolinyl)sulfonyl-N-[4-[(2-phenyl-5-methyloxazol-4-yl)methoxy]benzyl]amino]acetic acid

The procedure of Step 5 of Example 3 was repeated except for using 432 mg (0.75 mmol) of the compound obtained in Step 3 and 47 mg (1.13 mmol) of lithium hydroxide monohydrate to obtain the title compound (411 mg, 99%).

¹H-NMR (CDCl₃, 300 MHz): δ (ppm) 8.00-7.98 (m, 2H), 7.59 (d, J=8.1 Hz, 1H), 7.44-7.42 (m, 3H), 7.14-7.00 (m, 5H), 6.88 (d, J=8.4 Hz, 2H), 4.97 (s, 2H), 4.46 (s, 2H), 3.83 (s, 2H), 3.75 (t, J=5.9 Hz, 2H), 2.79 (t, J=6.6 Hz, 2H), 2.43 (s, 3H), 2.04 (s, 3H).

### EXAMPLE 58

### Preparation of [N-(1,2,3,4-tetraquinolinyl)sulfonyl-N-[4-[[2-(4-methylphenyl)-5-methyloxazol-4-yl]methoxy]benzyl]amino]acetic acid

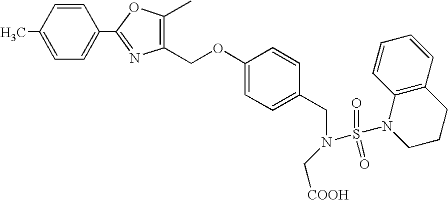

### Step 1: Preparation of [N-(1,2,3,4-tetraquinolinyl)sulfonyl-N-[4-[[2-(4-methyl phenyl)-5-methyloxazol-4-yl]methoxy]benzyl]amino]acetic acid ethyl ester

The procedure of Step 4 of Example 3 was repeated except for using 404 mg (1 mmol) of the compound obtained in Step 2 of Example 57, 266 mg (1.2 mmol) of [(4-chloromethyl-5-methyl-2-(4-methylphenyl))oxazol and 56 mg (1.4 mmol) of 60% sodium hydride to obtain the title compound (448 mg, 76%).

¹H-NMR (CDCl₃, 300 MHz): δ (ppm) 7.89 (d, J=8.2 Hz, 2H), 7.60 (d, J=8.2 Hz, 1H), 7.24 (d, J=8.6 Hz, 2H), 7.15-7.03 (m, 5H), 6.91 (d, J=8.6 Hz, 2H), 4.95 (s, 2H), 4.49 (s, 2H), 4.07 (q, J=7.2 Hz, 2H), 3.82 (s, 2H), 3.78 (t, J=5.8 Hz, 2H), 2.82 (t, J=6.7 Hz, 2H), 2.41 (s, 3H), 2.39 (s, 3H), 2.08-2.03 (m, 2H), 1.19 (t, J=7.2 Hz, 3H).

### Step 2: Preparation of [N-(1,2,3,4-tetraquinolinyl)sulfonyl-N-[4-[[2-(4-methyl phenyl)-5-methyloxazol-4-yl]methoxy]benzyl]amino]acetic acid

The procedure of Step 5 of Example 3 was repeated except for using 432 mg (0.76 mmol) of the compound obtained in Step 1 and 48 mg (1.14 mmol) of lithium hydroxide monohydrate to obtain the title compound (423 mg, 99%).

¹H-NMR (CDCl₃, 300 MHz): δ (ppm) 7.87 (d, J=8.2 Hz, 2H), 7.60 (d, J=8.2 Hz, 1H), 7.24 (d, J=8.6 Hz, 2H), 7.10-7.03 (m, 5H), 6.94 (d, J=8.6 Hz, 2H), 4.95 (s, 2H), 4.45 (s, 2H), 3.81 (s, 2H), 3.77 (t, J=5.8 Hz, 2H), 2.79 (t, J=6.7 Hz, 2H), 2.42 (s, 3H), 2.39 (s, 3H), 2.06-2.03 (m, 2H).

### EXAMPLE 59

### Preparation of [N-(1,2,3,4-tetrahydroquinolinyl)sulfonyl-N-[4-[[2-(4-trifluoromethylphenyl)-5-methyloxazol-4-yl]methoxy]benzyl]amino]acetic acid

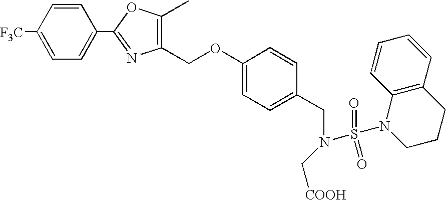

### Step 1: Preparation of [N-(1,2,3,4-tetrahydroquinolinyl)sulfonyl-N-[4-[[2-(4-trifluoromethylphenyl)-5-methyloxazol-4-yl]methoxy]benzyl]amino]acetic acid ethyl ester

The procedure of Step 4 of Example 3 was repeated except for using 404 mg (1 mmol) of the compound obtained in Step 2 of Example 57, 330 mg (1.2 mmol) of [4-chloromethyl-5-methyl-2-(4-trifluoromethylphenyl)]oxazol and 56 mg (1.4 mmol) of 60% sodium hydride to obtain the title compound (463 mg, 72%).

¹H-NMR (CDCl₃, 300 MHz): δ (ppm) 8.12 (d, J=8.4 Hz, 2H), 7.86 (d, J=8.4 Hz, 2H), 7.60 (d, J=8.2 Hz, 1H), 7.17-7.01 (m, 5H), 6.93 (d, J=8.6 Hz, 2H), 4.97 (s, 2H), 4.50 (s, 2H), 4.08 (q, J=7.2 Hz, 2H), 3.82 (s, 2H), 3.79 (t, J=5.8 Hz, 2H), 2.83 (t, J=6.6 Hz, 2H), 2.45 (s, 3H), 2.11-2.02 (m, 2H), 1.19 (t, J=7.2 Hz, 3H).

### Step 2: Preparation of [N-(1,2,3,4-tetrahydroquinolinyl)sulfonyl-N-[4-[[2-(4-trifluoromethylphenyl)-4-methyloxazol-4-yl]methoxy]benzyl]amino]acetic acid

The procedure of Step 5 of Example 3 was repeated except for using 463 mg (0.72 mmol) of the compound obtained in Step 1 and 45 mg (1.08 mmol) of lithium hydroxide monohydrate to obtain the title compound (439 mg, 99%).

¹H-NMR (CDCl₃, 200 MHz): δ (ppm) 8.10 (d, J=8.4 Hz, 2H), 7.69 (d, J=8.4 Hz, 2H), 7.53 (d, J=8.2 Hz, 1H), 7.10-6.91 (m, 5H), 6.84 (d, J=8.6 Hz, 2H), 4.90 (s, 2H), 4.40 (s, 2H), 3.84 (s, 2H), 3.74 (t, J=5.8 Hz, 2H), 2.75 (t, J=6.6 Hz, 2H), 2.40 (s, 3H), 2.08-2.02 (m, 2H).

### EXAMPLE 60

### Preparation of [N-(1,2,3,4-tetrahydroquinolinyl)sulfonyl-N-[4-[[2-(thiophen-2-yl)-5-methyloxazol-4-yl]methoxy]benzyl]amino]acetic acid

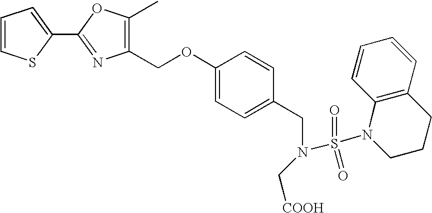

### Step 1: Preparation of [N-(1,2,3,4-tetrahydroquinolinyl)sulfonyl-N-[4-[[2-(thiophen-2-yl)-5-methyloxazol-4-yl]methoxy]benzyl]amino]acetic acid ethyl ester

The procedure of Step 4 of Example 3 was repeated except for using 404 mg (1 mmol) of the compound obtained in Step 2 of Example 57, 256 mg (1.2 mmol) of [4-chloromethyl-5-methyl-2-(thiophen-2-yl)]oxazol and 56 mg (1.4 mmol) of 60% sodium hydride to obtain the title compound (442 mg, 76%).

¹H-NMR (CDCl₃, 300 MHz): δ (ppm) 7.63-7.59 (m, 2H), 7.39 (d, J=5.9 Hz, 1H), 7.15-7.04 (m, 6H), 6.89 (d, J=8.6 Hz, 2H), 4.93 (s, 2H), 4.49 (s, 2H), 4.08 (q, J=7.2 Hz, 2H), 3.82 (s, 2H), 3.78 (t, J=5.8 Hz, 2H), 2.82 (t, J=6.7 Hz, 2H), 2.40 (s, 3H), 2.10-2.04 (m, 2H), 1.19 (t, J=7.2 Hz, 3H).

### Step 2: Preparation of [N-(1,2,3,4-tetrahydroquinolinyl)sulfonyl-N-[4-[[2-(thiophen-2-yl)-5-methyloxazol-4-yl]methoxy]benzyl]amino]acetic acid

The procedure of Step 5 of Example 3 was repeated except for using 442 mg (0.76 mmol) of the compound obtained in Step 1 and 48 mg (1.14 mmol) of lithium hydroxide monohydrate to obtain the title compound (417 mg, 99%).

¹H-NMR (CDCl₃, 300 MHz): δ (ppm) 8.82 (br s, 1H) 7.66-7.57 (m, 2H) 7.41 (d, J=5.9 Hz, 1H) 7.10-7.00 (m, 6H) 6.86 (d, J=8.6 Hz, 2H) 4.94 (s, 2H) 4.46 (s, 2H) 3.84 (s, 2H) 3.78 (t, J=5.8 Hz, 2H) 2.79 (t, J=6.7 Hz, 2H) 2.40 (s, 3H) 2.08-2.02 (m, 2H)

### EXAMPLE 61

### Preparation of [N—(N,N-dimethylamino)sulfonyl-N-[3-[2-(2-phenyl-5-methyloxazol-4-yl)ethoxy]benzyl]amino]acetic acid

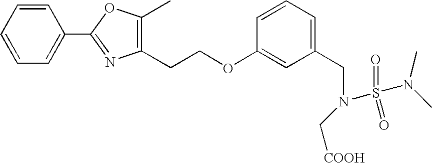

### Step 1: Preparation of [N—(N,N-dimethylamino)sulfonyl-N-[3-[2-(2-phenyl-5-methyloxazol-4-yl)ethoxy]benzyl]amino]acetic acid ethyl ester

302 mg (1 mmol) of the compound obtained in Step 3 of Example 3, 305 mg (1.5 mmol) of 2-[(5-methyl-2-phenyl)oxazol-4-yl]]ethanol and 446 mg (1.7 mmol) of triphenylphosphine were dissolved in 10 ml of toluene, and 344 mg (1.7 mmol) of diisopropyl azodicarboxylate was added thereto followed by stirring at room temperature for 12 hours. The resulting mixture was concentrated under a reduced pressure, and the residue was subjected to silica gel column chromatography (eluent: dichloromethane/ethylacetate=20/1 (v/v)) to obtain the title compound (466 mg, 93%).

¹H-NMR (CDCl₃, 200 MHz): δ (ppm) 8.00-7.95 (m, 2H), 7.43-7.39 (m, 3H), 7.28-7.21 (m, 1H), 6.89-6.87 (m, 3H), 4.51 (s, 2H), 4.24 (t, J=6.7 Hz, 2H), 4.15 (q, J=7.2 Hz, 2H), 3.83 (s, 2H), 2.98 (t, J=6.7 Hz, 2H), 2.86 (s, 6H), 2.34 (s, 3H), 1.23 (t, J=7.2 Hz, 3H).

### Step 2: Preparation of [N—(N,N-dimethylamino)sulfonyl-N-[3-[2-(2-phenyl-5-methyloxazol-4-yl)ethoxy]benzyl]amino]acetic acid

The procedure of Step 5 of Example 3 was repeated except for using 466 mg (0.93 mmol) of the compound obtained in Step 1 and 59 mg (1.4 mmol) of lithium hydroxide monohydrate to obtain the title compound (436 mg, 99%).

¹H-NMR (CDCl₃, 200 MHz): δ (ppm) 9.92 (br s, 1H), 7.98-7.93 (m, 2H), 7.44-7.39 (m, 3H), 7.25-7.18 (m, 1H), 6.97-6.86 (m, 3H), 4.49 (s, 2H), 4.23 (t, J=6.7 Hz, 2H), 3.89 (s, 2H), 2.97 (t, J=6.7 Hz, 2H), 2.84 (s, 6H), 2.37 (s, 3H).

### EXAMPLE 62

### Preparation of [N—(N,N-dimethylamino)sulfonyl-N-[3-[2-[2-(4-methylphenyl)-5-methyloxazol-4-yl]ethoxy]benzyl]amino]acetic acid

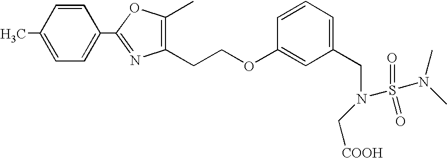

### Step 1: Preparation of [N—(N,N-dimethylamino)sulfonyl-N-[3-[2-[2-(4-methyl phenyl)-5-methyloxazol-4-yl]ethoxy]benzyl]amino]acetic acid ethyl ester

The procedure of Step 1 of Example 61 was repeated except for using 316 mg (1 mmol) of the compound obtained in Step 3 of Example 3, 326 mg (1.5 mmol) of 2-[5-methyl-2-(4-methylphenyl)oxazol-4-yl]ethanol, 466 mg (1.7 mmol) of triphenylphosphine and 344 mg (1.7 mmol) of diisopropyl azocarboxylate to obtain the title compound (469 mg, 91%).

¹H-NMR (CDCl₃, 200 MHz): δ (ppm) 7.85 (d, J=8.1 Hz, 2H), 7.27-7.19 (m, 3H), 6389-6.86 (m, 3H), 4.50 (s, 2H), 4.26-4.13 (m, 4H), 3.83 (s, 2H), 2.97 (t, J=6.7 Hz, 2H), 2.86 (s, 6H), 2.38 (s, 3H), 2.36 (s, 3H), 1.23 (t, J=7.2 Hz, 3H).

### Step 2: Preparation of [N—(N,N-dimethylamino)sulfonyl-N-[3-[2-[2-(4-methyl phenyl)-5-methyloxazol-4-yl]ethoxy]benzyl]amino]acetic acid

The procedure of Step 5 of Example 3 was repeated except for using 469 mg (0.91 mmol) of the compound obtained in Step 1 and 59 mg (1.4 mmol) of lithium hydroxide monohydrate to obtain the title compound (439 mg, 99%).

¹H-NMR (CDCl₃, 200 MHz): δ (ppm) 10.05 (br s, 1H), 7.84 (d, J=8.1 Hz, 2H), 7.25-7.18 (m, 3H), 6.97-6.86 (m, 3H), 4.50 (s, 2H), 4.22 (t, J=6.7 Hz, 2H), 3.89 (s, 2H), 2.96 (t, J=6.7 Hz, 2H), 2.85 (s, 6H), 2.38 (s, 3H), 2.37 (s, 3H).

### EXAMPLE 63

### Preparation of [N—(N,N-diethylamino)sulfonyl-N-[3-[2-[2-(4-methylphenyl)-5-methyloxazol-4-yl]ethoxy]benzyl]amino]acetic acid

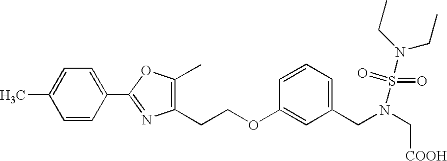

### Step 1: Preparation of [N—(N,N-diethylamino)sulfonyl-N-[3-[2-[2-(4-methyl phenyl)-5-methyloxazol-4-yl]ethoxy]benzyl]amino]acetic acid ethyl ester

The procedure of Step 2 of Example 3 was repeated except for using 380 mg (1 mmol) of [N-[3-[2-[2-(4-methylphenyl)-5-methyloxazol-4-yl]ethoxy]benzyl]amino]acetic acid ethyl ester, 189 mg (1.1 mmol) of N,N-diethylsulfamoyl chloride and 111 mg (1.1 mmol) of triethylamine to obtain the title compound (413 mg, 76%).

¹H-NMR (CDCl₃, 200 MHz): δ (ppm) 7.86 (d, J=8.3 Hz, 2H), 7.26-7.19 (m, 3H), 6.90-6.81 (m, 3H), 4.45 (s, 2H), 4.23 (t, J=6.5 Hz, 2H), 4.13 (q, J=7.1 Hz, 2H), 3.79 (s, 2H), 3.32 (q, J=7.1 Hz, 4H), 2.97 (t, J=6.5 Hz, 2H), 2.37 (s, 3H), 2.36 (s, 3H), 1.25-1.15 (m, 9H).

### Step 2: Preparation of [N—(N,N-diethylamino)sulfonyl-N-[3-[2-[2-(4-methyl phenyl)-5-methyloxazol-4-yl]ethoxy]benzyl]amino]acetic acid

The procedure of Step 5 of Example 3 was repeated except for using 413 mg (0.76 mmol) of the compound obtained in Step 1 and 48 mg (1.14 mmol) of lithium hydroxide monohydrate to obtain the title compound (388 mg, 99%).

¹H-NMR (CDCl₃, 200 MHz): δ (ppm) 9.79 (br s, 1H), 7.84 (d, J=8.3 Hz, 2H), 7.26-7.18 (m, 3H), 7.02-6.81 (m, 3H), 4.45 (s, 2H), 4.22 (t, J=6.5 Hz, 2H), 3.86 (s, 2H), 3.32 (q, J=7.1 Hz, 4H), 2.95 (t, J=6.5 Hz, 2H), 2.38 (s, 3H), 2.36 (s, 3H), 1.17 (t, J=7.1 Hz, 6H).

### EXAMPLE 64

### Preparation of [N—(N-isopropyl-N-methylamino)sulfonyl-N-[3-[2-[2-(4-methylphenyl)-5-methyloxazol-4-yl]ethoxy]benzyl]amino]acetic acid

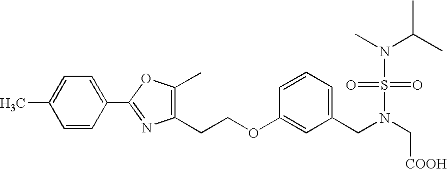

### Step 1: Preparation of [N—(N-isopropyl-N-methylamino)sulfonyl-N-[3-[2-[2-(4-methylphenyl)-5-methyloxazol-4-yl]ethoxy]benzyl]amino]acetic acid ethyl ester

The procedure of Step 2 of Example 3 was repeated except for using 380 mg (1 mmol) of [N-[3-[2-[2-(4-methylphenyl)-5-methyloxazol-4-yl]ethoxy]benzyl]amino]acetic acid ethyl ester, 189 mg (1.1 mmol) of (N-isopropyl-N-methylamino)sulfonyl chloride and 111 mg (1.1 mmol) of triethylamine to obtain the title compound (468 mg, 86%).

¹H-NMR (CDCl₃, 200 MHz): δ (ppm) 7.86 (d, J=8.1 Hz, 2H), 7.26-7.19 (m, 3H), 6.89-6.81 (m, 3H), 4.43 (s, 2H), 4.26-4.08 (m, 5H), 3.79 (s, 2H), 2.97 (t, J=6.5 Hz, 2H), 2.73 (s, 3H), 2.38 (s, 3H), 2.37 (s, 3H), 1.26-1.16 (m, 9H).

### Step 2: Preparation of [N—(N-isopropyl-N-methylamino)sulfonyl-N-[3-[2-[2-(4-methylphenyl)-5-methyloxazol-4-yl]ethoxy]benzyl]amino]acetic acid

The procedure of Step 5 of Example 3 was repeated except for using 468 mg (0.86 mmol) of the compound obtained in Step 1 and 54 mg (1.29 mmol) of lithium hydroxide monohydrate to obtain the title compound (439 mg, 99%).

¹H-NMR (CDCl₃, 200 MHz): δ (ppm) 9.91 (br s, 1H), 7.84 (d, J=8.1 Hz, 2H), 7.26-7.17 (m, 3H), 7.02-6.81 (m, 3H), 4.43 (s, 2H), 4.26-4.14 (m, 3H), 3.85 (s, 2H), 2.94 (t, J=6.5 Hz, 2H), 2.73 (s, 3H), 2.38 (s, 3H), 2.35 (s, 3H), 1.17 (s, 3H), 1.14 (s, 3H).

### EXAMPLE 65

### Preparation of [N—(N,N-dimethylamino)sulfonyl-N-[3-[2-[2-(4-trifluoromethylphenyl)-5-methyloxazol-4-yl]ethoxy]benzyl]amino]acetic acid

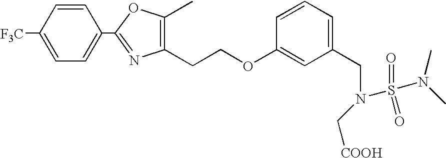

### Step 1: Preparation of [N—(N,N-dimethylamino)sulfonyl-N-[3-[2-[2-(4-trifluoromethylphenyl)-5-methyloxazol-4-yl]ethoxy]benzyl]amino]acetic acid ethyl ester

The procedure of Step 1 of Example 61 was repeated except for using 316 mg (1 mmol) of the compound obtained in Step 3 of Example 3, 407 mg (1.5 mmol) of 2-[5-methyl-2-(4-trifluoromethylphenyl)oxazol-4-yl]ethanol, 466 mg (1.7 mmol) of triphenylphosphine and 344 mg (1.7 mmol) of diisopropyl azocarboxylate to obtain the title compound (524 mg, 92%).

¹H-NMR (CDCl₃, 200 MHz): δ (ppm) 8.08 (d, J=8.1 Hz, 2H), 7.68 (d, J=6.1 Hz, 2H), 7.25-7.24 (m, 1H), 6.89-6.82 (m, 3H), 4.52 (s, 2H), 4.28-4.10 (m, 4H), 3.84 (s, 2H), 2.98 (t, J=6.7 Hz, 2H), 2.87 (s, 6H), 2.41 (s, 3H), 1.24 (t, J=7.2 Hz, 3H).

### Step 2: Preparation of [N—(N,N-dimethylamino)sulfonyl-N-[3-[2-[2-(4-trifluoro methylphenyl)-5-methyloxazol-4-yl]ethoxy]benzyl]amino]acetic acid

The procedure of Step 5 of Example 3 was repeated except for using 524 mg (0.92 mmol) of the compound obtained in Step 1 and 59 mg (1.4 mmol) of lithium hydroxide monohydrate to obtain the title compound (439 mg, 99%).

¹H-NMR (CDCl₃, 200 MHz): δ (ppm) 9.65 (br s, 1H), 8.07 (d, J=8.1 Hz, 2H), 7.67 (d, J=8.1 Hz, 2H), 7.23-7.19 (m, 1H), 6.95-6.87 (m, 3H), 4.49 (s, 2H), 4.24 (t, J=6.7 Hz, 2H), 3.89 (s, 2H), 2.98 (t, J=6.7 Hz, 2H), 2.85 (s, 6H), 2.40 (s, 3H).

### EXAMPLE 66

### Preparation of [N—(N-t-butylamino)sulfonyl-N-[3-[2-[2-(4-tri fluoromethylphenyl)-5-methyloxazol-4-yl]ethoxy]benzyl]amino]acetic acid

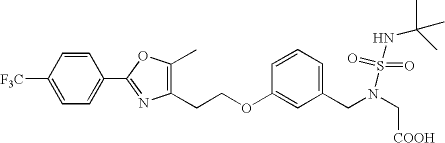

### Step 1: Preparation of [N—(N-t-butylamino)sulfonyl-N-[3-[2-[2-(4-trifluoromethylphenyl)-5-methyloxazol-4-yl]ethoxy]benzyl]amino]acetic acid ethyl ester

The procedure of Step 2 of Example 1 was repeated except for using 462 mg (1 mmol) of [N-[3-[2-[2-(4-trifluoromethylphenyl)-5-methyloxazol-4-yl]ethoxy]benzyl]amino]acetic acid ethyl ester, 0.11 g (1.1 mmol) of triethylamine and 0.21 g (12 mmol) of t-butylamino sulfonyl chloride to obtain the title compound (538 mg, 90%).

¹H-NMR (CDCl₃, 200 MHz): δ (ppm) 8.09 (d, J=8.7 Hz, 2H), 7.68 (d, J=8.7 Hz, 2H), 7.28-7.19 (m, 1H), 6.89-6.82 (m, 3H), 5.23 (s, 1H), 4.34 (s, 2H), 4.27-4.12 (m, 4H), 3.89 (s, 2H), 2.99 (t, J=6.5 Hz, 2H), 2.41 (s, 3H), 1.40 (s, 9H), 1.24 (t, J=7.2 Hz, 3H).

### Step 2: Preparation of [N—(N-t-butylamino)sulfonyl-N-[3-[2-[2-(4-trifluoro methylphenyl)-5-methyloxazol-4-yl]ethoxy]benzyl]amino]acetic acid

The procedure of Step 5 of Example 3 was repeated except for using 538 mg (0.9 mmol) of the compound obtained in Step 1 and 57 mg (1.35 mmol) of lithium hydroxide monohydrate to obtain the title compound (507 mg, 99%).

¹H-NMR (CDCl₃, 200 MHz): δ (ppm) 8.00 (d, J=8.7 Hz, 2H), 7.61 (d, J=8.7 Hz, 2H), 7.06-6.98 (m, 1H), 6.84-6.66 (m, 3H), 5.71 (br s, 1H), 4.30 (s, 2H), 4.11 (t, J=6.5 Hz, 2H), 3.69 (s, 2H), 2.88 (t, J=6.5 Hz, 2H), 2.31 (s, 3H), 1.17 (s, 9H).

### EXAMPLE 67

### Preparation of [N—(N,N-diethylamino)sulfonyl-N-[3-[2-[2-(4-trifluoromethylphenyl)-5-methyloxazol-4-yl]ethoxy]benzyl]amino]acetic acid

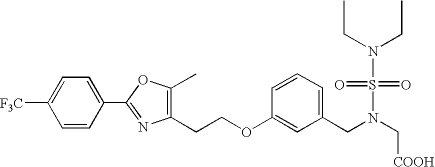

### Step 1: Preparation of [N—(N,N-diethylamino)sulfonyl-N-[3-[2-[2-(4-trifluoro methylphenyl)-5-methyloxazol-4-yl]ethoxy]benzyl]amino]acetic acid ethyl ester

The procedure of Step 2 of Example 3 was repeated except for using 462 mg (1 mmol) of [N-[3-[2-[2-(4-trifluoromethylphenyl)-5-methyloxazol-4-yl]ethoxy]benzyl]amino]acetic acid ethyl ester, 189 mg (1.1 mmol) of N,N-diethyl sulfamoyl chloride and 111 mg (1.1 mmol) of triethylamine to obtain the title compound (490 mg, 82%).

¹H-NMR (CDCl₃, 200 MHz): δ (ppm) 8.08 (d, J=8.1 Hz, 2H), 7.66 (d, J=8.1 Hz, 2H), 7.28-7.19 (m, 1H), 6.90-6.81 (m, 3H), 4.46 (s, 2H), 4.24 (t, J=6.5 Hz, 2H), 4.14 (q, J=7.1 Hz, 2H), 3.79 (s, 2H), 3.32 (q, J=7.1 Hz, 4H), 2.99 (t, J=6.5 Hz, 2H), 2.40 (s, 3H), 1.29-1.16 (m, 9H).

### Step 2: Preparation of [N—(N,N-diethylamino)sulfonyl-N-[3-[2-[2-(4-trifluoro methylphenyl)-5-methyloxazol-4-yl]ethoxy]benzyl]amino]acetic acid

The procedure of Step 5 of Example 3 was repeated except for using 490 mg (0.82 mmol) of the compound obtained in Step 1 and 53 mg (1.23 mmol) of lithium hydroxide monohydrate to obtain the title compound (462 mg, 99%).

¹H-NMR (CDCl₃, 200 MHz): δ (ppm) 8.99 (br s, 1H), 8.07 (d, J=8.1 Hz, 2H), 7.68 (d, J=8.1 Hz, 2H), 7.28-7.19 (m, 1H), 6.98-6.81 (m, 3H), 4.45 (s, 2H), 4.24 (t, J=6.5 Hz, 2H), 3.86 (s, 2H), 3.32 (q, J=7.1 Hz, 4H), 2.98 (t, J=6.5 Hz, 2H), 2.40 (s, 3H), 1.29-1.14 (m, 6H).

### EXAMPLE 68

### Preparation of [N—(N-isopropyl-N-methylanmo)sulfonyl-N-[3-[2-[2-(4-trifluoromethylphenyl)-5-methyloxazol-4-yl]ethoxy]benzyl]amino]acetic acid

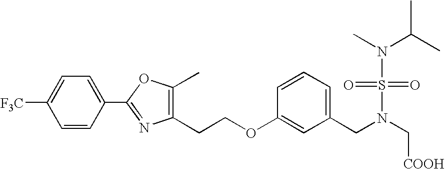

### Step 1: Preparation of [N—(N-isopropyl-N-methylamino)sulfonyl-N-[3-[2-[2-(4-trifluoromethylphenyl)-5-methyloxazol-4-yl]ethoxy]benzyl]amino]acetic acid ethyl ester

The procedure of Step 2 of Example 3 was repeated except for using 462 mg (1 mmol) of [N-[3-[2-[2-(4-trifluoromethylphenyl)-5-methyloxazol-4-yl]ethoxy]benzyl]amino]acetic acid ethyl ester, 189 mg (1.1 mmol) of [N-isopropyl-N-methylamino]sulfonyl chloride and 111 mg (1.1 mmol) of triethylamine to obtain the title compound (514 mg, 86%).

¹H-NMR (CDCl₃, 200 MHz): δ (ppm) 8.09 (d, J=8.1 Hz, 2H), 7.68 (d, J=8.1 Hz, 2H), 7.23-7.19 (m, 1H), 6.91-6.81 (m, 3H), 4.45 (s, 2H), 4.28-4.08 (m, 5H), 3.79 (s, 2H), 2.99 (t, J=6.5 Hz, 2H), 2.74 (s, 3H), 2.40 (s, 3H), 1.26-1.16 (m, 9H).

### Step 2: Preparation of [N—(N-isopropyl-N-methylamino)sulfonyl-N-[3-[2-[2-(4-trifluoromethylphenyl)-5-methyloxazol-4-yl]ethoxy]benzyl]amino]acetic acid

The procedure of Step 5 of Example 3 was repeated except for using 514 mg (0.86 mmol) of the compound obtained in Step 1 and 54 mg (1.29 mmol) of lithium hydroxide monohydrate to obtain the title compound (485 mg, 99%).

¹H-NMR (CDCl₃, 200 MHz): δ (ppm) 8.43 (br s, 1H), 8.07 (d, J=8.1 Hz, 2H), 7.68 (d, J=8.1 Hz, 2H), 7.26-7.19 (m, 1H), 7.00-6.82 (m, 3H), 4.43 (s, 2H), 4.27-4.13 (m, 3H), 3.85 (s, 2H), 2.97 (t, J=6.5 Hz, 2H), 2.73 (s, 3H), 2.40 (s, 3H), 1.18 (s, 3H), 1.15 (s, 3H).

### EXAMPLE 69

### Preparation of [N—(N-allyl-N-methylamino)sulfonyl-N-[3-[2-[2-(4-trifluoromethylphenyl)-5-methyloxazol-4-yl]ethoxy]benzyl]amino]acetic acid

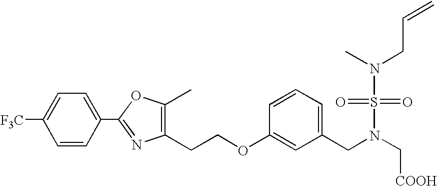

### Step 1: Preparation of [N—(N-allyl-N-methylamino)sulfonyl-N-[3-[2-[2-(4-trifluoromethylphenyl)-5-methyloxazol-4-yl]ethoxy]benzyl]amino]acetic acid ethyl ester

The procedure of Step 2 of Example 3 was repeated except for using 462 mg (1 mmol) of [N-[3-[2-[2-(4-trifluoromethylphenyl)-5-methyloxazol-4-yl]ethoxy]benzyl]amino]acetic acid ethyl ester, 187 mg (1.1 mmol) of (N-allyl-N-methylamino)sulfonyl chloride and 111 mg (1.1 mmol) of triethylamine to obtain the title compound (477 mg, 80%).

¹H-NMR (CDCl₃, 200 MHz): δ (ppm) 8.08 (d, J=8.1 Hz, 2H), 7.68 (d, J=8.1 Hz, 2H), 7.28-7.20 (m, 1H), 6.89-6.83 (m, 3H), 5.89-5.73 (m, 1H), 5.31-5.21 (m, 2H), 4.49 (s, 2H), 4.24 (t, J=6.5 Hz, 2H), 4.14 (q, J=7.1 Hz, 2H), 3.85-3.76 (m, 2H), 3.83 (s, 2H), 3.00 (t, J=6.5 Hz, 2H), 2.81 (s, 3H), 2.41 (s, 3H), 1.23 (t, J=7.1 Hz, 3H).

### Step 2: Preparation of [N—(N-allyl-N-methylamino)sulfonyl-N-[3-[2-[2-(4-trifluoromethylphenyl)-5-methyloxazol-4-yl]ethoxy]benzyl]amino]acetic acid

The procedure of Step 5 of Example 3 was repeated except for using 477 mg (0.8 mmol) of the compound obtained in Step 1 and 50 mg (1.2 mmol) of lithium hydroxide monohydrate to obtain the title compound (450 mg, 99%).

¹H-NMR (CDCl₃, 200 MHz): δ (ppm) 8.92 (br s, 1H), 8.08 (d, J=8.1 Hz, 2H), 7.68 (d, J=8.1 Hz, 2H), 7.28-7.20 (m, 1H), 7.00-6.83 (m, 3H), 5.88-5.71 (m, 1H), 5.29-5.19 (m, 2H), 4.48 (s, 2H), 4.25 (t, J=6.5 Hz, 2H), 3.89 (s, 2H), 3.80 (d, J=6.3 Hz, 2H), 2.98 (t, J=6.5 Hz, 2H), 2.80 (s, 3H), 2.41 (s, 3H).

### EXAMPLE 70

### Preparation of [N—(N-methyl-N-propargylamino)sulfonyl-N-[3-[2-[2-(4-trifluoromethylphenyl)-5-methyloxazol-4-yl]ethoxy]benzyl]amino]acetic acid

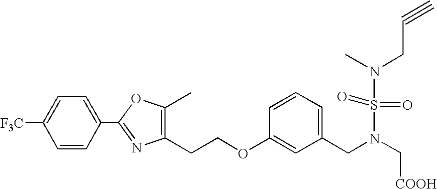

### Step 1: Preparation of [N—(N-methyl-N-propargylamino)sulfonyl-N-[3-[2-[2-(4-trifluoromethylphenyl)-5-methyloxazol-4-yl]ethoxy]benzyl]amino]acetic acid ethyl ester

The procedure of Step 2 of Example 3 was repeated except for using 462 mg (1 mmol) of [N-[3-[2-[2-(4-trifluoromethylphenyl)-5-methyloxazol-4-yl]ethoxy]benzyl]amino]acetic acid ethyl ester, 184 mg (1.1 mmol) of (N-methyl-N-propargylamino)sulfonyl chloride and 111 mg (1.1 mmol) of triethylamine to obtain the title compound (489 mg, 81%).

¹H-NMR (CDCl₃, 200 MHz): δ (ppm) 8.08 (d, J=8.1 Hz, 2H), 7.68 (d, J=8.1 Hz, 2H), 7.27-7.19 (m, 1H), 6.89-6.81 (m, 3H), 4.49 (s, 2H), 4.24 (t, J=6.5 Hz, 2H), 4.16 (q, J=7.1 Hz, 2H), 4.05 (d, J=2.4 Hz, 2H), 3.83 (s, 2H), 2.98 (t, J=6.5 Hz, 2H), 2.95 (s, 3H), 2.40 (s, 3H), 2.34 (t, J=2.4 Hz, 1H), 1.23 (t, J=7.1 Hz, 3H).

### Step 2: Preparation of [N—(N-methyl-N-propargylamino)sulfonyl-N-[3-[2-[2-(4-trifluoromethylphenyl)-5-methyloxazol-4-yl]ethoxy]benzyl]amino]acetic acid

The procedure of Step 5 of Example 3 was repeated except for using 489 mg (0.81 mmol) of the compound obtained in Step 1 and 53 mg (1.22 mmol) of lithium hydroxide monohydrate to obtain the title compound (454 mg, 99%).

¹H-NMR (CDCl₃, 200 MHz): δ (ppm) 8.23 (br s, 1H), 8.07 (d, J=8.1 Hz, 2H), 7.69 (d, J=8.1 Hz, 2H), 7.28-7.20 (m, 1H), 7.03-6.83 (m, 3H), 4.48 (s, 2H), 4.25 (t, J=6.5 Hz, 2H), 4.06 (d, J=2.4 Hz, 2H), 3.89 (s, 2H), 2.97 (t, J=6.5 Hz, 2H), 2.96 (s, 3H), 2.41 (s, 3H), 2.34 (t, J=2.4 Hz, 1H).

### EXAMPLE 71

### Preparation of [N-(piperidinyl)sulfonyl-N-[3-[2-[2-(4-trifluoro methylphenyl)-5-methyloxazol-4-yl]ethoxy]benzyl]amino]acetic acid

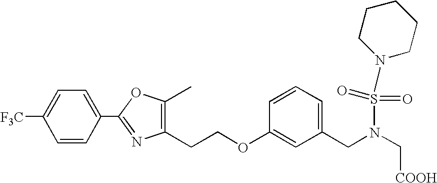

### Step 1: Preparation of [N-(piperidinyl)sulfonyl-N-[3-[2-[2-(4-trifluoromethyl phenyl)-5-methyloxazol-4-yl]ethoxy]benzyl]amino]acetic acid ethyl ester

The procedure of Step 2 of Example 3 was repeated except for using 462 mg (1 mmol) of [N-[3-[2-[2-(4-trifluoromethylphenyl)-5-methyloxazol-4-yl]ethoxy]benzyl]amino]acetic acid ethyl ester, 202 mg (1.1 mmol) of (piperidinyl)sulfonyl chloride and 111 mg (1.1 mmol) of triethylamine to obtain the title compound (518 mg, 85%).

¹H-NMR (CDCl₃, 200 MHz): δ (ppm) 8.08 (d, J=8.1 Hz, 2H), 7.67 (d, J=8.1 Hz, 2H), 7.28-7.19 (m, 1H), 6.89-6.80 (m, 3H), 4.51 (s, 2H), 4.24 (t, J=6.5 Hz, 2H), 4.14 (q, J=7.1 Hz, 2H), 3.81 (s, 2H), 3.28-3.17 (m, 4H), 2.98 (t, J=6.5 Hz, 2H), 2.40 (s, 3H), 1.68-1.43 (m, 6H), 1.23 (t, J=7.1 Hz, 3H).

### Step 2: Preparation of [N-(piperidinyl)sulfonyl-N-[3-[2-[2-(4-trifluoromethyl phenyl)-5-methyloxazol-4-yl]ethoxy]benzyl]amino]acetic acid

The procedure of Step 5 of Example 3 was repeated except for using 518 mg (0.85 mmol) of the compound obtained in Step 1 and 54 mg (1.28 mmol) of lithium hydroxide monohydrate to obtain the title compound (489 mg, 99%).

¹H-NMR (CDCl₃, 200 MHz): δ (ppm) 8.91 (br s, 1H), 8.08 (d, J=8.1 Hz, 2H), 7.68 (d, J=8.1 Hz, 2H), 7.28-7.19 (m, 1H), 6.99-6.82 (m, 3H), 4.49 (s, 2H), 4.25 (t, J=6.5 Hz, 2H), 3.89 (s, 2H), 3.26-3.17 (m, 4H), 2.97 (t, J=6.5 Hz, 2H), 2.41 (s, 3H), 1.68-1.43 (m, 6H).

### EXAMPLE 72

### Preparation of [N—(N,N-dimethylamino)sulfonyl-N-[3-[2-[2-(thiophen-2-yl)-5-methyloxazol-4-yl]ethoxy]benzyl]amino]acetic acid

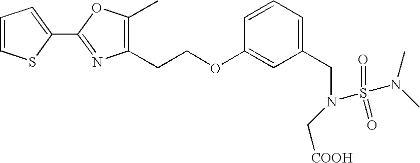

### Step 1: Preparation of [N—(N,N-dimethylamino)sulfonyl-N-[3-[2-[2-(thiophen-2-yl)-5-methyloxazol-4-yl]ethoxy]benzyl]amino]acetic acid ethyl ester

The procedure of Step 1 of Example 61 was repeated except for using 316 mg (1 mmol) of the compound obtained in Step 3 of Example 3, 314 mg (1.5 mmol) of 2-[5-methyl-2-(thiophen-2-yl)oxazol-4-yl]ethanol, 466 mg (1.7 mmol) of triphenylphosphine and 344 mg (1.7 mmol) of diisopropyl azocarboxylate to obtain the title compound (456 mg, 90%).

¹H-NMR (CDCl₃, 200 MHz): δ (ppm) 7.59-7.57 (m, 1H), 7.38-7.35 (m, 1H), 7.25-7.23 (m, 1H), 7.09-7.06 (m, 1H), 6.89-6.85 (m, 3H), 4.50 (s, 2H), 4.25-4.13 (m, 4H), 3.83 (s, 2H), 2.95 (t, J=6.7 Hz, 2H), 2.86 (s, 6H), 2.36 (s, 3H), 1.24 (t, J=7.2 Hz, 3H).

### Step 2: Preparation of [N—(N,N-dimethylamino)sulfonyl-N-[3-[2-[2-(thiophen-2-yl)-5-methyloxazol-4-yl]ethoxy]benzyl]amino]acetic acid

The procedure of Step 5 of Example 3 was repeated except for using 456 mg (0.9 mmol) of the compound obtained in Step 1 and 59 mg (1.4 mmol) of lithium hydroxide monohydrate to obtain the title compound (427 mg, 99%).

¹H-NMR (CDCl₃, 200 MHz): δ (ppm) 9.39 (br s, 1H), 7.65-7.62 (m, 1H), 7.40-7.37 (m, 1H), 7.22-7.18 (m, 1H), 7.10-7.06 (m, 1H), 6.96-6.86 (m, 3H), 4.49 (s, 2H), 4.21 (t, J=6.7 Hz, 2H), 3.89 (s, 2H), 2.94 (t, J=6.7 Hz, 2H), 2.85 (s, 6H), 2.35 (s, 3H).

### EXAMPLE 73

### Preparation of [N—[(N-methyl-N-phenyl)amino]sulfonyl-N-[3-[2-(2-phenyl-5-methyloxazol-4-yl)ethoxy]benzyl]amino]acetic acid

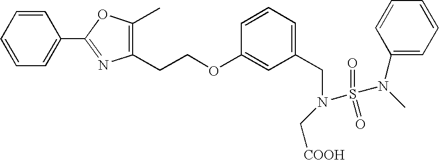

### Step 1: Preparation of [N—[(N-methyl-N-phenyl)amino]sulfonyl-N-[3-[2-(2-phenyl-5-methyloxazol-4-yl)ethoxy]benzyl]amino]acetic acid ethyl ester

The procedure of Step 1 of Example 61 was repeated except for using 316 mg (1 mmol) of the compound obtained in Step 2 of Example 14, 305 mg (1.5 mmol) of 2-[(5-methyl-2-phenyl)oxazol-4-yl]ethanol, 466 mg (1.7 mmol) of triphenylphosphine and 344 mg (1.7 mmol) of diisopropyl azocarboxylate to obtain the title compound (530 mg, 94%).

¹H-NMR (CDCl₃, 300 MHz): δ (ppm) 7.99-7.96 (m, 2H), 7.46-7.33 (m, 7H), 7.27-7.17 (m, 2H), 6.83-6.79 (m, 3H), 4.46 (s, 2H), 4.19 (t, J=6.6 Hz, 2H), 4.15 (q, J=7.2 Hz, 2H), 3.83 (s, 2H), 3.29 (s, 3H), 2.96 (t, J=6.6 Hz, 2H), 2.37 (s, 3H), 1.24 (t, J=7.2 Hz, 3H).

### Step 2: Preparation of [N—[(N-methyl-N-phenyl)amino]sulfonyl-N-[3-[2-(2-phenyl-5-methyloxazol-4-yl)ethoxy]benzyl]amino]acetic acid

The procedure of Step 5 of Example 3 was repeated except for using 530 mg (0.94 mmol) of the compound obtained in Step 1 and 59 mg (1.4 mmol) of lithium hydroxide monohydrate to obtain the title compound (498 mg, 99%).

¹H-NMR (CDCl₃, 200 MHz): δ (ppm) 7.98-7.93 (m, 2H), 7.46-7.13 (m, 9H), 6.92-6.78 (m, 3H), 4.46 (s, 2H), 4.19 (t, J=6.6 Hz, 2H), 3.86 (s, 2H), 3.29 (s, 3H), 2.94 (t, J=6.6 Hz, 2H), 2.36 (s, 3H).

### EXAMPLE 74

### Preparation of [N—[(N-methyl-N-phenyl)amino]sulfonyl-N-[3-[2-[2-(4-methylphenyl)-5-methyloxazol-4-yl]ethoxy]benzyl]amino]acetic acid

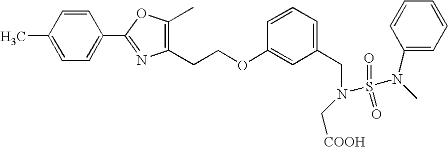

### Step 1: Preparation of [N—[(N-methyl-N-phenyl)amino]sulfonyl-N-[3-[2-[2-(4-methylphenyl)-5-methyloxazol-4-yl]ethoxy]benzyl]amino]acetic acid ethyl ester

The procedure of Step 1 of Example 61 was repeated except for using 316 mg (1 mmol) of the compound obtained in Step 2 of Example 14, 326 mg (1.5 mmol) of 2-[5-methyl-2-(4-methylphenyl)oxazol-4-yl]ethanol, 446 mg (1.7 mmol) of triphenylphosphine and 344 mg (1.7 mmol) of diisopropyl azocarboxylate to obtain the title compound (537 mg, 93%).

¹H-NMR (CDCl₃, 300 MHz): δ (ppm) 7.86 (d, J=8.1 Hz, 2H), 7.46-7.33 (m, 4H), 7.27-7.17 (m, 4H), 6.83-6.79 (m, 3H), 4.46 (s, 2H), 4.19 (t, J=6.6 Hz, 2H), 4.13 (q, J=7.2 Hz, 2H), 3.83 (s, 2H), 3.29 (s, 3H), 2.95 (t, J=6.6 Hz, 2H), 2.38 (s, 3H), 2.36 (s, 3H), 1.24 (t, J=7.2 Hz, 3H).

### Step 2: Preparation of [N—[(N-methyl-N-phenyl)amino]sulfonyl-N-[3-[2-[2-(4-methylphenyl)-5-methyloxazol-4-yl]ethoxy]benzyl]amino]acetic acid

The procedure of Step 5 of Example 3 was repeated except for using 537 mg (0.93 mmol) of the compound obtained in Step 1 and 59 mg (1.4 mmol) of lithium hydroxide monohydrate to obtain the title compound (506 mg, 99%).

¹H-NMR (CDCl₃, 200 MHz): δ (ppm) 7.84 (d, J=8.1 Hz, 2H), 7.47-7.14 (m, 8H), 6.97-6.96 (m, 1H), 6.85-6.78 (m, 2H), 6.25 (br s, 1H), 4.46 (s, 2H), 4.20 (t, J=6.6 Hz, 2H), 3.85 (s, 2H), 3.30 (s, 2H), 2.91 (t, J=6.6 Hz, 2H), 2.38 (s, 3H), 2.35 (s, 3H).

### EXAMPLE 75

### Preparation of [N—[(N-methyl-N-phenyl)amino]sulfonyl-N-[3-[2-[2-(4-trifluoromethylphenyl)-5-methyloxazol-4-yl]ethoxy]benzyl]amino]acetic acid

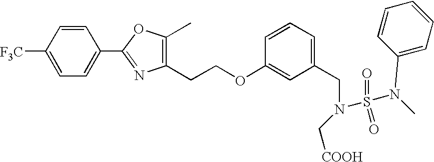

### Step 1: Preparation of [N—[(N-methyl-N-phenyl)amino]sulfonyl-N-[3-[2-[2-(4-trifluoromethylphenyl)-5-methyloxazol-4-yl]ethoxy]benzyl]amino]acetic acid ethyl ester

The procedure of Step 1 of Example 61 was repeated except for using 316 mg (1 mmol) of the compound obtained in Step 2 of Example 14, 407 mg (1.5 mmol) of 2-[5-methyl-2-[(4-trifluoromethyl)phenyl]oxazol-4-yl]ethanol, 446 mg (1.7 mmol) of triphenylphosphine and 344 mg (1.7 mmol) of diisopropyl azocarboxylate to obtain the title compound (575 mg, 91%).

¹H-NMR (CDCl₃, 300 MHz): δ (ppm) 8.07 (d, J=8.1 Hz, 2H), 7.67 (d, J=8.1 Hz, 2H), 7.47-7.44 (m, 2H), 7.38-7.33 (m, 2H), 7.28-7.19 (m, 2H), 6.83 6.78 (m, 3H), 4.46 (s, 2H), 4.20 (t, J=6.6 Hz, 2H), 4.14 (q, J=7.2 Hz, 2H), 3.83 (s, 2H), 3.29 (s, 3H), 2.97 (t, J=6.6 Hz, 2H), 2.39 (s, 3H), 1.24 (t, J=7.2 Hz, 3H).

### Step 2: Preparation of [N—[(N-methyl-N-phenyl)amino]sulfonyl-N-[3-[2-[2-(4-trifluoromethylphenyl)-5-methyloxazol-4-yl]ethoxy]benzyl]amino]acetic acid

The procedure of Step 5 of Example 3 was repeated except for using 575 mg (0.91 mmol) of the compound obtained in Step 1 and 59 mg (1.4 mmol) of lithium hydroxide monohydrate to obtain the title compound (544 mg, 99%).

¹H-NMR (CDCl₃, 200 MHz): δ (ppm) 8.06 (d, J=8.1 Hz, 2H), 7.65 (d, J=8.1 Hz, 2H), 7.46-7.15 (m, 6H), 6.93-6.77 (m, 3H), 5.41 (br s, 1H), 4.45 (s, 2H), 4.21 (t, J=6.6 Hz, 2H), 3.85 (s, 2H), 3.29 (s, 3H), 2.95 (t, J=6.6 Hz, 2H), 2.39 (s, 3H).

### EXAMPLE 76

### Preparation of [N—[(N-methyl-N-phenyl)amino]sulfonyl-N-[3-[2-[2-(thiophen-2-yl)-5-methyloxazol-4-yl]ethoxy]benzyl]amino]acetic acid

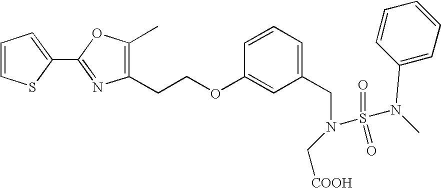

### Step 1: Preparation of [N—[(N-methyl-N-phenyl)amino]sulfonyl-N-[3-[2-[2-(thiophen-2-yl)-5-methyloxazol-4-yl]ethoxy]benzyl]amino]acetic acid ethyl ester

The procedure of Step 1 of Example 61 was repeated except for using 316 mg (1 mmol) of the compound obtained in Step 2 of Example 14, 314 mg (1.5 mmol) of 2-[5-methyl-2-(thiophen-2-yl)oxazol-4-yl]ethanol, 446 mg (1.7 mmol) of triphenylphosphine and 344 mg (1.7 mmol) of diisopropyl azocarboxylate to obtain the title compound (541 mg, 95%).

¹H-NMR (CDCl₃, 300 MHz): δ (ppm) 7.58-7.57 (m, 1H), 7.46-7.33 (m, 5H), 7.28-7.19 (m, 2H), 7.08-7.06 (m, 1H), 6.82-6.79 (m, 3H), 4.46 (s, 2H), 4.19-4.11 (m, 4H), 3.83 (s, 2H), 3.29 (s, 3H), 2.94 (t, J=6.6 Hz, 2H), 2.35 (s, 3H), 1.24 (t, J=7.2 Hz, 3H).

### Step 2: Preparation of [N—[(N-methyl-N-phenyl)amino]sulfonyl-N-[3-[2-[2-(thiophen-2-yl)-5-methyloxazol-4-yl]ethoxy]benzyl]amino]acetic acid

The procedure of Step 5 of Example 3 was repeated except for using 541 mg (0.95 mmol) of the compound obtained in Step 1 and 59 mg (1.4 mmol) of lithium hydroxide monohydrate to obtain the title compound (509 mg, 99%).

¹H-NMR (CDCl₃, 200 MHz): δ (ppm) 7.64-7.62 (m, 1H), 7.47-7.05 (m, 8H), 6.91-6.78 (m, 3H), 4.17 (br s, 1H), 4.46 (s, 2H), 4.18 (t, J=6.6 Hz, 2H), 3.86 (s, 2H), 3.29 (s, 3H), 2.91 (t, J=6.6 Hz, 2H), 2.34 (s, 3H).

### EXAMPLE 77

### Preparation of [N—[[N-methyl-N-(4-chlorophenyl)]amino]sulfonyl-N-[3-[2-(2-phenyl-5-methyloxazol-4-yl)ethoxy]benzyl]amino]acetic acid

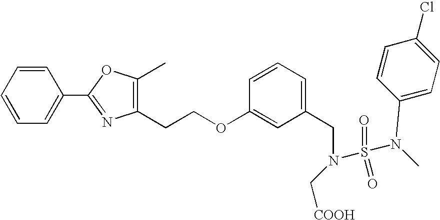

### Step 1: Preparation of [N—[[N-methyl-N-(4-chlorophenyl)]amino]sulfonyl-N-[3-[2-(2-phenyl-5-methyloxazol-4-yl)ethoxy]benzyl]amino]acetic acid ethyl ester

The procedure of Step 1 of Example 61 was repeated except for using 413 mg (1 mmol) of the compound obtained in Step 2 of Example 18, 305 mg (1.5 mmol) of 2-[(5-methyl-2-phenyl)oxazol-4-yl]ethanol, 446 mg (1.7 mmol) of triphenylphosphine and 344 mg (1.7 mmol) of diisopropyl azocarboxylate to obtain the title compound (556 mg, 93%).

¹H-NMR (CDCl₃, 300 MHz): δ (ppm) 7.99-7.96 (m, 2H), 7.42-7.20 (m, 8H), 6.81-6.78 (m, 3H), 4.44 (s, 2H), 4.21 (t, J=6.6 Hz, 2H), 4.14 (q, J=7.2 Hz, 2H), 3.84 (s, 2H), 3.26 (s, 3H), 2.96 (t, J=6.6 Hz, 2H), 2.37 (s, 3H), 1.23 (t, J=7.2 Hz, 3H).

### Step 2: Preparation of [N—[[N-methyl-N-(4-chlorophenyl)]amino]sulfonyl-N-[3-[2-(2-phenyl-5-methyloxazol-4-yl)ethoxy]benzyl]amino]acetic acid

The procedure of Step 5 of Example 3 was repeated except for using 556 mg (0.93 mmol) of the compound obtained in Step 1 and 59 mg (1.4 mmol) of lithium hydroxide monohydrate to obtain the title compound (525 mg, 99%).

¹H-NMR (CDCl₃, 300 MHz): δ (ppm) 8.02 (br s, 1H), 7.96-7.94 (m, 2H), 7.44-7.42 (m, 3H), 7.36 (d, J=8.7 Hz, 2H), 7.27 (d, J=8.7 Hz, 2H), 7.21-7.17 (m, 1H), 6.91 (s, 1H), 6.84-6.79 (m, 2H), 4.44 (s, 2H), 4.20 (t, J=6.6 Hz, 2H), 3.87 (s, 2H), 3.26 (s, 3H), 2.94 (t, J=6.6 Hz, 2H), 2.37 (s, 3H).

### EXAMPLE 78

### Preparation of [N—[[N-methyl-N-(4-chlorophenyl)]amino]sulfonyl-N-[3-[2-[2-(4-methylphenyl)-5-methyloxazol-4-yl]ethoxy]benzyl]amino]acetic acid

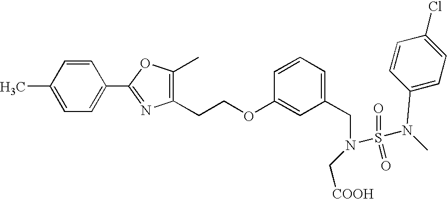

### Step 1: Preparation of [N—[[N-methyl-N-(4-chlorophenyl)]amino]sulfonyl-N-[3-[2-[2-(4-methylphenyl)-5-methyloxazol-4-yl]ethoxy]benzyl]amino]acetic acid ethyl ester

The procedure of Step 1 of Example 61 was repeated except for using 413 mg (1 mmol) of the compound obtained in Step 2 of Example 18, 326 mg (1.5 mmol) of 2-[5-methyl-2-(4-methylphenyl)oxazol-4-yl]ethanol, 446 mg (1.7 mmol) of triphenylphosphine and 344 mg (1.7 mmol) of diisopropyl azocarboxylate to obtain the title compound (575 mg, 94%).

¹H-NMR (CDCl₃, 300 MHz): δ (ppm) 7.85 (d, J=8.1 Hz, 2H), 7.39 (d, J=8.7 Hz, 2H), 7.31 (d, J=8.7 Hz, 2H), 7.25-7.17 (m, 3H), 6.84-6.78 (m, 3H), 4.44 (s, 2H), 4.20 (t, J=6.6 Hz, 2H), 4.14 (q, J=7.2 Hz, 2H), 3.83 (s, 2H), 3.27 (s, 3H), 2.97 (t, J=6.6 Hz, 2H), 2.38 (s, 3H), 2.36 (s, 3H), 1.23 (t, J=7.2 Hz, 3H).

### Step 2: Preparation of [N—[[N-methyl-N-(p-chlorophenyl)]amino]sulfonyl-N-[3-[2-[2-(4-methylphenyl)-5-methyloxazol-4-yl]ethoxy]benzyl]amino]acetic acid

The procedure of Step 5 of Example 3 was repeated except for using 575 mg (0.94 mmol) of the compound obtained in Step 1 and 59 mg (1.4 mmol) of lithium hydroxide monohydrate to obtain the title compound (544 mg, 99%).

¹H-NMR (CDCl₃, 300 MHz): δ (ppm) 8.62 (br s, 1H), 7.83 (d, J=8.1 Hz, 2H), 7.38-7.15 (m, 7H), 6.91 (s, 1H), 6.90-6.78 (m, 2H), 4.45 (s, 2H), 4.19 (t, J=6.6 Hz, 2H), 3.86 (s, 2H), 3.26 (s, 3H), 2.93 (t, J=6.6 Hz, 2H), 2.38 (s, 3H), 2.35 (s, 3H).

### EXAMPLE 79

### Preparation of [N—[[N-methyl-N-(4-chlorophenyl)]amino]sulfonyl-N-[3-[2-[2-(4-trifluoromethylphenyl)-5-methyloxazol-4-yl]ethoxy]benzyl]amino]acetic acid

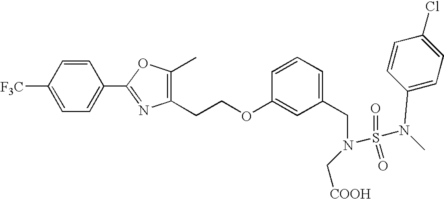

### Step 1: Preparation of [N—[[N-methyl-N-(4-chlorophenyl)]amino]sulfonyl-N-[3-[2-[2-(4-trifluoromethylphenyl)-5-methyloxazol-4-yl]ethoxy]benzyl]amino]acetic acid ethyl ester

The procedure of Step 1 of Example 61 was repeated except for using 413 mg (1 mmol) of the compound obtained in Step 2 of Example 18, 407 mg (1.5 mmol) of 2-[5-methyl-2-(4-trifluoromethylphenyl)oxazol-4-yl]ethanol, 446 mg (1.7 mmol) of triphenylphosphine and 344 mg (1.7 mmol) of diisopropyl azocarboxylate to obtain the title compound (613 mg, 92%).

¹H-NMR (CDCl₃, 300 MHz): δ (ppm) 8.07 (d, J=8.4 Hz, 2H), 7.67 (d, J=8.4 Hz, 2H), 7.41-7.30 (m, 4H), 7.23-7.18 (m, 1H), 6.84-6.78 (m, 3H), 4.44 (s, 2H), 4.21 (t, J=6.6 Hz, 2H), 4.14 (q, J=7.2 Hz, 2H), 3.84 (s, 2), 3.27 (s, 3H), 2.97 (t, J=6.6 Hz, 2H), 2.39 (s, 3H), 1.23 (t, J=7.2 Hz, 3H).

### Step 2: Preparation of [N—[[N-methyl-N-(4-chlorophenyl)]amino]sulfonyl-N-[3-[2-[2-(4-trifluoromethylphenyl)-5-methyloxazol-4-yl]ethoxy]benzyl]amino]acetic acid

The procedure of Step 5 of Example 3 was repeated except for using 613 mg (0.92 mmol) of the compound obtained in Step 1 and 59 mg (1.4 mmol) of lithium hydroxide monohydrate to obtain the title compound (581 mg, 99%).

¹H-NMR (CDCl₃, 300 MHz): δ (ppm) 8.06 (d, J=8.4 Hz, 2H), 7.67 (d, J=8.4 Hz, 2H), 7.38-7.17 (m, 6H), 6.89-6.78 (m, 3H), 4.44 (s, 2H), 4.21 (t, J=6.6 Hz, 2H), 3.87 (s, 2H), 3.26 (s, 3H), 2.96 (t, J=6.6 Hz, 2H), 2.39 (s, 3H).

### EXAMPLE 80

### Preparation of [N—(N-ethyl-N-m-tolylamino)sulfonyl-N-[3-[2-[2-(4-trifluoromethylphenyl)-5-methyloxazol-4-yl]ethoxy]benzyl]amino]acetic acid

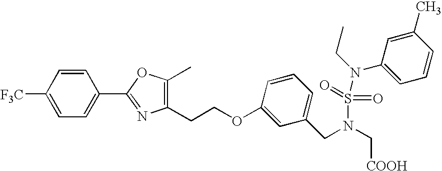

### Step 1: Preparation of [N—(N-ethyl-N-m-tolylamino)sulfonyl-N-[3-[2-[2-(4-trifluoromethylphenyl)-5-methyloxazol-4-yl]ethoxy]benzyl]amino]acetic acid ethyl ester

The procedure of Step 2 of Example 3 was repeated except for using 462 mg (1 mmol) of [N-[3-[2-[2-(4-trifluoromethylphenyl)-5-methyloxazol-4-yl]ethoxy]benzyl]amino]acetic acid ethyl ester, 257 mg (1.1 mmol) of (N-ethyl-N-m-tolylamino)sulfonyl chloride and 111 mg (1.1 mmol) of triethylamine to obtain the title compound (270 mg, 41%).

¹H-NMR (CDCl₃, 200 MHz): δ (ppm) 8.08 (d, J=8.1 Hz, 2H), 7.67 (d, J=8.1 Hz, 2H), 7.25-7.12 (m, 5H), 6.83-6.76 (m, 3H), 4.47 (s, 2H), 4.24-4.08 (m, 4H), 3.81 (s, 2H), 3.69 (q, J=7.2 Hz, 2H), 2.97 (t, J=6.5 Hz, 2H), 2.39 (s, 3H), 2.35 (s, 3H), 1.22 (t, J=7.2 Hz, 3H), 1.07 (t, J=7.2 Hz, 3H).

### Step 2: Preparation of [N—(N-ethyl-N-m-tolylamino)sulfonyl-N-[3-[2-[2-(4-trifluoromethylphenyl)-5-methyloxazol-4-yl]ethoxy]benzyl]amino]acetic acid

The procedure of Step 5 of Example 3 was repeated except for using 270 mg (0.41 mmol) of the compound obtained in Step 1 and 26 mg (0.62 mmol) of lithium hydroxide monohydrate to obtain the title compound (256 mg, 99%).

¹H-NMR (CDCl₃, 200 MHz): δ (ppm) 8.07 (d, J=8.1 Hz, 2H), 7.95 (br s, 1H), 7.68 (d, J=8.1 Hz, 2H), 7.30-7.09 (m, 5H), 6.95-6.75 (m, 3H), 4.45 (s, 2H), 4.21 (t, J=6.5 Hz, 2H), 3.83 (s, 2H), 3.71 (q, J=7.2 Hz, 2H), 2.94 (t, J=6.5 Hz, 2H), 2.39 (s, 3H), 2.34 (s, 3H), 1.08 (t, J=7.2 Hz, 3H).

### EXAMPLE 81

### Preparation of [N—(N-anisoyl-N-methylamino)sulfonyl-N-[3-[2-[2-(4-trifluoromethylphenyl)-5-methyloxazol-4-yl]ethoxy]benzyl]amino]acetic acid

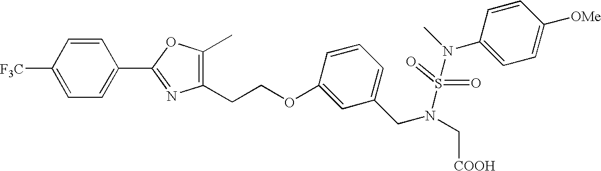

### Step 1: Preparation of [N—(N-anisoyl-N-methylamino)sulfonyl-N-[3-[2-[2-(4-trifluoromethylphenyl)-5-methyloxazol-4-yl]ethoxy]benzyl]amino]acetic acid ethyl ester

The procedure of Step 2 of Example 3 was repeated except for using 462 mg (1 mmol) of [N-[3-[2-[2-(4-trifluoromethylphenyl)-5-methyloxazol-4-yl]ethoxy]benzyl]amino]acetate ethyl ester, 259 mg (1.1 mmol) of (N-anisoyl-N-methylamino)sulfonyl chloride and 111 mg (1.1 mmol) of triethylamine to obtain the title compound (351 mg, 53%).

¹H-NMR (CDCl₃, 200 MHz): δ (ppm) 8.08 (d, J=8.1 Hz, 2H), 7.67 (d, J=8.1 Hz, 2H), 7.38 (d, J=8.9 Hz, 2H), 7.24-7.16 (m, 1H), 6.89-6.79 (m, 5H), 4.45 (s, 2H), 4.25-4.09 (m, 4H), 3.84 (s, 2H), 3.79 (s, 3H), 3.25 (s, 3H), 2.97 (t, J=6.5 Hz, 2H), 2.39 (s, 3H), 1.23 (t, J=7.2 Hz, 3H).

### Step 2: Preparation of [N—(N-anisoyl-N-methylamino)sulfonyl-N-[3-[2-[2-(4-trifluoromethylphenyl)-5-methyloxazol-4-yl]ethoxy]benzyl]amino]acetic acid

The procedure of Step 5 of Example 3 was repeated except for using 351 mg (0.53 mmol) of the compound obtained in Step 1 and 34 mg (0.8 mmol) of lithium hydroxide monohydrate to obtain the title compound (332 mg, 99%).

¹H-NMR (CDCl₃, 200 MHz): δ (ppm) 8.07 (d, J=8.1 Hz, 2H), 7.68 (d, J=8.1 Hz, 2H), 7.40-7.16 (m, 4H), 6.99-6.80 (m, 5H), 4.45 (s, 2H), 4.23 (t, J=6.5 Hz, 2H), 3.85 (s, 2H), 3.78 (s, 3H), 3.25 (s, 3H), 2.94 (t, J=6.5 Hz, 2H), 2.39 (s, 3H).

### EXAMPLE 82

### Preparation of [N—[N-(3-fluorophenyl)-N-methylamino]sulfonyl-N-[3-[2-[2-(4-trifluoromethylphenyl)-5-methyloxazol-4-yl]ethoxy]benzyl]amino]acetic acid

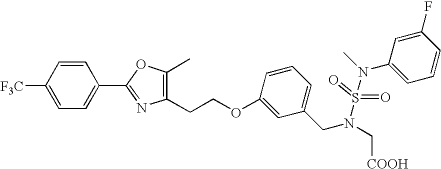

### Step 1: Preparation of [N—[N-(3-fluorophenyl)-N-methylamino]sulfonyl-N-[3-[2-[2-(4-trifluoromethylphenyl)-5-methyloxazol-4-yl]ethoxy]benzyl]amino]acetic acid ethyl ester

The procedure of Step 2 of Example 3 was repeated except for using 462 mg (1 mmol) of [N-[3-[2-[2-(4-trifluoromethylphenyl)-5-methyloxazol-4-yl]ethoxy]benzyl]amino]acetic acid ethyl ester, 246 mg (1.1 mmol) of [N-(3-fluorophenyl)-N-methylamino]sulfonyl chloride and 111 mg (1.1 mmol) of triethylamine to obtain the title compound (266 mg, 41%).

¹H-NMR (CDCl₃, 200 MHz): δ (ppm) 8.80 (d, J=8.1 Hz, 2H), 7.67 (d, J=8.1 Hz, 2H), 7.33-7.16 (m, 4H), 6.96-6.91 (m, 1H), 6.85-6.79 (m, 3H), 4.46 (s, 2H), 4.24-4.09 (m, 4H), 3.83 (s, 2H), 3.29 (s, 3H), 2.97 (t, J=6.5 Hz, 2H), 2.39 (s, 3H), 1.23 (t, J=7.2 Hz, 3H).

### Step 2: Preparation of [N—[N-(3-fluorophenyl)-N-methylamino]sulfonyl-N-[3-[2-[2-(4-trifluoromethylphenyl)-5-methyloxazol-4-yl]ethoxy]benzyl]amino]acetic acid

The procedure of Step 5 of Example 3 was repeated except for using 266 mg (0.41 mmol) of the compound obtained in Step 1 and 26 mg (0.62 mmol) of lithium hydroxide monohydrate to obtain the title compound (252 mg, 99%).

¹H-NMR (CDCl₃, 200 MHz): δ (ppm) 8.07 (d, J=8.1 Hz, 2H), 7.68 (d, J=8.1 Hz, 2H), 7.33-7.14 (m, 4H), 7.00-6.79 (m, 5H), 4.46 (s, 2H), 4.22 (t, J=6.5 Hz, 2H), 3.88 (s, 2H), 3.30 (s, 3H), 2.95 (t, J=6.5 Hz, 2H), 2.40 (s, 3H).

### EXAMPLE 83

### Preparation of [N—[[N-methyl-N-(4-chlorophenyl)]amino]sulfonyl-N-[3-[2-[2-(thiophen-2-yl)-5-methyloxazol-4-yl]ethoxy]benzyl]amino]acetic acid

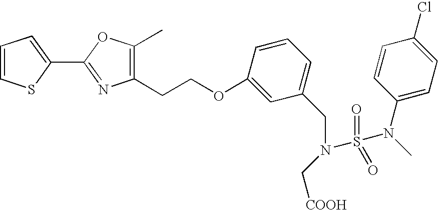

### Step 1 Preparation of [N—[[N-methyl-N-(4-chlorophenyl)]amino]sulfonyl-N-[3-[2-[2-(thiophen-2-yl)-5-methyloxazol-4-yl]ethoxy]benzyl]amino]acetic acid ethyl ester

The procedure of Step 1 of Example 61 was repeated except for using 413 mg (1 mmol) of the compound obtained in Step 2 of Example 18, 314 mg (1.5 mmol) of 2-[5-methyl-2-(thiophen-2-yl)oxazol-4-yl]ethanol, 446 mg (1.7 mmol) of triphenylphosphine and 344 mg (1.7 mmol) of diisopropyl azocarboxylate to obtain the title compound (556 mg, 92%).

¹H-NMR (CDCl₃, 300 MHz): δ (ppm) 7.58-7.57 (m, 1H), 7.41-7.20 (m, 6H), 7.08-7.06 (m, 1H), 6.82-6.78 (m, 3H), 4.44 (s, 2H), 4.20-4.11 (m, 4H), 3.84 (s, 2H), 3.27 (s, 3H), 2.94 (t, J=6.6 Hz, 2H), 2.35 (s, 3H), 1.23 (t, J=7.2 Hz, 3H).

### Step 2: Preparation of [N—[[N-methyl-N-(4-chlorophenyl)]amino]sulfonyl-N-[3-[2-[2-(thiophen-2-yl)-5-methyloxazol-4-yl]ethoxy]benzyl]amino]acetic acid

The procedure of Step 5 of Example 3 was repeated except for using 556 mg (0.92 mmol) of the compound obtained in Step 1 and 59 mg (1.4 mmol) of lithium hydroxide monohydrate to obtain the title compound (525 mg, 99%).

¹H-NMR (CDCl₃, 300 MHz): δ (ppm) 7.63-7.61 (m, 1H), 7.45 (br s, 1H), 7.39-7.19 (m, 6H), 7.07-7.06 (m, 1H), 6.89-6.79 (m, 3H), 4.44 (s, 2H), 4.18 (t, J=6.6 Hz, 2H), 3.89 (s, 2H), 3.27 (s, 3H), 2.91 (t, J=6.6 Hz, 2H), 2.35 (s, 3H).

### EXAMPLE 84

### Preparation of [N-(pyrrolidinyl)sulfonyl-N-[3-[2-[2-(4-methylphenyl)-5-methyloxazol-4-yl]ethoxy]benzyl]amino]acetic acid

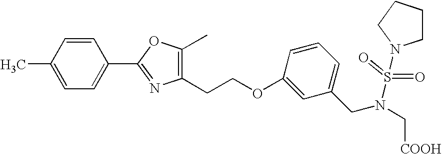

### Step 1: Preparation of [N-(pyrrolidinyl)sulfonyl-N-[3-[2-[2-(4-methylphenyl)-5-methyloxazol-4-yl]ethoxy]benzyl]amino]acetate methyl ester

The procedure of Step 1 of Example 61 was repeated except for using 328 mg (1 mmol) of [N-(pyrrolidinyl)sulfonyl-N-(3-benzyloxybenzyl)]amino]acetate methyl ester, 326 mg (1.5 mmol) of 2-[5-methyl-2-(4-methylphenyl)oxazol-4-yl]ethanol, 446 mg (1.7 mmol) of triphenylphosphine and 344 mg (1.7 mmol) of diisopropyl azocarboxylate to obtain the title compound (470 mg, 89%).

¹H-NMR (CDCl₃, 200 MHz): δ (ppm) 7.86 (d, J=8.3 Hz, 2H), 7.27-7.19 (m, 3H), 6.90-6.82 (m, 3H), 4.51 (s, 2H), 4.23 (t, J=6.7 Hz, 2H), 3.87 (s, 2H), 3.69 (s, 3H), 3.39-3.32 (m, 4H), 2.97 (t, J=6.7 Hz, 2H), 2.38 (s, 3H), 2.37 (s, 3H), 1.92-1.86 (m, 4H).

### Step 2: Preparation of [N-(pyrrolidinyl)sulfonyl-N-[3-[2-[2-(4-methylphenyl)-5-methyloxazol-4-yl]ethoxy]benzyl]amino]acetic acid

The procedure of Step 5 of Example 3 was repeated except for using 470 mg (0.89 mmol) of the compound obtained in Step 1 and 59 mg (1.4 mmol) of lithium hydroxide monohydrate to obtain the title compound (453 mg, 99%).

¹H-NMR (CDCl₃, 300 MHz): δ (ppm) 7.86 (d, J=8.1 Hz, 2H), 7.25 (d, J=8.1 Hz, 2H), 7.24-7.20 (m, 1H), 6.92-6.84 (m, 3H), 5.42 (s, 1H), 4.51 (s, 2H), 4.26 (t, J=7.5 Hz, 2H), 3.92 (s, 2H), 3.96-3.35 (m, 4H), 2.91 (t, J=7.5 Hz, 2H), 2.39 (s, 3H), 2.37 (s, 3H), 1.88-1.81 (m, 4H).

### EXAMPLE 85

### Preparation of [N-(pyrrolidinyl)sulfonyl-N-[3-[2-[2-(4-tri fluoromethylphenyl)-5-methyloxazol-4-yl]ethoxy]benzyl]amino]acetic acid

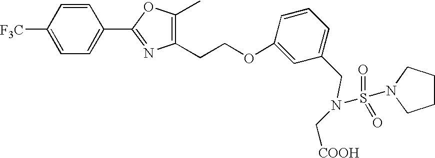

### Step 1: Preparation of [N-(pyrrolidinyl)sulfonyl-N-[3-[2-[2-(4-trifluoromethyl phenyl)-5-methyloxazol-4-yl]ethoxy]benzyl]amino]acetic acid ethyl ester

The procedure of Step 1 of Example 61 was repeated except for using 342 mg (1 mmol) of [N-(pyrrolidinyl)sulfonyl-N-(3-benzyloxybenzyl)]amino]acetic acid ethyl ester, 407 mg (1.5 mmol) of 2-[5-methyl-2-[(4-trifluoromethyl)phenyl]oxazol-4-yl]ethanol, 446 mg (1.7 mmol) of triphenylphosphine and 344 mg (1.7 mmol) of diisopropyl azocarboxylate to obtain the title compound (524 mg, 88%).

¹H-NMR (CDCl₃, 200 MHz): δ (ppm) 8.08 (d, J=7.8 Hz, 2H), 7.68 (d, J=7.8 Hz, 2H), 7.27-7.19 (m, 1H), 6.90-6.81 (m, 3H), 4.52 (s, 2H), 4.24 (t, J=6.5 Hz, 2H), 4.18 (q, J=7.2 Hz, 2H), 3.85 (s, 2H), 3.40-3.27 (m, 4H), 2.99 (t, J=6.5 Hz, 2H), 2.41 (s, 3H), 1.94-1.86 (m, 4H), 1.23 (t, J=7.3 Hz, 3H).

### Step 2: Preparation of [N-(pyrrolidinyl)sulfonyl-N-[3-[2-[2-(4-trifluoromethyl phenyl)-5-methyloxazol-4-yl]ethoxy]benzyl]amino]acetic acid

The procedure of Step 5 of Example 3 was repeated except for using 524 mg (0.88 mmol) of the compound obtained in Step 1 and 59 mg (1.4 mmol) of lithium hydroxide monohydrate to obtain the title compound (495 mg, 99%).

¹H-NMR (CDCl₃, 300 MHz): δ (ppm) 8.09 (d, J=8.3 Hz, 2H), 7.69 (d, J=8.3 Hz, 2H), 7.26-7.21 (m, 2H), 7.04 (s, 1H), 6.88-6.83 (m, 3H), 5.04 (br s, 1H), 4.50 (s, 2H), 4.25 (t, J=7.2 Hz, 2H), 3.92 (s, 2H), 3.38-3.29 (m, 4H), 2.96 (t, J=7.1 Hz, 2H), 2.41 (s, 3H), 1.93-1.85 (m, 4H).

### EXAMPLE 86

### Preparation of [N-(morpholinyl)sulfonyl-N-[3-[2-[2-(4-methylphenyl)-5-methyloxazol-4-yl]ethoxy]benzyl]amino]acetic acid

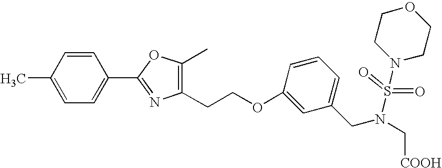

### Step 1: Preparation of [N-(morpholinyl)sulfonyl-N-[3-[2-[2-(4-methylphenyl)-5-methyloxazol-4-yl]ethoxy]benzyl]amino]acetic acid ethyl ester

The procedure of Step 1 of Example 61 was repeated except for using 344 mg (1 mmol) of N-(morpholinyl)sulfonyl-N-(3-hydroxybenzyl)]amino]acetate methyl ester, 326 mg (1.5 mmol) of 2-[5-methyl-2-(4-methylphenyl)oxazol-4-yl]ethanol, 446 mg (1.7 mmol) of triphenylphosphine and 344 mg (1.7 mmol) of diisopropyl azocarboxylate to obtain the title compound (473 mg, 87%).

¹H-NMR (CDCl₃, 200 MHz): δ (ppm) 7.85 (d, J=8.4 Hz, 2H), 7.26-7.20 (m, 3H), 6.89-6.83 (m, 3H), 4.51 (s, 2H), 4.23 (t, J=6.7 Hz, 2H), 3.86 (s, 2H), 3.73-3.71 (m, 4H), 3.70 (s, 3H), 3.30-3.26 (m, 4H), 3.76 (t, J=6.7 Hz, 2H), 2.38 (s, 3H), 2.36 (s, 3H).

### Step 2: Preparation of [N-(morpholinyl)sulfonyl-N-[3-[2-[2-(4-methylphenyl)-5-methyloxazol-4-yl]ethoxy]benzyl]amino]acetic acid

The procedure of Step 5 of Example 3 was repeated except for using 473 mg (0.87 mmol) of the compound obtained in Step 1 and 59 mg (1.4 mmol) of lithium hydroxide monohydrate to obtain the title compound (456 mg, 99%).

¹H-NMR (CDCl₃, 300 MHz): δ (ppm) 7.86 (d, J=8.1 Hz, 2H), 7.25 (d, J=8.1 Hz, 2H), 7.24-7.22 (m, 1H), 6.94 (s, 1H), 6.91-6.86 (m, 2H), 4.52 (s, 2H), 4.50 (br s, 1H), 4.25 (t, J=7.8 Hz, 2H), 3.92 (s, 2H), 3.69-3.66 (m, 4H), 3.33-3.29 (m, 4H), 2.91 (t, J=7.8 Hz, 2H), 2.39 (s, 3H), 2.38 (s, 3H).

### EXAMPLE 87

### Preparation of [N-(morpholinyl)sulfonyl-N-[3-[2-[2-(4-tri fluoromethylphenyl)-5-methyloxazol-4-yl]ethoxy]benzyl]amino]acetic acid

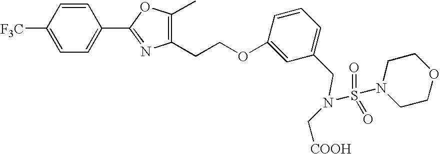

### Step 1: Preparation of [N-(morpholinyl)sulfonyl-N-[3-[2-[2-(4-trifluoromethyl phenyl)-5-methyloxazol-4-yl]ethoxy]benzyl]amino]acetic acid ethyl ester

The procedure of Step 1 of Example 61 was repeated except for using 358 mg (1 mmol) of [N-(morpholinyl)sulfonyl-N-(3-hydroxybenzyl)]amino]acetate ethyl ester, 407 mg (1.5 mmol) of 2-[5-methyl-2-[(4-trifluoromethyl)phenyl]oxazol-4-yl]ethanol, 446 mg (1.7 mmol) of triphenylphosphine and 344 mg (1.7 mmol) of diisopropyl azocarboxylate to obtain the title compound (538 mg, 88%).

¹H-NMR (CDCl₃, 200 MHz): δ (ppm) 8.08 (d, J=8.1 Hz, 2H), 7.68 (d, J=8.1 Hz, 2H), 7.28-7.20 (m, 1H), 6.88-6.83 (m, 3H), 4.53 (s, 2H), 4.25 (t, J=6.4 Hz, 2H), 4.16 (q, J=7.2 Hz, 2H), 3.85 (s, 2H), 3.74-3.69 (m, 4H), 3.31-3.26 (m, 4H), 2.99 (t, J=6.4 Hz, 2H), 2.41 (s, 3H), 1.24 (t, J=7.2 Hz, 3H).

### Step 2: Preparation of [N-(morpholinyl)sulfonyl-N-[3-[2-[2-(4-trifluoromethylphenyl)-5-methyloxazol-4-yl]ethoxy]benzyl]amino]acetic acid

The procedure of Step 5 of Example 3 was repeated except for using 538 mg (0.88 mmol) of the compound obtained in Step 1 and 59 mg (1.4 mmol) of lithium hydroxide monohydrate to obtain the title compound (508 mg, 99%).

¹H-NMR (CDCl₃, 300 MHz): δ (ppm) 8.09 (d, J=8.1 Hz, 1H), 7.71 (d, J=8.1 Hz, 2H), 7.28-7.23 (m, 2H), 7.04 (s, 1H), 6.93-6.89 (m, 2H), 4.52 (s, 2H), 4.26 (t, J=7.4 Hz, 2H), 3.92 (s, 2H), 3.71-3.68 (m, 4H), 3.32-3.29 (m, 4H), 2.95 (t, J=7.4 Hz, 2H), 2.42 (s, 3H).

### EXAMPLE 88

### Preparation of [N-(4-methyl-1-piperazinyl)sulfonyl-N-[3-[2-[2-(4-methylphenyl)-5-methyloxazol-4-yl]ethoxy]benzyl]amino]acetic acid

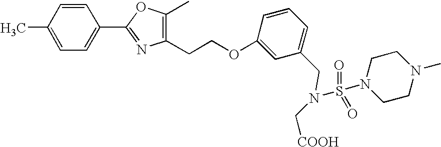

### Step 1: Preparation of [N-(4-methyl-1-piperazinyl)sulfonyl-N-[3-[2-[2-(4-methylphenyl)-5-methyloxazol-4-yl]ethoxy]benzyl]amino]acetate methyl ester

The procedure of Step 1 of Example 61 was repeated except for using 357 mg (1 mmol) of [N-(4-methyl-1-piperazinyl)sulfonyl-N-(3-hydroxybenzyl)]amino]acetate methyl ester, 326 mg (1.5 mmol) of 2-[5-methyl-2-(4-methylphenyl)oxazol-4-yl]ethanol, 446 mg (1.7 mmol) of triphenylphosphine and 344 mg (1.7 mmol) of diisopropyl azocarboxylate to obtain the title compound (507 mg, 91%).

¹H-NMR (CDCl₃, 200 MHz): δ (ppm) 7.86 (d, J=8.1 Hz, 2H), 7.27-7.23 (m, 1H), 7.22 (d, J=8.1 Hz, 2H), 6.89-6.83 (m, 3H), 4.49 (s, 2H), 4.23 (t, J=6.6 Hz, 2H), 3.84 (s, 2H), 3.70 (s, 3H), 3.35-3.32 (m, 4H), 2.97 (t, J=6.6 Hz, 2H), 2.47-2.43 (m, 4H), 2.38 (s. 3H), 2.36 (s, 3H), 2.29 (s, 3H).

### Step 2: Preparation of [N-(4-methyl-1-piperazinyl)sulfonyl-N-[3-[2-[2-(4-methylphenyl)-5-methyloxazol-4-yl]ethoxy]benzyl]amino]acetic acid

The procedure of Step 5 of Example 3 was repeated except for using 507 mg (0.91 mmol) of the compound obtained in Step 1 and 59 mg (1.4 mmol) of lithium hydroxide monohydrate to obtain the title compound (489 mg, 99%).

¹H-NMR (DMSO-d₆, 200 MHz): δ (ppm) 7.99 (d, J=8.1 Hz, 2H), 7.49 (d, J=8.1 Hz, 2H), 7.45-7.42 (m, 1H), 7.08-7.04 (m, 3H), 4.61 (s, 2H), 4.39 (t, J=6.6 Hz, 2H), 3.91 (s, 2H), 3.33-3.30 (m, 4H), 3.11 (t, J=6.6 Hz, 2H), 2.69 (s, 3H), 2.54 (s, 3H), 2.53-2.50 (m, 4H), 2.34 (s, 3H).

### EXAMPLE 89

### Preparation of [N-(4-methyl-1-piperazinyl)sulfonyl-N-[3-[2-[2-(4-trifluoromethylphenyl)-5-methyloxazol-4-yl]ethoxy]benzyl]amino]acetic acid

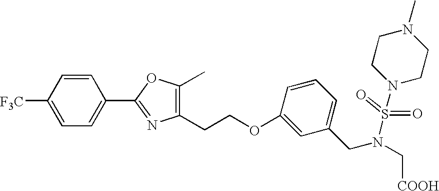

### Step 1: Preparation of [N-(4-methyl-1-piperazinyl)sulfonyl-N-[3-[2-[2-(4-trifluoromethylphenyl)-5-methyloxazol-4-yl]ethoxy]benzyl]amino]acetic acid ethyl ester

The procedure of Step 1 of Example 61 was repeated except for using 371 mg (1 mmol) of [N-(4-methyl-1-piperazinyl)sulfonyl-N-(3-hydroxybenzyl)]amino]acetate ethyl ester, 407 mg (1.5 mmol) of 2-[5-methyl-2-[(4-trifluoromethyl)phenyl]oxazol-4-yl]ethanol, 446 mg (1.7 mmol) of triphenylphosphine and 344 mg (1.7 mmol) of diisopropyl azocarboxylate to obtain the title compound (562 mg, 90%).

¹H-NMR (CDCl₃, 300 MHz): δ (ppm) 8.09 (d, J=8.1 Hz, 2H), 7.67 (d, J=8.1 Hz, 2H), 7.26-7.21 (m, 1H), 6.89-6.82 (m, 3H), 4.51 (s, 1H), 4.24 (t. J=6.6 Hz, 2H), 4.15 (q, J=7.2 Hz, 2H), 3.82 (s, 2H), 3.35-3.32 (m, 4H), 2.99 (t, J=6.6 Hz, 2H), 2.46-2.43 (m, 4H), 2.41 (s, 3H), 2.29 (s, 3H), 1.24 (t, J=7.2 Hz, 3H).

### Step 2: Preparation of [N-(4-methyl-1-piperazinyl)sulfonyl-N-[3-[2-[2-(4-trifluoromethylphenyl)-5-methyloxazol-4-yl]ethoxy]benzyl]amino]acetic acid

The procedure of Step 5 of Example 3 was repeated except for using 562 mg (0.91 mmol) of the compound obtained in Step 1 and 59 mg (1.4 mmol) of lithium hydroxide monohydrate to obtain the title compound (532 mg, 99%).

¹H-NMR (DMSO-d₆, 300 MHz): δ (ppm) 8.16 (d, J=8.2 Hz, 2H), 7.92 (d, J=8.2 Hz, 2H), 7.34-7.29 (m, 1H), 6.95-6.92 (m, 3H), 4.47 (s, 2H), 4.27 (t, J=6.6 Hz, 2H), 3.81 (s, 2H), 3.65 (br s, 1H), 3.23-3.19 (m, 4H), 3.02 (t, J=6.6 Hz, 2H), 2.55-2.50 (m, 4H), 2.45 (s, 3H), 2.30 (s, 3H).

### EXAMPLE 90

### Preparation of [N-(indolinyl)sulfonyl-N-[3-[2-(2-phenyl-5-methyloxazol-4-yl)ethoxy]benzyl]amino]acetic acid

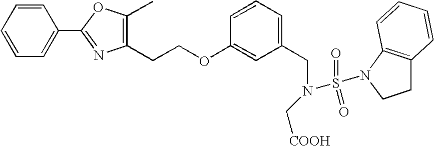

### Step 1: Preparation of [N-(indolinyl)sulfonyl-N-[3-[2-(2-phenyl-5-methyloxazol-4-yl)ethoxy]benzyl]amino]acetic acid ethyl ester

The procedure of Step 1 of Example 61 was repeated except for using 390 mg (1 mmol) of the compound obtained in Step 2 of Example 30, 305 mg (1.5 mmol) of 2-[(5-methyl-2-phenyl)oxazol-4-yl]ethanol, 446 mg (1.7 mmol) of triphenylphosphine and 344 mg (1.7 mmol) of diisopropyl azocarboxylate to obtain the title compound (553 mg, 96%).

¹H-NMR (CDCl₃, 200 MHz): δ (ppm) 8.02-7.99 (m, 2H), 7.43-7.41 (m, 4H), 7.25-7.12 (m, 3H), 6.95-6.82 (m, 4H), 4.88 (s, 2H), 4.62 (s, 2H), 4.11-3.89 (m, 6H), 3.12 (t, J=8.5 Hz, 2H), 2.43 (s, 3H), 1.12 (t, J=7.2 Hz, 3H).

### Step 2: Preparation of [N-(indolinyl)sulfonyl-N-[3-[2-(2-phenyl-5-methyloxazol-4-yl)ethoxy]benzyl]amino]acetic acid

The procedure of Step 5 of Example 3 was repeated except for using 553 mg (0.96 mmol) of the compound obtained in Step 1 and 59 mg (1.4 mmol) of lithium hydroxide monohydrate to obtain the title compound (520 mg, 99%).

¹H-NMR (CDCl₃, 200 MHz): δ (ppm) 8.00-7.96 (m, 2H), 7.45-7.39 (m, 4H), 7.14-7.08 (m, 3H), 6.91-6.88 (m, 4H), 5.29 (s, 1H), 4.85 (s, 2H), 4.47 (s, 2H), 3.96 (t, J=8.5 Hz, 2H), 3.93 (s, 2H), 3.05 (t, J=8.5 Hz, 2H), 2.43 (s, 3H).

### EXAMPLE 91

### Preparation of [N-(indolinyl)sulfonyl-N-[3-[2-[2-(4-methyl phenyl)-5-methyloxazol-4-yl]ethoxy]benzyl]amino]acetic acid

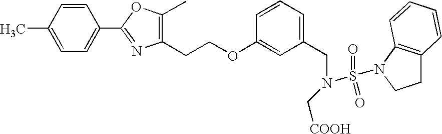

### Step 1: Preparation of [N-(indolinyl)sulfonyl-N-[3-[2-[2-(4-methylphenyl)-5-methyloxazol-4-yl]ethoxy]benzyl]amino]acetic acid ethyl ester

The procedure of Step 1 of Example 61 was repeated except for using 390 mg (1 mmol) of the compound obtained in Step 2 of Example 30, 326 mg (1.5 mmol) of 2-[5-methyl-2-(4-methylphenyl)oxazol-4-yl]ethanol, 446 mg (1.7 mmol) of triphenylphosphine and 344 mg (1.7 mmol) of diisopropyl azocarboxylate to obtain the title compound (554 mg, 94%).

¹H-NMR (CDCl₃, 200 MHz): δ (ppm) 7.90 (d, J=8.1 Hz, 2H), 7.41 (d, J=7.9 Hz, 1H), 7.25-7.13 (m, 5H), 6.95-6.82 (m, 4H), 4.88 (s, 2H), 4.62 (s, 2H), 4.10-3.94 (m, 4H), 3.88 (s, 2H), 3.12 (t, J=8.5 Hz, 2H), 2.42 (s, 3H), 2.39 (s, 3H), 1.25 (t, J=7.2 Hz, 3H).

### Step 2: Preparation of [N-(indolinyl)sulfonyl-N-[3-[2-[2-(4-methylphenyl)-5-methyloxazol-4-yl]ethoxy]benzyl]amino]acetic acid

The procedure of Step 5 of Example 3 was repeated except for using 554 mg (0.94 mmol) of the compound obtained in Step 1 and 59 mg (1.4 mmol) of lithium hydroxide monohydrate to obtain the title compound (523 mg, 99%).

¹H-NMR (CDCl₃, 200 MHz): δ (ppm) 7.86 (d, J=8.1 Hz, 2H), 7.42 (d, J=7.9 Hz, 1H), 7.25-7.08 (m, 5H), 6.94-6.76 (m, 4H), 5.37 (s, 1H), 4.87 (s, 2H), 4.54 (s, 2H), 4.00 (t, J=8.5 Hz, 2H), 3.92 (s, 2H), 3.04 (t, J=8.5 Hz, 2H), 2.41 (s, 3H), 2.39 (s, 3H).

### EXAMPLE 92

### Preparation of [N-(indolinyl)sulfonyl-N-[3-[2-[2-(4-trifluoro methylphenyl)-5-methyloxazol-4-yl]ethoxy]benzyl]amino]acetic acid

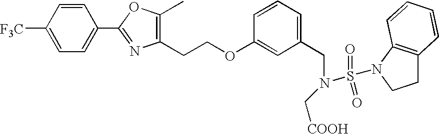

### Step 1: Preparation of [N-(indolinyl)sulfonyl-N-[3-[2-[2-(4-trifluoromethylphenyl)-5-methyloxazol-4-yl]ethoxy]benzyl]amino]acetic acid ethyl ester

The procedure of Step 1 of Example 61 was repeated except for using 390 mg (1 mmol) of the compound obtained in Step 2 of Example 30, 407 mg (1.5 mmol) of 2-[5-methyl-2-[(4-trifluoromethyl)phenyl]oxazol-4-yl]ethanol, 446 mg (1.7 mmol) of triphenylphosphine and 344 mg (1.7 mmol) of diisopropyl azocarboxylate to obtain the title compound (592 mg, 92%).

¹H-NMR (CDCl₃, 200 MHz): δ (ppm) 8.13 (d, J=8.1 Hz, 2H), 7.70 (d, J=8.1 Hz, 2H), 7.41 (d, J=9.1 Hz, 1H), 7.25-7.14 (m, 3H), 6.97-6.84 (m, 4H), 4.91 (s, 2H), 4.63 (s, 2H), 4.07 (t, J=8.5 Hz, 2H), 3.93 (q, J=7.2 Hz, 2H), 3.89 (s, 2H), 3.13 (t, J=8.5 Hz, 2H), 2.46 (s, 3H), 1.12 (t, J=7.2 Hz, 3H).

### Step 2: Preparation of [N-(indolinyl)sulfonyl-N-[3-[2-[2-(4-trifluoromethyl phenyl)-5-methyloxazol-4-yl]ethoxy]benzyl]amino]acetic acid

The procedure of Step 5 of Example 3 was repeated except for using 592 mg (0.92 mmol) of the compound obtained in Step 1 and 59 mg (1.4 mmol) of lithium hydroxide monohydrate to obtain the title compound (561 mg, 99%).

¹H-NMR (CDCl₃, 200 MHz): δ (ppm) 8.10 (d, J=8.7 Hz, 2H), 7.75 (d, J=8.7 Hz, 2H), 7.39 (d, J=9.1 Hz, 1H), 7.25-7.09 (m, 3H), 6.95-6.78 (m, 4H), 4.87 (s, 2H), 4.83 (s, 1H), 4.53 (s, 2H), 4.03 (t, J=8.3 Hz, 2H), 3.95 (s, 2H), 3.08 (t, J=8.3 Hz, 2H), 2.45 (s, 3H).

### EXAMPLE 93

### Preparation of [N-(indolinyl)sulfonyl-N-[3-[2-[2-(thiophen-2-yl)-5-methyloxazol-4-yl]ethoxy]benzyl]amino]acetic acid

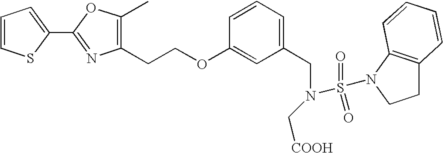

### Step 1: Preparation of [N-(indolinyl)sulfonyl-N-[3-[2-[2-(thiophen-2-yl)-5-methyloxazol-4-yl]ethoxy]benzyl]amino]acetic acid ethyl ester

The procedure of Step 1 of Example 61 was repeated except for using 390 mg (1 mmol) of the compound obtained in Step 2 of Example 30, 314 mg (1.5 mmol) of 2-[5-methyl-2-(thiophen-2-yl)oxazol-4-yl]ethanol, 446 mg (1.7 mmol) of triphenylphosphine and 344 mg (1.7 mmol) of diisopropyl azocarboxylate to obtain the title compound (529 mg, 91%).

¹H-NMR (CDCl₃, 200 MHz): δ (ppm) 7.64-7.62 (m, 1H), 7.41-7.38 (m, 2H), 7.22-7.07 (m, 4H), 6.93-6.86 (m, 4H), 4.86 (s, 2H), 4.62 (s, 2H), 4.12-3.95 (m, 4H), 3.89 (s, 2H), 3.12 (t, J=8.5 Hz, 2H), 2.41 (s, 3H), 1.13 (t, J=7.2 Hz, 3H).

### Step 2: Preparation of [N-(indolinyl)sulfonyl-N-[3-[2-[2-(thiophen-2-yl)-5-methyloxazol-4-yl]ethoxy]benzyl]amino]acetic acid

The procedure of Step 5 of Example 3 was repeated except for using 529 mg (0.91 mmol) of the compound obtained in Step 1 and 59 mg (1.4 mmol) of lithium hydroxide monohydrate to obtain the title compound (499 mg, 99%).

¹H-NMR (CDCl₃, 200 MHz): δ (ppm) 7.66-7.63 (m, 1H), 7.44-7.39 (m, 2H), 7.19-7.07 (m, 4H), 6.95-6.77 (m, 4H), 5.29 (s, 1H), 4.83 (s, 2H), 4.50 (s, 2H), 4.02 (t, J=8.3 Hz, 2H), 3.94 (s, 2H), 3.07 (t, J=8.3 Hz, 2H), 2.40 (s, 3H).

### EXAMPLE 94

### Preparation of [N-(1,2,3,4-tetrahydroquinolinyl)sulfonyl-N-[3-[2-(2-phenyl-5-methyloxazol-4-yl)ethoxy]benzyl]amino]acetic acid

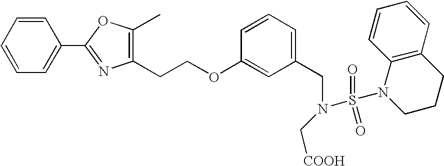

### Step 1: Preparation of [N-(1,2,3,4-tetrahydroquinolinyl)sulfonyl-N-[3-[2-(2-phenyl-5-methyloxazol-4-yl)ethoxy]benzyl]amino]acetic acid ethyl ester

The procedure of Step 1 of Example 61 was repeated except for using 404 mg (1 mmol) of the compound obtained in Step 2 of Example 34, 305 mg (1.5 mmol) of 2-[(5-methyl-2-phenyl)oxazol-4-yl]ethanol, 446 mg (1.7 mmol) of triphenylphosphine and 344 mg (1.7 mmol) of diisopropyl azocarboxylate to obtain the title compound (560 mg, 95%).

¹H-NMR (CDCl₃, 200 MHz): δ (ppm) 8.01-7.96 (m, 2H), 7.60 (d, J=7.9 Hz, 1H), 7.43-7.38 (m, 3H), 7.25-6.99 (m, 4H), 6.83-6.70 (m, 3H), 4.51 (s, 2H), 4.15-4.02 (m, 4H), 3.84 (s, 2H), 3.78 (t, J=6.8 Hz, 2H), 2.94 (t, J=6.6 Hz, 2H), 2.79 (t, J=6.8 Hz, 2), 2.37 (s, 3H), 2.07-2.01 (m, 2H), 1.18 (t, J=7.2 Hz, 3H).

### Step 2: Preparation of [N-(1,2,3,4-tetrahydroquinolinyl)sulfonyl-N-[3-[2-(2-phenyl-5-methyloxazol-4-yl)ethoxy]benzyl]amino]acetic acid

The procedure of Step 5 of Example 3 was repeated except for using 560 mg (0.95 mmol) of the compound obtained in Step 1 and 59 mg (1.4 mmol) of lithium hydroxide monohydrate to obtain the title compound (528 mg, 99%).

¹H-NMR (CDCl₃, 300 MHz): δ (ppm) 8.01-7.96 (m, 2H), 7.63 (d, J=7.9 Hz, 1H), 7.43-7.38 (m, 3H), 7.18-6.99 (m, 4H), 6.83-6.70 (m, 3H), 4.48 (s, 2H), 4.14 (t, J=6.6 Hz, 2H), 3.91 (s, 2H), 3.78 (t, J=5.8 Hz, 2H), 2.89 (t, J=6.6 Hz, 2H), 2.78 (t, J=6.8 Hz, 2H), 2.36 (s, 3H), 2.07-2.01 (m, 2H).

### EXAMPLE 95

### Preparation of [N-(1,2,3,4-tetraquinolinyl)sulfonyl-N-[3-[2-[2-(4-methylphenyl)-5-methyloxazol-4-yl]ethoxy]benzyl]amino]acetic acid

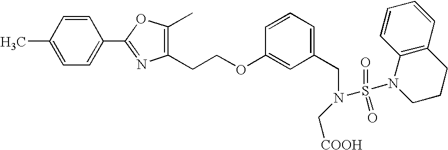

### Step 1: Preparation of [N-(1,2,3,4-tetraquinolinyl)sulfonyl-N-[3-[2-[2-(4-methyl phenyl)-5-methyloxazol-4-yl]ethoxy]benzyl]amino]acetic acid ethyl ester

The procedure of Step 1 of Example 61 was repeated except for using 404 mg (1 mmol) of the compound obtained in Step 2 of Example 34, 326 mg (1.5 mmol) of 2-[5-methyl-2-(4-methylphenyl)oxazol-4-yl]ethanol, 446 mg (1.7 mmol) of triphenylphosphine and 344 mg (1.7 mmol) of diisopropyl azocarboxylate to obtain the title compound (568 mg, 94%).

¹H-NMR (CDCl₃, 200 MHz): δ (ppm) 7.86 (d, J=8.3 Hz, 2H), 7.60 (d, J=8.3 Hz, 2H), 7.25-6.99 (m, 6H), 6.83-6.69 (m, 3H), 4.51 (s, 2H), 4.14-4.02 (m, 4H), 3.83 (s, 2H), 3.78 (t, J=5.8 Hz, 2H), 2.93 (t, J=6.6 Hz, 2H), 2.79 (t, J=6.8 Hz, 2H), 2.38 (s, 3H), 2.35 (s, 3H), 2.07-2.00 (m, 2H), 1.18 (t, J=7.2 Hz, 3H).

### Step 2: Preparation of [N-(1,2,3,4-tetraquinolinyl)sulfonyl-N-[3-[2-[2-(4-methyl phenyl)-5-methyloxazol-4-yl]ethoxy]benzyl]amino]acetic acid

The procedure of Step 5 of Example 3 was repeated except for using 568 mg (0.94 mmol) of the compound obtained in Step 1 and 59 mg (1.4 mmol) of lithium hydroxide monohydrate to obtain the title compound (523 mg, 99%).

¹H-NMR (CDCl₃, 300 MHz): δ (ppm) 7.83 (d, J=8.3 Hz, 2H), 7.63 (d, J=8.3 Hz, 1H), 7.36 (br s, 1H), 7.24-6.98 (m, 6H), 6.96 (s, 1H), 6.89-6.70 (m, 2H), 4.48 (s, 2H), 4.13 (t, J=6.6 Hz, 2H), 3.91 (s, 2H), 3.78 (t, J=5.8 Hz, 2H), 2.83 (t, J=6.6 Hz, 2H), 2.77 (t, J=6.8 Hz, 2H), 2.38 (s, 3H), 2.34 (s, 3H), 2.07-2.00 (m, 2H).

### EXAMPLE 96

### Preparation of [N-(1,2,3,4-tetrahydroquinolinyl)sulfonyl-N-[3-[2-[2-(4-trifluoromethylphenyl)-5-methyloxazol-4-yl]ethoxy]benzyl]amino]acetic acid

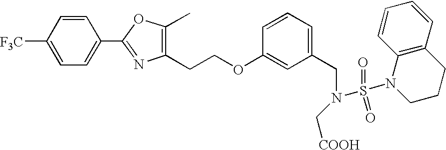

### Step 1: Preparation of [N-(1,2,3,4-tetrahydroquinolinyl)sulfonyl-N-[3-[2-[2-(4-trifluoromethylphenyl)-5-methyloxazol-4-yl]ethoxy]benzyl]amino]acetic acid ethyl ester

The procedure of Step 1 of Example 61 was repeated except for using 404 mg (1 mmol) of the compound obtained in Step 2 of Example 34, 407 mg (1.5 mmol) of 2-[5-methyl-2-[(4-trifluoromethyl)phenyl]oxazol-4-yl]ethanol, 446 mg (1.7 mmol) of triphenylphosphine and 344 mg (1.7 mmol) of diisopropyl azocarboxylate to obtain the title compound (605 mg, 92%).

¹H-NMR (CDCl₃, 200 MHz): δ (ppm) 8.09 (d, J=8.4 Hz, 2H), 7.68 (d, J=8.4 Hz, 2H), 7.61 (d, J=8.3 Hz, 1H), 7.22-7.00 (m, 4H), 6.83-6.72 (m, 3H), 4.52 (s, 2H), 4.16-4.02 (m, 4H), 3.84 (s, 2H), 3.78 (t, J=5.8 Hz, 2H), 2.96 (t, J=6.6 Hz, 2H), 2.81 (t, J=6.8 Hz, 2H), 2.39 (s, 3H), 2.08-2.02 (m, 2H), 1.18 (t, J=7.2 Hz, 3H).

### Step 2: Preparation of [N-(1,2,3,4-tetrahydroquinolinyl)sulfonyl-N-[3-[2-[2-(4-trifluoromethylphenyl)-5-methyloxazol-4-yl]ethoxy]benzyl]amino]acetic acid

The procedure of Step 5 of Example 3 was repeated except for using 605 mg (0.92 mmol) of the compound obtained in Step 1 and 59 mg (1.4 mmol) of lithium hydroxide monohydrate to obtain the title compound (573 mg, 99%).

¹H-NMR (CDCl₃, 300 MHz): δ (ppm) 8.04 (d, J=8.4 Hz, 2H), 7.67 (d, J=8.4 Hz, 2H), 7.62 (d, J=8.3 Hz, 1H), 7.19-7.00 (m, 4H), 6.97-6.71 (m, 3H), 6.53 (br s, 1H), 4.48 (s, 2H), 4.14 (t, J=6.6 Hz, 2H), 3.91 (s, 2H), 3.77 (t, 5.8 Hz, 2H), 2.93 (t, J=6.6 Hz, 2H), 2.79 (t, J=6.8 Hz, 2H), 2.39 (s, 3H), 2.08-2.02 (m, 2H).

### EXAMPLE 97

### Preparation of [N-(1,2,3,4-tetrahydroquinolinyl)sulfonyl-N-[3-[2-[2-(thiophen-2-yl)-5-methyloxazol-4-yl]ethoxy]benzyl]amino]acetic acid

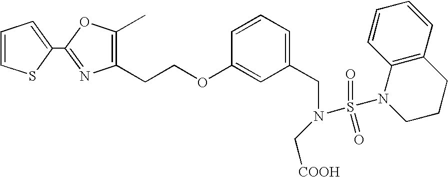

### Step 1: Preparation of [N-(1,2,3,4-tetrahydroquinolinyl)sulfonyl-N-[3-[2-[2-(thiophen-2-yl)-5-methyloxazol-4-yl]ethoxy]benzyl]amino]acetic acid ethyl ester

The procedure of Step 1 of Example 61 was repeated except for using 404 mg (1 mmol) of the compound obtained in Step 2 of Example 34, 314 mg (1.5 mmol) of 2-[5-methyl-2-(thiophen-2-yl)oxazol-4-yl]ethanol, 446 mg (1.7 mmol) of triphenylphosphine and 344 mg (1.7 mmol) of diisopropyl azocarboxylate to obtain the title compound (548 mg, 92%).

¹H-NMR (CDCl₃, 200 MHz): δ (ppm) 7.62-7.57 (m, 2H), 7.37-7.35 (m, 1H), 7.25-6.99 (m, 5H), 6.82-6.68 (m, 3H), 4.50 (s, 2H), 4.13-4.02 (m, 4H), 3.84 (s, 2H), 3.79 (d, J=5.8 Hz, 2H), 2.91 (t, J=6.6 Hz, 2H), 2.80 (t, J=6.8 Hz, 2H), 2.34 (s, 3H), 2.07-2.01 (m, 2H), 1.19 (t, J=7.2 Hz, 3H).

### Step 2: Preparation of [N-(1,2,3,4-tetrahydroquinolinyl)sulfonyl-N-[3-[2-[2-(thiophen-2-yl)-5-methyloxazol-4-yl]ethoxy]benzyl]amino]acetic acid

The procedure of Step 5 of Example 3 was repeated except for using 548 mg (0.92 mmol) of the compound obtained in Step 1 and 59 mg (1.4 mmol) of lithium hydroxide monohydrate to obtain the title compound (517 mg, 99%).

¹H-NMR (CDCl₃, 300 MHz): δ (ppm) 7.64-7.57 (m, 2H), 7.37-7.35 (m, 1H), 7.25-6.99 (m, 5H), 6.82-6.68 (m, 3H), 6.44 (br s, 1H), 4.48 (s, 2H), 4.12 (t, J=6.6 Hz, 2H), 3.91 (s, 2H), 3.79 (t, J=5.8 Hz, 2H), 2.87 (t, J=6.6 Hz, 2H), 2.78 (t, J=6.8 Hz, 2H), 2.34 (s, 3H), 2.07-2.01 (m, 2H).

### EXAMPLE 98

### Preparation of [N—(N,N-dimethylamino)sulfonyl-N-[4-[2-(2-phenyl-5-methyloxazol-4-yl)ethoxy]benzyl]amino]acetic acid

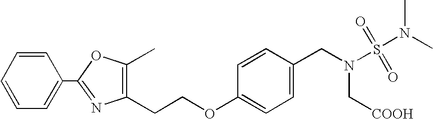

### Step 1: Preparation of [N—(N,N-dimethylamino)sulfonyl-N-[4-[2-(2-phenyl-5-methyloxazol-4-yl)ethoxy]benzyl]amino]acetic acid ethyl ester

The procedure of Step 1 of Example 61 was repeated except for using 316 mg (1 mmol) of the compound obtained in Step 3 of Example 38, 305 mg (1.5 mmol) of 2-[(5-methyl-2-phenyl)oxazol-4-yl]ethanol, 446 mg (1.7 mmol) of triphenylphosphine and 344 mg (1.7 mmol) of diisopropyl azocarboxylate to obtain the title compound (482 mg, 96%).

¹H-NMR (CDCl₃, 300 MHz): δ (ppm) 7.99-7.96 (m, 2H), 7.46-7.27 (m, 3H), 7.22 (d, J=8.7 Hz, 2H), 6.87 (d, J=8.7 Hz, 2H), 4.47 (s, 2H), 4.23 (t, J=7.5 Hz, 2H), 4.15 (q, J=7.2 Hz, 2H), 2.98 (t, J=7.5 Hz, 2H), 2.85 (s, 6H), 2.37 (s, 3H), 1.26 (t, J=7.1 Hz, 3H).

### Step 2: Preparation of [N—(N,N-dimethylamino)sulfonyl-N-[4-[2-(2-phenyl-5-methyloxazol-4-yl)ethoxy]benzyl]amino]acetic acid

The procedure of Step 5 of Example 3 was repeated except for using 482 mg (0.96 mmol) of the compound obtained in Step 1 and 59 mg (1.4 mmol) of lithium hydroxide monohydrate to obtain the title compound (450 mg, 99%).

¹H-NMR (CDCl₃, 200 MHz): δ (ppm) 7.98-7.92 (m, 2H), 7.68 (br s, 1H), 7.45-7.40 (m, 3H), 7.21 (d, J=8.5 Hz, 2H), 6.84 (d, J=8.5 Hz, 2H), 4.48 (s, 2H), 4.20 (t, J=6.5 Hz, 2H), 3.84 (s, 2H), 3.02 (t, J=6.5 Hz, 2H), 2.84 (s, 6H), 2.38 (s, 3H).

### EXAMPLE 99

### Preparation of [N—(N,N-dimethylamino)sulfonyl-N-[4-[2-[2-(4-methylphenyl)-5-methyloxazol-4-yl]ethoxy]benzyl]amino]acetic acid

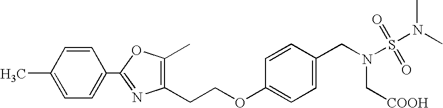

### Step 1: Preparation of [N—(N,N-dimethylamino)sulfonyl-N-[4-[2-[2-(4-methyl phenyl)-5-methyloxazol-4-yl]ethoxy]benzyl]amino]acetic acid ethyl ester

The procedure of Step 1 of Example 61 was repeated except for using 316 mg (1 mmol) of the compound obtained in Step 3 of Example 38, 326 mg (1.5 mmol) of 2-[5methyl-2-(4-methylphenyl)oxazol-4-yl]ethanol, 446 mg (1.7 mmol) of triphenylphosphine and 344 mg (1.7 mmol) of diisopropyl azocarboxylate to obtain the title compound (485 mg, 94%).

¹H-NMR (CDCl₃, 300 MHz): δ (ppm) 7.85 (d, J=8.1 Hz, 2H), 7.27-7.11 (m, 4H), 6.85 (d, J=8.6 Hz, 2H), 4.46 (s, 2H), 4.20 (t, J=6.5 Hz, 2H), 4.15 (q, J=7.2 Hz, 2H), 2.96 (t, J=6.5 Hz, 2H), 2.85 (s, 6H), 2.37 (s, 3H), 2.36 (s, 3H), 1.24 (t, J=7.2 Hz, 3H).

### Step 2: Preparation of [N—(N,N-dimethylamino)sulfonyl-N-[4-[2-[2-(4-methyl phenyl)-5-methyloxazol-4-yl]ethoxy]benzyl]amino]acetic acid

The procedure of Step 5 of Example 3 was repeated except for using 485 mg (0.94 mmol) of the compound obtained in Step 1 and 59 mg (1.4 mmol) of lithium hydroxide monohydrate to obtain the title compound (454 mg, 99%).

¹H-NMR (CDCl₃, 300 MHz): δ (ppm) 10.55 (br s, 1H), 7.83 (d, J=8.1 Hz, 2H), 7.26-7.11 (m, 4H), 6.77 (d, J=8.4 Hz, 2H), 4.48 (s, 2H), 4.18 (t, J=6.3 Hz, 2H), 3.84 (s, 2H), 3.02 (t, J=6.3 Hz, 2H), 2.84 (s, 6H), 2.38 (s, 6H).

### EXAMPLE 100

### Preparation of [N—(N-t-butylamino)sulfonyl-N-[4-[2-[2-(4-methylphenyl)-5-methyloxazol-4-yl]ethoxy]benzyl]amino]acetic acid

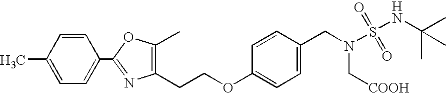

### Step 1: Preparation of [N—(N-t-butylamino)sulfonyl-N-[4-[2-[2-(4-methyl phenyl)-5-methyloxazol-4-yl]ethoxy]benzyl]amino]acetate methyl ester

The procedure of Step 2 of Example 3 was repeated except for using 394 mg (1 mmol) of N-[4-[2-[2-(4-methylphenyl)-5-methyloxazol-4-yl]ethoxy]benzyl]amino]acetate methyl ester, 210 mg (1.2 mmol) of t-butylamino sulfonyl chloride and 111 mg (1.1 mmol) of triethylamine to obtain the title compound (307 mg, 58%).

¹H-NMR (CDCl₃, 200 MHz): δ (ppm) 7.85 (d, J=8.1 Hz, 2H), 7.22 (m, 4H), 6.86 (d, J=8.9 Hz, 2H), 5.13 (s, 1H), 4.30 (s, 2H), 4.23 (t, J=6.9 Hz, 2H), 3.86 (s, 2H), 3.71 (s, 3H), 2.96 (t, J=6.9 Hz, 2H), 2.38 (s, 3H), 2.36 (s, 3H), 1.40 (s, 9H).

### Step 2: Preparation of [N—(N-t-butylamino)sulfonyl-N-[4-[2-[2-(4-methyl phenyl)-5-methyloxazol-4-yl]ethoxy]benzyl]amino]acetic acid

The procedure of Step 5 of Example 3 was repeated except for using 307 mg (0.58 mmol) of the compound obtained in Step 1 and 37 mg (0.87 mmol) of lithium hydroxide monohydrate to obtain the title compound (296 mg, 99%).

¹H-NMR (CDCl₃, 200 MHz): δ (ppm) 10.15 (br s, 1H), 7.83 (d, J=8.1 Hz, 2H), 7.25-7.20 (m, 4H), 6.83 (d, J=8.7 Hz, 2H), 5.36 (br s, 1H), 4.33 (s, 2H), 4.17 (t, J=6.9 Hz, 2H), 3.89 (s, 2H), 3.02 (t, J=6.9 Hz, 2H), 2.38 (s, 6H), 1.43 (s, 9H).

### EXAMPLE 101

### Preparation of [N—(N,N-diethylamino)sulfonyl-N-[4-[2-[2-(4-methylphenyl)-5-methyloxazol-4-yl]ethoxy]benzyl]amino]acetic acid

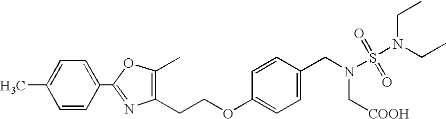

### Step 1: Preparation of [N—(N,N-diethylamino)sulfonyl-N-[4-[2-[2-(4-methyl phenyl)-5-methyloxazol-4-yl]ethoxy]benzyl]amino]acetate methyl ester

The procedure of Step 2 of Example 3 was repeated except for using 394 mg (1 mmol) of [N-[4-[2-[2-(4-methylphenyl)-5-methyloxazol-4-yl]ethoxy]benzyl]amino]acetate methyl ester, 193 mg (1.1 mmol) of N,N-diethyl sulfamoyl chloride and 111 mg (1.1 mmol) of triethylamine to obtain the title compound (456 mg, 86%).

¹H-NMR (CDCl₃, 200 MHz): δ (ppm) 7.85 (d, J=8.1 Hz, 2H), 7.24-7.19 (m, 4H), 6.86 (d, J=8.5 Hz, 2H), 4.41 (s, 2H), 4.23 (t, J=6.5 Hz, 2H), 3.78 (s, 2H), 3.67 (s, 3H), 3.30 (q, J=7.1 Hz, 4H), 2.95 (t, J=6.5 Hz, 2H), 2.37 (s, 3H), 2.35 (s, 3H), 1.19 (t, J=7.1 Hz, 6H).

### Step 2: Preparation of [N—(N,N-diethylamino)sulfonyl-N-[4-[2-[2-(4-methyl phenyl)-5-methyloxazol-4-yl]ethoxy]benzyl]amino]acetic acid

The procedure of Step 5 of Example 3 was repeated except for using 456 mg (0.81 mmol) of the compound obtained in Step 1 and 51 mg (1.22 mmol) of lithium hydroxide monohydrate to obtain the title compound (413 mg, 99%).

¹H-NMR (CDCl₃, 200 MHz): δ (ppm) 10.65 (br s, 1H), 7.83 (d, J=8.1 Hz, 2H), 7.24-7.20 (m, 4H), 6.83 (d, J=8.5 Hz, 2H), 4.43 (s, 2H), 4.19 (t, J=6.5 Hz, 2H), 3.79 (s, 2H), 3.30 (q, J=7.1 Hz, 4H), 3.00 (t, J=6.5 Hz, 2H), 2.36 (s, 6H), 1.17 (t, J=7.1 Hz, 6H).

### EXAMPLE 102

### Preparation of [N—(N-isopropyl-N-methylamino)sulfonyl-N-[4-[2-[2-(4-methylphenyl)-5-methyloxazol-4-yl]ethoxy]benzyl]amino]acetic acid

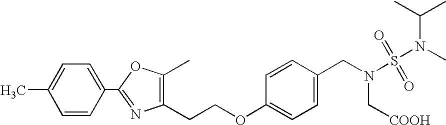

### Step 1: Preparation of [N—(N-isopropyl-N-methylamino)sulfonyl-N-[4-[2-[2-(4-methylphenyl)-5-methyloxazol-4-yl]ethoxy]benzyl]amino]acetate methyl ester

The procedure of Step 2 of Example 3 was repeated except for using 394 mg (1 mmol) of [N-[4-[2-[2-(4-methylphenyl)-5-methyloxazol-4-yl]ethoxy]benzyl]amino]acetate methyl ester, 193 mg (1.1 mmol) of (N-isopropyl-N-methylamino)sulfonyl chloride and 111 mg (1.1 mmol) of triethylamine to obtain the title compound (387 mg, 73%).

¹H-NMR (CDCl₃, 200 MHz): δ (ppm) 7.85 (d, J=8.1 Hz, 2H), 7.24-7.19 (m, 4H), 6.85 (d, J=8.5 Hz, 2H), 4.39 (s, 2H), 4.26-4.15 (m, 3H), 3.77 (s, 2H), 3.67 (s, 3H), 2.96 (t, J=6.5 Hz, 2H), 2.72 (s, 3H), 2.38 (s, 3H), 2.35 (s, 3H), 1.19 (s, 3H), 1.16 (s, 3H).

### Step 2: Preparation of [N—(N-isopropyl-N-methylamino)sulfonyl-N-[4-[2-[2-(4-methylphenyl)-5-methyloxazol-4-yl]ethoxy]benzyl]amino]acetic acid

The procedure of Step 5 of Example 3 was repeated except for using 387 mg (0.73 mmol) of the compound obtained in Step 1 and 46 mg (1.1 mmol) of lithium hydroxide monohydrate to obtain the title compound (373 mg, 99%).

¹H-NMR (CDCl₃, 200 MHz): δ (ppm) 10.99 (br s, 1H), 7.83 (d, J=8.1 Hz, 2H), 7.25-7.20 (m, 4H), 6.84 (d, J=8.5 Hz, 2H), 4.41 (s, 2H), 4.21-4.11 (m, 3H), 3.79 (s, 2H), 3.00 (t, J=6.5 Hz, 2H), 2.71 (s, 3H), 2.37 (s, 3H), 2.36 (s, 3H), 1.18 (s, 3H), 1.15 (s, 3H).

### EXAMPLE 103

### Preparation of [N—(N,N-dimethylamino)sulfonyl-N-[4-[2-[2-(4-trifluoromethylphenyl)-5-methyloxazol-4-yl]ethoxy]benzyl]amino]acetic acid

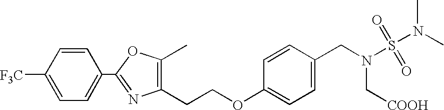

### Step 1: Preparation of [N—(N,N-dimethylamino)sulfonyl-N-[4-[2-[2-(4-trifluoromethylphenyl)-5-methyloxazol-4-yl]ethoxy]benzyl]amino]acetic acid ethyl ester

The procedure of Step 1 of Example 61 was repeated except for using 316 mg (1 mmol) of the compound obtained in Step 2 of Example 38, 446 mg (1.5 mmol) of 2-[5-methyl-2-[(4-trifluoromethyl)phenyl]oxazol-4-yl]ethanol, 446 mg (1.7 mmol) of triphenylphosphine and 344 mg (1.7 mmol) of diisopropyl azocarboxylate to obtain the title compound (518 mg, 91%).

¹H-NMR (CDCl₃, 300 MHz): δ (ppm) 8.07 (d, J=8.2 Hz, 2H), 7.67 (d, J=8.3 Hz, 2H), 7.25 (d, J=8.5 Hz, 2H), 6.87 (d, J=8.5 Hz, 2H), 4.48 (s, 2H), 4.24 (t, J=6.5 Hz, 2H), 4.15 (q, J=7.2 Hz, 2H), 3.80 (s, 2H), 2.99 (t, J=6.5 Hz, 2H), 2.86 (s, 6H), 2.39 (s, 3H), 1.24 (t, J=7.2 Hz, 3H).

### Step 2: Preparation of [N—(N,N-dimethylamino)sulfonyl-N-[4-[2-[2-(4-trifluoro methylphenyl)-5-methyloxazol-4-yl]ethoxy]benzyl]amino]acetic acid

The procedure of Step 5 of Example 3 was repeated except for using 518 mg (0.91 mmol) of the compound obtained in Step 1 and 59 mg (1.4 mmol) of lithium hydroxide monohydrate to obtain the title compound (488 mg, 99%).

¹H-NMR (CDCl₃, 300 MHz): δ (ppm) 10.25 (br s, 1H), 8.07 (d, J=8.1 Hz, 2H), 7.68 (d, J=8.1 Hz, 2H), 7.23 (d, J=8.4 Hz, 2H), 6.86 (d, J=8.4 Hz, 2H), 4.47 (s, 2H), 4.22 (t, J=6.3 Hz, 2H), 3.92 (s, 2H), 3.02 (t, J=6.3 Hz, 2H), 2.84 (s, 6H), 2.41 (s, 3H).

### EXAMPLE 104

### Preparation of [N—(N,N-dimethylamino)sulfonyl-N-[4-[2-[2-(thiophen-2-yl)-5-methyloxazol-4-yl]ethoxy]benzyl]amino]acetic acid

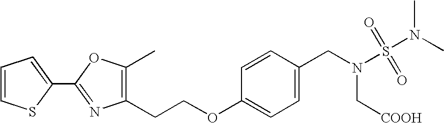

### Step 1: Preparation of [N—(N,N-dimethylamino)sulfonyl-N-[4-[2-[2-(thiophen-2-yl)-5-methyloxazol-4-yl]ethoxy]benzyl]amino]acetic acid ethyl ester

The procedure of Step 1 of Example 61 was repeated except for using 316 mg (1 mmol) of the compound obtained in Step 2 of Example 38, 314 mg (1.5 mmol) of 2-[5-methyl-2-(thiophen-2-yl)oxazol-4-yl]ethanol, 446 mg (1.7 mmol) of triphenylphosphine and 344 mg (1.7 mmol) of diisopropyl azocarboxylate to obtain the title compound (472 mg, 93%).

¹H-NMR (CDCl₃, 300 MHz): δ (ppm) 7.58-7.57 (m, 1H), 7.36-7.35 (m, 1H), 7.20 (d, J=8.4 Hz, 2H), 7.08-7.05 (m, 1H), 6.86 (d, J=8.4 Hz, 2H), 4.47 (s, 2H), 4.21 (t, J=6.6 Hz, 2H), 4.15 (q, J=7.2 Hz, 2H), 3.80 (s, 2H), 2.95 (t, J=6.6 Hz, 2H), 2.85 (s, 6H), 2.35 (s, 3H), 1.24 (t, J=7.2 Hz, 3H).

### Step 2: Preparation of [N—(N,N-dimethylamino)sulfonyl-N-[4-[2-[2-(thiophen-2-yl)-5-methyloxazol-4-yl]ethoxy]benzyl]amino]acetic acid

The procedure of Step 5 of Example 3 was repeated except for using 472 mg (0.93 mmol) of the compound obtained in Step 1 and 59 mg (1.4 mmol) of lithium hydroxide monohydrate to obtain the title compound (442 mg, 99%).

¹H-NMR (CDCl₃, 300 MHz): δ (ppm) 10.09 (br s, 1H), 7.63-7.62 (m, 1H), 7.39-7.38 (m, 1H), 7.27-7.15 (m, 2H), 7.12-7.06 (m, 1H), 6.85-6.79 (m, 2H), 4.47 (s, 2H), 4.18 (t, J=6.6 Hz, 2H), 4.09 (s, 2H), 2.98 (t, J=6.6 Hz, 2H), 2.84 (s, 6H), 2.34 (s, 3H).

### EXAMPLE 105

### Preparation of [N—[(N-methyl-N-phenyl)amino]sulfonyl-N-[4-[2-(2-phenyl-5-methyloxazol-4-yl)ethoxy]benzyl]amino]acetic acid

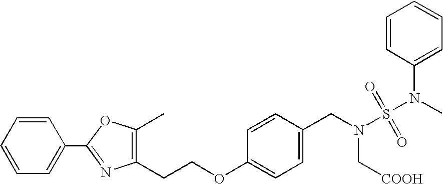

### Step 1: Preparation of [N—[(N-methyl-N-phenyl)amino]sulfonyl-N-[4-[2-(2-phenyl-5-methyloxazol-4-yl)ethoxy]benzyl]amino]acetic acid ethyl ester

The procedure of Step 1 of Example 61 was repeated except for using 378 mg (1 mmol) of the compound obtained in Step 2 of Example 45, 305 mg (1.5 mmol) of 2-[(5-methyl-2-phenyl)oxazol-4-yl]ethanol, 446 mg (1.7 mmol) of triphenylphosphine and 344 mg (1.7 mmol) of diisopropyl azocarboxylate to obtain the title compound (519 mg, 92%).

¹H-NMR (CDCl₃, 300 MHz): δ (ppm) 7.98-7.95 (m, 2H), 7.45-7.28 (m, 6H), 7.25-7.24 (m, 1H), 7.12 (d, J=8.6 Hz, 2H), 6.82 (d, J=8.6 Hz, 2H), 4.42 (s, 2H), 4.21 (t, J=6.6 Hz, 2H), 4.15 (q, J=7.2 Hz, 2H), 3.78 (s, 2H), 3.29 (s, 3H), 2.69 (t, J=6.6 Hz, 2H), 2.36 (s, 3H), 1.23 (t, J=7.2 Hz, 3H).

### Step 2: Preparation of [N—[(N-methyl-N-phenyl)amino]sulfonyl-N-[4-[2-(2-phenyl-5-methyloxazol-4-yl)ethoxy]benzyl]amino]acetic acid

The procedure of Step 5 of Example 3 was repeated except for using 519 mg (0.92 mmol) of the compound obtained in Step 1 and 59 mg (1.4 mmol) of lithium hydroxide monohydrate to obtain the title compound (488 mg, 99%).

¹H-NMR (CDCl₃, 300 MHz): δ (ppm) 8.60 (br s, 1H), 7.96-7.94 (m, 2H), 7.45-7.25 (m, 8H), 7.10 (d, J=8.4 Hz, 2H), 6.79 (d, J=8.4 Hz, 2H), 4.43 (s, 2H), 4.16 (t, J=6.4 Hz, 2H), 3.82 (s, 2H), 3.27 (s, 3H), 3.00 (t, J=6.4 Hz, 2H), 2.38 (s, 3H).

### EXAMPLE 106

### Preparation of [N—[(N-methyl-N-phenyl)amino]sulfonyl-N-[4-[2-[2-(4-methylphenyl)-5-methyloxazol-4-yl]ethoxy]benzyl]amino]acetic acid

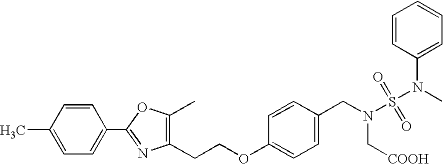

### Step 1: Preparation of [N—[(N-methyl-N-phenyl)amino]sulfonyl-N-[4-[2-[2-(4-methylphenyl)-5-methyloxazol-4-yl]ethoxy]benzyl]amino]acetic acid ethyl ester

The procedure of Step 1 of Example 61 was repeated except for using 378 mg (1 mmol) of the compound obtained in Step 2 of Example 45, 326 mg (1.5 mmol) of 2-[5-methyl-2-(4-methylphenyl)oxazol-4-yl]ethanol, 446 mg (1.7 mmol) of triphenylphosphine and 344 mg (1.7 mmol) of diisopropyl azocarboxylate to obtain the title compound (555 mg, 96%).

¹H-NMR (CDCl₃, 300 MHz): δ (ppm) 7.85 (d, J=8.1 Hz, 2H), 7.46-7.42 (m, 2H), 7.38-7.33 (m, 2H), 7.25-7.21 (m, 3H), 7.12 (d, J=8.4 Hz, 2H), 6.83 (d, J=8.4 Hz, 2H), 4.42 (s, 2H), 4.22 (t, J=6.6 Hz, 2H), 4.17 (q, J=7.2 Hz, 2H), 3.78 (s, 2H), 3.29 (s, 3H), 2.96 (t, J=6.6 Hz, 2H), 2.38 (s, 3H), 2.34 (s, 3H), 1.23 (t, J=7.2 Hz, 3H).

### Step 2: Preparation of [N—[(N-methyl-N-phenyl)amino]sulfonyl-N-[4-[2-[2-(4-methylphenyl)-5-methyloxazol-4-yl]ethoxy]benzyl]amino]acetic acid

The procedure of Step 5 of Example 3 was repeated except for using 555 mg (0.96 mmol) of the compound obtained in Step 1 and 59 mg (1.4 mmol) of lithium hydroxide monohydrate to obtain the title compound (522 mg, 99%).

¹H-NMR (CDCl₃, 300 MHz): δ (ppm) 7.83 (d, J=8.1 Hz, 2H), 7.46-7.43 (m, 2H), 7.37-7.32 (m, 2H), 7.28-7.21 (m, 3H), 7.10 (d, J=8.7 Hz, 2H), 6.80 (d, J=8.7 Hz, 2H), 4.43 (s, 2H), 4.17 (t, J=6.6 Hz, 2H), 3.82 (s, 2H), 3.28 (s, 3H), 3.00 (t, J=6.6 Hz, 2H), 2.37 (s, 6H).

### EXAMPLE 107

### Preparation of [N—[(N-methyl-N-phenyl)amino]sulfonyl-N-[4-[2-[2-(4-trifluoromethylphenyl)-5-methyloxazol-4-yl]ethoxy]benzyl]amino]acetic acid

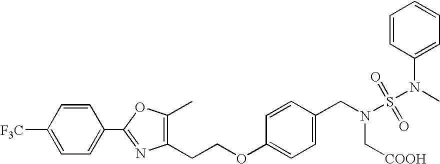

### Step 1: Preparation of [N—[(N-methyl-N-phenyl)amino]sulfonyl-N-[4-[2-[2-(4-trifluoromethylphenyl)-5-methyloxazol-4-yl]ethoxy]benzyl]amino]acetic acid ethyl ester

The procedure of Step 1 of Example 61 was repeated except for using 378 mg (1 mmol) of the compound obtained in Step 2 of Example 45, 407 mg (1.5 mmol) of 2-[5-methyl-2-[(4-trifluoromethyl)phenyl]oxazol-4-yl]ethanol, 446 mg (1.7 mmol) of triphenylphosphine and 344 mg (1.7 mmol) of diisopropyl azocarboxylate to obtain the title compound (581 mg, 92%).

¹H-NMR (CDCl₃, 300 MHz): δ (ppm) 8.07 (d, J=8.3 Hz, 2H), 7.67 (d, J=8.3 Hz, 2H), 7.46-7.44 (m, 2H), 7.38-7.33 (m, 2H), 7.28-7.24 (m, 1H), 7.13 (d, J=8.4 Hz, 2H), 6.83 (d, J=8.4 Hz, 2H), 4.42 (s, 2H), 4.22 (t, J=6.6 Hz, 2H), 4.14 (q, J=7.2 Hz, 2H), 3.78 (s, 2H), 3.29 (s, 3H), 2.98 (t, J=6.6 Hz, 2H), 2.39 (s, 3H), 1.24 (t, J=7.2 Hz, 3H).

### Step 2: Preparation of [N—[(N-methyl-N-phenyl)amino]sulfonyl-N-[4-[2-[2-(4-trifluoromethylphenyl)-5-methyloxazol-4-yl]ethoxy]benzyl]amino]acetic acid

The procedure of Step 5 of Example 3 was repeated except for using 581 mg (0.92 mmol) of the compound obtained in Step 1 and 59 mg (1.4 mmol) of lithium hydroxide monohydrate to obtain the title compound (550 mg, 99%).

¹H-NMR (CDCl₃, 300 MHz): δ (ppm) 8.06 (d, J=8.3 Hz, 2H), 7.67 (d, J=8.3 Hz, 2H), 7.44-7.40 (m, 2H), 7.37-7.32 (m, 2H), 7.28-7.23 (m, 2H), 7.11 (d, J=8.4 Hz, 2H), 6.80 (d, J=8.4 Hz, 2H), 5.54 (br s, 1H), 4.42 (s, 2H), 4.19 (t, J=6.6 Hz, 2H), 3.82 (s, 2H), 3.27 (s, 3H), 2.99 (t, J=6.6 Hz, 2H), 2.39 (s, 3H).

### EXAMPLE 108

### Preparation of [N—[(N-methyl-N-phenyl)amino]sulfonyl-N-[4-[2-[2-(thiophen-2-yl)-5-methoxyoxazol-4-yl]ethoxy]benzyl]amino]acetic acid

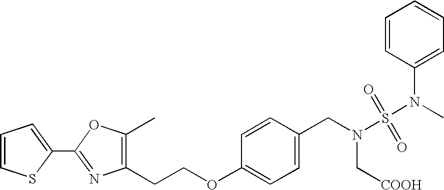

### Step 1: Preparation of [N—[(N-methyl-N-phenyl)amino]sulfonyl-N-[4-[2-[2-(thiophen-2-yl)-5-methoxyoxazol-4-yl]ethoxy]benzyl]amino]acetic acid ethyl ester

The procedure of Step 1 of Example 61 was repeated except for using 378 mg (1 mmol) of the compound obtained in Step 2 of Example 45, 314 mg (1.5 mmol) of 2-[5-methyl-2-(thiophen-2-yl)oxazol-4-yl]ethanol, 446 mg (1.7 mmol) of triphenylphosphine and 344 mg (1.7 mmol) of diisopropyl azocarboxylate to obtain the title compound (530 mg, 93%).

¹H-NMR (CDCl₃, 300 MHz): δ (ppm) 7.58-7.57 (m, 1H), 7.46-7.33 (m, 6H), 7.12 (d, J=8.4 Hz, 2H), 7.08-7.07 (m, 1H), 6.82 (d, J=8.4 Hz, 2H), 4.42 (s, 2H), 4.19 (t, J=6.6 Hz, 2H), 4.13 (q, J=7.2 Hz, 2H), 3.78 (s, 2H), 3.29 (s, 3H), 2.94 (t, J=6.6 Hz, 2H), 2.34 (s, 3H), 1.23 (t, J=7.2 Hz, 3H).

### Step 2: Preparation of [N—[(N-methyl-N-phenyl)amino]sulfonyl-N-[4-[2-[2-(thiophen-2-yl)-5-methoxyoxazol-4-yl]ethoxy]benzyl]amino]acetic acid

The procedure of Step 5 of Example 3 was repeated except for using 530 mg (0.93 mmol) of the compound obtained in Step 1 and 59 mg (1.4 mmol) of lithium hydroxide monohydrate to obtain the title compound (499 mg, 99%).

¹H-NMR (CDCl₃, 300 MHz): δ (ppm) 7.62-7.61 (m, 1H), 7.45-7.25 (m, 6H), 7.11 (d, J=8.4 Hz, 2H), 7.08-7.06 (m, 1H), 6.89 (d, J=8.4 Hz, 2H), 5.16 (br s, 1H), 4.43 (s, 2H), 4.16 (t, J=6.6 Hz, 2H), 3.82 (s, 2H), 3.27 (s, 3H), 2.97 (t, J=6.6 Hz, 2H), 2.35 (s, 3H).

### EXAMPLE 109

### Preparation of [N—[[N-methyl-N-(4-chlorophenyl)]amino]sulfonyl-N-[4-[2-(2-phenyl-5-methyloxazol-4-yl)ethoxy]benzyl]amino]acetic acid

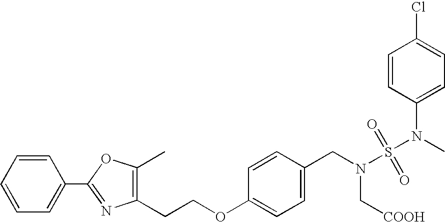

### Step 1: Preparation of [N—[[N-methyl-N-(4-chlorophenyl)]amino]sulfonyl-N-[4-[2-(2-phenyl-5-methyloxazol-4-yl)ethoxy]benzyl]amino]acetic acid ethyl ester

The procedure of Step 1 of Example 61 was repeated except for using 413 mg (1 mmol) of the compound obtained in Step 2 of Example 49, 305 mg (1.5 mmol) of 2-[(5-methyl-2-phenyl)oxazol-4-yl]ethanol, 446 mg (1.7 mmol) of triphenylphosphine and 344 mg (1.7 mmol) of diisopropyl azocarboxylate to obtain the title compound (556 mg, 93%).

¹H-NMR (CDCl₃, 300 MHz): δ (ppm) 7.98-7.95 (m, 2H), 7.43-7.28 (m, 7H), 7.05 (d, J=8.4 Hz, 2H), 6.74 (d, J=8.4 Hz, 2H), 4.39 (s, 2H), 4.22 (t, J=6.6 Hz, 2H), 4.15 (q, J=7.2 Hz, 2H), 3.90 (s, 2H), 3.25 (s, 3H), 2.96 (t, J=6.6 Hz, 2H), 2.37 (s, 3H), 1.26 (t, J=7.2 Hz, 3H).

### Step 2: Preparation of [N—[[N-methyl-N-(4-chlorophenyl)]amino]sulfonyl-N-[4-[2-(2-phenyl-5-methyloxazol-4-yl)ethoxy]benzyl]amino]acetic acid

The procedure of Step 5 of Example 3 was repeated except for using 556 mg (0.93 mmol) of the compound obtained in Step 1 and 59 mg (1.4 mmol) of lithium hydroxide monohydrate to obtain the title compound (525 mg, 99%).

¹H-NMR (CDCl₃, 300 MHz): δ (ppm) 7.97-7.94 (m, 2H), 7.42-7.29 (m, 7H), 7.05 (d, J=8.4 Hz, 2H), 6.75 (d, J=8.4 Hz, 2H), 4.38 (s, 2H), 4.15 (t, J=6.6 Hz, 2H), 3.79 (s, 2H), 3.20 (s, 3H), 2.99 (t, J=6.6 Hz, 2H), 2.37 (s, 3H).

### EXAMPLE 110

### Preparation of [N—[[N-methyl-N-(4-chlorophenyl)]amino]sulfonyl-N-[4-[2-[2-(4-methylphenyl)-5-methyloxazol-4-yl]ethoxy]benzyl]amino]acetic acid

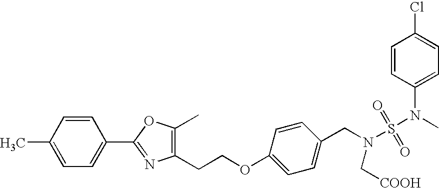

### Step 1: Preparation of [N—[[N-methyl-N-(4-chlorophenyl)]amino]sulfonyl-N-[4-[2-[2-(4-methylphenyl)-5-methyloxazol-4-yl]ethoxy]benzyl]amino]acetic acid ethyl ester

The procedure of Step 1 of Example 61 was repeated except for using 413 mg (1 mmol) of the compound obtained in Step 2 of Example 49, 326 mg (1.5 mmol) of 2-[5-methyl-2-(4-methylphenyl)oxazol-4-yl]ethanol, 446 mg (1.7 mmol) of triphenylphosphine and 344 mg (1.7 mmol) of diisopropyl azocarboxylate to obtain the title compound (582 mg, 95%).

¹H-NMR (CDCl₃, 300 MHz): δ (ppm) 7.85 (d, J=8.1 Hz, 2H), 7.39 (d, J=8.5 Hz, 2H), 7.32 (d, J=8.5 Hz, 2H), 7.22 (d, J=8.1 Hz, 2H), 7.20 (d, J=8.4 Hz, 2H), 6.83 (d, J=8.4 Hz, 2H), 4.40 (s, 2H), 4.22 (t, J=6.6 Hz, 2H), 4.16 (q, J=7.2 Hz, 2H), 3.79 (s, 2H), 3.26 (s, 3H), 2.96 (t, J=6.6 Hz, 2H), 2.38 (s, 3H), 2.36 (s, 3H), 1.23 (t, J=7.2 Hz, 3H).

### Step 2: Preparation of [N—[[N-methyl-N-(4-chlorophenyl)]amino]sulfonyl-N-[4-[2-[2-(4-methylphenyl)-5-methyloxazol-4-yl]ethoxy]benzyl]amino]acetic acid

The procedure of Step 5 of Example 3 was repeated except for using 582 mg (0.95 mmol) of the compound obtained in Step 1 and 59 mg (1.4 mmol) of lithium hydroxide monohydrate to obtain the title compound (549 mg, 99%).

¹H-NMR (CDCl₃, 300 MHz): δ (ppm) 7.83 (d, J=8.1 Hz, 2H), 7.39 (d, J=8.5 Hz, 2H), 7.29 (d, J=8.5 Hz, 2H), 7.23 (d, J=8.1 Hz, 2H), 7.13 (d, J=8.4 Hz, 2H), 6.81 (d, J=8.4 Hz, 2H), 4.42 (s, 2H), 4.16 (t, J=6.6 Hz, 2H), 3.84 (s, 2H), 3.26 (s, 3H), 3.03 (t, J=6.6 Hz, 2H), 2.38 (s, 3H), 2.37 (s, 3H).

### EXAMPLE 111

### Preparation of [N—(N-ethyl-N-m-tolylamino)sulfonyl-N-[4-[2-[2-(4-methylphenyl)-5-methyloxazol-4-yl]ethoxy]benzyl]amino]acetic acid

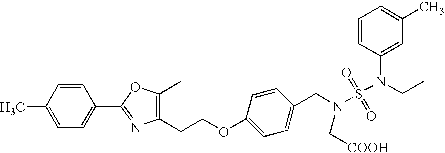

### Step 1: Preparation of [N—(N-ethyl-N-m-tolylamino)sulfonyl-N-[4-[2-[2-(4-methylphenyl)-5-methyloxazol-4-yl]ethoxy]benzyl]amino]acetate methyl ester

The procedure of Step 2 of Example 3 was repeated except for using 394 mg (1 mmol) of [N-[4-[2-[2-(4-methylphenyl)-5-methyloxazol-4-yl]ethoxy]benzyl]amino]acetate methyl ester, 257 mg (1.1 mmol) of (N-ethyl-N-m-tolylamino)sulfonyl chloride and 111 mg (1.1 mmol) of triethylamine to obtain the title compound (260 mg, 44%).

¹H-NMR (CDCl₃, 200 MHz): δ (ppm) 7.85 (d, J=8.1 Hz, 2H), 7.25-7.20 (m, 5H), 7.13-7.07 (m, 3H), 6.83-6.79 (m, 2H), 4.42 (s, 2H), 4.21 (t, J=6.5 Hz, 2H), 3.77 (s, 2H), 3.69 (q, J=7.2 Hz, 2H), 3.66 (s, 3H), 2.96 (t, J=6.5 Hz, 2H), 2.38 (s, 3H), 2.35 (s, 6H), 1.08 (t, J=7.2 Hz, 3H).

### Step 2: Preparation of [N—(N-ethyl-N-m-tolylamino)sulfonyl-N-[4-[2-[2-(4-methylphenyl)-5-methyloxazol-4-yl]ethoxy]benzyl]amino]acetic acid

The procedure of Step 5 of Example 3 was repeated except for using 260 mg (0.44 mmol) of the compound obtained in Step 1 and 28 mg (0.66 mmol) of lithium hydroxide monohydrate to obtain the title compound (252 mg, 99%).

¹H-NMR (CDCl₃, 200 MHz): δ (ppm) 7.83 (d, J=8.1 Hz, 2H), 7.26-7.12 (m, 6H), 7.09 (d, J=8.7 Hz, 2H), 6.78 (d, J=8.7 Hz, 2H), 4.43 (s, 2H), 4.17 (t, J=6.5 Hz, 2H), 3.79 (s, 2H), 3.69 (q, J=7.2 Hz, 2H), 3.01 (t, J=6.5 Hz, 2H), 2.37 (s, 6H), 2.34 (s, 3H), 1.07 (t, J=7.2 Hz, 3H).

### EXAMPLE 112

### Preparation of [N—(N-anisoyl-N-methylamino)sulfonyl-N-[4-[2-[2-(4-methylphenyl)-5-methyloxazol-4-yl]ethoxy]benzyl]amino]acetic acid

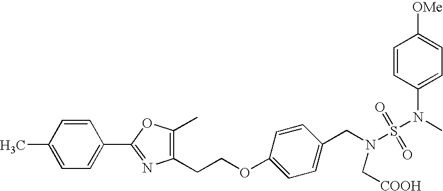

### Step 1: Preparation of [N—(N-anisoyl-N-methylamino)sulfonyl-N-[4-[2-[2-(4-methylphenyl)-5-methyloxazol-4-yl]ethoxy]benzyl]amino]acetate methyl ester

The procedure of Step 2 of Example 3 was repeated except for using 394 mg (1 mmol) of [N-[4-[2-[2-(4-methylphenyl)-5-methyloxazol-4-yl]ethoxy]benzyl]amino]acetate methyl ester, 259 mg (1.1 mmol) of (N-anisoyl-N-methylamino)sulfonyl chloride and 111 mg (1.1 mmol) of triethylamine to obtain the title compound (356 mg, 60%).

¹H-NMR (CDCl₃, 200 MHz): δ (ppm) 7.86 (d, J=8.1 Hz, 2H), 7.60 (d, J=8.1 Hz, 2H), 7.26-7.16 (m, 4H), 7.12-6.81 (m, 4H), 4.40 (s, 2H), 4.21 (t, J=6.5 Hz, 2H), 3.80 (s, 2H), 3.79 (s, 3H), 3.67 (s, 3H), 3.24 (s, 3H), 2.96 (t, J=6.5 Hz, 2H), 2.38 (s, 3H), 2.35 (s, 3H).

### Step 2: Preparation of [N—(N-anisoyl-N-methylamino)sulfonyl-N-[4-[2-[2-(4-methylphenyl)-5-methyloxazol-4-yl]ethoxy]benzyl]amino]acetic acid

The procedure of Step 5 of Example 3 was repeated except for using 356 mg (0.6 mmol) of the compound obtained in Step 1 and 38 mg (0.9 mmol) of lithium hydroxide monohydrate to obtain the title compound (344 mg, 99%).

¹H-NMR (CDCl₃, 200 MHz): δ (ppm) 7.83 (d, J=8.1 Hz, 2H), 7.38 (d, J=8.1 Hz, 2H), 7.26-7.12 (m,), 6.88-6.78 (m, 4H), 4.43 (s, 2H), 4.17 (t, J=6.5 Hz, 2H), 3.83 (s, 2H), 3.78 (s, 3H), 3.24 (s, 3H), 3.02 (t, J=6.5 Hz, 2H), 2.38 (s, 6H).

### EXAMPLE 113

### Preparation of [N—[N-(3-fluorophenyl)-N-methylamino]sulfonyl-N-[4-[2-[2-(4-methylphenyl)-5-methyloxazol-4-yl]ethoxy]benzyl]amino]acetic acid

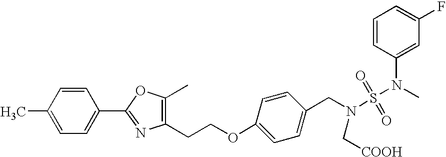

### Step 1: Preparation of [N—[N-(3-fluorophenyl)-N-methylamino]sulfonyl-N-[4-[2-[2-(4-methylphenyl)-5-methyloxazol-4-yl]ethoxy]benzyl]amino]acetate methyl ester

The procedure of Step 2 of Example 3 was repeated except for using 394 mg (1 mmol) of [N-[4-[2-[2-(4-methylphenyl)-5-methyloxazol-4-yl]ethoxy]benzyl]amino]acetate methyl ester, 246 mg (1.1 mmol) of [N-(3-fluorophenyl)-N-methylamino]sulfonyl chloride and 111 mg (1.1 mmol) of triethylamine to obtain the title compound (192 mg, 33%).

¹H-NMR (CDCl₃, 200 MHz): δ (ppm) 7.85 (d, J=8.1 Hz, 2H), 7.34-7.14 (m, 7H), 7.10-6.92 (m, 1H), 6.87 (d, J=8.9 Hz, 2H), 4.42 (s, 2H), 4.21 (t, J=6.5 Hz, 2H), 3.80 (s, 2H), 3.67 (s, 3H), 3.28 (s, 3H), 2.95 (t, J=6.5 Hz, 2H), 2.37 (s, 3H), 2.35 (s, 3H).

### Step 2: Preparation of [N—[N-(3-fluorophenyl)-N-methylamino]sulfonyl-N-[4-[2-[2-(4-methylphenyl)-5-methyloxazol-4-yl]ethoxy]benzyl]amino]acetic acid

The procedure of Step 5 of Example 3 was repeated except for using 192 mg (0.33 mmol) of the compound obtained in Step 1 and 23 mg (0.5 mmol) of lithium hydroxide monohydrate to obtain the title compound (185 mg, 99%).

¹H-NMR (CDCl₃, 200 MHz): δ (ppm) 7.83 (d, J=8.1 Hz, 2H), 7.35-7.14 (m, 7H), 7.10-6.89 (m, 1H), 6.79 (d, J=8.7 Hz, 2H), 4.43 (s, 2H), 4.16 (t, J=6.5 Hz, 2H), 3.84 (s, 2H), 3.27 (s, 3H), 3.03 (t, J=6.5 Hz, 2H), 2.38 (s, 6H).

### EXAMPLE 114

### Preparation of [N—[[N-methyl-N-(4-chlorophenyl)]amino]sulfonyl-N-[4-[2-[2-(4-trifluoromethylphenyl)-5-methyloxazol-4-yl]ethoxy]benzyl]amino]acetic acid

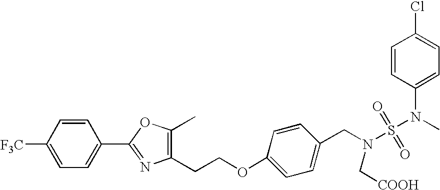

### Step 1: Preparation of [N—[[N-methyl-N-(4-chlorophenyl)]amino]sulfonyl-N-[4-[2-[2-(4-trifluoromethylphenyl)-5-methyloxazol-4-yl]ethoxy]benzyl]amino]acetic acid

The procedure of Step 1 of Example 61 was repeated except for using 413 mg (1 mmol) of the compound obtained in Step 2 of Example 49, 446 mg (1.5 mmol) of 2-[5-methyl-2-[(4-trifluoromethyl)phenyl]oxazol-4-yl]ethanol, 446 mg (1.7 mmol) of triphenylphosphine and 344 mg (1.7 mmol) of diisopropyl azocarboxylate to obtain the title compound (613 mg, 92%).

¹H-NMR (CDCl₃, 300 MHz): δ (ppm) 8.07 (d, J=8.3 Hz, 2H), 7.67 (d, J=8.3 Hz, 2H), 7.39 (d, J=8.7 Hz, 2H), 7.32 (d, J=8.7 Hz, 2H), 7.13 (d, J=8.5 Hz, 2H), 6.83 (d, J=8.5 Hz, 2H), 4.40 (s, 2H), 4.23 (t, J=6.6 Hz, 2H), 4.15 (q, J=7.2 Hz, 2H), 3.79 (s, 2H), 3.26 (s, 3H), 2.98 (t, J=6.6 Hz, 2H), 2.39 (s, 3H), 1.24 (t, J=7.2 Hz, 3H).

### Step 2: Preparation of [N—[[N-methyl-N-(4-chlorophenyl)]amino]sulfonyl-N-[4-[2-[2-(4-trifluoromethylphenyl)-5-methyloxazol-4-yl]ethoxy]benzyl]amino]acetic acid

The procedure of Step 5 of Example 3 was repeated except for using 613 mg (0.92 mmol) of the compound obtained in Step 1 and 59 mg (1.4 mmol) of lithium hydroxide monohydrate to obtain the title compound (581 mg, 99%).

¹H-NMR (CDCl₃, 300 MHz): δ (ppm) 8.06 (d, J=8.3 Hz, 2H), 7.68 (d, J=8.3 Hz, 2H), 7.36 (d, J=8.7 Hz, 2H), 7.29 (d, J=8.7 Hz, 2H), 7.12 (d, J=8.5 Hz, 2H), 6.82 (d, J=8.5 Hz, 2H), 4.41 (s, 2H), 4.19 (t, J=6.6 Hz, 2H), 3.84 (s, 2H), 3.25 (s, 3H), 3.01 (t, J=6.6 Hz, 2H), 2.41 (s, 3H).

### EXAMPLE 115

### Preparation of [N—[[N-methyl-N-(4-chlorophenyl)]amino]sulfonyl-N-[4-[2-[2-(thiophen-2-yl)-5-methyloxazol-4-yl]ethoxy]benzyl]amino]acetic acid

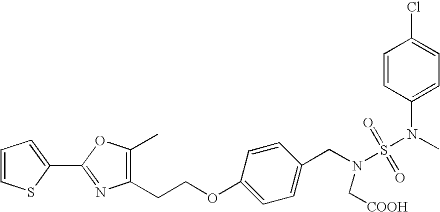

### Step 1: Preparation of [N—[[N-methyl-N-(4-chlorophenyl)]amino]sulfonyl-N-[4-[2-[2-(thiophen-2-yl)-5-methyloxazol-4-yl]ethoxy]benzyl]amino]acetic acid ethyl ester

The procedure of Step 1 of Example 61 was repeated except for using 413 mg (1 mmol) of the compound obtained in Step 2 of Example 49, 314 mg (1.5 mmol) of 2-[5-methyl-2-(thiophen-2-yl)oxazol-4-yl]ethanol, 446 mg (1.7 mmol) of triphenylphosphine and 344 mg (1.7 mmol) of diisopropyl azocarboxylate to obtain the title compound (544 mg, 90%).

¹H-NMR (CDCl₃, 300 MHz): δ (ppm) 7.58-7.57 (m, 1H), 7.41-7.30 (m, 5H), 7.12 (d, J=8.5 Hz, 2H), 7.09-7.06 (m, 1H), 6.83 (d, J=8.5 Hz, 2H), 4.40 (s, 2H), 4.20 (t, J=6.6 Hz, 2H), 4.14 (q, J=7.2 Hz, 2H), 3.79 (s, 2H), 3.26 (s, 3H), 2.94 (t, J=6.6 Hz, 2H), 2.35 (s, 3H), 1.24 (t, J=7.2 Hz, 3H).

### Step 2: Preparation of [N—[[N-methyl-N-(4-chlorophenyl)]amino]sulfonyl-N-[4-[2-[2-(thiophen-2-yl)-5-methyloxazol-4-yl]ethoxy]benzyl]amino]acetic acid

The procedure of Step 5 of Example 3 was repeated except for using 544 mg (0.9 mmol) of the compound obtained in Step 1 and 59 mg (1.4 mmol) of lithium hydroxide monohydrate to obtain the title compound (513 mg, 99%).

¹H-NMR (CDCl₃, 300 MHz): δ (ppm) 7.62-7.61 (m, 1H), 7.39-7.28 (m, 5H), 7.13 (d, J=8.5 Hz, 2H), 7.09-7.07 (m, 1H), 6.80 (d, J=8.5 Hz, 2H), 4.42 (s, 2H), 4.17 (t, J=6.6 Hz, 2H), 3.84 (s, 2H), 3.26 (s, 3H), 2.99 (t, J=6.6 Hz, 2H), 2.37 (s, 3H).

### EXAMPLE 116

### Preparation of [N-(indolinyl)sulfonyl-N-[4-[2-[(2-phenyl-5-methyloxazol-4-yl)ethoxy]benzyl]amino]acetic acid

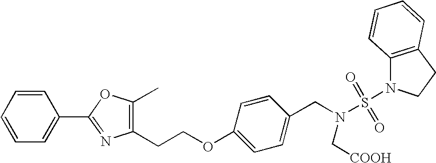

### Step 1: Preparation of [N-(indolinyl)sulfonyl-N-[4-[2-[(2-phenyl-5-methyl oxazol-4-yl)ethoxy]benzyl]amino]acetic acid ethyl ester

The procedure of Step 1 of Example 61 was repeated except for using 390 mg (1 mmol) of the compound obtained in Step 2 of Example 53, 305 mg (1.5 mmol) of 2-[(5-methyl-2-phenyl)oxazol-4-yl]ethanol, 446 mg (1.7 mmol) of triphenylphosphine and 344 mg (1.7 mmol) of diisopropyl azocarboxylate to obtain the title compound (553 mg, 96%).

¹H-NMR (CDCl₃, 300 MHz): δ (ppm) 7.98-7.95 (m, 2H), 7.43-7.39 (m, 4H), 7.15-6.96 (m, 4H), 6.94-6.81 (m, 1H), 6.79 (d, J=8.4 Hz, 2H), 4.55 (s, 2H), 4.21 (t, J=6.6 Hz, 2H), 4.05 (t, J=8.7 Hz, 2H), 3.96 (s, 2H), 3.83 (s, 2H), 3.11 (t, J=8.4 Hz, 2H), 2.96 (t, J=6.6 Hz, 2H), 2.37 (s, 3H), 1.10 (t, J=7.2 Hz, 3H).

### Step 2: Preparation of [N-(indolinyl)sulfonyl-N-[4-[2-[(2-phenyl-5-methyl oxazol-4-yl)ethoxy]benzyl]amino]acetic acid

The procedure of Step 5 of Example 3 was repeated except for using 553 mg (0.96 mmol) of the compound obtained in Step 1 and 59 mg (1.4 mmol) of lithium hydroxide monohydrate to obtain the title compound (520 mg, 99%).

¹H-NMR (CDCl₃, 300 MHz): δ (ppm) 8.81 (br s, 1H), 7.95-7.92 (m, 2H), 7.44-7.39 (m, 4H), 7.12-7.07 (m, 4H), 6.94-6.92 (m, 1H), 6.78 (d, J=8.4 Hz, 2H), 4.49 (s, 2H), 4.14 (t, J=6.3 Hz, 2H), 4.02 (q, J=7.2 Hz, 2H), 3.90 (s, 2H), 3.07 (t, J=8.4 Hz, 2H), 2.98 (t, J=6.3 Hz, 2H), 2.38 (s, 3H).

### EXAMPLE 117

### Preparation of [N-(indolinyl)sulfonyl-N-[4-[2-[2-(4-methyl phenyl)-5-methyloxazol-4-yl]ethoxy]benzyl]amino]acetic acid

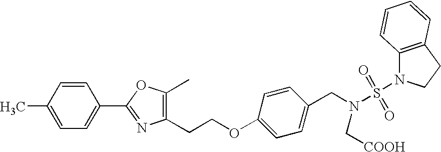

### Step 1: Preparation of [N-(indolinyl)sulfonyl-N-[4-[2-[2-(4-methylphenyl)-5-methyloxazol-4-yl]ethoxy]benzyl]amino]acetic acid ethyl ester

The procedure of Step 1 of Example 61 was repeated except for using 390 mg (1 mmol) of the compound obtained in Step 2 of Example 53, 326 mg (1.5 mmol) of 2-[5-methyl-2-(4-methylphenyl)oxazol-4-yl]ethanol, 446 mg (1.7 mmol) of triphenylphosphine and 344 mg (1.7 mmol) of diisopropyl azocarboxylate to obtain the title compound (554 mg, 94%).

¹H-NMR (CDCl₃, 200 MHz): δ (ppm) 7.85 (d, J=8.1 Hz, 2H), 7.42 (d, J=7.9 Hz, 1H), 7.25-7.09 (m, 6H), 6.98-6.90 (m, 1H), 6.82 (d, J=8.4 Hz, 2H), 4.55 (s, 2H), 4.21 (t, J=6.7 Hz, 2H), 4.05 (t, J=8.5 Hz, 2H), 3.95 (q, J=7.2 Hz, 2H), 3.83 (s, 2H), 3.11 (t, J=8.5 Hz, 2H), 2.95 (t, J=6.7 Hz, 2H), 2.38 (s, 3H), 2.35 (s, 3H), 1.12 (t, J=7.2 Hz, 3H).

### Step 2: Preparation of [N-(indolinyl)sulfonyl-N-[4-[2-[2-(4-methylphenyl)-5-methyloxazol-4-yl]ethoxy]benzyl]amino]acetic acid

The procedure of Step 5 of Example 3 was repeated except for using 554 mg (0.94 mmol) of the compound obtained in Step 1 and 59 mg (1.4 mmol) of lithium hydroxide monohydrate to obtain the title compound (523 mg, 99%).

¹H-NMR (CDCl₃, 300 MHz): δ (ppm) 7.82 (d, J=8.4 Hz, 2H), 7.43 (d, J=8.4 Hz, 2H), 7.25-7.08 (m, 6H), 6.96-6.91 (m, 1H), 6.79 (d, J=8.4 Hz, 2H), 4.51 (s, 2H), 4.16 (t, J=6.6 Hz, 2H), 4.04 (t, J=8.5 Hz, 2H), 3.09 (t, J=8.5 Hz, 2H), 2.98 (t, J=6.6 Hz, 2H), 2.38 (s, 3H), 2.37 (s, 3H).

### EXAMPLE 118

### Preparation of [N-(indolinyl)sulfonyl-N-[4-[2-[2-(4-trifluoro methylphenyl)-5-methyloxazol-4-yl]ethoxy]benzyl]amino]acetic acid

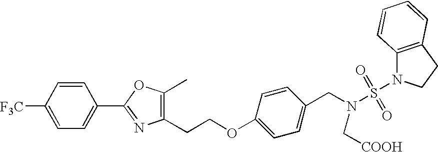

### Step 1: Preparation of [N-(indolinyl)sulfonyl-N-[4-[2-[2-(4-trifluoromethyl phenyl)-5-methyloxazol-4-yl]ethoxy]benzyl]amino]acetic acid ethyl ester

The procedure of Step 1 of Example 61 was repeated except for using 390 mg (1 mmol) of the compound obtained in Step 2 of Example 53, 407 mg (1.5 mmol) of 2-[5-methyl-2-[(4-trifluoromethyl)phenyl]oxazol-4-yl]ethanol, 446 mg (1.7 mmol) of triphenylphosphine and 344 mg (1.7 mmol) of diisopropyl azocarboxylate to obtain the title compound (599 mg, 93%).

¹H-NMR (CDCl₃, 200 MHz): δ (ppm) 8.09 (d, J=8.7 Hz, 2H), 7.67 (d, J=8.7 Hz, 2H), 7.43 (d, J=7.9 Hz, 1H), 7.25-7.09 (m, 4H), 6.97-6.90 (m, 1H), 6.82 (d, J=8.4 Hz, 2H), 4.56 (s, 2H), 4.22 (t, J=6.5 Hz, 2H), 4.09-3.89 (m, 4H), 3.83 (s, 2H), 3.12 (t, J=8.3 Hz, 2H), 2.98 (t, J=6.5 Hz, 2H), 2.39 (s, 3H), 1.11 (t, J=7.2 Hz, 3H).

### Step 2: Preparation of [N-(indolinyl)sulfonyl-N-[4-[2-[2-(4-trifluoromethyl phenyl)-5-methyloxazol-4-yl]ethoxy]benzyl]amino]acetic acid

The procedure of Step 5 of Example 3 was repeated except for using 599 mg (0.93 mmol) of the compound obtained in Step 1 and 59 mg (1.4 mmol) of lithium hydroxide monohydrate to obtain the title compound (567 mg, 99%).

¹H-NMR (CDCl₃, 200 MHz): δ (ppm) 8.06 (d, J=8.7 Hz, 2H), 7.67 (d, J=8.7 Hz, 2H), 7.43 (d, J=7.9 Hz, 1H), 7.25-7.09 (m, 4H), 6.97-6.90 (m, 1H), 6.80 (d, J=8.4 Hz, 2H), 4.50 (s, 2H), 4.18 (t, J=6.5 Hz, 2H), 4.02 (t, J=8.5 Hz, 2H), 3.89 (s, 2H), 3.08 (t, J=8.3 Hz, 2H), 2.98 (t, J=6.5 Hz, 2H), 2.39 (s, 3H).

### EXAMPLE 119

### Preparation of [N-(indolinyl)sulfonyl-N-[4-[2-[2-(thiophen-2-yl)-5-methyloxazol-4-yl]ethoxy]benzyl]amino]acetic acid

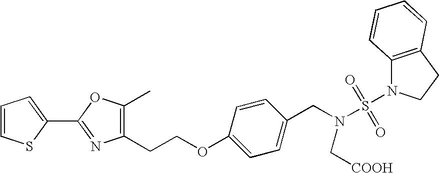

### Step 1: Preparation of [N-(indolinyl)sulfonyl-N-[4-[2-[2-(thiophen-2-yl)-5-methyloxazol-4-yl]ethoxy]benzyl]amino]acetic acid ethyl ester

The procedure of Step 1 of Example 61 was repeated except for using 390 mg (1 mmol) of the compound obtained in Step 2 of Example 53, 314 mg (1.5 mmol) of 2-[5-methyl-2-(thiophen-2-yl)oxazol-4-yl]ethanol, 446 mg (1.7 mmol) of triphenylphosphine and 344 mg (1.7 mmol) of diisopropyl azocarboxylate to obtain the title compound (547 mg, 94%).

¹H-NMR (CDCl₃, 200 MHz): δ (ppm) 7.58-7.56 (m, 1H), 7.44-7.25 (m, 2H), 7.16-7.05 (m, 5H), 6.97-6.90 (m, 1H), 6.80 (d, J=8.4 Hz, 2H), 4.55 (s, 2H), 4.19 (t, J=6.7 Hz, 2H), 4.09-3.89 (m, 4H), 3.83 (s, 2H), 3.11 (t, J=8.5 Hz, 2H), 2.94 (t, J=6.6 Hz, 2H), 2.34 (s, 3H), 1.12 (t, J=7.2 Hz, 3H).

### Step 2: Preparation of [N-(indolinyl)sulfonyl-N-[4-[2-[2-(thiophen-2-yl)-5-methyloxazol-4-yl]ethoxy]benzyl]amino]acetic acid

The procedure of Step 5 of Example 3 was repeated except for using 547 mg (0.94 mmol) of the compound obtained in Step 1 and 59 mg (1.4 mmol) of lithium hydroxide monohydrate to obtain the title compound (515 mg, 99%).

¹H-NMR (CDCl₃, 300 MHz): δ (ppm) 7.60-7.59 (m, 1H), 7.45-7.37 (m, 2H), 7.16-7.06 (m, 5H), 6.97-6.94 (m, 1H), 6.79 (d, J=8.4 Hz, 2H), 4.51 (s, 2H), 4.16 (t, J=6.7 Hz, 2H), 4.03 (t, J=8.5 Hz, 2H), 3.90 (s, 2H), 3.10 (t, J=8.5 Hz, 2H), 2.96 (t, J=6.5 Hz, 2H), 2.36 (s, 3H).

### EXAMPLE 120

### Preparation of [N-(1,2,3,4-tetrahydroquinolinyl)sulfonyl-N-[4-[2-(2-phenyl-5-methyloxazol-4-yl)ethoxy]benzyl]amino]acetic acid

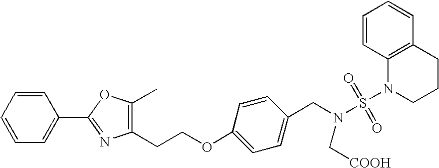

### Step 1: Preparation of [N-(1,2,3,4-tetrahydroquinolinyl)sulfonyl-N-[4-[2-(2-phenyl-5-methyloxazol-4-yl)ethoxy]benzyl]amino]acetic acid ethyl ester

The procedure of Step 1 of Example 61 was repeated except for using 404 mg (1 mmol) of the compound obtained in Step 2 of Example 57, 305 mg (1.5 mmol) of 2-[(5-methyl-2-phenyl)oxazol-4-yl]ethanol, 446 mg (1.7 mmol) of triphenylphosphine and 344 mg (1.7 mmol) of diisopropyl azocarboxylate to obtain the title compound (554 mg, 94%).

¹H-NMR (CDCl₃, 200 MHz): δ (ppm) 7.97 (m, 2H), 7.59 (d, J=8.1 Hz, 2H), 7.42 (m, 3H), 7.03 (m, 5H), 6.79 (d, J=8.7 Hz, 2H), 4.47 (s, 2H), 4.21 (t, J=6.7 Hz, 2H), 4.08 (q, J=7.2 Hz, 2H), 3.80 (m, 4H), 2.98 (t, J=6.4 Hz, 2H), 2.79 (t, J=6.7 Hz, 2H), 2.37 (s, 3H), 2.07 (m, 2H), 1.19 (t, J=7.2 Hz, 3H).

### Step 2: Preparation of [N-(1,2,3,4-tetrahydroquinolinyl)sulfonyl-N-[4-[2-(2-phenyl-5-methyloxazol-4-yl)ethoxy]benzyl]amino]acetic acid

The procedure of Step 5 of Example 3 was repeated except for using 554 mg (0.94 mmol) of the compound obtained in Step 1 and 59 mg (1.4 mmol) of lithium hydroxide monohydrate to obtain the title compound (523 mg, 99%).

¹H-NMR (CDCl₃, 300 MHz): δ (ppm), 9.61 (br s, 1H), 7.96-7.93 (m, 2H), 7.59 (d, J=8.1 Hz, 1H), 7.39-7.38 (m, 3H), 7.15-6.99 (m, 5H), 6.77 (d, J=8.7 Hz, 2H), 4.46 (s, 2H), 4.13 (t, J=7.2 Hz, 2H), 3.84 (s, 2H), 3.76 (t, J=5.9 Hz, 2H), 2.99 (t, J=6.5 Hz, 2H), 2.79 (t, J=6.6 Hz, 2H), 2.37 (s, 3H), 2.09 (m, 2H).

### EXAMPLE 121

### Preparation of [N-(1,2,3,4-tetrahydroquinolinyl)sulfonyl-N-[4-[2-[2-(4-methylphenyl)-5-methyloxazol-4-yl]ethoxy]benzyl]amino]acetic acid

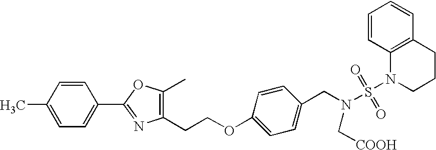

### Step 1: Preparation of [N-(1,2,3,4-tetrahydroquinolinyl)sulfonyl-N-[4-[2-[2-(4-methylphenyl)-5-methyloxazol-4-yl]ethoxy]benzyl]amino]acetic acid ethyl ester

The procedure of Step 1 of Example 61 was repeated except for using 404 mg (1 mmol) of the compound obtained in Step 2 of Example 57, 326 mg (1.5 mmol) of 2-[5-methyl-2-(4-methylphenyl)oxazol-4-yl]ethanol, 446 mg (1.7 mmol) of triphenylphosphine and 344 mg (1.7 mmol) of diisopropyl azocarboxylate to obtain the title compound (574 mg, 95%).

¹H-NMR (CDCl₃, 300 MHz): δ (ppm) 7.84 (d, J=8.1 Hz, 2H), 7.59 (d, J=8.1 Hz, 1H), 7.25-7.01 (m, 7H), 6.79 (d, J=8.4 Hz, 2H), 4.47 (s, 2H), 4.20 (t, J=6.6 Hz, 2H), 4.07 (q, J=7.2 Hz, 2H), 3.79-3.76 (m, 4H), 2.95 (t, J=6.3 Hz, 2H), 2.81 (t, J=6.6 Hz, 2H), 2.38 (s, 3H), 2.35 (s, 3H), 2.07-2.03 (m, 2H), 1.19 (t, J=7.2 Hz, 3H).

### Step 2: Preparation of [N-(1,2,3,4-tetrahydroquinolinyl)sulfonyl-N-[4-[2-[2-(4-methylphenyl)-5-methyloxazol-4-yl]ethoxy]benzyl]amino]acetic acid

The procedure of Step 5 of Example 3 was repeated except for using 574 mg (0.95 mmol) of the compound obtained in Step 1 and 59 mg (1.4 mmol) of lithium hydroxide monohydrate to obtain the title compound (541 mg, 99%).

¹H-NMR (CDCl₃, 300 MHz): δ (ppm) 7.83 (d, J=8.1 Hz, 2H), 7.59 (d, J=8.1 Hz, 2H), 7.25-6.99 (m, 7H), 6.76 (d, J=8.4 Hz, 2H), 5.04 (br s, 1H), 4.46 (s, 2H), 4.16 (t, J=6.6 Hz, 2H), 3.84 (s, 2H), 3.76 (t, J=5.7 Hz, 2H), 2.98 (t, J=6.3 Hz, 2H), 2.80 (t, J=6.6 Hz, 2H), 2.37 (s, 3H), 2.33 (s, 3H), 2.08-1.99 (m, 2H).

### EXAMPLE 122

### Preparation of [N-(1,2,3,4-tetrahydroquinolinyl)sulfonyl-N-[4-[2-[2-(4-trifluoromethylphenyl)-5-methyloxazol-4-yl]ethoxy]benzyl]amino]acetic acid

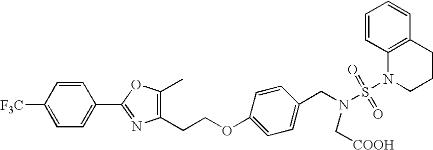

### Step 1: Preparation of [N-(1,2,3,4-tetrahydroquinolinyl)sulfonyl-N-[4-[2-[2-(4-trifluoromethylphenyl)-5-methyloxazol-4-yl]ethoxy]benzyl]amino]acetic acid ethyl ester

The procedure of Step 1 of Example 61 was repeated except for using 404 mg (1 mmol) of the compound obtained in Step 2 of Example 57, 407 mg (1.5 mmol) of 2-[5-methyl-2-[(4-trifluoromethyl)phenyl]oxazol-4-yl]ethanol, 446 mg (1.7 mmol) of triphenylphosphine and 344 mg (1.7 mmol) of diisopropyl azocarboxylate to obtain the title compound (605 mg, 92%).

¹H-NMR (CDCl₃, 300 MHz): δ (ppm) 8.08 (d, J=8.4 Hz, 2H), 7.68 (d, J=8.4 Hz, 2H), 7.60 (d, J=8.1 Hz, 1H), 7.17-7.01 (m, 5H), 6.79 (d, J=8.4 Hz, 2H), 4.48 (s, 2H), 4.22 (t, J=6.6 Hz, 2H), 4.06 (q, J=7.2 Hz, 2H), 3.79-3.76 (m, 4H), 2.97 (t, J=6.3 Hz, 2H), 2.82 (t, J=6.6 Hz, 2H), 2.39 (s, 3H), 2.09-2.01 (m, 2H), 1.18 (t, J=7.2 Hz, 3H).

### Step 2: Preparation of [N-(1,2,3,4-tetrahydroquinolinyl)sulfonyl-N-[4-[2-[2-(4-trifluoromethylphenyl)-5-methyloxazol-4-yl]ethoxy]benzyl]amino]acetic acid

The procedure of Step 5 of Example 3 was repeated except for using 605 mg (0.92 mmol) of the compound obtained in Step 1 and 59 mg (1.4 mmol) of lithium hydroxide monohydrate to obtain the title compound (573 mg, 99%).

¹H-NMR (CDCl₃, 300 MHz): δ (ppm) 8.06 (d, J=8.4 Hz, 2H), 7.67 (d, J=8.4 Hz, 2H), 7.58 (d, J=8.1 Hz, 1H), 7.16-6.99 (m, 5H), 6.77 (d, J=8.4 Hz, 2H), 5.89 (br s, 1H), 4.45 (s, 2H), 4.19 (t, J=6.6 Hz, 2H), 3.84 (s, 2H), 3.76 (t, J=5.8 Hz, 2H), 2.98 (t, J=6.3 Hz, 2H), 2.80 (t, J=6.6 Hz, 2H), 2.39 (s, 3H), 2.08-1.99 (m, 2H).

### EXAMPLE 123

### Preparation of [N-(1,2,3,4-tetrahydroquinolinyl)sulfonyl-N-[4-[2-[2-(thiophen-2-yl)-5-methoxyoxazol-4-yl]ethoxy]benzyl]amino]acetic acid

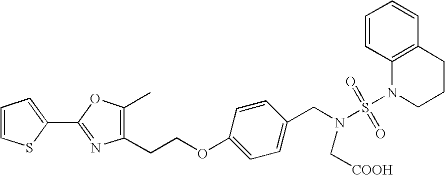

### Step 1: Preparation of [N-(1,2,3,4-tetrahydroquinolinyl)sulfonyl-N-[4-[2-[2-(thiophen-2-yl)-5-methoxyoxazol-4-yl]ethoxy]benzyl]amino]acetic acid ethyl ester

The procedure of Step 1 of Example 61 was repeated except for using 404 mg (1 mmol) of the compound obtained in Step 2 of Example 57, 314 mg (1.5 mmol) of 2-[5-methyl-2-(thiophen-2-yl)oxazol-4-yl]ethanol, 446 mg (1.7 mmol) of triphenylphosphine and 344 mg (1.7 mmol) of diisopropyl azocarboxylate to obtain the title compound (554 mg, 93%).

¹H-NMR (CDCl₃, 300 MHz): δ (ppm) 7.61-7.57 (m, 2H), 7.37-7.35 (m, 1H), 7.14-7.01 (m, 6H), 6.78 (d, J=8.4 Hz, 2H), 4.46 (s, 2H), 4.18 (t, J=6.6 Hz, 2H), 4.07 (q, J=7.2 Hz, 2H), 3.79-3.76 (m, 4H), 2.94 (t, J=6.3 Hz, 2H), 2.82 (t, J=6.6 Hz, 2H), 2.34 (s, 3H), 2.09-2.01 (m, 2H), 1.19 (t, J=7.2 Hz, 3H).

### Step 2: Preparation of [N-(1,2,3,4-tetrahydroquinolinyl)sulfonyl-N-[4-[2-[2-(thiophen-2-yl)-5-methoxyoxazol-4-yl]ethoxy]benzyl]amino]acetic acid

The procedure of Step 5 of Example 3 was repeated except for using 554 mg (0.93 mmol) of the compound obtained in Step 1 and 595 mg (1.4 mmol) of lithium hydroxide monohydrate to obtain the title compound (523 mg, 99%).

¹H-NMR (CDCl₃, 300 MHz): δ (ppm) 7.61-7.57 (m, 2H), 7.36-7.35 (m, 1H), 7.07-6.99 (m, 6H), 6.76 (d, J=8.4 Hz, 2H), 5.03 (br s, 1H), 4.45 (s, 2H), 4.15 (t, J=6.6 Hz, 2H), 3.84 (s, 2H), 3.76 (t, J=5.7 Hz, 2H), 2.95 (t, J=6.3 Hz, 2H), 2.80 (t, J=6.6 Hz, 2H), 2.34 (s, 3H), 2.09-2.01 (m, 2H).

### EXAMPLE 124

### Preparation of (S)-2-[N—(N,N-dimethylamino)sulfonyl-N-[3-[(5-methyl-2-p-tolyloxazol-4-yl)methoxy]benzyl]amino]propionic acid

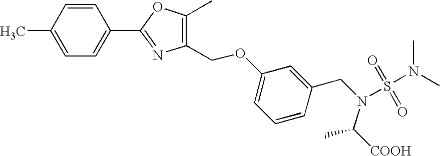

### Step 1: Preparation of (S)-2-[N—(N,N-dimethylamino)sulfonyl-N-[3-[(5-methyl-2-p-tolyloxazol-4-yl)methoxy]benzyl]amino]propionate ethyl ester

The procedure of Step 2 of Example 3 was repeated except for using 408 mg (1 mmol) of (S)-[[2-[3-[(5-methyl-2-p-tolyloxazol-4-yl)methoxy]benzyl]amino]propionate ethyl ester, 158 mg (1.1 mmol) of N,N-dimethyl sulfamoyl chloride and 111 mg (1.1 mmol) of triethylamine to obtain the title compound (170 mg, 33%).

¹H-NMR (CDCl₃, 200 MHz): δ (ppm) 7.90 (d, J=8.1 Hz, 2H), 7.29-7.21 (m, 3H), 7.08-689 (m, 3H), 4.98 (s, 2H), 4.61-4.07 (m, 5H), 2.77 (s, 6H), 2.42 (s, 3H), 2.39 (s, 3H), 1.40 (d, J=6.9 Hz, 3H), 1.26 (t, J=7.2 Hz, 3H).

### Step 2: Preparation of (S)-2-[N—(N,N-dimethylamino)sulfonyl-N-[3-[(5-methyl-2-p-tolyloxazol-4-yl)methoxy]benzyl]amino]propionic acid

The procedure of Step 5 of Example 3 was repeated except for using 170 mg (0.33 mmol) of the compound obtained in Step 1 and 23 mg (0.5 mmol) of lithium hydroxide monohydrate to obtain the title compound (159 mg, 99%).

¹H-NMR (CDCl₃, 200 MHz): δ (ppm) 9.74 (br s, 1H), 7.87 (d, J=8.1 Hz, 2H), 7.26-7.17 (m, 4H), 6.97-6.85 (m, 2H), 5.02 (s, 2H), 4.42-4.29 (m, 3H), 2.73 (s, 6H), 2.43 (s, 3H), 2.39 (s, 3H), 1.39 (d, J=6.9 Hz, 3H).

### EXAMPLE 125

### Preparation of (S)-3-methyl-2-[N—(N,N-dimethylamino) sulfonyl-N-[3-[(5-methyl-2-p-tolyloxazol-4-yl)methoxy]benzyl]amino]butyric acid

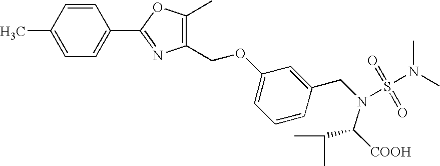

### Step 1: Preparation of (S)-3-methyl-2-[N—(N,N-dimethylamino)sulfonyl-N-[3-[(5-methyl-2-p-tolyloxazol-4-yl)methoxy]benzyl]amino]butyrate methyl ester

The procedure of Step 2 of Example 3 was repeated except for using 423 mg (1 mmol) of (S)-3-methyl-2-[3-[(5-methyl-2-p-tolyloxazol-4-yl)methoxy]benzyl]amino]butyrate methyl ester, 158 mg (1.1 mmol) of N,N-dimethyl sulfamoyl chloride and 111 mg (1.1 mmol) of triethylamine to obtain the title compound (344 mg, 65%).

¹H-NMR (CDCl₃, 300 MHz): δ (ppm) 7.90 (d, J=8.1 Hz, 2H), 7.26-7.20 (m, 3H), 7.09 (s, 1H), 7.03-7.01 (m, 1H), 6.93-6.89 (m, 1H), 4.97 (s, 2H), 4.60 (d, J=2.4 Hz, 2H), 3.97 (d, J=10.6 Hz, 1H), 3.72 (s, 3H), 2.65 (s, 6H), 2.42 (s, 3H), 2.39 (s, 3H), 2.13-2.05 (m, 1H), 0.87-0.83 (m, 6H).

### Step 2: Preparation of (S)-3-methyl-2-[N—(N,N-dimethylamino)sulfonyl-N-[3-[(5-methyl-2-p-tolyloxazol-4-yl)methoxy]benzyl]amino]butyric acid

The procedure of Step 5 of Example 3 was repeated except for using 344 mg (0.65 mmol) of the compound obtained in Step 1 and 41 mg (0.98 mmol) of lithium hydroxide monohydrate to obtain the title compound (332 mg, 99%).

¹H-NMR (CDCl₃, 200 MHz): δ (ppm) 10.18 (br s, 1H), 7.86 (d, J=8.1 Hz, 2H), 7.25-7.18 (m, 4H), 7.14-7.11 (m, 1H), 7.00-6.82 (m, 1H), 5.05 (s, 2H), 4.48 (s, 2H), 4.04 (d, J=10.7 Hz, 1H), 2.60 (s, 6H), 2.43 (s, 3H), 2.38 (s, 3H), 2.20-2.08 (m, 1H), 0.98-0.85 (m, 6H).

### EXAMPLE 126

### Preparation of [N—(N,N-dimethylamino)sulfonyl-N-[1-[[3-(5-methyl-2-p-tolyloxazol-4-yl)methoxy]phenyl]ethyl]amino]acetic acid

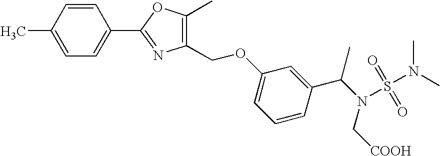

### Step 1: Preparation of [N—(N,N-dimethylamino)sulfonyl-N-[1-[[3-(5-methyl-2-p-tolyloxazol-4-yl)methoxy]phenyl]ethyl]amino]acetate methyl ester

The procedure of Step 2 of Example 3 was repeated except for using 394 mg (1 mmol) of [[1-[3-[(5-methyl-2-p-tolyloxazol-4-yl)methoxy]phenyl]ethyl]amino]acetate methyl ester, 158 mg (1.1 mmol) of N,N-dimethyl sulfamoyl chloride and 111 mg (1.1 mmol) of triethylamine to obtain the title compound (221 mg, 44%).

¹H-NMR (CDCl₃, 200 MHz): δ (ppm) 7.90 (d, J=8.1 Hz, 2H), 7.32-7.22 (m, 3H), 7.13-6.92 (m, 3H), 5.06 (q, J=6.9 Hz, 1H), 4.98 (s, 2H), 3.71 (q, J=18.3 Hz, J=22.7 Hz, 2H), 3.61 (s, 2H), 2.89 (s, 6H), 2.43 (s, 3H), 2.38 (s, 3H), 1.57 (d, J=6.9 Hz, 3H)

### Step 2: Preparation of [N—(N,N-dimethylamino)sulfonyl-N-[1-[[3-(5-methyl-2-p-tolyloxazol-4-yl)methoxy]phenyl]ethyl]amino]acetic acid

The procedure of Step 5 of Example 3 was repeated except for using 221 mg (0.44 mmol) of the compound obtained in Step 1 and 28 mg (0.66 mmol) of lithium hydroxide monohydrate to obtain the title compound (212 mg, 99%).

¹H-NMR (CDCl₃, 200 MHz): δ (ppm) 8.94 (br s, 1H), 7.86 (d, J=8.1 Hz, 2H), 7.28-7.14 (m, 4H), 7.03-6.92 (m, 1H), 6.88-6.87 (m, 1H), 5.06 (q, J=7.3 Hz, 1H), 4.99 (s, 2H), 3.74 (q, J=10.9 Hz, J=18.3 Hz, 2H), 2.86 (s, 6H), 2.42 (s, 3H), 2.38 (s, 3H), 1.57 (d, J=7.3 Hz, 3H).

### EXAMPLE 127

### Preparation of [N-(pyrrolidinyl)sulfonyl-N-[1-[[3-(5-methyl-2-p-tolyloxazol-4-yl)methoxy]phenyl]ethyl]amino]acetic acid

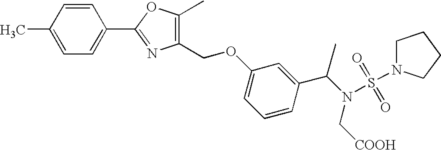

### Step 1: Preparation of [N-(pyrrolidinyl)sulfonyl-N-[1-[[3-(5-methyl-2-p-tolyloxazol-4-yl)methoxy]phenyl]ethyl]amino]acetate methyl ester

The procedure of Step 2 of Example 3 was repeated except for using 394 mg (1 mmol) of [[1-[[3-(5-methyl-2-p-tolyloxazol-4-yl)methoxy]phenyl]ethyl]amino]acetate methyl ester, 187 mg (1.1 mmol) of (pyrrolidinyl)sulfonyl chloride and 111 mg (1.1 mmol) of triethylamine to obtain the title compound (269 mg, 51%).

¹H-NMR (CDCl₃, 200 MHz): δ (ppm) 7.90 (d, J=8.1 Hz, 2H), 7.31-7.22 (m, 3H), 7.14-6.92 (m, 3H), 5.10 (q, J=7.3 Hz, 1H), 4.98 (s, 2H), 3.72 (q, J=18.0 Hz, J=24.4 Hz, 2H), 3.61 (s, 3H), 3.59-3.35 (m, 4H), 2.43 (s, 3H), 2.38 (s, 3H), 1.93-1.86 (m, 4H), 1.58 (d, J=7.3 Hz, 3H).

### Step 2: Preparation of [N-(pyrrolidinyl)sulfonyl-N-[1-[[3-(5-methyl-2-p-tolyl oxazol-4-yl)methoxy]phenyl]ethyl]amino]acetic acid

The procedure of Step 5 of Example 3 was repeated except for using 269 mg (0.51 mmol) of the compound obtained in Step 1 and 33 mg (0.77 mmol) of lithium hydroxide monohydrate to obtain the title compound (259 mg, 99%).

¹H-NMR (CDCl₃, 200 MHz): δ (ppm) 9.08 (br s, 1H), 7.87 (d, J=8.1 Hz, 2H), 7.29-7.14 (m, 4H), 7.03-6.92 (m, 1H), 6.89-6.87 (m, 1H), 5.10 (q, J=7.3 Hz, 1H), 4.99 (s, 2H), 3.76 (q, J=18.3 Hz, J=14.2 Hz, 2H), 3.39-3.33 (m, 4H), 2.42 (s, 3H), 2.38 (s, 3H), 1.89-1.82 (m, 4H), 1.57 (d, J=7.3 Hz, 3H).

### EXAMPLE 128

### Preparation of [N—(N,N-diethylamino)sulfonyl-N-[1-[[3-(5-methyl-2-p-tolyloxazol-4-yl)methoxy]phenyl]ethyl]amino]acetic acid

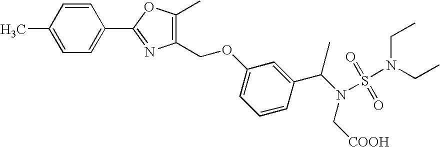

### Step 1: Preparation of [N—(N,N-diethylamino)sulfonyl-[1-[[3-(5-methyl-2-p-tolyloxazol-4-yl)methoxy]phenyl]ethyl]amino]acetate methyl ester

The procedure of Step 2 of Example 3 was repeated except for using 394 mg (1 mmol) of [[1-[3-[(5-methyl-2-p-tolyloxazol-4-yl)methoxy]phenyl]ethyl]amino]acetate methyl ester, 189 mg (1.1 mmol) of (N,N-diethylamino)sulfonyl chloride and 111 mg (1.1 mmol) of triethylamine to obtain the title compound (159 mg, 30%).

¹H-NMR (CDCl₃, 200 MHz): δ (ppm) 7.90 (d, J=8.2 Hz, 2H), 7.31-7.22 (m, 3H), 7.14-6.96 (m, 3H), 5.02 (q, 1H), 4.98 (s, 2H), 3.70 (q, J=18.3 Hz, 2H), 3.61 (s, 3H), 3.37 (q, J=7.1 Hz, 4H), 2.43 (s, 3H), 2.39 (s, 3H), 1.57 (d, J=7.1 Hz, 3H), 1.19 (t, J=7.1 Hz, 6H).

### Step 2: Preparation of [N—(N,N-diethylamino)sulfonyl-N-[1-[[3-(5-methyl-2-p-tolyloxazol-4-yl)methoxy]phenyl]ethyl]amino]acetic acid

The procedure of Step 5 of Example 3 was repeated except for using 159 mg (0.30 mmol) of the compound obtained in Step 1 and 20 mg (0.45 mmol) of lithium hydroxide monohydrate to obtain the title compound (153 mg, 99%).

¹H-NMR (CDCl₃, 200 MHz): δ (ppm) 7.87 (d, J=8.1 Hz, 2H), 7.59 (br s, 1H), 7.29-7.21 (m, 4H), 7.17-6.99 (m, 1H), 6.92-6.87 (m, 1H), 5.04 (q, 1H), 4.99 (s, 2H), 3.73 (d, J=4.9 Hz, 2H), 3.34 (q, J=7.1 Hz, 4H), 2.43 (s, 3H), 2.39 (s, 3H), 1.57 (d, J=7.1 Hz, 3H), 1.17 (t, J=7.1 Hz, 6H).

### EXAMPLE 129

### Preparation of [N—(N-isopropyl-N-methylamino)sulfonyl-N-[1-[[3-(5-methyl-2-p-tolyloxazol-4-yl)methoxy]phenyl]ethyl]amino]acetic acid

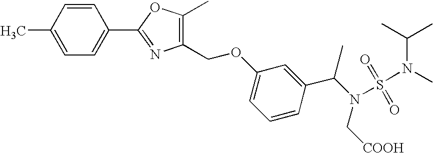

### Step 1: Preparation of [N—(N-isopropyl-N-methylamino)sulfonyl-[1-[[3-(5-methyl-2-p-tolyloxazol-4-yl)methoxy]phenyl]ethyl]amino]acetate methyl ester

The procedure of Step 2 of Example 3 was repeated except for using 394 mg (1 mmol) of [[1-[[3-(5-methyl-2-p-tolyloxazol-4-yl)methoxy]phenyl]ethyl]amino]acetate methyl ester, 189 mg (1.1 mmol) of (N-isopropyl-N-methylamino)sulfonyl chloride and 111 mg (1.1 mmol) of triethylamine to obtain the title compound (132 mg, 25%).

¹H-NMR (CDCl₃, 200 MHz): δ (ppm) 7.89 (d, J=8.1 Hz, 2H), 7.31-7.22 (m, 3H), 7.14-6.92 (m, 3H), 5.00 (q, 1H), 4.98 (s, 2H), 4.32-4.24 (m, 1H), 3.68 (q, J=18.3 Hz, 2H), 3.61 (s, 3H), 2.76 (s, 3H), 2.43 (s, 3H), 2.39 (s, 3H), 1.57 (d, J=7.1 Hz, 3H), 1.20 (s, 3H), 1.17 (s, 3H).

### Step 2: Preparation of [N—(N-isopropyl-N-methylamino)sulfonyl-[1-[[3-(5-methyl-2-p-tolyloxazol-4-yl)methoxy]phenyl]ethyl]amino]acetic acid

The procedure of Step 5 of Example 3 was repeated except for using 132 mg (0.25 mmol) of the compound obtained in Step 1 and 16 mg (0.38 mmol) of lithium hydroxide monohydrate to obtain the title compound (128 mg, 99%).

¹H-NMR (CDCl₃, 200 MHz): δ (ppm) 7.87 (d, J=8.1 Hz, 2H), 7.49 (br s, 1H), 7.29-7.17 (m, 4H), 7.03-6.87 (m, 2H), 5.02 (q, 1H), 4.99 (s, 2H), 4.28-4.22 (m, 1H), 3.72 (d, J=3.3 Hz, 2H), 2.72 (s, 3H), 2.43 (s, 3H), 2.39 (s, 3H), 1.57 (d, J=6.9 Hz, 3H), 1.19 (s, 3H), 1.15 (s, 3H).

### EXAMPLE 130

### Preparation of 3-[N—(N,N-dimethylamino)sulfonyl-N-[3-[(5-methyl-2-p-tolyloxazol-4-yl)methoxy]benzyl]amino]propionic acid

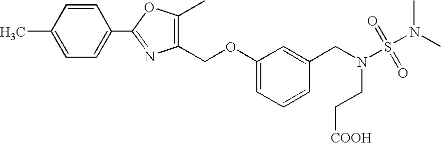

### Step 1: Preparation of 3-[N—(N,N-dimethylamino)sulfonyl-N-[3-[(5-methyl-2-p-tolyloxazol-4-yl)methoxy]benzyl]amino]propionate ethyl ester

The procedure of Step 2 of Example 3 was repeated except for using 408 mg (1 mmol) of 3-[[3-[(5-methyl-2-p-tolyloxazol-4-yl)methoxy]benzyl]amino]propionate ethyl ester, 158 mg (1.1 mmol) of N,N-dimethyl sulfamoyl chloride and 111 mg (1.1 mmol) of triethylamine to obtain the title compound (474 mg, 92%).

¹H-NMR (CDCl₃, 200 MHz): δ (ppm) 7.90 (d, J=8.1 Hz, 2H), 7.28-7.22 (m, 3H), 7.04-6.93 (m, 3H), 4.98 (s, 2H), 4.35 (s, 2H), 4.07 (q, J=7.2 Hz, 2H), 3.45 (t, J=6.5 Hz, 2H), 2.79 (s, 6H), 2.54 (t, J=6.5 Hz, 2H), 2.43 (s, 3H), 2.39 (s, 3H), 1.22 (t, J=7.2 Hz, 3H).

### Step 2: Preparation of 3-[N—(N,N-dimethylamino)sulfonyl-N-[3-[(5-methyl-2-p-tolyloxazol-4-yl)methoxy]benzyl]amino]propionic acid

The procedure of Step 5 of Example 3 was repeated except for using 474 mg (0.92 mmol) of the compound obtained in Step 1 and 58 mg (1.38 mmol) of lithium hydroxide monohydrate to obtain the title compound (444 mg, 99%).

¹H-NMR (CDCl₃, 300 MHz): δ (ppm) 10.39 (br s, 1H), 7.87 (d, J=8.1 Hz, 2H), 7.28-7.22 (m, 3H), 7.03-6.90 (m, 3H), 5.00 (s, 2H), 4.33 (s, 2H), 3.44 (t, J=6.5 Hz, 2H), 2.78 (s, 6H), 2.55 (t, J=6.5 Hz, 2H), 2.42 (s, 3H), 2.38 (s, 3H).

### EXAMPLE 131

### Preparation of [N—(N,N-dimethylarino)sulfonyl-N-[3-[(2-phenyloxazol-4-yl)ethoxy]benzyl]amino]acetic acid

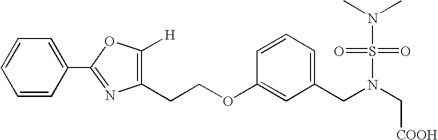

### Step 1: Preparation of [N—(N,N-dimethylamino)sulfonyl-N-[3-[(2-phenyloxazol-4-yl)ethoxy]benzyl]amino]acetic acid ethyl ester

The procedure of Step 2 of Example 3 was repeated except for using 380 mg (1 mmol) of [[3-[2-(2-phenyloxazol-4-yl)ethoxy]benzyl]amino]acetic acid ethyl ester, 158 mg (1.1 mmol) of N,N-dimethyl sulfamoyl chloride and 111 mg (1.1 mmol) of triethylamine to obtain the title compound (375 mg, 77%).

¹H-NMR (CDCl₃, 200 MHz): δ (ppm) 8.04-8.00 (m, 2H), 7.58 (s, 1H), 7.45-7.43 (m, 3H), 7.28-7.22 (m, 1H), 6.91-6.85 (m, 3H), 4.52 (s, 2H), 4.28 (t, J=6.5 Hz, 2H), 4.16 (q, J=7.2 Hz, 2H), 3.85 (s, 2H), 3.08 (t, J=6.5 Hz, 2H), 2.87 (s, 6H), 1.24 (t, J=7.2 Hz, 3H).

### Step 2: Preparation of [N—(N,N-dimethylamino)sulfonyl-N-[3-[(2-phenyloxazol-4-yl)ethoxy]benzyl]amino]acetic acid

The procedure of Step 5 of Example 3 was repeated except for using 375 mg (0.77 mmol) of the compound obtained in Step 1 and 49 mg (1.16 mmol) of lithium hydroxide monohydrate to obtain the title compound (350 mg, 99%).

¹H-NMR (CDCl₃, 200 MHz): δ (ppm) 9.37 (br s, 1H), 8.01-7.89 (m, 2H), 7.58 (s, 1H), 7.46-7.44 (m, 3H), 7.28-7.22 (m, 1H), 6.98-6.85 (m, 3H), 4.51 (s, 2H), 4.28 (t, J=6.5 Hz, 2H), 3.91 (s, 2H), 3.07 (t, J=6.5 Hz, 2H), 2.86 (s, 6H).

### EXAMPLE 132

### Preparation of [N—(N,N-dimethylamino)sulfonyl-N-[4-[(2-phenyloxazol-4-yl)ethoxy]benzyl]amino]acetic acid

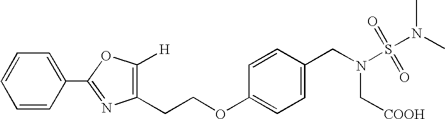

### Step 1: Preparation of [N—(N,N-dimethylamino)sulfonyl-N-[4-[(2-phenyloxazol-4-yl)ethoxy]benzyl]amino]acetic acid ethyl ester

The procedure of Step 2 of Example 3 was repeated except for using 380 mg (1 mmol) of [[4-[2-(2-phenyloxazol-4-yl)ethoxy]benzyl]amino]acetic acid ethyl ester, 158 mg (1.1 mmol) of N,N-dimethyl sulfamoyl chloride and 111 mg (1.1 mmol) of triethylamine to obtain the title compound (419 mg, 86%).

¹H-NMR (CDCl₃, 200 MHz): δ (ppm) 8.04-7.99 (m, 2H), 7.57 (s, 1H), 7.46-7.43 (m, 3H), 7.24 (d, J=8.6 Hz, 2H), 6.88 (d, J=8.6 Hz, 2H), 4.48 (s, 2H), 4.28 (t, J=6.5 Hz, 2H), 4.18 (q, J=7.2 Hz, 2H), 3.81 (s, 2H), 3.08 (t, J=6.5 Hz, 2H), 2.86 (s, 6H), 1.24 (t, J=7.2 Hz, 3H).

### Step 2: Preparation of [N—(N,N-dimethylamino)sulfonyl-N-[4-[(2-phenyloxazol-4-yl)ethoxy]benzyl]amino]acetic acid

The procedure of Step 5 of Example 3 was repeated except for using 419 mg (0.86 mmol) of the compound obtained in Step 1 and 54 mg (1.29 mmol) of lithium hydroxide monohydrate to obtain the title compound (391 mg, 99%).

¹H-NMR (CDCl₃, 200 MHz): δ (ppm) 11.32 (br s, 1H), 8.03-7.97 (m, 2H), 7.58 (s, 1H), 7.47-7.42 (m, 3H), 7.24 (d, J=8.6 Hz, 2H), 6.88 (d, J=8.6 Hz, 2H), 4.48 (s, 2H), 4.25 (t, J=6.5 Hz, 2H), 3.87 (s, 2H), 3.10 (t, J=6.5 Hz, 2), 2.85 (s, 6H)

### EXAMPLE 133

### Preparation of [N—[[N-(4-chlorophenyl)-N-methyl]amino]sulfonyl-N-[4-[(2-phenyloxazol-4-yl)ethoxy]benzyl]amino]acetic acid

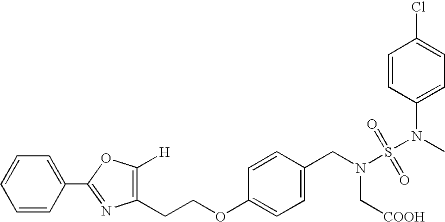

### Step 1: Preparation of [N—[[N-(4-chlorophenyl)-N-methyl]amino]sulfonyl-N-[4-[(2-phenyloxazol-4-yl)ethoxy]benzyl]amino]acetic acid ethyl ester

The procedure of Step 2 of Example 3 was repeated except for using 380 mg (1 mmol) of [[4-[2-(2-phenyloxazol-4-yl)ethoxy]benzyl]amino]acetic acid ethyl ester, 264 mg (1.1 mmol) of [N-[(4-chlorophenyl)-N-methyl]amino]sulfonyl chloride and 111 mg (1.1 mmol) of triethylamine to obtain the title compound (456 mg, 78%).

¹H-NMR (CDCl₃, 200 MHz): δ (ppm) 8.02 (m, 2H), 7.56 (s, 1H), 7.46-7.29 (m, 7H), 7.15 (d, J=8.6 Hz, 2H), 6.86 (d, J=8.6 Hz, 2H), 4.42 (s, 2H), 4.27 (t, J=6.5 Hz, 2H), 4.15 (q, J=7.2 Hz, 2H), 3.81 (s, 2H), 3.27 (s, 3H), 3.08 (t, J=6.5 Hz, 2H), 1.24 (t, J=7.2 Hz, 3H).

### Step 2: Preparation of [N—[[N-(4-chlorophenyl)-N-methyl]amino]sulfonyl-N-[4-[(2-phenyloxazol-4-yl)ethoxy]benzyl]amino]acetic acid

The procedure of Step 5 of Example 3 was repeated except for using 456 mg (0.78 mmol) of the compound obtained in Step 1 and 49 mg (1.17 mmol) of lithium hydroxide monohydrate to obtain the title compound (429 mg, 99%).

¹H-NMR (CDCl₃, 200 MHz): δ (ppm) 9.46 (br s, 1H), 8.02-7.95 (m, 2H), 7.58 (s, 1H), 7.48-7.25 (m, 6H), 7.14 (d, J=8.6 Hz, 2H), 6.84 (d, J=8.6 Hz, 2H), 4.42 (s, 2H), 4.24 (t, J=6.5 Hz, 2H), 3.85 (s, 2H), 3.25 (s, 3H), 3.09 (t, J=6.5 Hz, 2H).

### EXAMPLE 134

### Preparation of [N—[(N,N-dimethylamino)sulfonyl]-N-[4-[(2-phenyl-5-isopropyloxazol-4-yl)methoxy]benzyl]amino]acetic acid

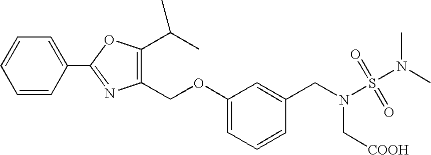

### Step 1: Preparation of [N—[(N,N-dimethylamino)sulfonyl]-N-[4-[(2-phenyl-5-isopropyloxazol-4-yl)methoxy]benzyl]amino]acetic acid ethyl ester

The procedure of Step 2 of Example 3 was repeated except for using 408 mg (1 mmol) of [[3-[(5-isopropyl-2-phenyloxazol-4-yl)methoxy]benzyl]amino]acetic acid ethyl ester, 158 mg (1.1 mmol) of N,N-dimethyl sulfamoyl chloride and 111 mg (1.1 mmol) of triethylamine to obtain the title compound (402 mg, 78%).

¹H-NMR (CDCl₃, 200 MHz): δ (ppm) 8.05-7.98 (m, 2H), 7.47-7.40 (m, 3H), 7.31-7.23 (m, 1H), 6.99-6.91 (m, 3H), 4.99 (s, 2H), 4.54 (s, 2H), 4.16 (q, J=7.1 Hz, 2H), 3.85 (s, 2H), 3.29-3.15 (m, 1H), 2.87 (s, 6H), 1.36 (s, 3H), 1.32 (s, 3H), 1.25 (t, J=7.1 Hz, 3H).

### Step 2: Preparation of [N—[(N,N-dimethylamino)sulfonyl]-N-[4-[(2-phenyl-5-isopropyloxazol-4-yl)methoxy]benzyl]amino]acetic acid

The procedure of Step 5 of Example 3 was repeated except for using 402 mg (0.78 mmol) of the compound obtained in Step 1 and 49 mg (1.17 mmol) of lithium hydroxide monohydrate to obtain the title compound (371 mg, 99%).

¹H-NMR (CDCl₃, 200 MHz): δ (ppm) 9.96 (br s, 1H), 8.03-7.97 (m, 2H), 7.47-7.41 (m, 3H), 7.28-7.19 (m, 1H), 6.99-6.87 (m, 3H), 5.03 (s, 2H), 4.47 (s, 2H), 3.88 (s, 2H), 3.27-3.17 (m, 1H), 2.83 (s, 6H), 1.36 (s, 3H), 1.32 (s, 3H).

### TEST EXAMPLE 1

### PPAR α Agonist Assay

Step 1) Exogeneous PPAR α Agonist Assay

Cross-activation assay of the compounds of Examples 1 to 134 against human PPAR α was carried out using a DNA construct of PPAR α including its ligand binding domain and GAL4 DNA binding domain (Sher et al., Biochemistry 32, 5598-5604, 1993) to analyze whether any of the compounds binds to the ligand binding domain of PPAR α expressed by the construct to affect the PPAR α activity. When the GAL4 DNA binding domainm which is fused with PPAR α ligand binding domain, binds to UAS (upstream activation sequence) of firefly-luciferase, the luciferase is expressed. Therefore, the effect of each of the compounds of Examples on PPAR α activation can be evaluated by analyzing the activation of luciferase.

Specifically, 2×10⁴ of HepG2 (ATCC HB-8065) or 293T cells (ATCC CRL-1573) were aliquotted into each well of a 96-well plate and cultured for overnight at 37° C. in a CO₂ incubator (culture condition: DMEM, 10% FBS and 1% antibiotics).

The transformation of PPAR α DNA was traced as follows. First, a mixture of the following ingredients in amounts per well; Solution A (Promega) containing 50 ng of full length human PPAR α DNA, 50 ng of firefly-luciferase construct, 5 ng of Renilla-luciferase construct, 1.0 μl of PLUS™ reagent, and 8.3 μl of a serum-free medium containing no antibiotics; was prepared and kept at room temperature for 15 min. Solution B, which contained 0.5 μl of Lipofectamine™ reagent and 9.5 μl of serum-free medium without antibiotics per well, and Solution C, which contained 50 μl of serum-free medium per well), were prepared. Solutions A, B and C were mixed together to prepare lipofectamin-DNA complex, and the mixture was kept at room temperature for 15 min. Single layer of cells on each well of a 96-well plate were washed with serum-free DMEM, 70 μl of lipofectamine-DNA complex prepared above was added thereto, the plate was incubated at 37° C. in a 5% CO₂ incubator for 3 hours to transform the cells, and 50 μM of a diluted solution of each of the compounds of Examples 1 to 134 per well was added to each well to a final concentration 25 μM, and incubated at 37° C. in a CO₂ incubator for 48 hours. Then, the medium was removed, 50 μl of passive lysis buffer (Promega) was added thereto, and the plate was shook at room temperature for 20 min to lysis cells. 40 μl of Luciferase Assay reagent of Dual Luciferase Kit (Invitrogen) per 20 μl of cell lysis buffer was added to the plate, followed by reacting at 20° C. for 8 seconds, and 40 μl of Stop & Glo reagent (Promega) as a stop solution was added thereto to quench the reaction. The luminescence intensities of F-luciferase and R-luciferase were measured with MicroLumat Plus (Berthord Technology). The result was analyzed in terms of activity increase when 25 μM of one of the compounds of Examples was treated, relative to the control; and EC₅₀, the concentration at which the activity of the test compound increased by 50% over that of control (Tables 1a to 1c, wherein ND means not treating the inventive compound).

Step 2) Endogeneous PPAR α Agonist Assay

The procedure of Step 1 was repeated except for using a firefly-luciferase DNA construct containing Renilla-luciferase construct, full length human PPAR α, RXR (retinoid X receptor) and PPRE (peroxisome proliferator-responsive element) (Sher et al., Biochemistry 32, 5598-5604, 1993; and Fleischhauer et al., Hum Genet 90, 505-510, 1993) to transform 293T cells, to conduct endogenous PPAR CL agonist assay. Each of the compounds of Examples was used in such a treatment which lasted for 48 hours, and the luciferase activity of the resulting heterodimer, a R/R (endogenous binding partner of PPAR) bound to a full length PPAR CL with the compounds, was measured in accordance with the same method of Step 1. The measured values are showed in Tables 1a to 1c, wherein ND means not treated with any of the inventive compound.

As shown in Tables 1a to 1c, the inventive compounds have a useful effect on significant increase of PPAR, a lipid metabolism related enzyme.

TABLE 1a

| | Activity at 25 μM | | EC₅₀ (μM) | | |
|---|---|---|---|---|---|
| | Step 1 | | Step 1 | | Step 2 |
| Example | 293T | HepG2 | 293T | HepG2 | Endogenous |
| No. | cell | cell | cell | cell | gene activity |
| 2 | 6 | 25 | 5 | 5 | 1 |
| 3 | 2 | ND | 5 | 5 | 25 |
| 4 | 3 | ND | 1 | 1 | 25 |
| 5 | 2 | ND | 0.2 | 0.2 | 5 |
| 6 | 2 | ND | 0.2 | 1 | 25 |
| 7 | 3 | 54 | ND | 0.2 | ND |
| 8 | 4 | 31 | 5 | 0.2 | ND |
| 9 | 3 | 52 | 0.2 | 1 | ND |
| 10 | 3 | 45 | 0.2 | 1 | ND |
| 11 | 4 | 44 | 0.2 | 1 | ND |
| 12 | 2 | ND | 25 | 25 | 25 |
| 13 | 2 | ND | 5 | 25 | 25 |
| 14 | 2 | ND | ND | 25 | 25 |
| 15 | 2 | ND | ND | 5 | 25 |
| 18 | 2 | ND | 5 | 25 | 25 |
| 19 | 2 | ND | 5 | 5 | 25 |
| 20 | 2 | ND | 5 | 5 | 25 |
| 21 | 5 | 40 | 1 | 5 | ND |
| 22 | 4 | 49 | 1 | 1 | ND |
| 23 | 5 | 34 | 1 | 5 | ND |
| 25 | 2 | ND | 5 | 5 | 100 |
| 26 | 2 | ND | 25 | 100 | 25 |
| 27 | 1 | ND | ND | 25 | 25 |
| 28 | 3 | ND | 5 | 25 | ND |
| 29 | 2 | ND | ND | 100 | ND |
| 30 | 2 | ND | 25 | 25 | 100 |
| 31 | 3 | ND | 5 | 25 | ND |
| 32 | 2 | ND | 1 | 5 | ND |
| 34 | 2 | ND | ND | 100 | 100 |
| 35 | 2 | ND | 5 | 5 | 100 |
| 36 | 2 | ND | ND | 25 | ND |
| 38 | 2 | ND | 25 | 25 | 100 |
| 39 | 2 | ND | 25 | 25 | ND |

TABLE 1b

| | Activity at 25 μM | | EC₅₀ (μM) | | |
|---|---|---|---|---|---|
| | Step 1 | | Step 1 | | Step 2 |
| Example | 293T | HepG2 | 293T | HepG2 | Endogenous |
| No. | cell | cell | cell | cell | gene activity |
| 40 | 2 | ND | 25 | 25 | ND |
| 42 | 3 | ND | 5 | 25 | ND |
| 43 | 2 | ND | 0.2 | 100 | 100 |
| 44 | 3 | ND | 25 | 100 | ND |
| 46 | 2 | ND | ND | 25 | 25 |
| 47 | 2 | ND | 25 | 5 | 25 |
| 48 | 2 | ND | ND | 25 | 100 |
| 49 | 3 | ND | ND | 25 | 100 |
| 50 | 2 | ND | 5 | 25 | 25 |
| 53 | 2 | ND | ND | 5 | 100 |
| 54 | 2 | ND | ND | 5 | 100 |
| 55 | 2 | ND | ND | 5 | 100 |
| 58 | 4 | ND | 25 | 5 | 100 |
| 59 | 3 | ND | ND | 25 | 100 |
| 61 | 2 | ND | 5 | 1 | 5 |
| 62 | 2 | ND | 0.2 | 0.2 | 1 |
| 63 | 3 | 33 | ND | 0.2 | ND |
| 64 | 2 | 30 | 0.2 | 0.2 | ND |
| 65 | 1 | ND | 0.2 | 0.2 | 1 |
| 66 | 1 | ND | ND | 1 | 100 |
| 67 | 2 | 50 | ND | 0.2 | ND |
| 68 | 2 | 11 | 5 | ND | ND |
| 69 | 2 | 40 | 0.2 | 0.2 | ND |
| 70 | 3 | 34 | 0.2 | 0.2 | ND |
| 71 | 2 | 44 | 0 | 0.2 | ND |
| 72 | 2 | ND | 5 | 1 | 25 |
| 80 | 5 | 30 | 5 | 5 | 1 |
| 81 | 3 | 33 | 5 | 1 | 5 |
| 82 | 2 | 26 | 5 | 1 | 0.2 |
| 84 | 1 | ND | 0.2 | 5 | 25 |
| 85 | 1 | ND | 0.2 | 5 | 25 |
| 86 | 2 | ND | 5 | ND | 5 |
| 87 | 1 | ND | ND | 100 | 25 |
| 88 | 1 | ND | ND | 5 | ND |
| 89 | 1 | ND | ND | 5 | ND |
| 94 | 2 | ND | ND | ND | ND |

TABLE 1c

| | Activity at 25 μM | | EC₅₀ (μM) | | |
|---|---|---|---|---|---|
| | Step 1 | | Step 1 | | Step 2 |
| Example | 293T | HepG2 | 293T | HepG2 | Endogenous |
| No. | cell | cell | cell | cell | gene activity |
| 98 | 1 | ND | 5 | 25 | 25 |
| 99 | 2 | ND | 25 | 5 | 25 |
| 100 | 2 | ND | 0.2 | 25 | ND |
| 101 | 4 | 45 | 5 | 1 | ND |
| 102 | 4 | 46 | 5 | 0.2 | ND |
| 103 | 2 | ND | 25 | 5 | 25 |
| 104 | 1 | ND | ND | 25 | 100 |
| 105 | 2 | ND | ND | 25 | ND |
| 106 | 2 | ND | 5 | 5 | 100 |
| 108 | 2 | ND | ND | ND | ND |
| 109 | 3 | ND | 25 | 5 | 100 |
| 110 | 2 | ND | 1 | 5 | 25 |
| 111 | 5 | 25 | 0.2 | 1 | 5 |
| 112 | 6 | 30 | 1 | 1 | 5 |
| 113 | 4 | 29 | 1 | 1 | 5 |
| 114 | 2 | ND | 1 | ND | 25 |
| 115 | 2 | ND | 25 | 25 | 25 |
| 116 | 2 | ND | 25 | 25 | ND |
| 117 | 3 | ND | 1 | 5 | 25 |
| 118 | 2 | ND | 1 | 5 | 25 |
| 119 | 2 | ND | 25 | ND | ND |
| 120 | 2 | ND | ND | 25 | ND |
| 121 | 2 | ND | 5 | 25 | 100 |
| 122 | 2 | ND | ND | 25 | ND |
| 123 | 2 | ND | ND | ND | ND |
| 124 | 5 | 40 | 5 | 5 | 0.2 |
| 125 | 3 | 22 | ND | 5 | 5 |
| 126 | 4 | 62 | 5 | 1 | ND |
| 127 | 3 | 50 | 25 | 1 | ND |
| 128 | 2 | 25 | ND | ND | ND |
| 129 | 3 | 43 | 1 | 1 | ND |
| 130 | 4 | 36 | 0.2 | 5 | ND |
| 131 | 5 | 41 | 5 | 5 | 5 |
| 132 | 5 | 32 | ND | 25 | 5 |
| 133 | 4 | 34 | 5 | 5 | 25 |
| 134 | 2 | 36 | ND | 25 | ND |

### TEST EXAMPLE 2

### Anti-Obesity Test

In order to evaluate the anti-obesity activity of the inventive compound, B6.V-Lepob/J mice (laboratory animal team of Korea Research Institute of Chemical Technology) were used for tests and lean mice as a control. Ob/ob mice are model animals for insulin-independent diabetes, suitable for studying the effect of an anti-obesity drug.

42 mice weighing 38.6±1.4 g were selected randomly from a pool of ob/ob mice (7-9 weeks old), and divided into groups of 7 mice each. The test was carried out at a constant temperature (24±0.5° C.) and humidity (55±5%) using ventilation system (10-12 times/hour), under a specific pathogen free (SPF) condition created in an automatic on-off cycle of 12 hours.

The groups were divided to a control group treated only with an excipient, a positive control group treated with fenofibrate, and test groups treated with each of the compounds of Examples 5, 62 and 65, at dosage 100 mg/10 ml/kg body weight. Each of the test and control compounds was diluted with 0.5% CMC to a concentration to match the above dosage, and administered orally using a syringe into the stomach twice a day (9:00 and 18:00) for a period of 4 weeks, and the following characteristics were measured.

1) General Symptoms and Observation of Dead Animal

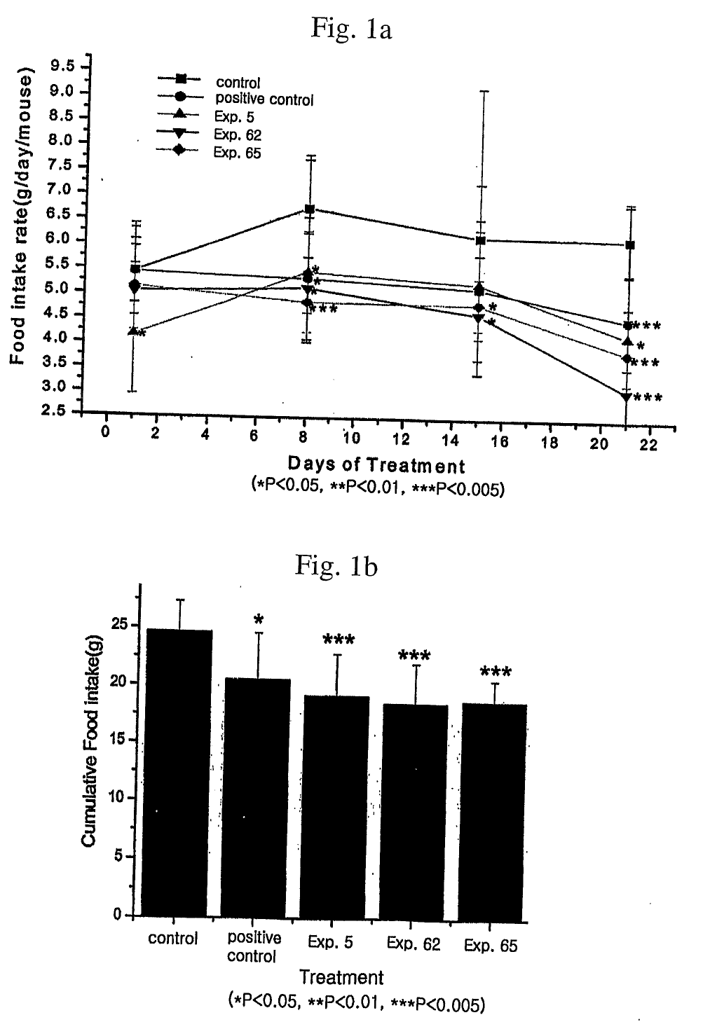

The conditions, behavioral changes, addictive symptoms and the death rate were observed constantly during the test period. As shown in FIGS. 1a and 1b, the daily feed intakes of the test groups treated with the compounds of Examples 5, 62 and 65 were significantly reduced for the first week of the test as compared to the control group (P<0.005 significance level, FIG. 1a), and the cumulative amounts of total feed intake showed a similar result (P<0.05 significance level, FIG. 1b).

Therefore, the compound of the present invention exerts a positive effect on reducing feed intake.

2) Body Weight Measurement

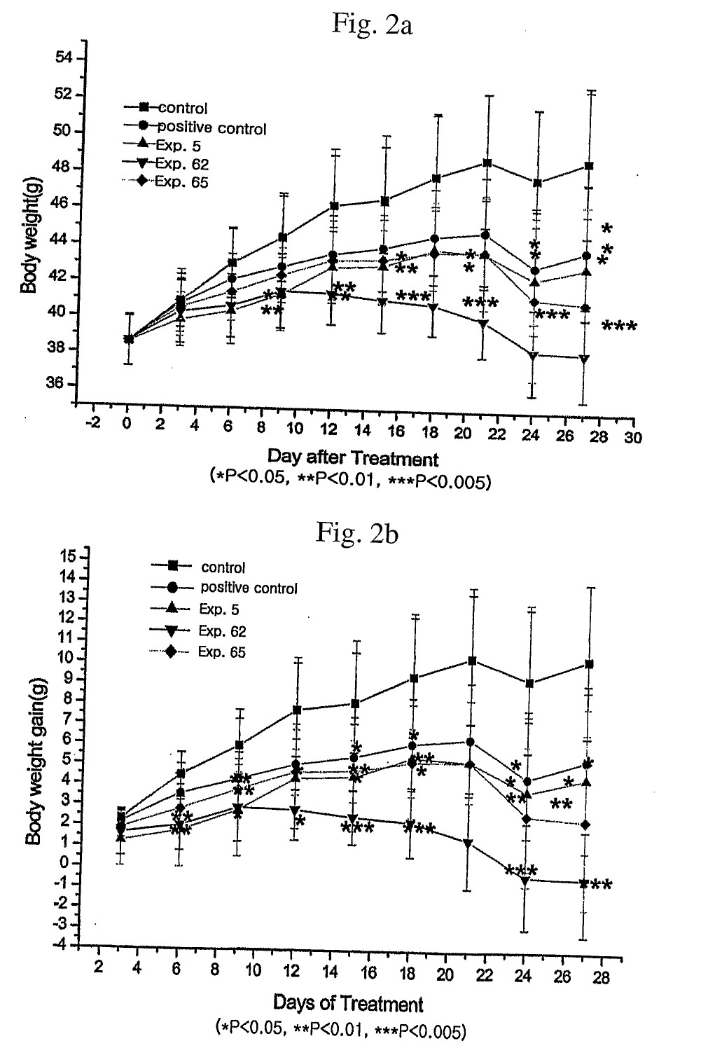

The average body weight of each group was measured every 3 days after the administration, and the changes in the average body weight and body weight gain (measured body weight—initial body weight) were analyzed. As shown in FIGS. 2a (the change in the average body weight) and 2b (the change in the body weight gain), the body weight of each of the test groups was significantly reduced as compared to that of the control group. Particularly, the change in the average body weight of the test group treated with the compound of Example 62 was markedly reduced at day 10 from the administration as compared to the control group (P<0.01, P<0.005 significance level, FIG. 2), and the change in the body weight gain of the compound was evidently lower at day 7 from the administration as compared to the control group (P<0.01 significance level, FIG. 2). The test group treated with the compound of Example 62 showed an improved effect as compared to the positive control group treated with fenofibrate, the compounds of Examples 5 and 65 showing similar results.

3) Measurement of Lipid Metabolism-Related Materials and Insulin Concentration in Blood

28 days after the administration, experimental mice were fasted for 16 hours, and the concentrations of lipid metabolism-related materials such as triglyceride, free fatty acid and cholesterol, and insulin in blood were measured. Specifically, a blood sample was collected from the ocular venous plexus of each mouse and centrifuged to separate plasma. The glucose (GLU), HDL-cholesterol (HDL), LDL-cholesterol (LDL), triglyceride (TG) and free fatty acid (NEFA) levels were measured by enzymatic assay using 2 kinds of Selectra (Vital Lab), an automatic biochemical analyzer, and the insulin concentration was measured by ELISA using an insulin detection kit (Shibayagi). The result is showed in Table 2.

TABLE 2

| | unit | excipient | fenofibrate | Example 5 | Example 62 | Example 65 |
|---|---|---|---|---|---|---|
| TG | mg/dl | 57.0 ± 14.5 | 49.4 ± 6.1 | 47.3 ± 12.4 | 37.9 ± 6.6* | 45.9 ± 4.7 |
| HDL | mg/dl | 273.6 ± 32.4 | 280.5 ± 28.5 | 292.1 ± 24.7 | 381.8 ± 40.1*** | 321.4 ± 31.8* |
| GLU | mg/dl | 309.4 ± 107.2 | 223.1 ± 77.2** | 218.1 ± 125.5* | 190.4 ± 77.6** | 158.8 ± 44.0*** |
| NEFA | UEq/l | 979 ± 204 | 1,003 ± 80 | 880 ± 273 | 976 ± 238 | 1,006 ± 203 |
| LDL | mg/dl | 34.3 ± 8.6 | 27.0 ± 4.7 | 24.3 ± 4.3* | 37.8 ± 5.2 | 33.0 ± 9.3 |

Significance as compared to a control treated with excipient:
*P < 0.05,
**P < 0.01,
***P < 0.005

As shown in Table 2, the free fatty acid concentration was similar among the groups, and the triglyceride concentration was significantly lower for the group treated with the compound of Example 62 as compared to a control (P<0.05 significance level). The LDL concentration of the group treated with the compound of Example 5 (P<0.05 significance level) was low. The HDL concentration was high for groups treated with the compounds of Example 62 and 65, respectively, and the GLU concentrations of the groups treated with the compounds of Example 5, 62 and 65, respectively, were much lower than that of the control or positive control group.

4) Oral Glucose Tolerance Test (OGTT)

28 days after the administration, each of the experimental mice was fasted for 16 hours, 2 g/kg of glucose was administered thereto, a blood sample was collected from the ocular venous plexus thereof at 0, 15, 30, 60 and 120 min from the administration, and the glucose concentration and AUC (area under the curve, Skrumsager et al., J Clin Pharmacol. 43, 1244-1256, 2003) were determined.

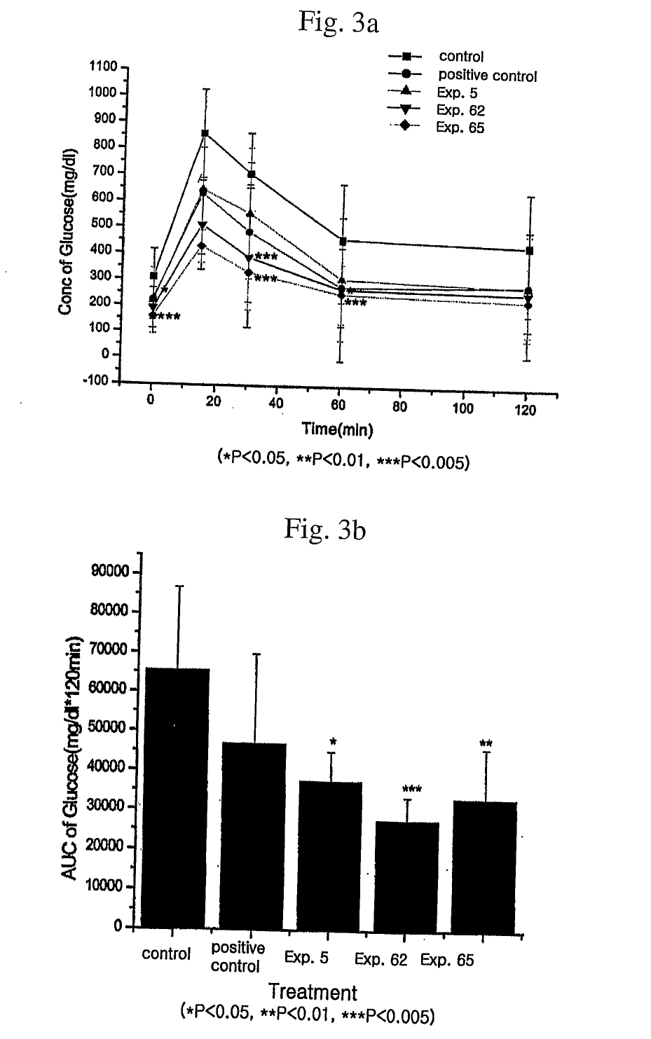

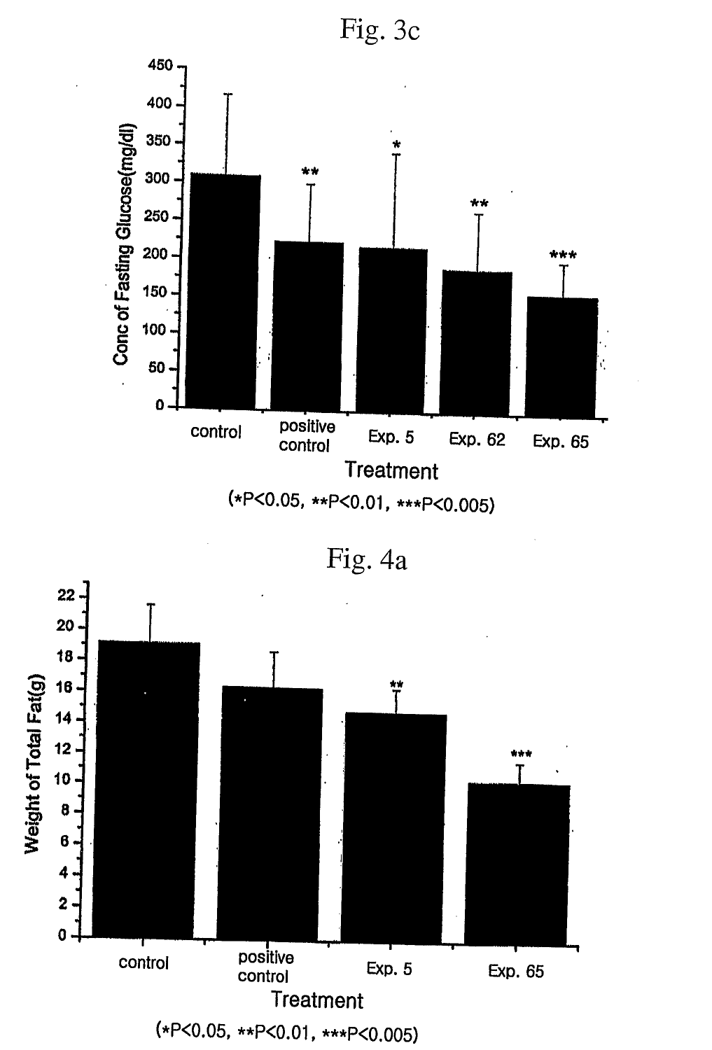

As shown in FIGS. 3a to 3c, the glucose concentration during the fast was observed to the significantly low for the test groups treated with the compounds of Examples 5, 62 and 65, respectively, as compared to the control group (P<0.05 significance level, FIG. 3c). The glucose concentration became significantly low at 30 and 60 min after the glucose administration for the test groups treated with the compounds of Examples 62 and 65, respectively, as compared to the control group (P<0.01, P<0.05 significance level, FIG. 3a), and the total changes in the glucose concentration represented by AUC were much lower for the test groups treated with the compounds of Examples 5, 62 and 65, respectively, than that of the control group (P<0.05, P<0.005 and P<0.01 significance level, FIG. 3b).

Thus, the inventive compounds can confer glucose tolerance to an animal.

5) Dissection

After completing all tests, each of the mice in test treated with the compounds of Examples 5 and 62 and the positive control group treated with fenofibrate was sacrificed, the whole blood, liver, muscle and white adipose tissue thereof were extracted. White adipose tissued was divided into visceral fat and subcutaneous fat, and the amounts of the total fats and liver tissue were weighed separately.

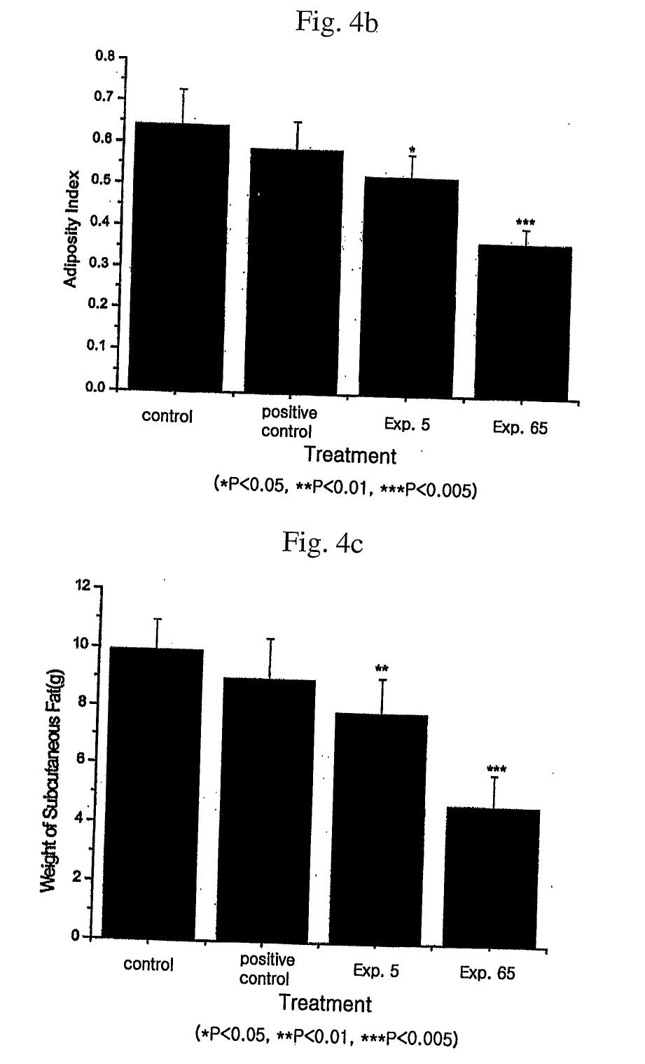

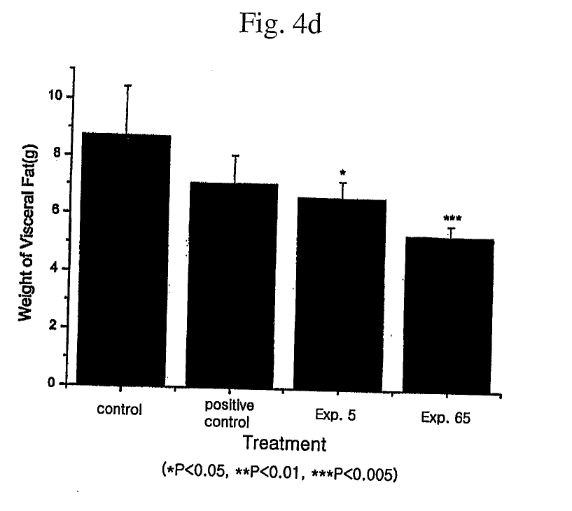

As shown in FIGS. 4a to 4d, the total fat weight observed for each of the test groups treated with the compounds of Examples 5 and 62 was low (P<0.01, P<0.005 significance level, FIG. 4a). The adiposity index (fat weight/(body weight—fat weight)) (FIG. 4b), and the weights of visceral fat and subcutaneous fat (FIG. 4d) were each reduced.

While the invention has been described with respect to the above specific embodiments, it should be recognized that various modifications and changes may be made and also fall within the scope of the invention as defined by the claims that follow.

What is claimed is:

1. A sulfamide derivative of formula (I) or a pharmaceutically acceptable salt, hydrate or solvate thereof. 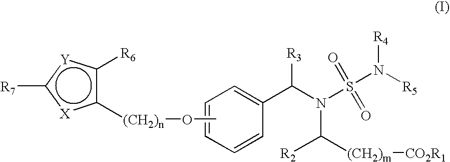
wherein,
R₁, R₂ and R₃ are each independently hydrogen or C₁₋₃ alkyl;
R₄ and R₅ are each independently hydrogen; C₁₋₅ alkyl; C₃₋₅ alkenyl; C₃₋₅ alkynyl; phenyl which is unsubstituted or substituted with C₁₋₃ alkyl, C₁₋₃ alkoxy, C₁₋₃ halogenated alkyl or halogen; or are fused together with the nitrogen atom to which they are attached to form a heterocyclic ring comprising a nitrogen atom;
R₆ is hydrogen or C₁₋₃ alkyl;
R₇ is phenyl which is unsubstituted or substituted with C₁₋₃ alkyl, C₁₋₃ halogenated alkyl or C₁₋₃ alkoxy; or thiophen;
X is nitrogen when Y is oxygen, and X is oxygen when Y is nitrogen;
m is 0 or 1; and
n is 1 or 2.

2. The derivative of claim 1, which is selected from the group consisting of:
1) [N-(aminosulfonyl)-N-[[4-(2-phenyl-5-methyloxazol-4-yl)ethoxy]benzyl]amino]acetic acid;
2) (S)-3-methyl-2-[N-(sulfamoyl)-N-[3-[(5-methyl-2-p-tolyloxazol-4-yl)methoxy]benzyl]amino]butyric acid;
3) [N—[(N,N-dimethylamino)sulfonyl]-N-[3-[(2-phenyl-5-methyloxazol-4-yl)methoxy]benzyl]amino]acetic acid;
4) [N—[(N,N-dimethylamino)sulfonyl]-N-[3-[[2-(4-methylphenyl)-5-methyloxazol-4-yl]methoxy]benzyl]amino]acetic acid;
5) [N—(N,N-dimethylamino)sulfonyl-N-[3-[[2-(4-trifluoromethylphenyl)-5-methyloxazol-4-yl]methoxy]benzyl]amino]acetic acid;
6) [N—(N-t-butylamino)sulfonyl-N-[3-[[2-(4-trifluoromethylphenyl)-5-methyloxazol-4-yl]methoxy]benzyl]amino]acetic acid;
7) [N—(N,N-diethylamino)sulfonyl-N-[3-[[2-(4-trifluoromethylphenyl)-5-methyloxazol-4-yl]methoxy]benzyl]amino]acetic acid;
8) [N—(N-isopropyl-N-methylamino)sulfonyl-N-[3-[[2-(4-trifluoromethyl phenyl)-5-methyloxazol-4-yl]methoxy]benzyl]amino]acetic acid;
9) [N—(N-allyl-N-methylamino)sulfonyl-N-[3-[[2-(4-trifluoromethyl phenyl)-5-methyloxazol-4-yl]methoxy]benzyl]amino]acetic acid;
10) [N—(N-methyl-N-propargyl)sulfonyl-N-[3-[[2-(4-trifluoromethyl phenyl)-5-methyloxazol-4-yl]methoxy]benzyl]amino]acetic acid;
11) [N-(piperidinyl)sulfonyl-N-[3-[[2-(4-trifluoromethylphenyl)-5-methyloxazol-4-yl]methoxy]benzyl]amino]acetic acid;
12) [N—(N,N-dimethylamino)sulfonyl-N-[3-[[2-(thiophen-2-yl)-5-methyl oxazol-4-yl]methoxy]benzyl]amino]acetic acid;
13) [N—(N,N-dimethylamino)sulfonyl-N-[3-[[2-(4-trifluoromethylphenyl)-4-methyloxazol-5-yl]methoxy]benzyl]amino]acetic acid;
14) [N—[(N-methyl-N-phenyl)amino]sulfonyl-N-[3-[(2-phenyl-5-methyl oxazol-4-yl)methoxy]benzyl]amino]acetic acid;
15) [N—[(N-methyl-N-phenyl)amino]sulfonyl-N-[3-[[2-(4-methylphenyl)-5-methyloxazol-4-yl]methoxy]benzyl]amino]acetic acid;
16) [N—[(N-methyl-N-phenyl)amino]sulfonyl-N-[3-[[2-(4-trifluoromethyl phenyl)-5-methyloxazol-4-yl]methoxy]benzyl]amino]acetic acid;
17) [N—[(N-methyl-N-phenyl)amino]sulfonyl-N-[3-[[2-(thiophen-2-yl)-5-methyloxazol-4-yl]methoxy]benzyl]amino]acetic acid;
18) [N—[[N-methyl-N-(4-chlorophenyl)]amino]sulfonyl-N-[3-[(2-phenyl-5-methyloxazol-4-yl)methoxy]benzyl]amino]acetic acid;
19) [N—[[N-methyl-N-(4-chlorophenyl)]amino]sulfonyl-N-[3-[[2-(4-methylphenyl)-5-methyloxazol-4-yl]methoxy]benzyl]amino]acetic acid;
20) [N—[[N-methyl-N-(4-chlorophenyl)]amino]sulfonyl-N-[3-[[2-(4-tri fluoromethylphenyl)-5-methyloxazol-4-yl]methoxy]benzyl]amino]acetic acid;
21) [N—(N-ethyl-N-m-tolylamino)sulfonyl-N-[3-[[2-(4-trifluoromethyl phenyl)-5-methyloxazol-4-yl]methoxy]benzyl]amino]acetic acid;
22) [N—(N-p-anisoyl-N-methylamino)sulfonyl-N-[3-[[2-(4-trifluoro methylphenyl)-5-methyloxazol-4-yl]methoxy]benzyl]amino]acetic acid;
23) [N—[N-(3-fluorophenyl)-N-methylamino]sulfonyl-N-[3-[[2-(4-tri fluoromethylphenyl)-5-methyloxazol-4-yl]methoxy]benzyl]amino]acetic acid;
24) [N—[[N-methyl-N-(p-chlorophenyl)]amino]sulfonyl-N-[3-[[2-(thiophen-2-yl)-5-methyloxazol-4-yl]methoxy]benzyl]amino]acetic acid;
25) [N-(pyrrolidinyl)sulfonyl-N-[3-[[2-(4-trifluoromethylphenyl)-5-methyloxazol-4-yl]methoxy]benzyl]amino]acetic acid;
26) [N-(pyrrolidinyl)sulfonyl-N-[3-[[2-(4-trifluoromethylphenyl)-4-methyloxazol-5-yl]methoxy]benzyl]amino]acetic acid;
27) [N-(4-methyl-1-piperazinyl)sulfonyl-N-[3-[[2-(4-trifluoromethyl phenyl)-5-methyloxazol-4-yl]methoxy]benzyl]amino]acetic acid;
28) [N-(morpholinyl)sulfonyl-N-[3-[[2-(4-methylphenyl)-5-methyl oxazol-4-yl]methoxy]benzyl]amino]acetic acid;
29) [N-(morpholinyl)sulfonyl-N-[3-[[2-(4-trifluoromethylphenyl)-4-methyloxazol-5-yl]methoxy]benzyl]amino]acetic acid;
30) [N-(indolinyl)sulfonyl-N-[3-[(2-phenyl-5-methyloxazol-4-yl)methoxy]benzyl]amino]acetic acid;
31) [N-(indolinyl)sulfonyl-N-[3-[[2-(4-methylphenyl)-5-methyloxazol-4-yl]methoxy]benzyl]amino]acetic acid;
32) [N-(indolinyl)sulfonyl-N-[3-[[2-(4-trifluoromethylphenyl)-5-methyl oxazol-4-yl]methoxy]benzyl]amino]acetic acid;
33) [N-(indolinyl)sulfonyl-N-[3-[[2-(thiophen-2-yl)-5-methyloxazol-4-yl]methoxy]benzyl]amino]acetic acid;
34) [N-(1,2,3,4-tetrahydroquinolinyl)sulfonyl-N-[3-[(2-phenyl-5-methyl oxazol-4-yl)methoxy]benzyl]amino]acetic acid;
35) [N-(1,2,3,4-tetrahydroquinolinyl)sulfonyl-N-[3-[[2-(4-methylphenyl)-5-methyloxazol-4-yl]methoxy]benzyl]amino]acetic acid;
36) [N-(1,2,3,4-tetraquinolinyl)sulfonyl-N-[3-[[2-(4-trifluoromethyl phenyl)-5-methyloxazol-4-yl]methoxy]benzyl]amino]acetic acid;
37) [N-(1,2,3,4-tetraquinolinyl)sulfonyl-N-[3-[[2-(thiophen-2-yl)-5-methyloxazol-4-yl]methoxy]benzyl]amino]acetic acid;
38) [N—(N,N-dimethylamino)sulfonyl-N-[4-[(2-phenyl-5-methyloxazol-4-yl)methoxy]benzyl]amino]acetic acid;
39) [N—(N,N-dimethylamino)sulfonyl-N-[4-[[2-(4-methylphenyl)-5-methyloxazol-4-yl]methoxy]benzyl]amino]acetic acid;
40) [N—(N,N-dimethylamino)sulfonyl-N-[4-[[2-(4-trifluoromethylphenyl)-5-methyloxazol-4-yl]methoxy]benzyl]amino]acetic acid;
41) [N—(N,N-dimethylamino)sulfonyl-N-[4-[[2-(thiophen-2-yl)-5-methyl oxazol-4-yl]methoxy]benzyl]amino]acetic acid;
42) [N-(pyrrolidinyl)sulfonyl-N-[4-[[2-(4-methylphenyl)-5-methyloxazol-4-yl]methoxy]benzyl]amino]acetic acid;
43) [N-(4-methyl-1-piperazinyl)sulfonyl-N-[4-[[2-(4-methylphenyl)-5-methyloxazol-4-yl]methoxy]benzyl]amino]acetic acid;
44) [N-(morpholinyl)sulfonyl-N-[4-[[2-(4-methylphenyl)-5-methyloxazol-4-yl]methoxy]benzyl]amino]acetic acid;
45) [N—[(N-methyl-N-phenyl)amino]sulfonyl-N-[4-[(2-phenyl-5-methyl oxazol-4-yl)methoxy]benzyl]amino]acetic acid;
46) [N—[(N-methyl-N-phenyl)amino]sulfonyl-N-[4-[[2-(4-methylphenyl)-5-methyloxazol-4-yl]methoxy]benzyl]amino]acetic acid;
47) [N—[(N-methyl-N-phenyl)amino]sulfonyl-N-[4-[[2-(4-trifluoromethyl phenyl)-5-methyloxazol-4-yl]methoxy]benzyl]amino]acetic acid;
48) [N—[(N-methyl-N-phenyl)amino]sulfonyl-N-[4-[[2-(thiophen-2-yl)-5-methyloxazol-4-yl]methoxy]benzyl]amino]acetic acid;
49) [N—[[N-methyl-N-(4-chlorophenyl)]amino]sulfonyl-N-[4-[(2-phenyl-5-methyloxazol-4-yl)methoxy]benzyl]amino]acetic acid;
50) [N—[[N-methyl-N-(4-chlorophenyl)]amino]sulfonyl-N-[4-[[2-(4-methylphenyl)-5-methyloxazol-4-yl]methoxy]benzyl]amino]acetic acid;
51) [N—[[N-methyl-N-(4-chlorophenyl)]amino]sulfonyl-N-[4-[[2-(4-tri fluoromethylphenyl)-5-methyloxazol-4-yl]methoxy]benzyl]amino]acetic acid;
52) [N—[[N-methyl-N-(4-chlorophenyl)]amino]sulfonyl-N-[4-[[2-(thiophen-2-yl)-5-methyloxazol-4-yl]methoxy]benzyl]amino]acetic acid;
53) [N-(indolinyl)sulfonyl-N-[4-[(2-phenyl-5-methyloxazol-4-yl)methoxy]benzyl]amino]acetic acid;
54) [N-(indolinyl)sulfonyl-N-[4-[[2-(4-methylphenyl)-5-methyloxazol-4-yl]methoxy]benzyl]amino]acetic acid;
55) [N-(indolinyl)sulfonyl-N-[4-[[2-(4-trifluoromethylphenyl)-5-methyl oxazol-4-yl]methoxy]benzyl]amino]acetic acid;
56) [N-(indolinyl)sulfonyl-N-[4-[[2-(thiophen-2-yl)-5-methyloxazol-4-yl]methoxy]benzyl]amino]acetic acid;
57) [N-(1,2,3,4-tetrahydroquinolinyl)sulfonyl-N-[4-[(2-phenyl-5-methyl oxazol-4-yl)methoxy]benzyl]amino]acetic acid;
58) [N-(1,2,3,4-tetraquinolinyl)sulfonyl-N-[4-[[2-(4-methylphenyl)-5-methyloxazol-4-yl]methoxy]benzyl]amino]acetic acid;
59) [N-(1,2,3,4-tetrahydroquinolinyl)sulfonyl-N-[4-[[2-(4-trifluoro methylphenyl)-5-methyloxazol-4-yl]methoxy]benzyl]amino]acetic acid;
60) [N-(1,2,3,4-tetrahydroquinolinyl)sulfonyl-N-[4-[[2-(thiophen-2-yl)-5-methyloxazol-4-yl]methoxy]benzyl]amino]acetic acid;
61) [N—(N,N-dimethylamino)sulfonyl-N-[3-[2-(2-phenyl-5-methyl oxazol-4-yl)ethoxy]benzyl]amino]acetic acid;
62) [N—(N,N-dimethylamino)sulfonyl-N-[3-[2-[2-(4-methylphenyl)-5-methyloxazol-4-yl]ethoxy]benzyl]amino]acetic acid;
63) [N—(N,N-diethylamino)sulfonyl-N-[3-[2-[2-(4-methylphenyl)-5-methyloxazol-4-yl]ethoxy]benzyl]amino]acetic acid;
64) [N—(N-isopropyl-N-methylamino)sulfonyl-N-[3-[2-[2-(4-methyl phenyl)-5-methyloxazol-4-yl]ethoxy]benzyl]amino]acetic acid;
65) [N—(N,N-dimethylamino)sulfonyl-N-[3-[2-[2-(4-trifluoromethyl phenyl)-5-methyloxazol-4-yl]ethoxy]benzyl]amino]acetic acid;
66) [N—(N-t-butylamino)sulfonyl-N-[3-[2-[2-(4-trifluoromethylphenyl)-5-methyloxazol-4-yl]ethoxy]benzyl]amino]acetic acid;
67) [N—(N,N-diethylamino)sulfonyl-N-[3-[2-[2-(4-trifluoromethylphenyl)-5-methyloxazol-4-yl]ethoxy]benzyl]amino]acetic acid;
68) [N—(N-isopropyl-N-methylamino)sulfonyl-N-[3-[2-[2-(4-trifluoro methylphenyl)-5-methyloxazol-4-yl]ethoxy]benzyl]amino]acetic acid;
69) [N—(N-allyl-N-methylamino)sulfonyl-N-[3-[2-[2-(4-trifluoromethyl phenyl)-5-methyloxazol-4-yl]ethoxy]benzyl]amino]acetic acid;
70) [N—(N-methyl-N-propargylamino)sulfonyl-N-[3-[2-[2-(4-trifluoro methylphenyl)-5-methyloxazol-4-yl]ethoxy]benzyl]amino]acetic acid;
71) [N-(piperidinyl)sulfonyl-N-[3-[2-[2-(4-trifluoromethylphenyl)-5-methyloxazol-4-yl]ethoxy]benzyl]amino]acetic acid;
72) [N—(N,N-dimethylamino)sulfonyl-N-[3-[2-[2-(thiophen-2-yl)-5-methyloxazol-4-yl]ethoxy]benzyl]amino]acetic acid;
73) [N—[(N-methyl-N-phenyl)amino]sulfonyl-N-[3-[2-(2-phenyl-5-methyloxazol-4-yl)ethoxy]benzyl]amino]acetic acid;
74) [N—[(N-methyl-N-phenyl)amino]sulfonyl-N-[3-[2-[2-(4-methyl phenyl)-5-methyloxazol-4-yl]ethoxy]benzyl]amino]acetic acid;
75) [N—[(N-methyl-N-phenyl)amino]sulfonyl-N-[3-[2-[2-(4-trifluoro methylphenyl)-5-methyloxazol-4-yl]ethoxy]benzyl]amino]acetic acid;
76) [N—[(N-methyl-N-phenyl)amino]sulfonyl-N-[3-[2-[2-(thiophen-2-yl)-5-methyloxazol-4-yl]ethoxy]benzyl]amino]acetic acid;
77) [N—[[N-methyl-N-(4-chlorophenyl)]amino]sulfonyl-N-[3-[2-(2-phenyl-5-methyloxazol-4-yl)ethoxy]benzyl]amino]acetic acid;
78) [N—[[N-methyl-N-(4-chlorophenyl)]amino]sulfonyl-N-[3-[2-[2-(4-methylphenyl)-5-methyloxazol-4-yl]ethoxy]benzyl]amino]acetic acid;
79) [N—[[N-methyl-N-(4-chlorophenyl)]amino]sulfonyl-N-[3-[2-[2-(4-trifluoromethylphenyl)-5-methyloxazol-4-yl]ethoxy]benzyl]amino]acetic acid;
80) [N—(N-ethyl-N-m-tolylamino)sulfonyl-N-[3-[2-[2-(4-trifluoro methylphenyl)-5-methyloxazol-4-yl]ethoxy]benzyl]amino]acetic acid;
81) [N—(N-anisoyl-N-methylamino)sulfonyl-N-[3-[2-[2-(4-trifluoro methylphenyl)-5-methyloxazol-4-yl]ethoxy]benzyl]amino]acetic acid;
82) [N—[N-(3-fluorophenyl)-N-methylamino]sulfonyl-N-[3-[2-[2-(4-tri fluoromethylphenyl)-5-methyloxazol-4-yl]ethoxy]benzyl]amino]acetic acid;
83) [N—[[N-methyl-N-(4-chlorophenyl)]amino]sulfonyl-N-[3-[2-[2-(thiophen-2-yl)-5-methyloxazol-4-yl]ethoxy]benzyl]amino]acetic acid;
84) [N-(pyrrolidinyl)sulfonyl-N-[3-[2-[2-(4-methylphenyl)-5-methyl oxazol-4-yl]ethoxy]benzyl]amino]acetic acid;
85) [N-(pyrrolidinyl)sulfonyl-N-[3-[2-[2-(4-trifluoromethylphenyl)-5-methyloxazol-4-yl]ethoxy]benzyl]amino]acetic acid;
86) [N-(morpholinyl)sulfonyl-N-[3-[2-[2-(4-methylphenyl)-5-methyl oxazol-4-yl]ethoxy]benzyl]amino]acetic acid;
87) [N-(morpholinyl)sulfonyl-N-[3-[2-[2-(4-trifluoromethylphenyl)-5-methyloxazol-4-yl]ethoxy]benzyl]amino]acetic acid;
88) [N-(4-methyl-1-piperazinyl)sulfonyl-N-[3-[2-[2-(4-methylphenyl)-5-methyloxazol-4-yl]ethoxy]benzyl]amino]acetic acid;
89) [N-(4-methyl-1-piperazinyl)sulfonyl-N-[3-[2-[2-(4-trifluoromethyl phenyl)-5-methyloxazol-4-yl]ethoxy]benzyl]amino]acetic acid;
90) [N-(indolinyl)sulfonyl-N-[3-[2-(2-phenyl-5-methyloxazol-4-yl)ethoxy]benzyl]amino]acetic acid;
91) [N-(indolinyl)sulfonyl-N-[3-[2-[2-(4-methylphenyl)-5-methyloxazol-4-yl]ethoxy]benzyl]amino]acetic acid;
92) [N-(indolinyl)sulfonyl-N-[3-[2-[2-(4-trifluoromethylphenyl)-5-methyloxazol-4-yl]ethoxy]benzyl]amino]acetic acid;
93) [N-(indolinyl)sulfonyl-N-[3-[2-[2-(thiophen-2-yl)-5-methyloxazol-4-yl]ethoxy]benzyl]amino]acetic acid;
94) [N-(1,2,3,4-tetrahydroquinolinyl)sulfonyl-N-[3-[2-(2-phenyl-5-methyloxazol-4-yl)ethoxy]benzyl]amino]acetic acid;
95) [N-(1,2,3,4-tetraquinolinyl)sulfonyl-N-[3-[2-[2-(4-methylphenyl)-5-methyloxazol-4-yl]ethoxy]benzyl]amino]acetic acid;
96) [N-(1,2,3,4-tetrahydroquinolinyl)sulfonyl-N-[3-[2-[2-(4-trifluoro methylphenyl)-5-methyloxazol-4-yl]ethoxy]benzyl]amino]acetic acid;
97) [N-(1,2,3,4-tetrahydroquinolinyl)sulfonyl-N-[3-[2-[2-(thiophen-2-yl)-5-methyloxazol-4-yl]ethoxy]benzyl]amino]acetic acid;
98) [N—(N,N-dimethylamino)sulfonyl-N-[4-[2-(2-phenyl-5-methyl oxazol-4-yl)ethoxy]benzyl]amino]acetic acid;
99) [N—(N,N-dimethylamino)sulfonyl-N-[4-[2-[2-(4-methylphenyl)-5-methyloxazol-4-yl]ethoxy]benzyl]amino]acetic acid;
100) [N—(N-t-butylamino)sulfonyl-N-[4-[2-[2-(4-methylphenyl)-5-methyl oxazol-4-yl]ethoxy]benzyl]amino]acetic acid;
101) [N—(N,N-diethylamino)sulfonyl-N-[4-[2-[2-(4-methylphenyl)-5-methyloxazol-4-yl]ethoxy]benzyl]amino]acetic acid;
102) [N—(N-isopropyl-N-methylamino)sulfonyl-N-[4-[2-[2-(4-methyl phenyl)-5-methyloxazol-4-yl]ethoxy]benzyl]amino]acetic acid;
103) [N—(N,N-dimethylamino)sulfonyl-N-[4-[2-[2-(4-trifluoromethyl phenyl)-5-methyloxazol-4-yl]ethoxy]benzyl]amino]acetic acid;
104) [N—(N,N-dimethylamino)sulfonyl-N-[4-[2-[2-(thiophen-2-yl)-5-methyloxazol-4-yl]ethoxy]benzyl]amino]acetic acid;
105) [N—[(N-methyl-N-phenyl)amino]sulfonyl-N-[4-[2-(2-phenyl-5-methyloxazol-4-yl)ethoxy]benzyl]amino]acetic acid;
106) [N—[(N-methyl-N-phenyl)amino]sulfonyl-N-[4-[2-[2-(4-methyl phenyl)-5-methyloxazol-4-yl]ethoxy]benzyl]amino]acetic acid;
107) [N—[(N-methyl-N-phenyl)amino]sulfonyl-N-[4-[2-[2-(4-trifluoro methylphenyl)-5-methyloxazol-4-yl]ethoxy]benzyl]amino]acetic acid;
108) [N—[(N-methyl-N-phenyl)amino]sulfonyl-N-[4-[2-[2-(thiophen-2-yl)-5-methyloxazol-4-yl]ethoxy]benzyl]amino]acetic acid;
109) [N—[[N-methyl-N-(4-chlorophenyl)]amino]sulfonyl-N-[4-[2-(2-phenyl-5-methyloxazol-4-yl)ethoxy]benzyl]amino]acetic acid;
110) [N—[[N-methyl-N-(4-chlorophenyl)]amino]sulfonyl-N-[4-[2-[2-(4-methylphenyl)-5-methyloxazol-4-yl]ethoxy]benzyl]amino]acetic acid;
111) [N—(N-ethyl-N-m-tolylamino)sulfonyl-N-[4-[2-[2-(4-methylphenyl)-5-methyloxazol-4-yl]ethoxy]benzyl]amino]acetic acid;
112) [N—(N-anisoyl-N-methylamino)sulfonyl-N-[4-[2-[2-(4-methyl phenyl)-5-methyloxazol-4-yl]ethoxy]benzyl]amino]acetic acid;
113) [N—[N-(3-fluorophenyl)-N-methylamino]sulfonyl-N-[4-[2-[2-(4-methylphenyl)-5-methyloxazol-4-yl]ethoxy]benzyl]amino]acetic acid;
114) [N—[[N-methyl-N-(4-chlorophenyl)]amino]sulfonyl-N-[4-[2-[2-(4-trifluoromethylphenyl)-5-methyloxazol-4-yl]ethoxy]benzyl]amino]acetic acid;
115) [N—[[N-methyl-N-(4-chlorophenyl)]amino]sulfonyl-N-[4-[2-[2-(thiophen-2-yl)-5-methyloxazol-4-yl]ethoxy]benzyl]amino]acetic acid;
116) [N-(indolinyl)sulfonyl-N-[4-[2-[(2-phenyl-5-methyloxazol-4-yl)ethoxy]benzyl]amino]acetic acid;
117) [N-(indolinyl)sulfonyl-N-[4-[2-[2-(4-methylphenyl)-5-methyl oxazol-4-yl]ethoxy]benzyl]amino]acetic acid;
118) [N-(indolinyl)sulfonyl-N-[4-[2-[2-(4-trifluoromethylphenyl)-5-methyloxazol-4-yl]ethoxy]benzyl]amino]acetic acid;
119) [N-(indolinyl)sulfonyl-N-[4-[2-[2-(thiophen-2-yl)-5-methyloxazol-4-yl]ethoxy]benzyl]amino]acetic acid;
120) [N-(1,2,3,4-tetrahydroquinolinyl)sulfonyl-N-[4-[2-(2-phenyl-5-methyloxazol-4-yl)ethoxy]benzyl]amino]acetic acid;
121) [N-(1,2,3,4-tetrahydroquinolinyl)sulfonyl-N-[4-[2-[2-(4-methyl phenyl)-5-methyloxazol-4-yl]ethoxy]benzyl]amino]acetic acid;
122) [N-(1,2,3,4-tetrahydroquinolinyl)sulfonyl-N-[4-[2-[2-(4-trifluoro methylphenyl)-5-methyloxazol-4-yl]ethoxy]benzyl]amino]acetic acid;
123) [N-(1,2,3,4-tetrahydroquinolinyl)sulfonyl-N-[4-[2-[2-(thiophen-2-yl)-5-methoxyoxazol-4-yl]ethoxy]benzyl]amino]acetic acid;
124) (S)-2-[N—(N,N-dimethylamino)sulfonyl-N-[3-[(5-methyl-2-p-tolyl oxazol-4-yl)methoxy]benzyl]amino]propionic acid;
125) (S)-3-methyl-2-[N—(N,N-dimethylamino)sulfonyl-N-[3-[(5-methyl-2-p-tolyloxazol-4-yl)methoxy]benzyl]amino]butyric acid;
126) [N—(N,N-dimethylamino)sulfonyl-N-[1-[[3-(5-methyl-2-p-tolyl oxazol-4-yl)methoxy]phenyl]ethyl]amino]acetic acid;
127) [N-(pyrrolidinyl)sulfonyl-N-[1-[[3-(5-methyl-2-p-tolyloxazol-4-yl)methoxy]phenyl]ethyl]amino]acetic acid;
128) [N—(N,N-diethylamino)sulfonyl-N-[1-[[3-(5-methyl-2-p-tolyloxazol-4-yl)methoxy]phenyl]ethyl]amino]acetic acid;
129) [N—(N-isopropyl-N-methylamino)sulfonyl-N-[1-[[3-(5-methyl-2-p-tolyloxazol-4-yl)methoxy]phenyl]ethyl]amino]acetic acid;
130) 3-[N—(N,N-dimethylamino)sulfonyl-N-[3-[(5-methyl-2-p-tolyl oxazol-4-yl)methoxy]benzyl]amino]propionic acid;
131) [N—(N,N-dimethylamino)sulfonyl-N-[3-[(2-phenyloxazol-4-yl)ethoxy]benzyl]amino]acetic acid;
132) [N—(N,N-dimethylamino)sulfonyl-N-[4-[(2-phenyloxazol-4-yl)ethoxy]benzyl]amino]acetic acid;
133) [N—[[N-(4-chlorophenyl)-N-methyl]amino]sulfonyl-N-[4-[(2-phenyl oxazol-4-yl)ethoxy]benzyl]amino]acetic acid; and
134) [N—[(N,N-dimethylamino)sulfonyl]-N-[4-[(2-phenyl-5-isopropyl oxazol-4-yl)methoxy]benzyl]amino]acetic acid.

3. The derivative of claim 1, wherein R₁ is hydrogen; R₂ is hydrogen or methyl; R₃ is hydrogen or C₁₋₃ alkyl; R₄ and R₅ are each independently hydrogen, methyl, ethyl, isopropyl, propargyl, piperidinyl, or phenyl which is unsubstituted or substituted with methyl, methoxy, fluoro or chloro; or are fused together with the nitrogen atom to which they are attached to form piperidine or quinolone ring; R₆ is methyl; R₇ is phenyl, which is unsubstituted or substitutied with CF₃ or CH₃; X is nitrogen; Y is oxygen; m is 0 or 1; and n is 1 or 2.

4. The derivative of claim 3, which is selected from the group consisting of:
2) (S)-3-methyl-2-[N-(sulfamoyl)-N-[3-[(5-methyl-2-p-tolyloxazol-4-yl)methoxy]benzyl]amino]butyric acid;
7) [N—(N,N-diethylamino)sulfonyl-N-[3-[[2-(4-trifluoromethylphenyl)-5-methyloxazol-4-yl]methoxy]benzyl]amino]acetic acid;
8) [N—(N-isopropyl-N-methylamino)sulfonyl-N-[3-[[2-(4-trifluoromethyl phenyl)-5-methyloxazol-4-yl]methoxy]benzyl]amino]acetic acid;
9) [N—(N-allyl-N-methylamino)sulfonyl-N-[3-[[2-(4-trifluoromethyl phenyl)-5-methyloxazol-4-yl]methoxy]benzyl]amino]acetic acid;
10) [N—(N-methyl-N-propargyl)sulfonyl-N-[3-[[2-(4-trifluoromethyl phenyl)-5-methyloxazol-4-yl]methoxy]benzyl]amino]acetic acid;
11) [N-(piperidinyl)sulfonyl-N-[3-[[2-(4-trifluoromethylphenyl)-5-methyloxazol-4-yl]methoxy]benzyl]amino]acetic acid;
21) [N—(N-ethyl-N-m-tolylamino)sulfonyl-N-[3-[[2-(4-trifluoromethyl phenyl)-5-methyloxazol-4-yl]methoxy]benzyl]amino]acetic acid;
22) [N—(N-p-anisoyl-N-methylamino)sulfonyl-N-[3-[[2-(4-trifluoro methylphenyl)-5-methyloxazol-4-yl]methoxy]benzyl]amino]acetic acid;
23) [N—[N-(3-fluorophenyl)-N-methylamino]sulfonyl-N-[3-[[2-(4-tri fluoromethylphenyl)-5-methyloxazol-4-yl]methoxy]benzyl]amino]acetic acid;
58) [N-(1,2,3,4-tetraquinolinyl)sulfonyl-N-[4-[[2-(4-methylphenyl)-5-methyloxazol-4-yl]methoxy]benzyl]amino]acetic acid;
62) [N—(N,N-dimethylamino)sulfonyl-N-[3-[2-[2-(4-methylphenyl)-5-methyloxazol-4-yl]ethoxy]benzyl]amino]acetic acid;
63) [N—(N,N-diethylamino)sulfonyl-N-[3-[2-[2-(4-methylphenyl)-5-methyloxazol-4-yl]ethoxy]benzyl]amino]acetic acid;
65) [N—(N,N-dimethylamino)sulfonyl-N-[3-[2-[2-(4-trifluoromethyl phenyl)-5-methyloxazol-4-yl]ethoxy]benzyl]amino]acetic acid;
67) [N—(N,N-diethylamino)sulfonyl-N-[3-[2-[2-(4-trifluoromethylphenyl)-5-methyloxazol-4-yl]ethoxy]benzyl]amino]acetic acid;
70) [N—(N-methyl-N-propargylamino)sulfonyl-N-[3-[2-[2-(4-trifluoro methylphenyl)-5-methyloxazol-4-yl]ethoxy]benzyl]amino]acetic acid;
71) [N-(piperidinyl)sulfonyl-N-[3-[2-[2-(4-trifluoromethylphenyl)-5-methyloxazol-4-yl]ethoxy]benzyl]amino]acetic acid;
80) [N—(N-ethyl-N-m-tolylamino)sulfonyl-N-[3-[2-[2-(4-trifluoromethyl phenyl)-5-methyloxazol-4-yl]ethoxy]benzyl]amino]acetic acid;
81) [N—(N-anisoyl-N-methylamino)sulfonyl-N-[3-[2-[2-(4-trifluoro methylphenyl)-5-methyloxazol-4-yl]ethoxy]benzyl]amino]acetic acid;
82) [N—[N-(3-fluorophenyl)-N-methylamino]sulfonyl-N-[3-[2-[2-(4-tri fluoromethylphenyl)-5-methyloxazol-4-yl]ethoxy]benzyl]amino]acetic acid;
101) [N—(N,N-diethylamino)sulfonyl-N-[4-[2-[2-(4-methylphenyl)-5-methyloxazol-4-yl]ethoxy]benzyl]amino]acetic acid;
102) [N—(N-isopropyl-N-methylamino)sulfonyl-N-[4-[2-[2-(4-methyl phenyl)-5-methyloxazol-4-yl]ethoxy]benzyl]amino]acetic acid;
111) [N—(N-ethyl-N-m-tolylamino)sulfonyl-N-[4-[2-[2-(4-methylphenyl)-5-methyloxazol-4-yl]ethoxy]benzyl]amino]acetic acid;
112) [N—(N-anisoyl-N-methylamino)sulfonyl-N-[4-[2-[2-(4-methyl phenyl)-5-methyloxazol-4-yl]ethoxy]benzyl]amino]acetic acid;
113) [N—[N-(3-fluorophenyl)-N-methylamino]sulfonyl-N-[4-[2-[2-(4-methylphenyl)-5-methyloxazol-4-yl]ethoxy]benzyl]amino]acetic acid;
124) (S)-2-[N—(N,N-dimethylamino)sulfonyl-N-[3-[(5-methyl-2-p-tolyl oxazol-4-yl)methoxy]benzyl]amino]propionic acid;
125) (S)-3-methyl-2-[N—(N,N-dimethylamino)sulfonyl-N-[3-[(5-methyl-2-p-tolyloxazol-4-yl)methoxy]benzyl]amino]butyric acid;
126) [N—(N,N-dimethylamino)sulfonyl-N-[1-[[3-(5-methyl-2-p-tolyl oxazol-4-yl)methoxy]phenyl]ethyl]amino]acetic acid;
127) [N-(pyrrolidinyl)sulfonyl-N-[1-[[3-(5-methyl-2-p-tolyloxazol-4-yl)methoxy]phenyl]ethyl]amino]acetic acid;
129) [N—(N-isopropyl-N-methylamino)sulfonyl-N-[1-[[3-(5-methyl-2-p-tolyloxazol-4-yl)methoxy]phenyl]ethyl]amino]acetic acid;
130) 3-[N—(N,N-dimethylamino)sulfonyl-N-[3-[(5-methyl-2-p-tolyl oxazol-4-yl)methoxy]benzyl]amino]propionic acid;
131) [N—(N,N-dimethylamino)sulfonyl-N-[3-[(2-phenyloxazol-4-yl)ethoxy]benzyl]amino]acetic acid;
132) [N—(N,N-dimethylamino)sulfonyl-N-[4-[(2-phenyloxazol-4-yl)ethoxy]benzyl]amino]acetic acid; and
133) [N—[[N-(4-chlorophenyl)-N-methyl]amino]sulfonyl-N-[4-[(2-phenyloxazol-4-yl)ethoxy]benzyl]amino]acetic acid.

5. A pharmaceutical composition for upregulation of lipid metabolism comprising the derivative of the claim 1 or a pharmaceutically acceptable salt, hydrate or solvate thereof as an active ingredient.

6. The composition of claim 5, which is used for the treatment and prevention of obesity, NIDDM (non-insulin dependent diabetes mellitus), hyperlipidemia, arteriosclerosis, steatosis of the liver or muscle, and fatty liver.
